(19) United States
(12) Patent Application Publication
(10) Pub. No.: US 2025/0205373 A1
(43) Pub. Date: Jun. 26, 2025

(54) ENRICHED AND STABLE RADIOLIGAND THERAPY FORMULATIONS AND PHARMACEUTICAL COMPOSITIONS COMPRISING SAME

(71) Applicant: Mariana Oncology Inc., Watertown, MA (US)

(72) Inventors: Julie CUPIDO, Hudson, NH (US); Alex SUTTON, Waltham, MA (US); Henry MOK, Londonderry, NH (US); Bernard LAMBERT, Fishers, IN (US); Wei ZHANG, Belmont, MA (US)

(21) Appl. No.: 18/968,695

(22) Filed: Dec. 4, 2024

Related U.S. Application Data
(60) Provisional application No. 63/606,327, filed on Dec. 5, 2023.
(60) Provisional application No. 63/606,335, filed on Dec. 5, 2023.

(51) Int. Cl. A61K 51/08; A61K 47/20; A61K 47/22; A61K 47/26; A61P 35/00

(52) U.S. Cl. CPC A61K 51/088; A61K 47/20; A61K 47/22; A61K 47/26; A61P 35/00; A61K 2121/00

(57) ABSTRACT

The present disclosure relates to stable compositions comprising a radiolabeled compound, and related pharmaceutical compositions and methods of manufacture. Such compositions are useful in both the diagnostic imaging of and the treatment of diseases (e.g., cancer).

### RELATED APPLICATIONS

This application claims priority to U.S. Provisional Application No. 63/606,327 filed on Dec. 5, 2023 and U.S. Provisional Application No. 63/606,335 filed on Dec. 5, 2023, the entire contents of which are hereby incorporated by reference in their entirety.

### BACKGROUND

With the advancement of quantitative detection techniques, scientists are able to more accurately quantify the level of low-expressing targets in cancer cells. However, the relationship between cancer cells and low-expressing targets remains unknown. Existing treatment methods are the same for cancers characterized by low-expressing targets as cancers characterized by high-expressing targets. However, these treatment methods are inefficient for cancers characterized by low-expressing targets (Cancers 2022, 14, 3774). The development of targeted radioligand therapies for low-expressing targets remains an unexplored, yet potentially effective method for cancer treatment.

Additional consideration must be given to radioligand therapies (RLTs) designed for low-expressing targets. This is because most radioligand therapy formulations contain a large amount of unlabeled compound that compete with their radioactive counterparts for target binding. For low-expressing targets, the presence of these non-radioactive entities can limit cellular internalization of radioactivity to such an extent as to limit cell killing.

Chromatographic separation of RLTs to remove excess, non-radiolabeled starting materials (e.g., non-radiolabeled peptide) remains a challenge in the field because the non-radiolabeled precursor often coelutes with the radiolabeled product of interest (e.g., radiolabeled peptide) due to the similarities in chemical structure and properties, and the minute difference in molecular weight. Existing formulations that are not enriched in radiolabeled product are particularly disadvantageous for low-expressing targets.

One added complication of RLTs is the production and shelf-life of the radioactive component of the therapy. Decay of the radioactive component occurs constantly during manufacture and storage and the released high energy emissions induce the cleavage of the chemical bonds of the molecules in the radioligand, resulting in reduced radiochemical purity (RCP). Most common RLT production processes require the use of ICH Class 2 solvents that cannot be injected into humans, which further necessitates additional chemical processing and reformulation steps. However, these added steps often result in degradation of the radioligand therapy in the form of radiolysis, resulting in reduced RCP. Additionally, because radioactive isotopes have a short half-life, and undergo auto-radiolysis, the time between production and administration of the therapy must be minimized. Production of a pure and highly radioactive pharmaceutical that quickly tracks to a tumor will improve the odds of radiation sufficiently reaching the tumor to induce cell death.

Thus, there remains a need to develop stable radioligand therapies that are radioactively enriched and processes for preparing the same.

### SUMMARY

Provided herein are stable compositions comprising a radiolabeled compound, and related pharmaceutical compositions and methods of manufacture.

Thus, in an aspect, provided herein is a method of making a stabilized composition of a radiolabeled compound, comprising:
a. eluting a mixture comprising the radiolabeled compound and a corresponding unlabeled compound through a chromatography column, thereby separating the radiolabeled compound from the unlabeled compound;
b. collecting an eluate containing the separated radiolabeled compound in a vessel containing a stabilizing solution to afford the stabilized composition of the radiolabeled compound.

In some embodiments, the stabilized composition is a pharmaceutical composition.

In another aspect, provided herein is a method of preparing a composition enriched in a radiolabeled peptide, wherein the method comprises:
(a) combining a peptide that is linked to a chelator, a stabilizer, a buffer, and a radionuclide to form a reaction mixture, wherein a radiolabeled peptide is formed;
(b) optionally adding a quencher to the reaction mixture;
(c) separating the radiolabeled peptide from non-radiolabeled peptide by chromatography; and
(d) isolating the composition enriched in the radiolabeled peptide.

In another aspect, provided herein is a pharmaceutical composition comprising a radiolabeled compound and a stabilizing solution, wherein, in certain embodiments, the radiolabeled compound comprises (i) a peptide comprising a chelator, and (ii) a radioisotope; and wherein, in certain embodiments, the pharmaceutical composition (which comprises the stabilizing solution) comprises ascorbic acid, or a pharmaceutically acceptable salt thereof, and a polysorbate.

In certain embodiments, the pharmaceutical composition has a RCP of >90% after 72 hours at 2-8° C.

In yet another aspect, provided herein is a pharmaceutical composition comprising:
(a) a radiolabeled peptide;
(b) a stabilizer;
(c) a surfactant; and
(d) a buffer,
wherein the composition is enriched in the radiolabeled peptide.

In still another aspect, provided herein is a pharmaceutical composition comprising a radiolabeled compound, ascorbic acid or a pharmaceutically acceptable salt thereof, and a polysorbate, wherein the ascorbic acid, or salt thereof, and the polysorbate are present in amounts sufficient to maintain a RCP of at least 90% for at least 168 hours at 2-8° C.

In yet an aspect, provided herein is a method of treating cancer in a subject in need thereof, comprising administering to the subject a stabilized composition prepared by the methods disclosed herein.

### BRIEF DESCRIPTION OF THE DRAWINGS

FIG. 1 and FIG. 2 depict the process disclosed herein that is used to prepare enriched and stable RLT formulations and pharmaceutical compositions.

FIG. 3 is a representative UV chromatogram of [²²⁵Ac]Ac-153 purification at 220 nm overlaid with radioactivity chromatogram.

FIG. 4 is a representative UV chromatogram of [²²⁵Ac]Ac-447 purification at 220 nm overlaid with radioactivity chromatogram.

FIG. 5 and FIG. 6 are a comparison of dose-dependent and time-dependent cell killing in SHP77 with high and regular specific activity [²²⁵Ac]Ac-447.

FIG. 7 is an operation flowchart that depicts the production of [¹¹¹In]In-447.

FIG. 8 is a comparison of dose-dependent anti-cancer activity in CDX mouse models with high and regular specific activity [¹⁷⁷Lu]Lu-496. FIG. 8A shows data for a SHP77 cell model. FIG. 8B shows data for a NCI-H1650 model.

FIG. 9 is an RP-HPLC chromatogram of small-scale HSA [²²⁵Ac]Ac-496 (Run #1).

FIG. 10 is an RP-HPLC chromatogram of small-scale HSA [²²⁵Ac]Ac-496 (Run #2).

FIG. 11A and FIG. 11B show tumor volume over time after a single IV administration of vehicle, [²²⁵Ac]Ac-496 in Study 1 (A), and Study 2 (B) in NCI-H1650 Tumor-Bearing Mice (MEAN±SEM).

FIG. 12 shows HPLC separation of DOTA-TATE and [^{nal}La]La-DOTA-TATE following co-injection.

FIG. 13 shows HPLC separation of a [²²⁵Ac]Ac-DOTA-TATE reaction mixture with collected fractions indicated.

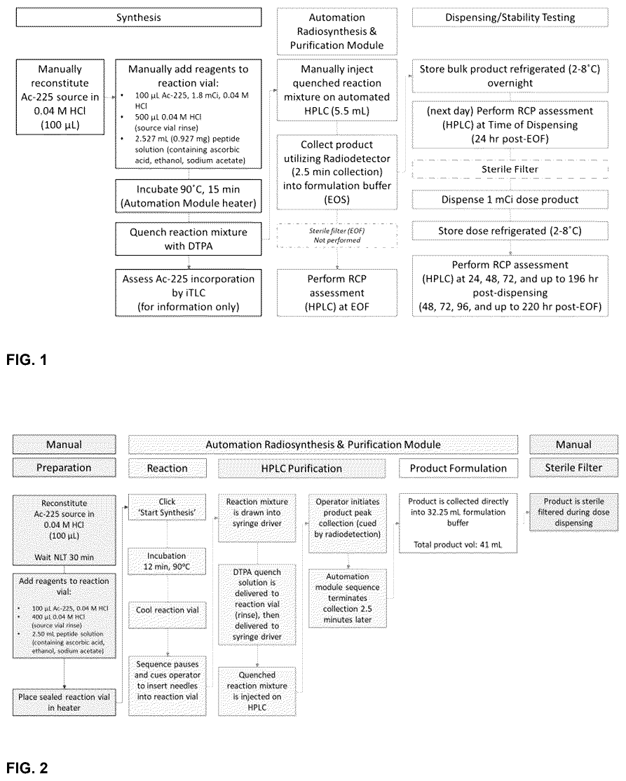

### DETAILED DESCRIPTION

Provided herein are stable compositions comprising a radiolabeled compound, and related pharmaceutical compositions and methods of manufacture. These compositions are useful in both the diagnostic imaging of and the treatment of diseases (e.g., cancer), particularly cancers characterized as having low expression of targeted cell surface receptors.

### Definitions

Listed below are definitions of various terms used to describe the compounds and compositions disclosed herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, 1%, and ±0.1% from the specified value, as such variations are appropriate to perform the disclosed methods.

As used herein, the term “enriched” refers to a radiolabeled peptide composition, in which the ratio of radiolabeled peptide is higher than the ratio of the non-radiolabeled peptide starting material. In some embodiments, the radiolabeled peptide composition is about 100%, at least about 95%, at least about 90%, at least about 85%, at least about 80%, at least about 75%, at least about 70%, at least about 60%, at least about 65%, at least about 60%, at least about 55%, or at least about 50% enriched in radiolabeled peptide as compared to non-radiolabeled peptide. In some embodiments, the radiolabeled peptide composition is about 100%, 95%, 90%, 85%, 80%, 75%, 70%, 60%, 65%, 60%, 55%, or 50% enriched in radiolabeled peptide as compared to non-radiolabeled peptide. In an embodiment, these percentages are based on weight percent.

As used herein, the term “stabilizing solution” refers to a solution comprising a radiolytic stabilizer. Radiolytic stabilizers are substances that prevent or decrease radiolysis, i.e., the degradation of a target molecule by the energy of radioactive decay.

As used herein, “surfactant” refers to an excipient that preferentially adsorbs to an interface between two immiscible phases, such as the interface between water and an organic polymer solution, a water/air interface or organic solvent/air interface. Suitable surfactants include, but are not limited to, fatty alcohols such as polyethylene glycols (PEGs) and cetyl alcohol. In a non-limiting embodiment, the surfactant is a polysorbate. Polysorbates are products of the esterification of ethoxylated sorbitan (a derivative of sorbitol) with fatty acids, and include (but are not limited to): polyoxyethylene (20) sorbitan monolaurate, polyoxyethylene (20) sorbitan monopalmitate, polyoxyethylene (20) sorbitan monostearate, and polyoxyethylene (20) sorbitan monooleate. In certain embodiments provided herein, the polysorbate is polyoxyethylene (20) sorbitan monolaurate (Tween 20).

As used herein, the terms “quencher” or “quenching agent” refers to a molecule or group of molecules that can scavenge excess amounts of radionuclide starting material in the synthesis of radiolabeled compounds including radiolabeled peptides. In a non-limiting example, the quencher is diethylenetriaminepentaacetic acid (DTPA).

The term “buffer” refers to a solution where the pH does not change significantly on dilution or on addition of an acid or base at a constant temperature.

As used herein, the terms “radionuclide” and “radioisotope” are used interchangeably and refer to a nuclide that has excess nuclear energy that is used in one of three ways: emitted from the nucleus as gamma radiation; transferred to one of its electrons to release it as a conversion electron; or used to create and emit a new particle (alpha particle or beta particle) from the nucleus.

As used herein, the term “radiochemical purity” or “RCP” is defined as the percent of total radioactivity within a composition that corresponds to the radiolabeled drug substance (radiolabeled compound).

As used herein, the term “half-life” or “t₁₁₂” refers to the time it takes for half of the radioactive atoms of a radionuclide to decay.

As used herein, the term “chromatography” refers to a process in which a chemical mixture carried by a liquid or gas is separated into components as a result of differential distribution of the chemical entities as they flow around or over a stationary liquid or solid phase.

As used herein, the phrase “liquid chromatography” or “LC” means a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., mobile phase), as this fluid moves relative to the stationary phase(s). Examples of “liquid chromatography” include reverse phase liquid chromatography (RPLC), high performance liquid chromatography (HPLC), and turbulent flow liquid chromatography (TFLC) (sometimes known as high turbulence liquid chromatography (HTLC) or high throughput liquid chromatography).

The mobile phase composition may be constant (i.e., “isocratic elution” as used herein) or may be changed (i.e., “gradient elution” as used herein) during the separation process. In order to separate compositions comprising components that have very different chemical natures, additional solvents may be introduced to the column, typically by increasing the proportion of solvent in the mobile phase. In a non-limiting example, the stationary phase is C18 bonded to silica. In a non-limiting example the mobile phase includes an aqueous mixture of water and organic solvent, for example ethanol.

In some embodiments, liquid chromatography is ultra-performance liquid chromatography (UPLC; the term “ultra high performance liquid chromatography” or UHPLC may be used interchangeably herein). UPLC is known in the art as an LC technique that relies upon a column with reduced particle size (e.g., less than 2 μm) and increased flow velocity to improve chromatographic resolution, efficiency, peak capacity, and sensitivity (see, e.g., Plumb, R. et al. (2004) Rapid Commun. Mass Spectrom. 18:2331-2337). In some embodiments, UPLC refers to the use of a column with a particle size less than 2 μm in liquid chromatography. In some embodiments, UPLC refers to the use of a high linear solvent velocity (e.g., as observed when operating at 6000 psi or higher) in liquid chromatography. Exemplary UPLC instruments are commercially available, e.g., Waters ACQUITY Premier UPLC H Class (Premier QSM, Premier SM-FTN, CM-A, PDA eA detector & SQD 2 mass analyzer).

As used herein, the phrase “high performance liquid chromatography” or “HPLC” (sometimes known as “high pressure liquid chromatography”) refers to liquid chromatography in which the degree of separation is increased by forcing the mobile phase under pressure through a stationary phase, typically a densely packed column.

As used herein, the terms “extraction column” and “column” are used interchangeably to refer to a chromatography column having sufficient chromatographic plates to affect a separation of materials in a sample that elute. The general purpose of an extraction column is for separating or extracting retained material from non-retained materials in order to obtain a purified sample for further analysis. Such columns are often distinguished from “analytical columns,” which allow for the determination of the presence or amount of an analyte. As used in this context, the term “about” means ±10%. In a preferred embodiment the analytical column contains particles of about 5 μm in diameter.

As used herein, the term “alkyl,” when used alone or as part of a larger moiety, such as “haloalkyl”, “hydroxyalkyl” and the like, means saturated straight chain or branched monovalent hydrocarbon radical having, unless otherwise specified, from 1 to 20 carbon atoms such as C1-10, C1-6, or C1-4. A C1-6 alkyl includes e.g. methyl, ethyl, propyl (e.g., n-propyl, isopropyl), butyl (e.g., n-butyl, isobutyl, tert-butyl, sec-butyl), pentyl (e.g., n-pentyl, 3-pentanyl, amyl, neopentyl, 3-methyl-2-butanyl, tertiary amyl), and hexyl (e.g., n-hexyl). It will be understood that when specified, optional substituents on an alkyl group may be present on any substitutable position.

The term “alkylene,” employed alone or in combination with other terms, refers to a divalent alkyl linking group. An alkylene group formally corresponds to an alkane with two C—H bonds replaced by points of attachment of the alkylene group to the remainder of the compound. The term “Cₙ₋ₘ alkylene” refers to an alkylene group having n to m carbon atoms. Examples of alkylene groups include, but are not limited to, methylene, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “amino acid” includes the residues of the natural amino acids as well as unnatural amino acids. The 20 natural proteinogenic amino acids are identified and referred to herein by either the one-letter or three-letter designations as follows: aspartic acid (Asp: D), isoleucine (Ile: I), threonine (Thr: T), leucine (Leu: L), serine (Ser: S), tyrosine (Tyr: Y), glutamic acid (Glu: E), phenylalanine (Phe: F), proline (Pro: P), histidine (His: H), glycine (Gly: G), lysine (Lys: K), alanine (Ala: A), arginine (Arg: R), cysteine (Cys: C), tryptophan (Trp: W), valine (Val: V), glutamine (Gln: Q) methionine (Met: M), asparagine (Asn: N). Naturally occurring amino acids exist in their levorotary (L) stereoisomeric forms. Amino acids referred to herein are L-stereoisomers except where otherwise indicated. The term “amino acid” also includes amino acids bearing a conventional amino protecting group (e.g., acetyl or benzyloxycarbonyl), as well as natural and unnatural amino acids protected at the carboxy terminus (e.g., as a (C1-C6) alkyl, phenyl or benzyl ester or amide; or as an alpha-methylbenzyl amide). Other suitable amino and carboxy protecting groups are known to those skilled in the art (See for example, Greene, T. W.; Wutz, P. G. M., Protecting Groups In Organic Synthesis; second edition, 1991, New York, John Wiley & sons, Inc., and documents cited therein, the contents of each of which are herein incorporated by reference in their entirety). Peptides and/or peptide compositions of the present disclosure may also include modified amino acids.

“Unnatural” amino acids have side chains or other features not present in the 20 naturally-occurring amino acids listed above and include, but are not limited to: N-methyl amino acids, N-alkyl amino acids, alpha, alpha substituted amino acids, beta-amino acids, alpha-hydroxy amino acids, D-amino acids, and other unnatural amino acids known in the art (See, e.g., Josephson et al., (2005) J. Am. Chem. Soc. 127: 11727-11735; Forster, A. C. et al. (2003) Proc. Natl. Acad. Sci. USA 100: 6353-6357; Subtelny et al., (2008) J. Am. Chem. Soc. 130: 6131-6136; Hartman, M. C. T. et al. (2007) PLoS ONE 2:e972; and Hartman et al., (2006) Proc. Natl. Acad. Sci. USA 103:4356-4361). Further unnatural amino acids useful for the optimization of peptides and/or peptide compositions of the present disclosure include, but are not limited to, 1,2,3,4-tetrahydroisoquinoline-1-carboxylic acid, 1-amino-2,3-hydro-1H-indene-1-carboxylic acid, homolysine, homoarginine, homoserine, 2-aminoadipic acid, 3-aminoadipic acid, beta-alanine, aminopropionic acid, 2-aminobutyric acid, 4-aminobutyric acid, 5-aminopentanoic acid, 5-afminohexanoic acid, 6-aminocaproic acid, 2-aminoheptanoic acid, 2-aminoisobutyric acid, 3-aminoisobutyric acid, 2-aminopimelic acid, desmosine, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylpentylglycine, naphthylalanine, ornithine, pentylglycine, thioproline, norvaline, tert-butylglycine (also known as tert-leucine), phenylglycine, azatryptophan, 5-azatryptophan, 7-azatryptophan, 4-fluorophenylalanine, penicillamine, sarcosine, homocysteine, 1-aminocyclopropanecarboxylic acid, 1-aminocyclobutanecarboxylic acid, 1-aminocyclopentanecarboxylic acid, 1-aminocyclohexanecarboxylic acid, 4-aminotetrahydro-2H-pyran-4-carboxylic acid, (S)-2-amino-3-(1H-tetrazol-5-yl)propanoic acid, cyclopentylglycine, cyclohexylglycine, cyclopropylglycine, n-w-methyl-arginine, 4-chlorophenylalanine, 3-chlorotyrosine, 3-fluorotyrosine, 5-fluorotryptophan, 5-chlorotryptophan, citrulline, 4-chloro-homophenylalanine, homophenylalanine, 4-aminomethyl-phenylalanine, 3-aminomethyl-phenylalanine, octylglycine, norleucine, tranexamic acid, 2-amino pentanoic acid, 2-amino hexanoic acid, 2-amino heptanoic acid, 2-amino octanoic acid, 2-amino nonanoic acid, 2-amino decanoic acid, 2-amino undecanoic acid, 2-amino dodecanoic acid, aminovaleric acid, and 2-(2-aminoethoxy)acetic acid, pipecolic acid, 2-carboxy azetidine, hexafluoroleucine, 3-Fluorovaline, 2-amino-4,4-difluoro-3-methylbutanoic acid, 3-fluoro-isoleucine, 4-fluoroisoleucine, 5-fluoroisoleucine, 4-methyl-phenylglycine, 4-ethyl-phenylglycine, 4-isopropyl-phenylglycine, (S)-2-amino-5-azidopentanoic acid (also referred to herein as “X02”), (S)-2-aminohept-6-enoic acid (also referred to herein as “X30”), (S)-2-aminopent-4-ynoic acid (also referred to herein as “X31”), (S)-2-aminopent-4-enoic acid (also referred to herein as “X12”), (S)-2-amino-5-(3-methylguanidino) pentanoic acid, (S)-2-amino-3-(4-(aminomethyl)phenyl)propanoic acid, (S)-2-amino-3-(3-(aminomethyl)phenyl)propanoic acid, (S)-2-amino-4-(2-aminobenzo[d]oxazol-5-yl)butanoic acid, (S)-leucinol, (S)-valinol, (S)-tert-leucinol, (R)-3-methylbutan-2-amine, (S)-2-methyl-1-phenylpropan-1-amine, and (S)—N,2-dimethyl-1-(pyridin-2-yl)propan-1-amine, (S)-2-amino-3-(oxazol-2-yl)propanoic acid, (S)-2-amino-3-(oxazol-5-yl)propanoic acid, (S)-2-amino-3-(1,3,4-oxadiazol-2-yl)propanoic acid, (S)-2-amino-3-(1,2,4-oxadiazol-3-yl)propanoic acid, (S)-2-amino-3-(5-fluoro-1H-indazol-3-yl)propanoic acid, and (S)-2-amino-3-(1H-indazol-3-yl)propanoic acid, (S)-2-amino-3-(oxazol-2-yl)butanoic acid, (S)-2-amino-3-(oxazol-5-yl) butanoic acid, (S)-2-amino-3-(1,3,4-oxadiazol-2-yl) butanoic acid, (S)-2-amino-3-(1,2,4-oxadiazol-3-yl) butanoic acid, (S)-2-amino-3-(5-fluoro-1H-indazol-3-yl) butanoic acid, and (S)-2-amino-3-(1H-indazol-3-yl) butanoic acid, 2-(2′MeOphenyl)-2-amino acetic acid, tetrahydro 3-isoquinolinecarboxylic acid and stereoisomers thereof (including, but not limited, to D and L isomers).

Additional unnatural amino acids that are useful in the optimization of peptides or peptide compositions of the disclosure include but are not limited to halogenated amino acids wherein one or more carbon bound hydrogen atoms are replaced by one or more halogen atoms. The number of halogen atoms included can range from 1 up to and including all of the hydrogen atoms.

In some embodiments, unnatural amino acids that are useful in the optimization of peptides or peptide compositions of the disclosure include but are not limited to fluorinated amino acids wherein one or more carbon bound hydrogen atoms are replaced by one or more fluorine atoms. The number of fluorine atoms included can range from 1 up to and including all of the hydrogen atoms. Examples of such amino acids include but are not limited to 3-fluoroproline, 3,3-difluoroproline, 4-fluoroproline, 4,4-difluoroproline, 3,4-difluroproline, 3,3,4,4-tetrafluoroproline, 4-fluorotryptophan, 5-flurotryptophan, 6-fluorotryptophan, 7-fluorotryptophan, and stereoisomers thereof.

In some embodiments, unnatural amino acids that are useful in the optimization of peptides or peptide compositions of the disclosure include but are not limited to chlorinated amino acids wherein one or more carbon bound hydrogen atoms are replaced by one or more chlorine atoms. The number of chlorine atoms included can range from 1 up to and including all of the hydrogen atoms.

Further unnatural amino acids that are useful in the optimization of peptides of the disclosure include but are not limited to those that are disubstituted at the α-carbon. These include amino acids in which the two substituents on the α-carbon are the same, for example α-amino isobutyric acid, and 2-amino-2-ethyl butanoic acid, as well as those where the substituents are different, for example α-methylphenylglycine and α-methylproline. Further the substituents on the α-carbon may be taken together to form a ring, for example 1-aminocyclopentanecarboxylic acid, 1-aminocyclobutanecarboxylic acid, 1-aminocyclohexanecarboxylic acid, 3-aminotetrahydrofuran-3-carboxylic acid, 3-aminotetrahydropyran-3-carboxylic acid, 4-aminotetrahydropyran-4-carboxylic acid, 3-aminopyrrolidine-3-carboxylic acid, 3-aminopiperidine-3-carboxylic acid, 4-aminopiperidinnne-4-carboxylix acid, and stereoisomers thereof.

Additional unnatural amino acids that are useful in the optimization of peptides or peptide compositions of the disclosure include but are not limited to analogs of tryptophan in which the indole ring system is replaced by another 9 or 10 membered bicyclic ring system comprising 0, 1, 2, 3 or 4 heteroatoms independently selected from N, O, or S. Each ring system may be saturated, partially unsaturated, or fully unsaturated. The ring system may be substituted by 0, 1, 2, 3, or 4 substituents at any substitutable atom. Each substituent may be independently selected from H, F, Cl, Br, CN, COOR, CONRR′, oxo, OR, NRR′. Each R and R′ may be independently selected from H, C₁-C₂₀ alkyl, or C₁-C₂₀ alkyl-O—C₁₋₂₀ alkyl.

In some embodiments, analogs of tryptophan (also referred to herein as “tryptophan analogs”) may be useful in the optimization of peptides or peptide compositions of the disclosure. Tryptophan analogs may include, but are not limited to, 5-fluorotryptophan [(5-F)W], 5-methyl-O-tryptophan [(5-MeO)W], 1-methyltryptophan [(1-Me-W) or (1-Me)W], D-tryptophan (D-Trp), azatryptophan (including, but not limited to 4-azatryptophan, 7-azatryptophan and 5-azatryptophan) 5-chlorotryptophan, 4-fluorotryptophan, 6-fluorotryptophan, 7-fluorotryptophan, and stereoisomers thereof. Except where indicated to the contrary, the term “azatryptophan” and its abbreviation, “azaTrp,” as used herein, refer to 7-azatryptophan.

As used herein, the term “peptide” refers to a compound comprising two or more amino acids, as defined below, linked by a peptide bond (i.e., an amide bond linking the amine of one amino acid to the carboxyl of another). In a non-limiting example, peptide refers to a polypeptide that comprises 20-49 amino acids linked by a peptide bond. In a non-limiting example, peptide refers to a cyclic peptide that includes a “cyclic loop” formed when two amino acids are connected by a bridging moiety. The cyclic loop comprises the amino acids along the peptide present between the bridged amino acids. Cyclic loops may comprise 2, 3, 4, 5, 6, 7, 8, 9, 10 or more amino acids. In some embodiments, peptides of the present disclosure are cyclic peptides. In some embodiments, the cyclic peptides comprise a disulfide bond. In some embodiments, peptides of the present disclosure are linear peptides prior to the cyclization step. In some embodiments, peptides of the present disclosure are linear peptides prior to the formation of a disulfide bond.

Generally, disulfide bond formation involves a reaction between the sulfhydryl (SH) side chains of two cysteine residues. Proper disulfide bonds provide stability to a protein, decreasing further entropic choices that facilitate folding progression toward the native state by limiting unfolded or improperly folded conformations.

One method of protecting a peptide from proteolytic degradation involves chemical modification or “capping” of the amino and/or carboxy terminus of the peptides. As used herein, the terms “chemically modified” or “capped” are used interchangeably to refer to the introduction of a blocking group at the end or both ends of the compound by covalent modification. Suitable blocking groups serve to block the ends of the peptides without decreasing the biological activity of the peptides. Any residue located at the amino or carboxy terminus, or both of the described compounds can be chemically modified. In some embodiments, peptides of the present disclosure comprise an N-terminal and/or C-terminal modification.

In one embodiment, the amino end of the compound is chemically modified by acetylation to produce an N-acetylated peptide (which may be represented by “Ac—” in the structure or formula of the present disclosure). In another embodiment, the carboxy terminus of the described peptides is chemically modified by amidation to give the primary carboxamide at the C-terminus (which may be represented as “amide” in the peptide sequence, structure or claims of the present disclosure). In some embodiments, both the amino end and the carboxy end are chemically modified by acetylation and amidation, respectively. However, other capping groups are possible. For example, the amino end can be capped by acylation with groups such as an acetyl group, a benzoyl group, or natural or non-natural amino acids, such as beta-alanine, capped by an acetyl group; or by alkylation with groups such as a benzyl group or a butyl group, or by sulfonylation to produce sulfonamides. Similarly, the carboxy terminus can be esterified or converted to a secondary amide and acylsulfonamide or the like.

In some embodiments, the N-terminal capping function is in a linkage to the terminal amino group and may be selected from the group: formyl; alkanoyl, having from 1 to 10 carbon atoms, such as acetyl, propionyl, butyryl; alkenoyl, having from 1 to 10 carbon atoms, such as hex-3-enoyl; alkynoyl, having from 1 to 10 carbon atoms, such as hex-5-ynoyl; aroyl, such as benzoyl or 1-naphthoyl; heteroaroyl, such as 3-pyrroyl or 4-quinoloyl; alkylsulfonyl, such as methanesulfonyl; arylsulfonyl, such as benzenesulfonyl or sulfanilyl; heteroarylsulfonyl, such as pyridine-4-sulfonyl; substituted alkanoyl, having from 1 to 10 carbon atoms, such as 4-aminobutyryl; substituted alkenoyl, having from 1 to 10 carbon atoms, such as 6-hydroxy-hex-3-enoyl; substituted alkynoyl, having from 1 to 10 carbon atoms, such as 3-hydroxy-hex-5-ynoyl; substituted aroyl, such as 4-chlorobenzoyl or 8-hydroxy-naphth-2-oyl; substituted heteroaroyl, such as 2,4-dioxo-1,2,3,4-tetrahydro-3-methyl-quinazolin-6-oyl; substituted alkylsulfonyl, such as 2-aminoethanesulfonyl; substituted arylsulfonyl, such as 5-dimethylamino-1-naphthalenesulfonyl; substituted heteroarylsulfonyl, such as 1-methoxy-6-isoquinolinesulfonyl; carbamoyl or thiocarbamoyl; substituted carbamoyl (R′—NH—CO) or substituted thiocarbamoyl (R′—NH—CS) wherein R′ is alkyl, alkenyl, alkynyl, aryl, heteroaryl, substituted alkyl, substituted alkenyl, substituted alkynyl, substituted aryl, or substituted heteroaryl; substituted carbamoyl (R′—NH—CO) and substituted thiocarbamoyl (R′—NH—CS) wherein R′ is alkanoyl, alkenoyl, alkynoyl, aroyl, heteroaroyl, substituted alkanoyl, substituted alkenoyl, substituted alkynoyl, substituted aroyl, or substituted heteroaroyl, all as above defined; Lys-(Gly)n, where n=1-8; or Tyr-(Gly)n where n=1-8.

In some embodiments, the C-terminal capping function can either be in an amide bond with the terminal carboxyl or in an ester bond with the terminal carboxyl. Capping functions that provide for an amide bond are designated as NR1 R2 wherein each R1 and R2 may be independently selected from the following group: hydrogen; alkyl, having from 1 to 10 carbon atoms, such as methyl, ethyl, isopropyl; alkenyl, preferably having from 1 to 10 carbon atoms, such as prop-2-enyl; alkynyl, preferably having from 1 to 10 carbon atoms, such as prop-2-ynyl; substituted alkyl having from 1 to 10 carbon atoms, such as hydroxyalkyl, alkoxyalkyl, mercaptoalkyl, alkylthioalkyl, halogenoalkyl, cyanoalkyl, aminoalkyl, alkylaminoalkyl, dialkylaminoalkyl, alkanoylalkyl, carboxyalkyl, carbamoylalkyl; substituted alkenyl having from 1 to 10 carbon atoms, such as hydroxyalkenyl, alkoxyalkenyl, mercaptoalkenyl, alkylthioalkenyl, halogenoalkenyl, cyanoalkenyl, aminoalkenyl, alkylaminoalkenyl, dialkylaminoalkenyl, alkanoylalkenyl, carboxyalkenyl, carbamoylalkenyl; substituted alkynyl having from 1 to 10 carbon atoms, such as hydroxyalkynyl, alkoxyalkynyl, mercaptoalkynyl, alkylthioalkynyl, halogenoalkynyl, cyanoalkynyl, aminoalkynyl, alkylaminoalkynyl, dialkylaminoalkynyl, alkanoylalkynyl, carboxyalkynyl, carbamoylalkynyl; aroylalkyl having up to 10 carbon atoms, such as phenacyl or 2-benzoylethyl; aryl, such as phenyl or 1-naphthyl; heteroaryl, such as 4-quinolyl; alkanoyl having from 1 to 10 carbon atoms, such as acetyl or butyryl; aroyl, such as benzoyl; heteroaroyl, such as 3-quinoloyl; OR′ or NR′R″ where R′ and R″ each are independently hydrogen, alkyl, aryl, heteroaryl, acyl, aroyl, sulfonyl, sulfinyl, SO2-R′″ or SO—R′″ where R′″ is substituted or unsubstituted alkyl, aryl, heteroaryl, alkenyl, or alkynyl.

In some embodiments, capping functions that provide for an ester bond are designated as OR, wherein R may be: alkoxy; aryloxy; heteroaryloxy; aralkyloxy; heteroaralkyloxy; substituted alkoxy; substituted aryloxy; substituted heteroaryloxy; substituted aralkyloxy; or substituted heteroaralkyloxy.

In some embodiments, peptides of the present disclosure can comprise modifications to the C-terminus of the peptide sequence with one or more of the following moieties: NH₂, NH—CH₃; NH—CH₂—CH₃; NH—CH—(CH₃)₂, NH—CH₂—CH₂—CH₃, NH—CH₂—CH—(CH₃)₂, N(CH₃)₂, N(CH₂—CH₃)₂, or OH.

In some embodiments, peptides of the present disclosure can comprise modifications to the N-terminus of the peptide sequence with one or more peptide-based moieties. In some embodiments, peptides of the present disclosure can comprise modifications to the C-terminus of the peptide sequence with one or more peptide-based moieties. In some embodiments, peptides of the present disclosure can comprise modifications to both the N-terminus of the peptide sequence and the C-terminus of the peptide sequence with one or more peptide-based moieties.

In some embodiments, peptides of the present disclosure can comprise modifications to the N-terminus of the peptide sequence with one or more non-peptide-based moieties. In some embodiments, peptides of the present disclosure can comprise modifications to the C-terminus of the peptide sequence with one or more non-peptide-based moieties. In some embodiments, peptides of the present disclosure can comprise modifications to both the N-terminus of the peptide sequence and the C-terminus of the peptide sequence with one or more non-peptide-based moieties.

In some embodiments, peptides of the present disclosure can comprise modifications to the N-terminus which comprise a string of 5 or 6 Glu amino acids. In some embodiments, peptides of the present disclosure can comprise modifications to the N-terminus which comprise a string of 5 or 6 Lys amino acids. In some embodiments, peptides of the present disclosure can comprise modifications to the N-terminus which comprise a string of 5 or 6 amino acids, each independently selected from Glu or Lys.

In some embodiments, peptides of the present disclosure comprise an N-terminal peptide consisting of a chain of about 15 to about 400 identical amino acids. In some embodiments, the N-terminal peptide comprises about 25 to about 300 identical amino acids, about 50 to about 200 identical amino acids, about 75 to about 150 identical amino acids, about 90 to about 120 identical amino acids, or about 100 or 110 identical amino acids. In some embodiments, the N-terminal peptide comprises: poly(glutamic acid) peptides (PGa), poly(aspartic acid) peptides (PAs), poly(lysine) peptides (PLy), poly(arginine) peptides (PAr), poly(histidine) peptides (PHi), poly(ornithine) peptides (POr), or combinations thereof.

Peptides of the disclosure may be peptidomimetics. A “peptidomimetic” or “peptide mimetic” is a peptide in which the molecule contains structural elements that are not found in natural peptides (i.e., peptides comprised of only the 20 proteinogenic amino acids). In some embodiments, peptidomimetics are capable of recapitulating or mimicking the biological action(s) of a natural peptide. A peptidomimetic may differ in many ways from natural peptides, for example through changes in backbone structure or through the presence of amino acids that do not occur in nature. In some cases, peptidomimetics may include amino acids with side chains that are not found among the known 20 proteinogenic amino acids; non-peptide-based bridging moieties used to effect cyclization between the ends or internal portions of the molecule; substitutions of the amide bond hydrogen moiety by methyl groups (N-methylation) or other alkyl groups; substitutions of the amino acid alpha hydrogen moiety by methyl groups (alpha-methylation) or other alkyl groups; replacement of a peptide bond with a chemical group or bond that is resistant to chemical or enzymatic treatments; N- and C-terminal modifications; and/or conjugation with a non-peptidic extension (such as polyethylene glycol, lipids, carbohydrates, nucleosides, nucleotides, nucleoside bases, various small molecules, or phosphate or sulfate groups).

Modified amino acid residues useful for the optimization of peptides and/or peptide compositions of the present disclosure include, but are not limited to, those which are chemically blocked (reversibly or irreversibly); chemically modified on their N-terminal amino group or their side chain groups; chemically modified in the amide backbone, as for example, N-methylated, D (unnatural amino acids) and L (natural amino acids) stereoisomers; or residues wherein the side chain functional groups are chemically modified to another functional group. In some embodiments, modified amino acids include without limitation, methionine sulfoxide; methionine sulfone; aspartic acid-(beta-methyl ester), a modified amino acid of aspartic acid; N-ethylglycine, a modified amino acid of glycine; alanine carboxamide; and/or a modified amino acid of alanine. Unnatural amino acids may be purchased from Sigma-Aldrich (St. Louis, MO), Bachem (Torrance, CA) or other suppliers. Unnatural amino acids may further include any of those listed in Table 2 of US patent publication US 2011/0172126, the contents of which are incorporated herein by reference in their entirety.

In some embodiments, amino acids for use in the present disclosure are modified using an organic proteinaceous or non-proteinaceous derivatizing agent. In some embodiments, amino acids for use in the present disclosure are modified using post-translational modification. In some embodiments, modifications are introduced by reacting targeted amino acid residues of the peptide with an organic derivatizing agent that is capable of reacting with selected side-chains or terminal residues. In some embodiments, modifications are introduced by harnessing mechanisms of post-translational modifications that function in selected recombinant host cells. Certain post-translational modifications are the result of the action of recombinant host cells on an expressed peptide. As one example, glutaminyl and asparaginyl residues are frequently post-translationally deamidated to the corresponding glutamyl and aspartyl residues under certain post-translational conditions (e.g., under mildly acidic conditions). Other post-translational modifications include: hydroxylation of proline and lysine; phosphorylation of hydroxyl groups of tyrosinyl, seryl or threonyl residues; and methylation of the alpha-amino groups of lysine, arginine, and histidine side chains (see, e.g, Creighton et al., Proteins: Structure and Molecular Properties, W.H. Freeman & Co., San Francisco, 1983, pp. 79-86).

In some embodiments, amino acid modifications include the bonding of non-proteinaceous polymers to peptides of the present disclosure. Examples of non-proteinaceous polymers include hydrophilic synthetic polymers (i.e., non-natural polymers), such as hydrophilic polyvinyl polymers (e.g., polyvinylalcohol and polyvinylpyrrolidone). The examples of non-proteinaceous polymers also include polyethylene glycol, polypropylene glycol and polyoxyalkylenes. In some embodiments, amino acid modifications include the bonding of non-proteinaceous polymers to peptides of the present disclosure, as described in U.S. Pat. Nos. 4,640,835, 4,496,689, 4,301,144, 4,670,417, 4,791,192, and 4,179,337; the contents of which are each incorporated herein by reference in their entirety, as related to amino acid modifications for us in the present disclosure.

Examples of bridging moieties/peptide staples for use with compounds of the present disclosure include, but are not limited to: amide-based (e.g., lactam) bridges; aromatic-ring-based bridges; hydrocarbon chains; alkene-based hydrocarbon bridges (e.g., using Fmoc-S-2-(2′-pentenyl)alanine); triazole-based Click bridges, such as copper(I)-catalyzed Huisgen 1,3-dipolar cycloaddition reactions between side chain azido and alkynyl moieties (e.g., Fmoc-L-Nle(εN3) and Fmoc-D-Pra) (see S. Kawamoto, et al., J. Med. Chem. 2012, 55(3), 1137-1146); dialkynyl staples (e.g., 1,4-diethynylbenzene, diethynylpentane, diethynylamines) for stapling linear diazido-peptides; sulfide-bonded disulfide, thioether and bis-thioether bridges; perfluorobenzene bridges; or combinations thereof.

In some embodiments, bridging moieties comprise an amide bond between an amine functionality and a carboxylate functionality, each present in an amino acid, unnatural amino acid or non-amino acid residue side chain. In some embodiments, the amine or carboxylate functionalities are part of a non-amino acid residue or unnatural amino acid residue. In some embodiments, the bridging moiety comprises an amide bond produced by the reaction of the side chains of the following pairs of amino acids: lysine and glutamate; lysine and aspartate; ornithine and glutamate; ornithine and aspartate; homolysine and glutamic acid; homolysine and aspartic acid; and other combinations of amino acids, unnatural amino acids or non-amino acid residues comprising a primary amine and a carboxylic acid. In some embodiments, bridging moieties are formed through cyclization reactions using olefin metathesis.

In some embodiments, the bridging moiety comprises a disulfide bond formed between two thiol containing residues. In some embodiments, the bridging moiety comprises one or more thioether bonds. Such thioether bonds may include those found in cyclo-thioalkyl compounds. These bonds can be formed during a chemical cyclization reaction between chloro acetic acid N-terminal modified groups and cysteine residues. In some embodiments, bridging moieties comprise one or more triazole ring.

In some embodiments, bridging moieties comprise one or more hydrocarbon chains (linear or branched), and/or hydrocarbon rings (cyclic, heterocyclic, aromatic, heteroaromatic). In some embodiments, hydrocarbon bridging moieties may be introduced by reaction with reagents containing multiple reactive halides, including, but not limited to poly(bromomethyl)benzenes, poly(bromomethyl)pyridines, poly(bromomethyl)alkyl benzenes and/or (E)-1,4-dibromobut-2-ene. Examples of Poly(bromomethyl)benzene molecules of the present disclosure can include 1,2-bis(bromomethyl)benzene; 1,3-bis(bromomethyl)benzene; and 1,4-bis(bromomethyl)benzene.

In some embodiments, the thiol group of a cysteine residue is cross-linked with another cysteine residue to form a disulfide bond. In some embodiments, thiol groups of cysteine residues react with bromomethyl groups of poly(bromomethyl)benzene molecules to form stable linkages (see, e.g., Timmerman et al., ChemBioChem (2005) 6:821-824, the contents of which are incorporated herein by reference in their entirety).

In some embodiments, bis-, tris- and tetrakis(bromomethyl)benzene molecules can be used to generate bridging moieties to produce peptides with one, two or three loops, respectively. Bromomethyl groups of a poly(bromomethyl)benzene molecule may be arranged on the benzene ring on adjacent ring carbons (ortho- or o-), with a ring carbon separating the two groups (meta- or m-) or on opposite ring carbons (para- or p-). In some embodiments, m-bis(bromomethyl)benzene (i.e., m-dibromoxylene), o-bis(bromomethyl)benzene (i.e., o-dibromoxylene) and/or p-bis(bromomethyl)benzene (i.e., p-dibromoxylene) are used to form cyclic peptides. In some embodiments, thiol groups of cysteine residues react with other reagents comprising one or more bromo functional groups to form stable linkages. Such reagents may include, but are not limited to, poly(bromomethyl) pyridines (e.g., 2,6-bis(bromomethyl) pyridine), poly(bromomethyl)alkyl benzenes (e.g., 1,2-bis(bromomethyl)-4-alkylbenzene) and/or (E)-1,4-dibromobut-2-ene.

In some embodiments, a side chain amino group and a terminal amino group are cross-linked with disuccinimidyl glutarate (see, e.g., Millward et al., J. Am. Chem. Soc. (2005) 127:14142-14143). In some embodiments, an enzymatic method is used which relies on the reaction between (1) a cysteine and (2) a dehydroalanine or dehydrobutyrine group, catalyzed by a lantibiotic synthetase, to create the thioether bond (see, e.g., Levengood et al., Bioorg. and Med. Chem. Lett. (2008) 18:3025-3028). The dehydro functional group can also be generated chemically by the oxidation of selenium containing amino acid side chains incorporated during translation (see, e.g., Seebeck et al., J. Am. Chem. Soc. 2006).

In some embodiments, bridging moieties comprise an aromatic, 6-membered ring (e.g., benzene). In some embodiments, bridging moieties comprise a heterocyclic, 6-membered ring which includes one nitrogen atoms (e.g., pyridine). In some embodiments, bridging moieties comprise a heterocyclic, 6-membered ring which includes two nitrogen atoms (e.g., pyridazine, pyrimidine, pyrazine). In some embodiments, bridging moieties comprise a heterocyclic, 6-membered ring which includes three nitrogen atoms (e.g., triazanes). In some embodiments, bridging moieties comprise a heterocyclic, 5-membered ring which includes one nitrogen atoms (e.g., pyrrole). In some embodiments, bridging moieties comprise a heterocyclic, 5-membered ring which includes two nitrogen atoms (e.g., imidazole, pyrazole). In some embodiments, bridging moieties comprise a heterocyclic, 5-membered ring which includes three nitrogen atoms (e.g., triazoles).

As used herein, the term “cyclic” refers to the presence of a continuous loop. Cyclic molecules need not be circular, only joined to form an unbroken chain of subunits. Cyclic peptides may include a “cyclic loop,” formed when two amino acids are connected by a bridging moiety. The cyclic loop comprises the amino acids along the peptide present between the bridged amino acids. Cyclic loops may comprise 2, 3, 4, 5, 6, 7, 8, 9, 10 or more amino acids.

Peptides of the present disclosure may be cyclized through the carboxy terminus, the amino terminus, or through any other convenient point of attachment, such as, for example, through the sulfur of a cysteine (e.g., through the formation of disulfide bonds between two cysteine residues in a sequence) or any side-chain of an amino acid residue. Further linkages forming cyclic loops may include, but are not limited to, maleimide linkages, amide linkages, ester linkages, ether linkages, thiol ether linkages, hydrazone linkages, or acetamide linkages.

In some embodiments, peptides of the disclosure are formed using a lactam moiety. Such cyclic peptides may be formed, for example, by synthesis on a solid support Wang resin using standard Fmoc chemistry. In some cases, Fmoc-ASP(allyl)-OH and Fmoc-LYS(alloc)-OH are incorporated into peptides to serve as precursor monomers for lactam bridge formation.

As used herein, the term “lactam bridge” refers to an amide bond that forms a bridge between chemical groups in a molecule. In some cases, lactam bridges are formed between amino acids in a peptide.

As used herein, the terms “associated with,” “conjugated,” “linked,” “attached,” and “tethered,” when used with respect to two or more entities, means that the entities are physically associated or connected with one another, either directly or via one or more moieties that serve as linking agents, to form a structure that is sufficiently stable so that the entities remain physically associated, e.g., under working conditions, e.g., under physiological conditions. An “association” need not be through covalent chemical bonding and may include other forms of association or bonding sufficiently stable such that the “associated” entities remain physically associated, e.g., ionic or hydrogen bonding or a hybridization-based connectivity.

As used herein, the terms “linker” or “linkers” refer to any chemical structure that connects two or more entities or domains. Linkers may include one or more chemical bonds, atoms, groups of atoms, and/or chemical groups. Examples of chemical groups that can be included in linkers include, but are not limited to, alkyl, alkenyl, alkynyl, amido, amino, ether, thioether, ester, alkylene, heteroalkylene, aryl, or heterocyclyl chemical groups, each of which can be optionally substituted, as described herein. Linkers may include one or more of unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers. Linkers may include amino acids, peptides, peptides, and/or proteins.

Linkers may include carbon chains. Linker carbon chain lengths may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more atoms long. Linker carbon chains may contain heteroatoms (e.g., nitrogen, oxygen, sulfur, etc.).

Entities or domains joined by linkers may include, but are not limited to, atoms, chemical groups, nucleosides, nucleotides, nucleobases, sugars, nucleic acids, amino acids, peptides, peptides, proteins, protein complexes, cargo, therapeutic agents, and detectable labels. Linkers may be used for multiple purposes, including, but not limited to, forming multimers or conjugates.

Linkers may include cleavable elements, for example, disulfide (—S—S—) bonds or azo (—N═N—) bonds, which can be cleaved using reducing agents or photolysis. Selectively cleavable bonds may include amido bonds which may be cleaved for example by photolysis or by using tris(2-carboxyethyl)phosphine (TCEP) or other reducing agents. Selectively cleavable bonds may include ester bonds which may be cleaved, for example, by acidic or basic hydrolysis.

Linkers may include, but are not limited to, pH-sensitive linkers, protease cleavable peptide linkers, nuclease sensitive nucleic acid linkers, lipase sensitive lipid linkers, glycosidase sensitive carbohydrate linkers, hypoxia sensitive linkers, photo-cleavable linkers, heat-labile linkers, enzyme cleavable linkers (e.g., esterase cleavable linker), ultrasound-sensitive linkers, and x-ray cleavable linkers.

As used herein, the terms “chelating agent(s)” and “chelator(s)” may include metal chelating agents that associate with metal cargo (e.g., metallic nuclide cargo). Chelating agents may include macromolecular compounds. In some embodiments, the chelating agents include acyclic or macrocyclic compounds. Non-limiting examples of the chelating agents include 1,4,7,10-tetraazacyclododecane-1,4,7,10-tetraacetic acid (DOTA); DOTA derivative: DO3A; diethylenetriamine-N,N,N′,N″,N″-pentaacetic acid (DTPA); DTPA derivatives: 2-(p-SCN-Bz)-6-methyl-DTPA, CHX-A″-DTPA, and the cyclic anhydride of DTPA (CA-DTPA); 1,4,7,10-Tetraazacyclododecane-1,4,7,10-tetraacetic acid, α-(2-carboxyethyl) (DOTAGA), 1,4,7-triazacyclononane-1,4-7-triacetic acid (NOTA); NOTA derivatives (e.g., BCNOTA, p-NCS-Bz-NOTA, BCNOT); 6-hydrazinonicotinamide (HYNIC); ethylenediamine tetraacetic acid (EDTA); N,N′-ethylene-di-L-cysteine; N,N′-bis(2,2-dimethyl-2-mercaptoethyl)ethylenediamine-N,N′-diacetic acid (6SS); 1-(4-carboxymethoxybenzyl)-N—N′-bis[(2-mercapto-2,2-dimethyl)ethyl]-1,2-ethylenediamine-N,N′-diacetic acid (B6SS); Deferoxamine (DFO); 1,1,1-tris(aminomethyl)ethane (TAME); tris(aminomethyl)ethane-N,N,N′,N′,N″,N″-hexaacetic acid (TAME Hex); O-hydroxybenzyl iminodiacetic acid; 1,4,7-triazacyclononane (TACN); 1,4,7,10-tretraazacyclododecane (cyclen); 1,4,7-triazacyclononane-1-succinic acid-4,7-diacetic acid (NODASA); 1-(1-carboxy-3-carboxypropyl)-4,7-bis-(carboxymethyl)-1,4,7-triazacyclononane (NODAGA); 1,4,7-tris(2-mercaptoethyl)-1,4,7-triazacylclonane (triazacyclononane-TM); 1,4,7-triazacyclononane-N,N′,N″-tris(methylenephosphonic)acid (NOTP); 1, 4, 8, 11-tetraazacyclotetradecane-N,N′,N″,N′″-tetraacetic acid (TETA); 1,4,7,10,13-pentaazacyclopentadecane-N,N′,N″,N′″,N″″-pentaacetic acid (PEPA), 1,4,7,10,13,16-hexaazacyclohexadecane-N,N′,N″,N′″,N″″,N′″″-hexaacetic acid (HEHA); 1,4,7,10-tetrakis(carbamoylmethyl)-1,4,7,10-tetraazacyclododecane (TCMC); and derivatives or analogs thereof.

In an embodiment, the chelator is independently selected from the group consisting of ethylenediamine tetraacetic acid (EDTA), diethylenetriamine pentaacetic acid (DTPA), 1,4,7,10-tetra-azacylcododecane-N,N′,N″,N′″-tetraacetic acid (DOTA), 6-((16-((6-Carboxypyridin-2-yl)methyl)-1,4,10,13-tetraoxa-7,16-diazacyclooctadecan-7-yl)methyl)-4-isothiocyanatopicolinic acid (Macropa), Macrodipa, 2,2′,2″,2′″-(1,10-dioxa-4,7,13,16-tetraazacyclooctadecane-4,7,13,16-tetrayl)tetraacetic acid) (Crown), 1,4,7,10-Tetraazacyclododecane-1,4,7,10-tetraacetic acid, α-(2-carboxyethyl) (DOTAGA), 1,4,7-Triazacyclononane-N,N′,N″-triacetic acid (NOTA), 1,4,7,10-tetraazacyclododecane-N,N′,N′,N′″-tetraacetic acid (TETA), 1,4,7,10,13-pentaazacyclopentadecane-N,N′,N″,N′″,N″″-pentaacetic acid (PEPA), and 1,4,7,10,13,16-hexaazacyclohexadecane-N,N′,N″,N′″,N″″,N′″″-hexaacetic acid (HEHA).

In some embodiments, the chelating agents are polyaminocarboxylate agents, such as ethylenediamine tetraacetic acid (EDTA), diethylenetriamine pentaacetic acid (DTPA), 1,4,7,10-tetra-azacylcododecane-N,N′,N″,N′″-tetraacetic acid (DOTA), or derivatives thereof. They can coordinate with metals such as Fe, In, Ga, Zr, Y, Bi, Pb, or Ac.

In some embodiments, the chelator is EDTA:

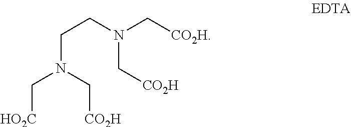 [SMILES: O=C(O)CN(CCN(CC(=O)O)CC(=O)O)CC(=O)O]

In some embodiments, the cheating agents are macrocyclic agents: 1,4,7-Triazacyclononane-N,N′,N″-triacetic acid (NOTA), 1,4,7,10-tetraazacyclododecane-N,N′,N″,N′″-tetraacetic acid (TETA), 1,4,7,10,13-pentaazacyclopentadecane-N,N′,N″,N′″,N″″-pentaacetic acid (PEPA), 1,4,7,10,13,16-hexaazacyclohexadecane-N,N′,N″,N′″,N″″,N′″″-hexaacetic acid (HEHA), or derivatives thereof.

Non-limiting examples of DTPA and derivatives thereof are:

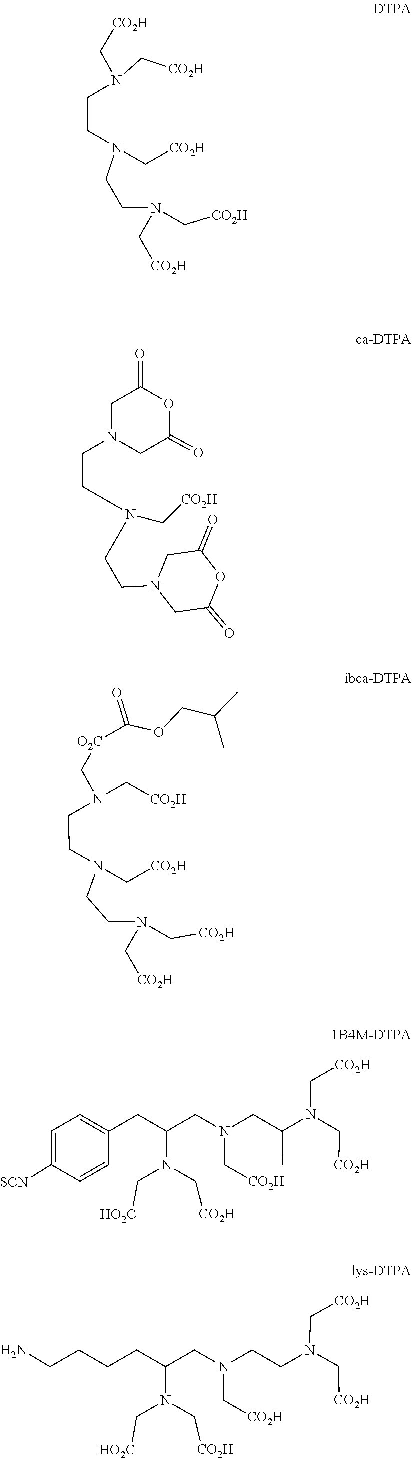

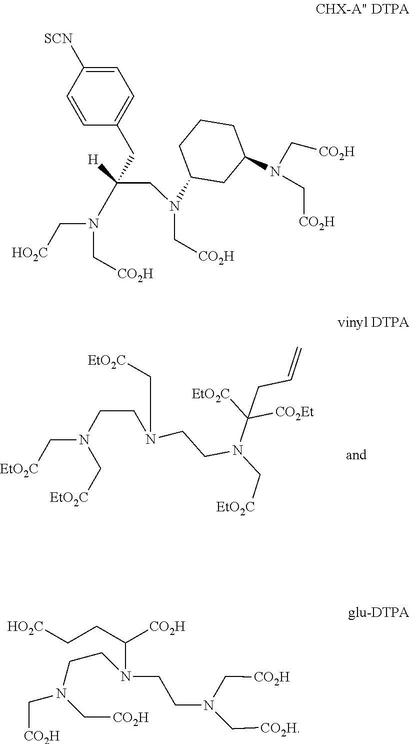

Non-limiting examples of DOTA and derivatives thereof are:

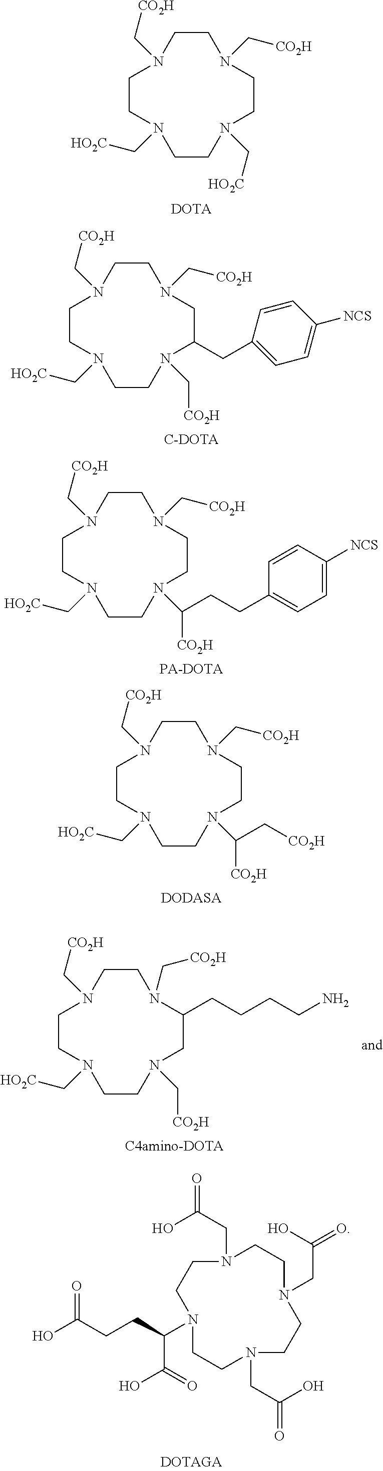

In some embodiments, the chelating agents of the present disclosure include DOTA, DOTAGA, or any derivative/analog thereof. Any chelating agent disclosed in Eisenwiener et al., Bioorg Med Chem Lett., vol. 10(18):2133 (2000), the contents of which are incorporated herein by reference in their entirety, can be used as a chelating agent.

Other non-limiting examples of the chelating agents include:

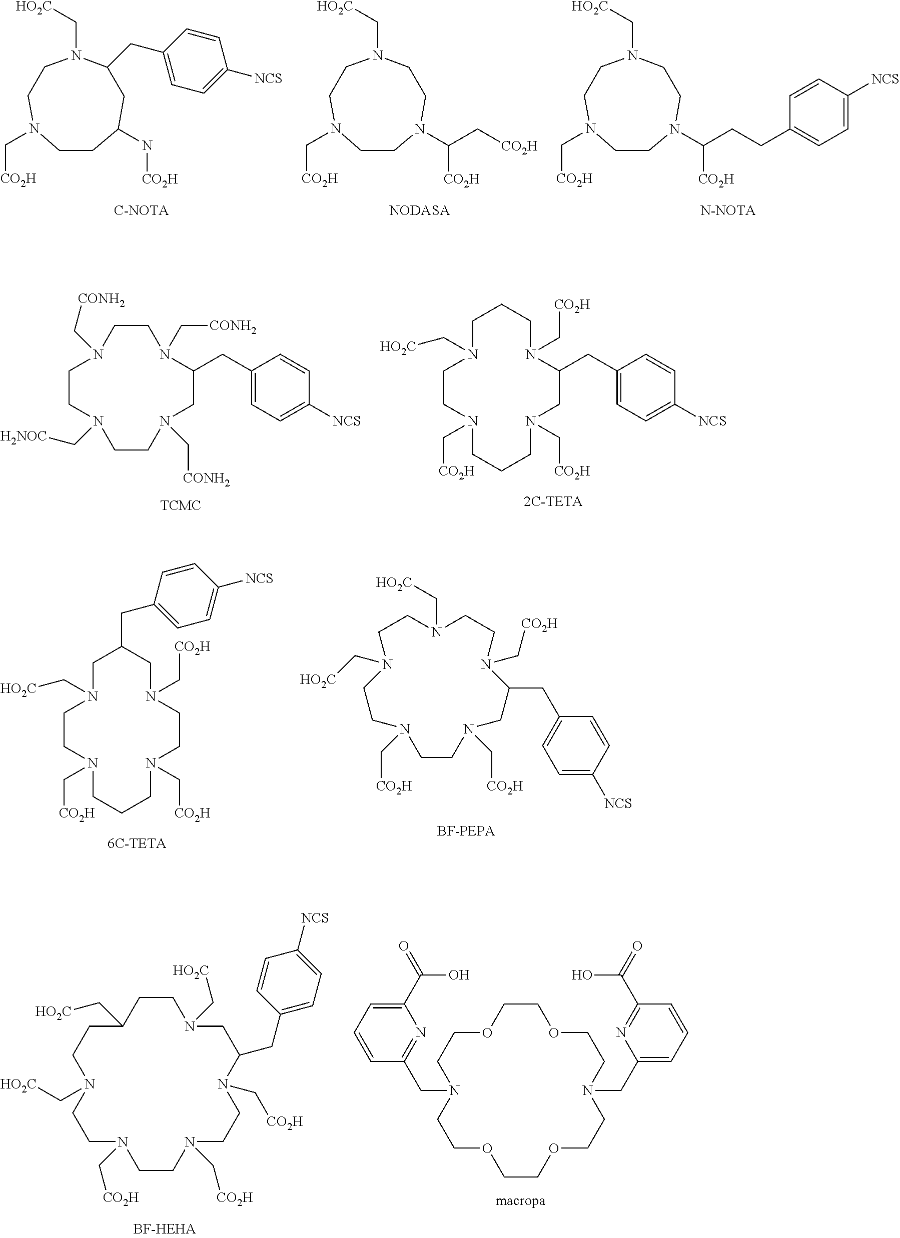

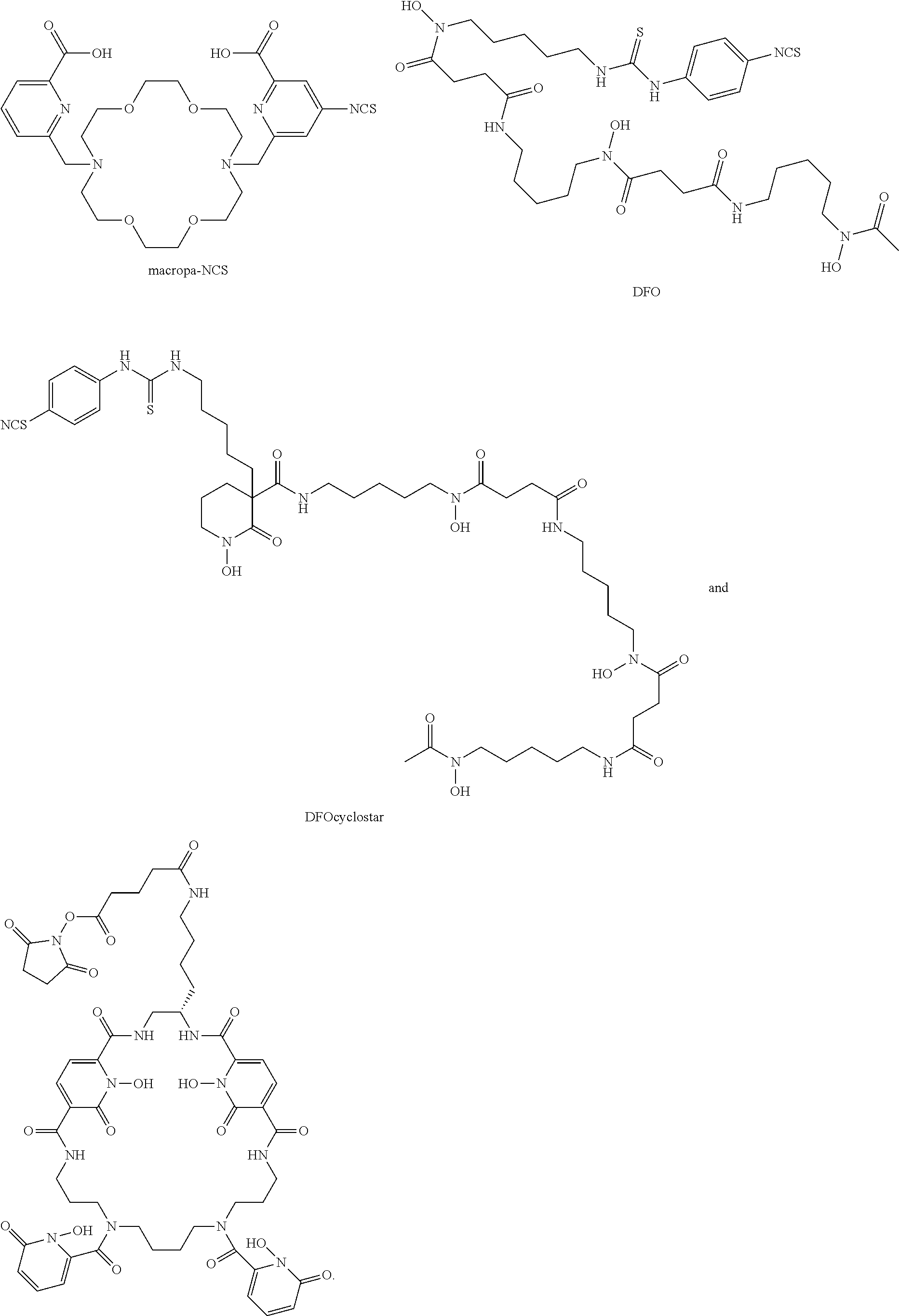

As used herein, the term “compound,” refers to a distinct chemical entity. Constructs, targeting constructs, targeting moieties, cargo, chelators, or other construct components, together with any fragments or variants of the foregoing, may be referred to independently or collectively as compounds. Compounds described herein may be provided as salts and may be prepared in combination with solvent or water molecules to form solvates and hydrates by routine methods. As used herein, “compound” is used interchangeably with “peptide.”

As used herein, the term “construct” refers to an artificially manipulated molecule. Some constructs may include nucleic acids and/or peptides, which may be products of recombinant technology and may be artificially synthesized or expressed from a recombinant nucleic acid sequence. Constructs may be combinations of nucleic acids, peptides, and/or other compounds.

Compounds may exist in one or more isomeric or isotopic forms (including, but not limited to stereoisomers, geometric isomers, tautomers, and isotopes). Compounds may be provided or utilized in singular form or as a mixture of two or more forms (including, but not limited to racemic mixtures of stereoisomers). Some compounds may exist in different forms, which may exhibit different properties and/or activities (including, but not limited to biological activities). For example, compounds containing asymmetrically substituted carbon atoms may be isolated in optically active or racemic forms.

The absolute stereochemistry is specified herein according to the Cahn-Ingold-Prelog R—S system. Chiral centers, of which the absolute configurations are known, are labelled by prefixes R and S, assigned by the standard sequence-rule procedure, and preceded when necessary by the appropriate locants (Pure & Appl. Chem. 45, 1976, 11-30). Certain examples contain chemical structures that are depicted or labelled as an (R*) or (S*). When (R*) or (S*) is used in the name of a compound or in the chemical representation of the compound, it is intended to convey that the compound is a pure single isomer at that stereocenter; however, absolute configuration of that stereocenter has not been established. Thus, a compound designated as (R*) refers to a compound that is a pure single isomer at that stereocenter with an absolute configuration of either (R) or (S), and a compound designated as (S*) refers to a compound that is a pure single isomer at that stereocenter with an absolute configuration of either (R) or (S).

The compounds described herein may be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C=N double bonds, and the like can also be present in the compounds described herein, and all such stable isomers are contemplated in the present disclosure. Cis and trans geometric isomers of compounds of the present disclosure may be isolated as a mixture of isomers or as separated isomeric forms.

Tautomeric compound forms result from the swapping of a single bond with an adjacent double bond and concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds provided herein can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

In the compounds provided herein, any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Also, unless otherwise stated, when a position is designated specifically as “D” or “deuterium,” the position is understood to have deuterium at an abundance that is at least 3000 times greater than the natural abundance of deuterium, which is 0.015% (i.e., at least 45% incorporation of deuterium).

As used herein, the terms “radioligand therapy” and “RLT” refer to a composition comprising a radiolabeled compound. In an embodiment, the radiolabeled compound is a radiolabeled peptide. In a non-limiting embodiment, the radiolabeled peptide is [²²⁵Ac]Ac-DOTA-TATE.

As used herein, the term “pharmaceutical composition” refers to a composition comprising at least one active ingredient in a form and amount that permits the active ingredient to be therapeutically effective and a pharmaceutically acceptable carrier or excipient.

The term “pharmaceutically acceptable,” as used herein, refers to compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio (e.g., in accordance with the guidelines of government agencies or other regulatory bodies, for example, the U.S. Food and Drug Administration).

As used herein, the phrase “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, the term “pharmaceutically acceptable salt” refers to salts of acidic or basic groups that are substantially nontoxic and non-inflammatory in subjects. Compounds that are basic in nature are capable of forming a variety of salts with various inorganic and organic acids. Acids that may be used to prepare pharmaceutically acceptable acid addition salts of basic compounds are those that form non-toxic acid addition salts, i.e., salts containing pharmacologically acceptable anions, including but not limited to sulfate, citrate, malate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds including amino moieties may form pharmaceutically acceptable salts with various amino acids, in addition to the acids mentioned above. Compounds that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the terms “patient,” “subject,” and “individual” refer to a subject seeking treatment, in need of treatment, requiring treatment, receiving treatment, expecting treatment, or that are under the care of a trained (e.g., licensed) professional for a particular disease, disorder, or condition. Patients may include any organism. Patient treatments may include, but are not limited to, experimental, diagnostic, prophylactic, and/or therapeutic treatments. Typical patients include, but are not limited to, animals (e.g., mammals such as mice, rats, rabbits, non-human primates, and humans).

The term “administration” or the like as used herein refers to the providing a therapeutic agent to a subject. Multiple techniques of administering a therapeutic agent exist in the art including, but not limited to, intravenous, oral, aerosol, parenteral, ophthalmic, pulmonary, and topical administration.

As used herein, the term “target” refers to an object or entity to be affected by an action or refers to activity associated with an agent that is directed to the object or entity (e.g., an agent that “targets” an object or entity). In some embodiments, targets refer to antigens, epitopes, or other structures to which antibodies or other compounds bind or that are selected and/or used in the design, development, or isolation of antigen-specific antibodies or other compounds. Targets may include molecular structures that include, but are not limited to, nucleic acids, peptides, proteins, haptens, receptors, carbohydrates, glycans, enzymes, lipids, cells, and fragments or complexes of any of the foregoing.

When used to refer to activity of an agent directed to objects or entities, the term “target(s)” may be used to describe binding activity of agents (e.g., antibodies, cyclic peptides, or related structures) with such objects or entities (e.g., antigens or epitopes). For example, an antibody that binds to a specific antigen may be said to “target” or be “directed to” the particular antigen. Similarly, a compound (e.g., a targeting construct) that exhibits activity (e.g., therapeutic or cytotoxic activity) toward a specific cell or tissue may be said to “target” the cell or tissue.

Targets may include cells (referred to herein as “target cells”). Target cells may be in vivo or in vitro. Target cells may include, for example, blood cells, lymph cells, cells lining the alimentary canal, such as the oral and pharyngeal mucosa, cells forming the villi of the small intestine, cells lining the large intestine, cells lining the respiratory system (nasal passages/lungs) of an animal, dermal/epidermal cells, cells of the vagina and rectum, cells of internal organs, cells of the placenta, and cells of the blood-brain barrier. In some embodiments, target cells may be cancer cells, including, but not limited to those found in leukemias or tumors (e.g., tumors of the brain, lung (small cell and non-small cell), ovary, prostate, breast, and colon, as well as other carcinomas and sarcomas). In still other embodiments, target cells may be part of a tissue. Tissues with target cells or other target structures are referred to herein as target tissues. Target tissues may include, but are not limited to, neuronal tissues, intestinal tissues, pancreatic tissues, liver tissues, kidney tissues, prostate tissues, ovary tissues, lung tissues, bone marrow tissues, and breast tissue tissues.

As used herein, an “epitope” refers to a surface or region on one or more entities that is capable of interacting with an antibody or other binding biomolecule. For example, a protein epitope may contain one or more amino acids and/or post-translational modifications (e.g., phosphorylated residues) which interact with an antibody. In some embodiments, an epitope may be a “conformational epitope,” which refers to an epitope involving a specific three-dimensional arrangement of the entity(ies) having or forming the epitope. For example, conformational epitopes of proteins may include combinations of amino acids and/or post-translational modifications from folded, non-linear stretches of amino acid chains.

As used herein, the term “cancer” refers to a disease characterized by abnormal cell growth and division.

As used herein, the term “cancer cell” refers to a cell that grows and divides in an abnormal and uncontrolled manner. See, e.g., Stedman's Medical Dictionary, 25th ed.; Hensyl ed.; Williams & Wilkins: Philadelphia, 1990.

As used herein, the term “tumor” refers to a group of cells forming in solid tissue as a result of abnormal cell growth and division. Benign or “noncancerous” tumors remain isolated while malignant or “cancerous” tumors include cells capable of proliferating to surrounding tissues.

As used herein, the term “therapeutically effective amount” means an amount of an agent to be delivered that is sufficient, when administered to a subject suffering from or susceptible to a disease, disorder, and/or condition, to treat, improve symptoms of, diagnose, prevent, and/or delay the onset of the disease, disorder, and/or condition.

As used herein the terms “treat,” “treatment,” and the like, refer to any actions taken to offer relief from or alleviation of pathological processes. As it relates to any of the therapeutic indications recited herein, the terms “treat,” “treatment,” and the like mean to relieve or alleviate at least one symptom associated with such indications, or to slow or reverse the progression or anticipated progression of such indications.

The term “prevent,” “preventing,” or “prevention” as used herein, comprises the prevention of at least one symptom associated with or caused by the state, disease or disorder being prevented.

As used herein, the following abbreviations are defined by the structures in Table 1.

TABLE 1

| Abbreviation | Structure |
|---|---|
| (Cyx)2 | 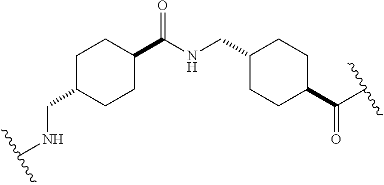 |
| (D-gE) | 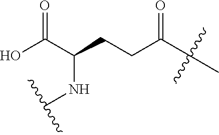 [SMILES: *N[C@H](CCC(*)=O)C(=O)O] |
| (D-gE)3 | 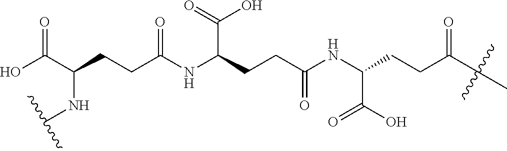 [SMILES: *N[C@H](CCC(=O)N[C@H](CCC(=O)N[C@H](CCC(*)=O)C(=O)O)C(=O)O)C(=O)O] |
| (gE)3 | 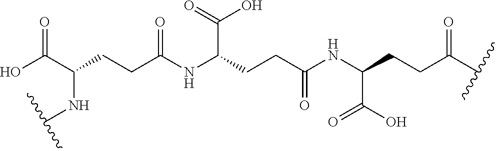 [SMILES: *N[C@@H](CCC(=O)N[C@@H](CCC(=O)N[C@@H](CCC(*)=O)C(=O)O)C(=O)O)C(=O)O] |
| (Sar)5 | 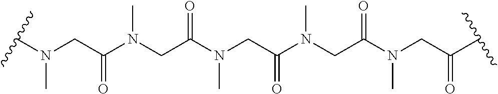 [SMILES: *C(=O)CN(C)C(=O)CN(C)C(=O)CN(C)C(=O)CN(C)C(=O)CN(*)C] |
| 1Me-Trp (1Me-W) | 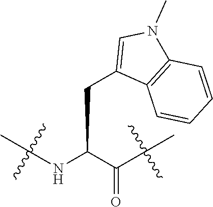 [SMILES: *N[C@@H](Cc1cn(C)c2ccccc12)C(*)=O] |
| 1Nal | 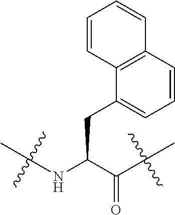 [SMILES: *N[C@@H](Cc1cccc2ccccc12)C(*)=O] |
| 2CF3-Phe (2CF3- F) | 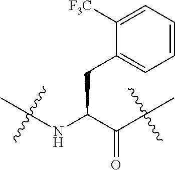 [SMILES: *N[C@@H](Cc1ccccc1C(F)(F)F)C(*)=O] |
| 2Nal | 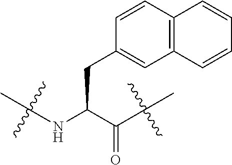 [SMILES: *N[C@@H](Cc1ccc2ccccc2c1)C(*)=O] |
| 2PhEt-Ala (2PhEt-A) | 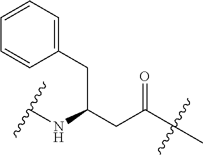 [SMILES: *N[C@H](CC(*)=O)Cc1ccccc1] |
| 3-(1-morpholinyl)- Ala | 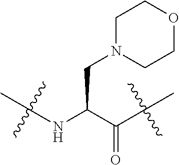 [SMILES: *N[C@@H](CN1CCOCC1)C(*)=O] |
| 3-(4-piperidinyl)- Ala | 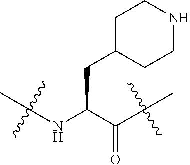 [SMILES: *N[C@@H](CC1CCNCC1)C(*)=O] |
| 3Me2-Aze | 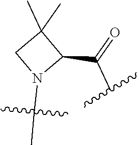 [SMILES: *C(=O)[C@H]1N(*)CC1(C)C] |
| 3Pya | 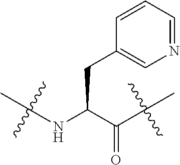 [SMILES: *N[C@@H](Cc1cccnc1)C(*)=O] |
| 4CF3-Phe (4CF3- F) | 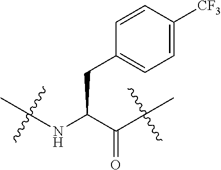 [SMILES: *N[C@@H](Cc1ccc(C(F)(F)F)cc1)C(*)=O] |
| 4F-Phe (4Fluoro- F) | 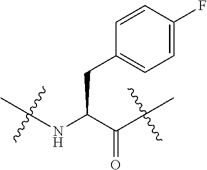 [SMILES: *N[C@@H](Cc1ccc(F)cc1)C(*)=O] |
| 4Pya | 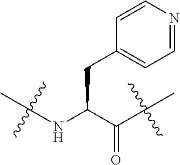 [SMILES: *N[C@@H](Cc1ccncc1)C(*)=O] |
| 5,5-diMe-Pro (5,5- diMe-P) | 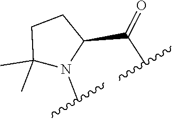 [SMILES: *C(=O)[C@@H]1CCC(C)(C)N1*] |
| 5F-Trp (5Fluoro- W) | 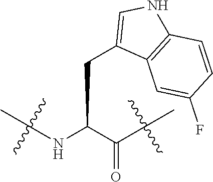 [SMILES: *N[C@@H](Cc1c[nH]c2ccc(F)cc12)C(*)=O] |
| 5OH-Trp (5OH- W) | 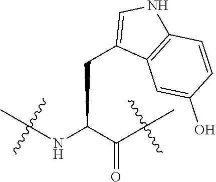 [SMILES: *N[C@@H](Cc1c[nH]c2ccc(O)cc12)C(*)=O] |
| 5OMe-Trp (5OMe-W) | 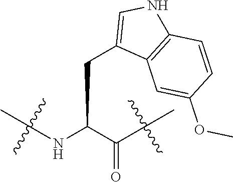 [SMILES: *N[C@@H](Cc1c[nH]c2ccc(OC)cc12)C(*)=O] |
| 5Qui | 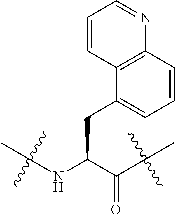 [SMILES: *N[C@@H](Cc1cccc2ncccc12)C(*)=O] |
| 7Aza-Trp (7Aza- W) | 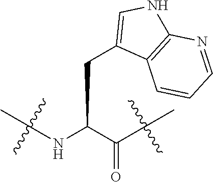 [SMILES: *N[C@@H](Cc1c[nH]c2ncccc12)C(*)=O] |
| 7Cl-Trp (7Cl-W) | 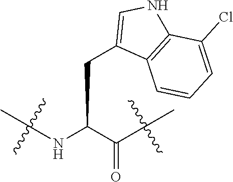 [SMILES: *N[C@@H](Cc1c[nH]c2c(Cl)cccc12)C(*)=O] |
| 7MeO-Trp (7MeO-W) | 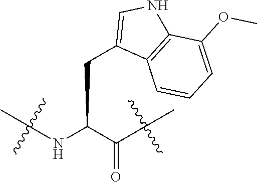 [SMILES: *N[C@@H](Cc1c[nH]c2c(OC)cccc12)C(*)=O] |
| 7Me-Trp (7Me-W) | 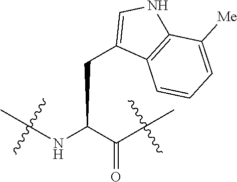 [SMILES: *N[C@@H](Cc1c[nH]c2c(C)cccc12)C(*)=O] |
| ACl | 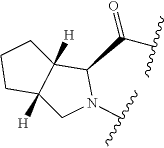 [SMILES: *C(=O)[C@@H]1[C@H]2CCC[C@H]2CN1*] |
| Ahx | 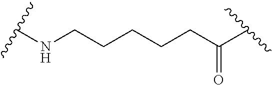 [SMILES: *NCCCCCC(*)=O] |
| Allo-lle | 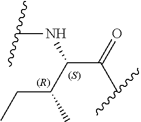 |
| AMBX | 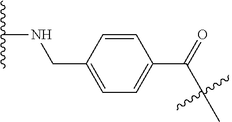 [SMILES: *NCc1ccc(C(*)=O)cc1] |
| alpha-Me-Aze | 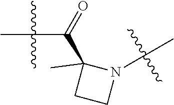 [SMILES: *C(=O)[C@]1(C)CCN1*] |
| alpha-Me-Asp (aMe-D) | 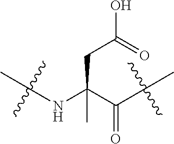 [SMILES: *N[C@](C)(CC(=O)O)C(*)=O] |
| alpha-Me-lle (aMe-I) | 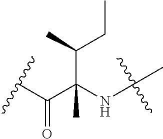 [SMILES: *N[C@](C)(C(*)=O)[C@@H](C)CC] |
| alpha-Me-Pro (aMe-P) | 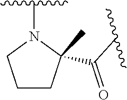 [SMILES: *C(=O)[C@]1(C)CCCN1*] |
| alpha-Me-Thr (aMe-T) | 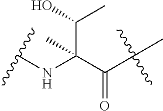 [SMILES: *N[C@](C)(C(*)=O)[C@@H](C)O] |
| alpha-Me-Trp (aMe-W) | 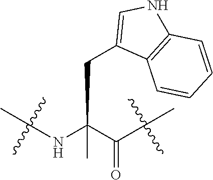 [SMILES: *N[C@](C)(Cc1c[nH]c2ccccc12)C(*)=O] |
| Aze | 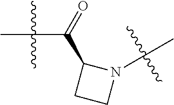 [SMILES: *C(=O)[C@@H]1CCN1*] |
| BIP | 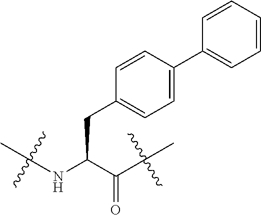 [SMILES: *N[C@@H](Cc1ccc(-c2ccccc2)cc1)C(*)=O] |
| C12OH | 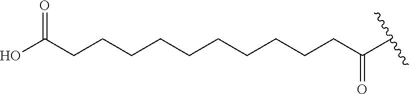 [SMILES: *C(=O)CCCCCCCCCCC(=O)O] |
| C14 | 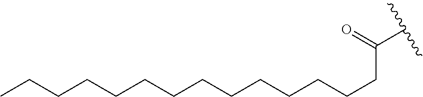 [SMILES: *C(=O)CCCCCCCCCCCCCC] |
| C15 | 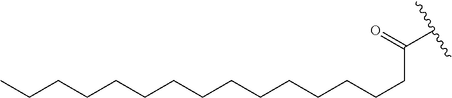 [SMILES: *C(=O)CCCCCCCCCCCCCCC] |
| C15-(L-gE)- PEG12 | 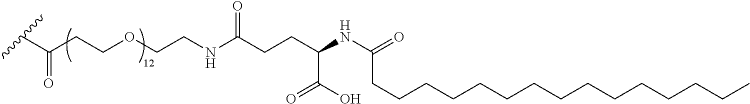 |
| COC16 | 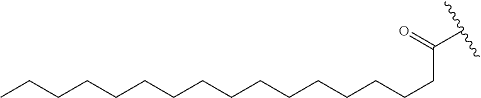 [SMILES: *C(=O)CCCCCCCCCCCCCCCC] |
| CBA | 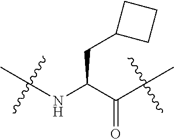 [SMILES: *N[C@@H](CC1CCC1)C(*)=O] |
| CHA | 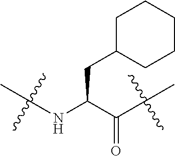 [SMILES: *N[C@@H](CC1CCCCC1)C(*)=O] |
| Chg | 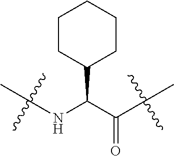 [SMILES: *N[C@H](C(*)=O)C1CCCCC1] |
| cis4Fluoro-Pro (cis4Fluoro-P) | 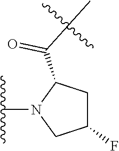 [SMILES: *C(=O)[C@@H]1C[C@H](F)CN1*] |
| cis4NH2-Pro (cis4NH2-P) | 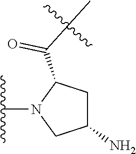 [SMILES: *C(=O)[C@@H]1C[C@H](N)CN1*] |
| cis4OH-Pro (cis4OH-P) | 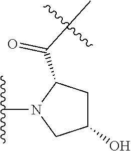 [SMILES: *C(=O)[C@@H]1C[C@H](O)CN1*] |
| CyHex | 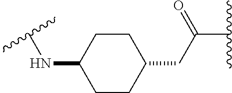 [SMILES: *N[C@H]1CC[C@H](CC(*)=O)CC1] |
| DAB-4- NHCOC5H11 | 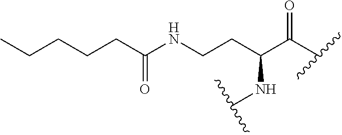 [SMILES: *N[C@@H](CCNC(=O)CCCCC)C(*)=O] |
| DAB-4- NHCOC7H15 | 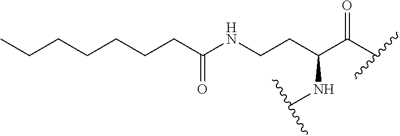 [SMILES: *N[C@@H](CCNC(=O)CCCCCCC)C(*)=O] |
| D-Ala | 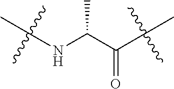 [SMILES: *N[C@H](C)C(*)=O] |
| D-Lys((D-Pro)3- PEG8- ) | 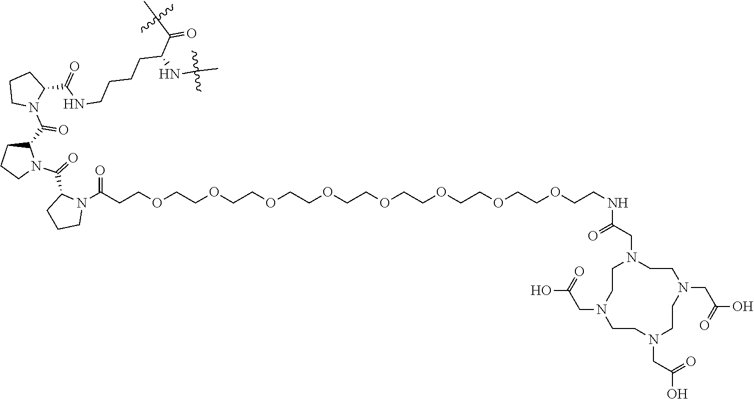 |
| D-Lys((D-Pro)- PEG8-DOTA) | 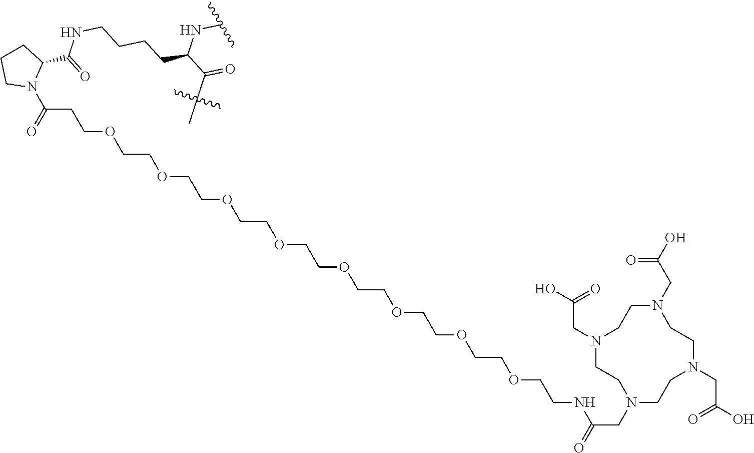 [SMILES: *N[C@H](CCCCNC(=O)[C@H]1CCCN1C(=O)CCOCCOCCOCCOCCOCCOCCOCCOCCNC(=O)CN1CCN(CC(=O)O)CCN(CC(=O)O)CCN(CC(=O)O)CC1)C(*)=O] |
| D-Lys(Ahx-DOTA) | 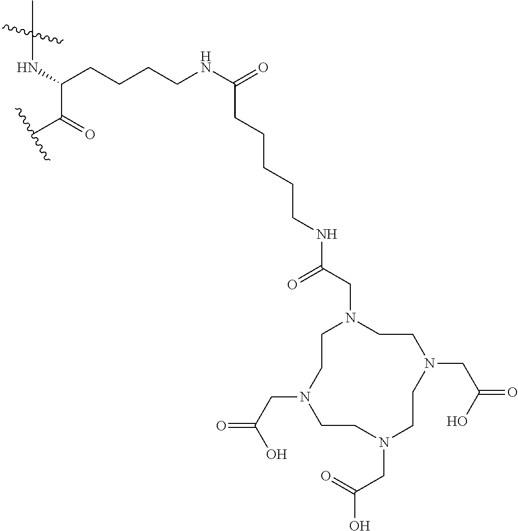 [SMILES: *N[C@H](CCCCNC(=O)CCCCCNC(=O)CN1CCN(CC(=O)O)CCN(CC(=O)O)CCN(CC(=O)O)CC1)C(*)=O] |
| D-Lys(Chx- DOTA) | 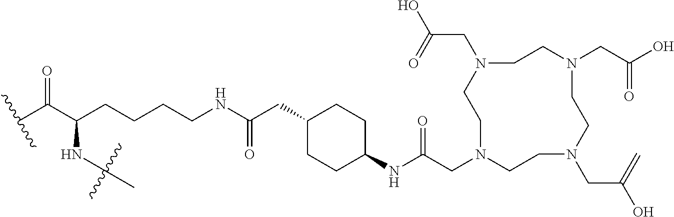 [SMILES: *N[C@H](CCCCNC(=O)C[C@H]1CC[C@H](NC(=O)CN2CCN(CC(=C)O)CCN(CC(=O)O)CCN(CC(=O)O)CC2)CC1)C(*)=O] |
| D-Lys(DOTA) | 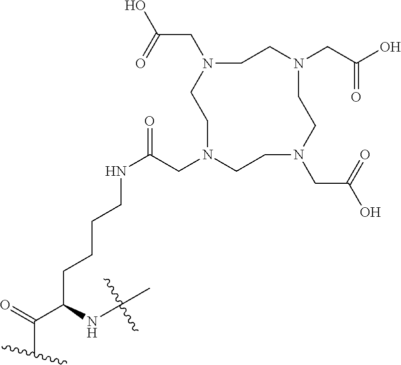 [SMILES: *N[C@H](CCCCNC(=O)CN1CCN(CC(=O)O)CCN(CC(=O)O)CCN(CC(=O)O)CC1)C(*)=O] |
| D-Lys(gE-DOTA) | 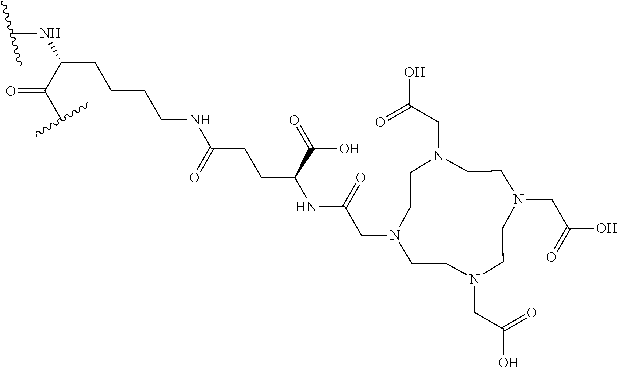 [SMILES: *N[C@H](CCCCNC(=O)CC[C@H](NC(=O)CN1CCN(CC(=O)O)CCN(CC(=O)O)CCN(CC(=O)O)CC1)C(=O)O)C(*)=O] |
| D-Lys(gE-gE-gE- DOTA) | 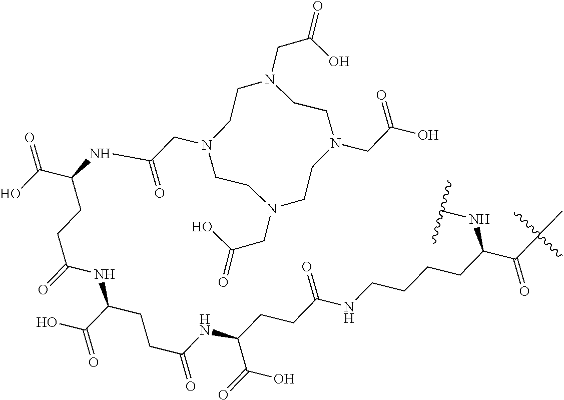 [SMILES: *N[C@H](CCCCNC(=O)CC[C@H](NC(=O)CC[C@H](NC(=O)CC[C@H](NC(=O)CN1CCN(CC(=O)O)CCN(CC(=O)O)CCN(CC(=O)O)CC1)C(=O)O)C(=O)O)C(=O)O)C(*)=O] |
| D-Lys(Gly-Gly- Ser-Gly-Gly-Ser- DOTA) | 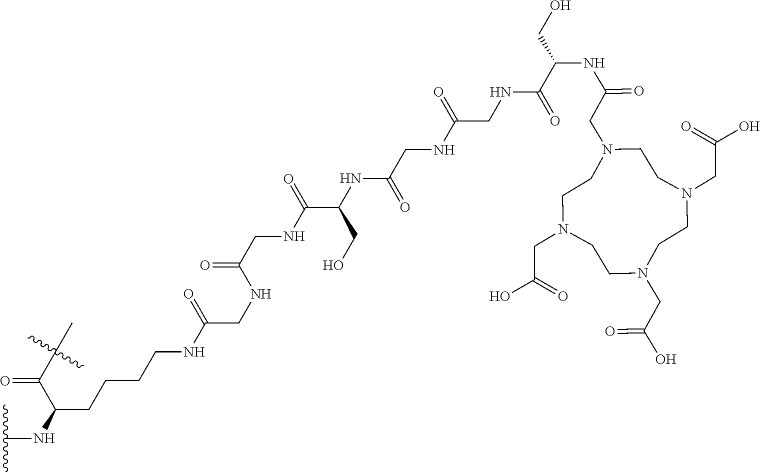 [SMILES: *N[C@H](CCCCNC(=O)CNC(=O)CNC(=O)[C@H](CO)NC(=O)CNC(=O)CNC(=O)[C@H](CO)NC(=O)CN1CCN(CC(=O)O)CCN(CC(=O)O)CCN(CC(=O)O)CC1)C(*)=O] |
| D-Lys(Gly-Gly- TTDS-DOTA) | 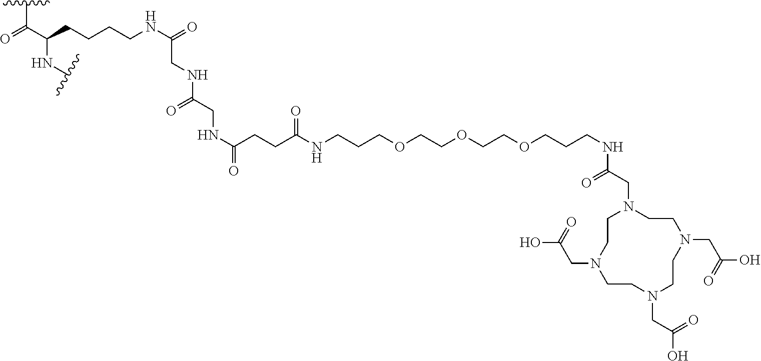 [SMILES: *N[C@H](CCCCNC(=O)CNC(=O)CNC(=O)CCC(=O)NCCCOCCOCCOCCCNC(=O)CN1CCN(CC(=O)O)CCN(CC(=O)O)CCN(CC(=O)O)CC1)C(*)=O] |
| D-Lys(Gly-Ser- Gly-Ser-Gly-Ser- DOTA) | 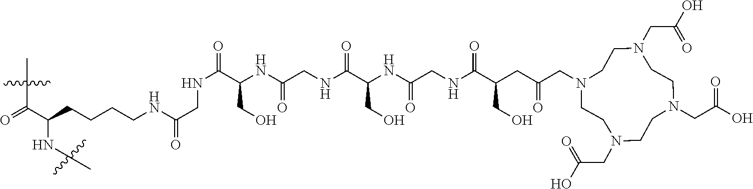 [SMILES: *N[C@H](CCCCNC(=O)CNC(=O)[C@H](CO)NC(=O)CNC(=O)[C@H](CO)NC(=O)CNC(=O)[C@H](CO)CC(=O)CN1CCN(CC(=O)O)CCN(CC(=O)O)CCN(CC(=O)O)CC1)C(*)=O] |
| D-Lys(PEG4- DOTA) | 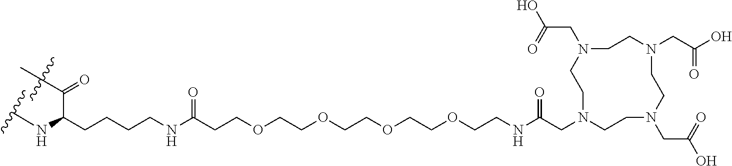 [SMILES: *N[C@H](CCCCNC(=O)CCOCCOCCOCCOCCNC(=O)CN1CCN(CC(=O)O)CCN(CC(=O)O)CCN(CC(=O)O)CC1)C(*)=O] |
| D-Lys(PEG8- COCH2Ph-4Br) | 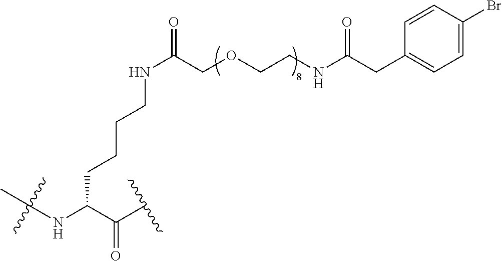 |
| D-Lys(PEG8- COCH2Ph-4I) | 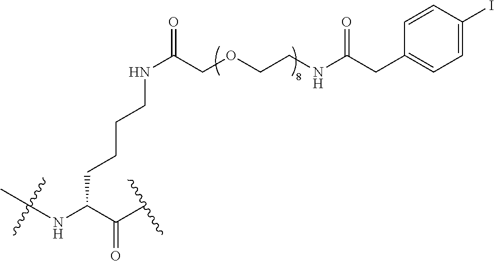 |
| D-Lys(PEG8- DOTA) | 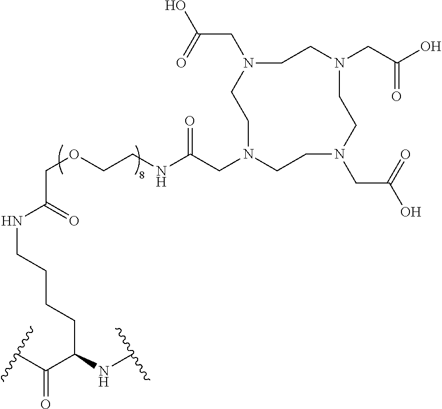 |
| D-Lys(SP6- PEG8-DOTA) | 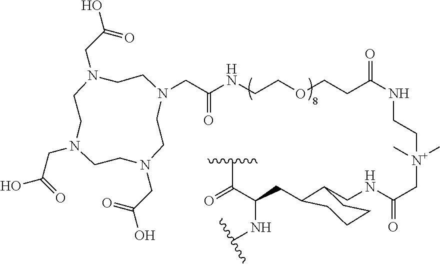 |
| D-Lys[PEG8-D- Lys(DOTA)- PEG12- COCH2Ph-4Br] | 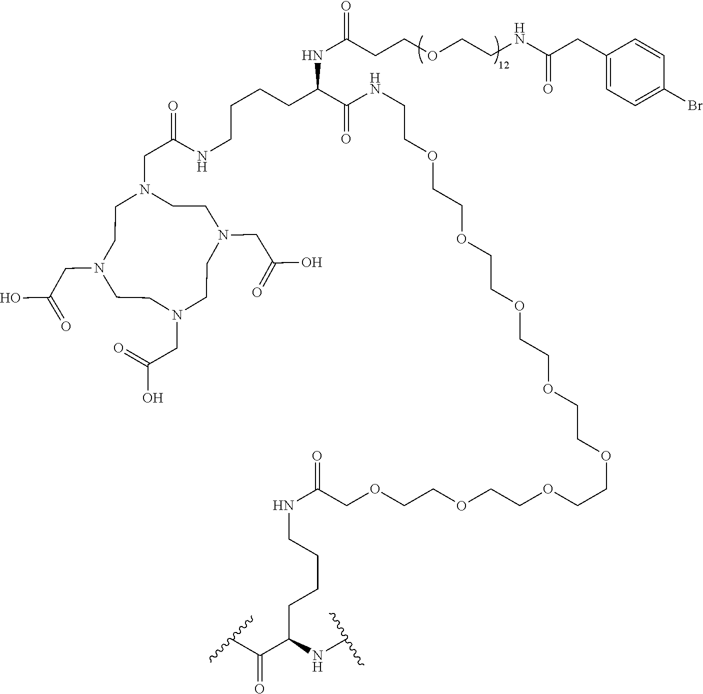 |
| D-Lys[PEG8-D- Lys(DOTA)- PEG24- COCH2Ph-4Br] | 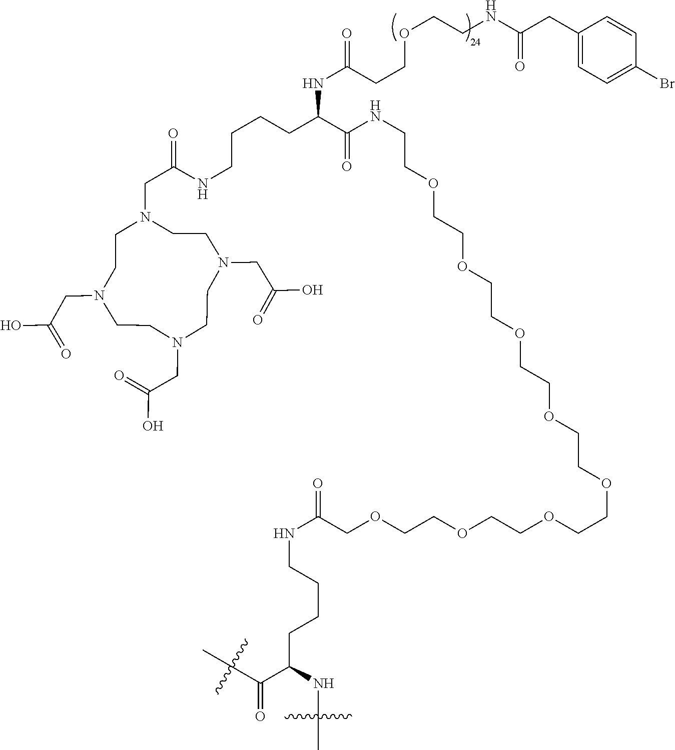 |
| D-Lys[PEG8-D- Lys(DOTA)- PEG8-COCH2Ph- 4Br] | 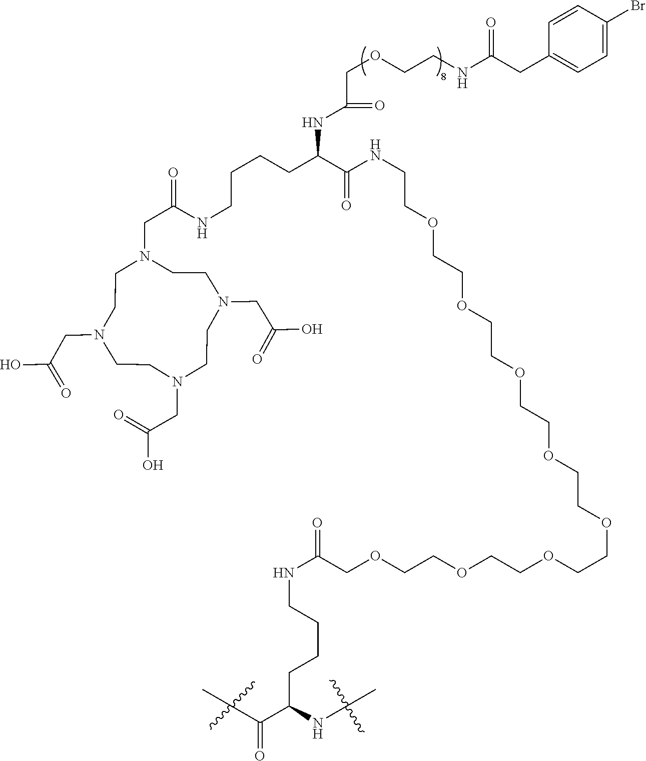 |
| D-Nle | 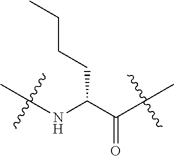 [SMILES: *N[C@H](CCCC)C(*)=O] |
| D-Pro (dPro) | 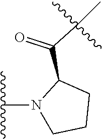 [SMILES: *C(=O)[C@H]1CCCN1*] |
| Env | 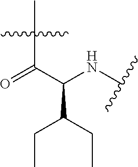 [SMILES: *N[C@H](C(*)=O)C(CC)CC] |
| gE | 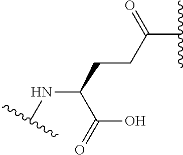 [SMILES: *N[C@@H](CCC(*)=O)C(=O)O] |
| Glu[gLys(OH)- Val-Met-AmBz- DOTA] | 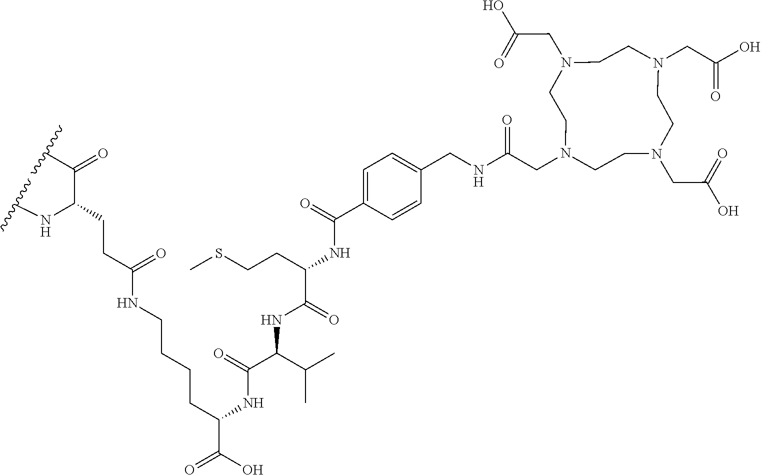 [SMILES: *N[C@@H](CCC(=O)NCCCC[C@H](NC(=O)[C@@H](NC(=O)[C@H](CCSC)NC(=O)c1ccc(CNC(=O)CN2CCN(CC(=O)O)CCN(CC(=O)O)CCN(CC(=O)O)CC2)cc1)C(C)C)C(=O)O)C(*)=O] |
| Lys(C12) | 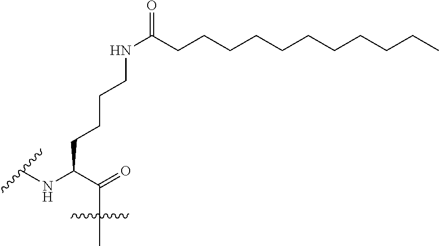 [SMILES: *N[C@@H](CCCCNC(=O)CCCCCCCCCCC)C(*)=O] |
| Lys(C14) | 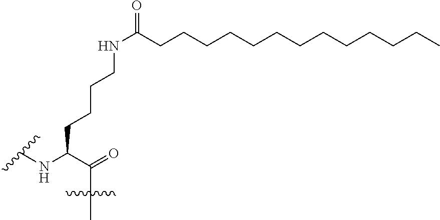 [SMILES: *N[C@@H](CCCCNC(=O)CCCCCCCCCCCCC)C(*)=O] |
| Lys(C16) | 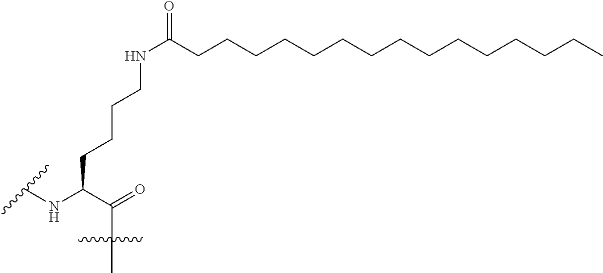 [SMILES: *N[C@@H](CCCCNC(=O)CCCCCCCCCCCCCCC)C(*)=O] |
| Lys(Me)3 | 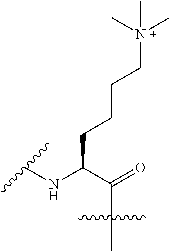 [SMILES: *N[C@@H](CCCC[N+](C)(C)C)C(*)=O] |
| Lys(DOTA) | 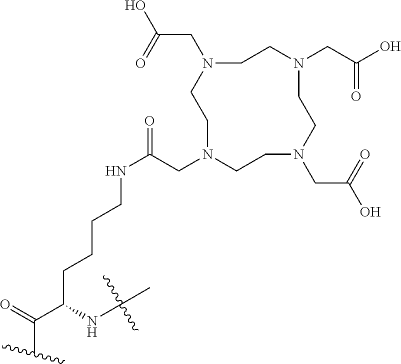 [SMILES: *N[C@@H](CCCCNC(=O)CN1CCN(CC(=O)O)CCN(CC(=O)O)CCN(CC(=O)O)CC1)C(*)=O] |
| Lys(Chx-DOTA) | 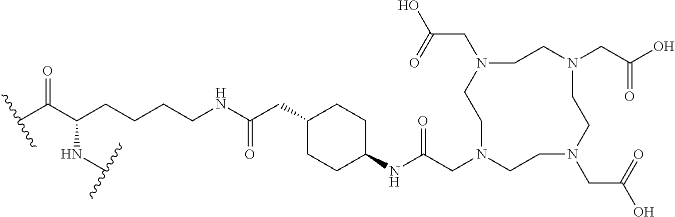 [SMILES: *N[C@@H](CCCCNC(=O)C[C@H]1CC[C@H](NC(=O)CN2CCN(CC(=O)O)CCN(CC(=O)O)CCN(CC(=O)O)CC2)CC1)C(*)=O] |
| Lys(Gly-Gly- TTDS-DOTA) | 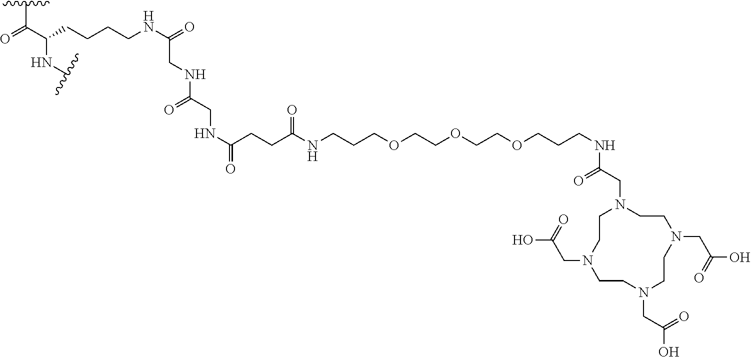 [SMILES: *N[C@@H](CCCCNC(=O)CNC(=O)CNC(=O)CCC(=O)NCCCOCCOCCOCCCNC(=O)CN1CCN(CC(=O)O)CCN(CC(=O)O)CCN(CC(=O)O)CC1)C(*)=O] |
| Lys(Gly-Ser-Gly- Ser-Gly-Ser- DOTA) | 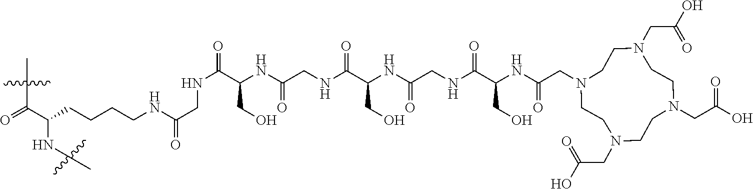 [SMILES: *N[C@@H](CCCCNC(=O)CNC(=O)[C@H](CO)NC(=O)CNC(=O)[C@H](CO)NC(=O)CNC(=O)[C@H](CO)NC(=O)CN1CCN(CC(=O)O)CCN(CC(=O)O)CCN(CC(=O)O)CC1)C(*)=O] |
| MAF | 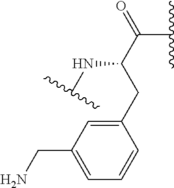 [SMILES: *N[C@@H](Cc1cccc(CN)c1)C(*)=O] |
| Nle | 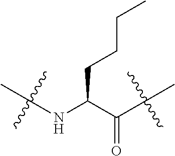 [SMILES: *N[C@@H](CCCC)C(*)=O] |
| NMe-Ala (NMeA) | 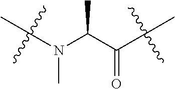 [SMILES: *C(=O)[C@H](C)N(*)C] |
| NMe-Asp (NMeD) | 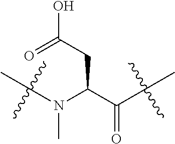 [SMILES: *C(=O)[C@H](CC(=O)O)N(*)C] |
| NMe-His (NMeH) | 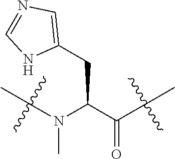 [SMILES: *C(=O)[C@H](Cc1cnc[nH]1)N(*)C] |
| NMeK(DOTA) | 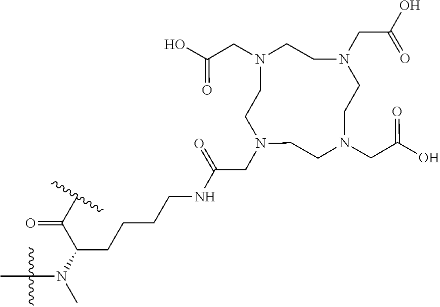 [SMILES: *C(=O)[C@H](CCCCNC(=O)CN1CCN(CC(=O)O)CCN(CC(=O)O)CCN(CC(=O)O)CC1)N(*)C] |
| NMe-Leu (NMeL) | 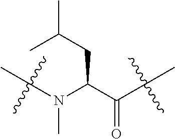 [SMILES: *C(=O)[C@H](CC(C)C)N(*)C] |
| NMe-Asn (NMeN) | 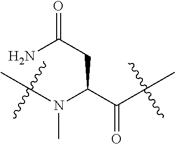 [SMILES: *C(=O)[C@H](CC(N)=O)N(*)C] |
| NMe-Nle | 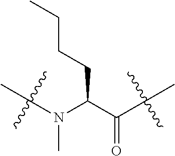 [SMILES: *C(=O)[C@H](CCCC)N(*)C] |
| NMe-Ser (NMeS) | 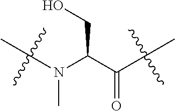 [SMILES: *C(=O)[C@H](CO)N(*)C] |
| NMe-Thr (NMeT) | 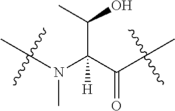 [SMILES: *C(=O)[C@H]([C@@H](C)O)N(*)C] |
| NMe-tBuAla | 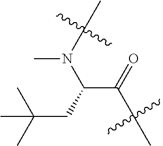 [SMILES: *C(=O)[C@H](CC(C)(C)C)N(*)C] |
| NMe-Trp (NMeW) | 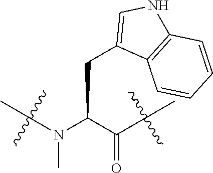 [SMILES: *C(=O)[C@H](Cc1c[nH]c2ccccc12)N(*)C] |
| OAF | 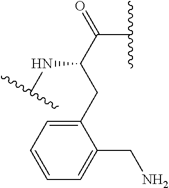 [SMILES: *N[C@@H](Cc1ccccc1CN)C(*)=O] |
| Orn | 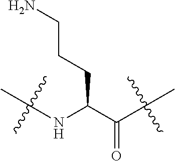 [SMILES: *N[C@@H](CCCN)C(*)=O] |
| PAF | 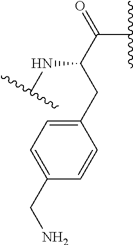 [SMILES: *N[C@@H](Cc1ccc(CN)cc1)C(*)=O] |
| PEG4 | 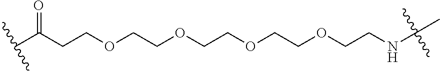 [SMILES: *NCCOCCOCCOCCOCCC(*)=O] |
| PEG8 | 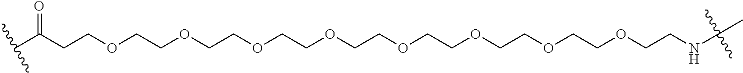 [SMILES: *NCCOCCOCCOCCOCCOCCOCCOCCOCCC(*)=O] |
| Pip | 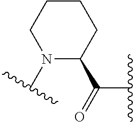 [SMILES: *C(=O)[C@@H]1CCCCN1*] |
| R-3Me-Aze | 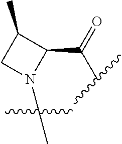 [SMILES: *C(=O)[C@@H]1[C@H](C)CN1*] |
| Sar | 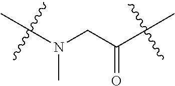 [SMILES: *C(=O)CN(*)C] |
| SP6 | 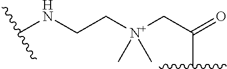 [SMILES: *NCC[N+](C)(C)CC(*)=O] |
| Tbg | 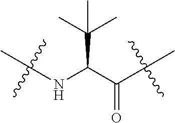 [SMILES: *N[C@H](C(*)=O)C(C)(C)C] |
| t-Bu-Ala | 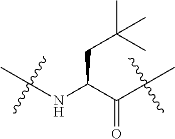 [SMILES: *N[C@@H](CC(C)(C)C)C(*)=O] |
| THPG | 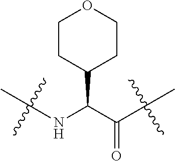 [SMILES: *N[C@H](C(*)=O)C1CCOCC1] |
| trans4Fluoro-Pro (trans4Fluoro-P) | 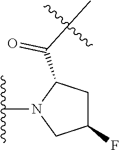 [SMILES: *C(=O)[C@@H]1C[C@@H](F)CN1*] |
| trans4NH2-Pro (trans4NH2-P) | 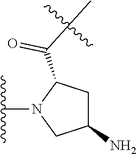 [SMILES: *C(=O)[C@@H]1C[C@@H](N)CN1*] |
| trans4OH-Pro (trans4OH-P) | 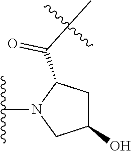 [SMILES: *C(=O)[C@@H]1C[C@@H](O)CN1*] |
| TTDS-Gly-Gly | 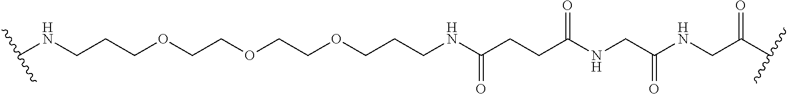 [SMILES: *NCCCOCCOCCOCCCNC(=O)CCC(=O)NCC(=O)NCC(*)=O] |
| α-tert-amylGly | 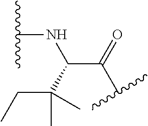 [SMILES: *N[C@H](C(*)=O)C(C)(C)CC] |
| DapN3 | 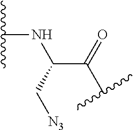 [SMILES: *N[C@@H](CN=[N+]=[N-])C(*)=O] |
| Pra | 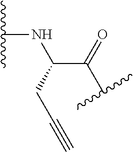 [SMILES: *N[C@@H](CC#C)C(*)=O] |
| Dap | 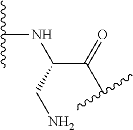 [SMILES: *N[C@@H](CN)C(*)=O] |
| L-2,4-Dab | 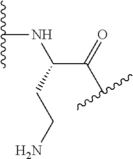 [SMILES: *N[C@@H](CCN)C(*)=O] |
| AllylG | 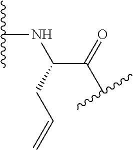 [SMILES: *N[C@@H](CC=C)C(*)=O] |
| Lys(PEG24- [gE(C16)]-OH) | 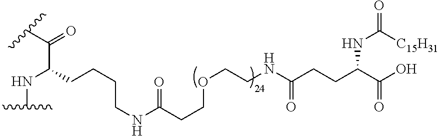 |
| Macrodipa | 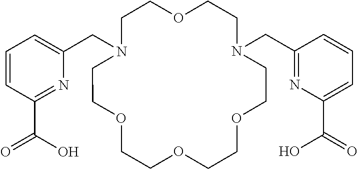 [SMILES: O=C(O)c1cccc(CN2CCOCCOCCOCCN(Cc3cccc(C(=O)O)n3)CCOCC2)n1] |
| D-Lys(Ac) | 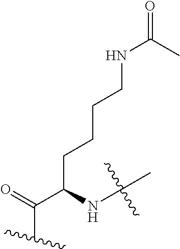 [SMILES: *N[C@H](CCCCNC(C)=O)C(*)=O] |
| C14OH | 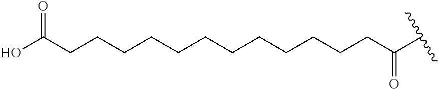 [SMILES: *C(=O)CCCCCCCCCCCCC(=O)O] |
| C16OH | 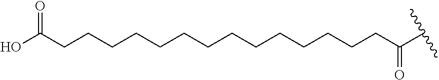 [SMILES: *C(=O)CCCCCCCCCCCCCCC(=O)O] |
| alpha-Me-Cys | 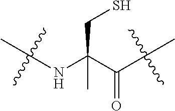 [SMILES: *N[C@](C)(CS)C(*)=O] |
| NMe-Cys | 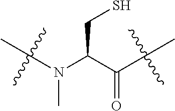 [SMILES: *C(=O)[C@H](CS)N(*)C] |
| D-Cys | 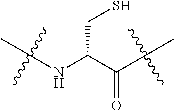 [SMILES: *N[C@H](CS)C(*)=O] |
| Pip(CH₂CO₂H)Ala | 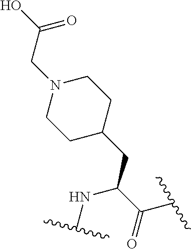 [SMILES: *N[C@@H](CC1CCN(CC(=O)O)CC1)C(*)=O] |
| Pip(PEGNMe₃)Ala | 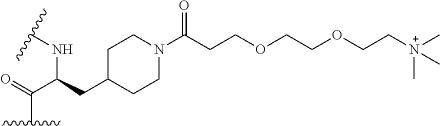 [SMILES: *N[C@@H](CC1CCN(C(=O)CCOCCOCC[N+](C)(C)C)CC1)C(*)=O] |
| Pip(GAE- DOTA)Ala | 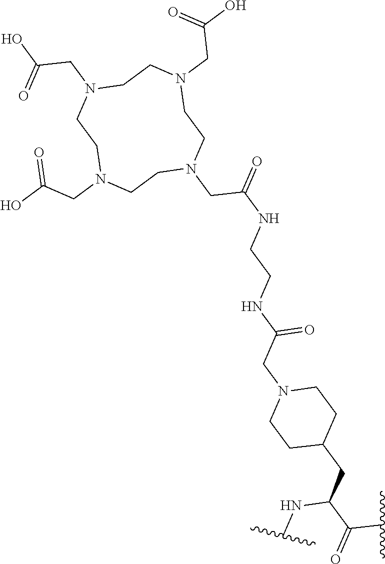 [SMILES: *N[C@@H](CC1CCN(CC(=O)NCCNC(=O)CN2CCN(CC(=O)O)CCN(CC(=O)O)CCN(CC(=O)O)CC2)CC1)C(*)=O] |

### Methods of Making Stabilized Compositions of Radiolabeled Compounds

Radiopharmaceuticals and radiopharmaceutical compositions are typically prepared at radio-pharmacies or PET Centers, where they also undergo quality control testing. Once a drug product has passed the quality control (00) process and has been released for administration to a patient, it may be administered to a patient on site, where PET scanning facilities exist, or it may be shipped to a PET scanning facility.

Regulatory standards for RLTs require that they are sterile, pyrogen-free, safe, and effective. One of the key tests in the 00 process is quantifying chemical impurities and the RCP of the drug product. In the United States, the FDA sets the minimum threshold for all radiopharmaceuticals, including RLTs, at 90% RCP. Maintaining a high RCP up to the point of administration is critical.

RCP analysis is routinely carried out by high pressure liquid chromatography (HPLC). HPLC achieves the separation of the various components of a liquid composition due to the different interactions of the components with the stationary phase (usually a silica-based column) and the mobile phase, or eluent, that passes through the column. The chemical nature of the components determines their affinity for the stationary phase, based on the intermolecular interactions, and the time spent on the column before eluting.

The above analysis is ensures that the RLT will remain pure and highly radioactive up to the point of administration. To ensure that this process is as efficient as possible, the production of RLTs with high stability is critical.

Thus, provided herein is a method of making a stabilized composition of a radiolabeled compound, comprising:
a. eluting a mixture comprising the radiolabeled compound and a corresponding unlabeled compound through a chromatography column, thereby separating the radiolabeled compound from the unlabeled compound;
b. collecting an eluate containing the separated radiolabeled compound in a vessel containing a stabilizing solution to afford the stabilized composition of the radiolabeled compound.

In some embodiments, the product of step (b) is a pharmaceutical composition.

In some embodiments, the method further comprises sterile filtration of the stabilized composition to afford a pharmaceutical composition of the radiolabeled compound. In another embodiment, the method further comprises sterile filtration through a filter having a pore size of 0.01-0.1 microns, 0.1-0.2 microns, or 0.2-0.3 microns. In yet another embodiment, the pore size is about 0.22 microns.

The use of pharmaceutically acceptable techniques and materials advantageously allows the stabilized compositions and pharmaceutical compositions provided herein to be packaged, distributed, and/or administered without further manipulation or delay. Accordingly, in some embodiments, eluting the mixture comprising the radiolabeled compound and unlabeled compound through a chromatography column comprises the use of mobile phase solvents, wherein the mobile phase solvents are pharmaceutically acceptable. In an embodiment, the mobile phase solvents are selected from ethanol, aqueous buffer, and combinations thereof.

A stoichiometric excess of compound (i.e., unlabeled compound) in relation to radionuclide may be used in the radiolabeling reaction as needed to achieve the desired level of radionuclide incorporation, thereby resulting in a mixture of radiolabeled compound and unlabeled compound following the radiolabeling reaction. Accordingly, in some embodiments, the molar ratio of radiolabeled compound to unlabeled compound is in the range of about 1:5 to about 1:15000. In a particular embodiment, the molar ratio is in the range of about 1:5 to about 1:10000. In a particular embodiment, the molar ratio is in the range of about 1:9 to about 1:10000.

In a particular embodiment, the radiolabeled compound is radiolabeled with ¹⁷⁷Lu and the molar ratio of radiolabeled compound to unlabeled compound is in the range of about 1:9 to about 1:100. In a particular embodiment, the radiolabeled compound is radiolabeled with ¹⁷⁷Lu and the molar ratio of radiolabeled compound to unlabeled compound is about 1:9. In a particular embodiment, the radiolabeled compound is radiolabeled with ¹⁷⁷Lu and the molar ratio of radiolabeled compound to unlabeled compound is about 1:19. In a particular embodiment, the radiolabeled compound is radiolabeled with ¹⁷⁷Lu and the molar ratio of radiolabeled compound to unlabeled compound is about 1:99.

In a particular embodiment, the radiolabeled compound is radiolabeled with ¹¹¹In and the molar ratio of radiolabeled compound to unlabeled compound is in the range of about 1:10 to about 1:500. In a particular embodiment, the radiolabeled compound is radiolabeled with ¹¹¹In and the molar ratio of radiolabeled compound to unlabeled compound is about 9:91. In a particular embodiment, the radiolabeled compound is radiolabeled with ¹¹¹In and the molar ratio of radiolabeled compound to unlabeled compound is about 3:47.

In a particular embodiment, the radiolabeled compound is radiolabeled with ²²⁵Ac and the molar ratio of radiolabeled compound to unlabeled compound is in the range of about 1:400 to about 1:11000. In a particular embodiment, the radiolabeled compound is radiolabeled with ²²⁵Ac and the molar ratio of radiolabeled compound to unlabeled compound is in the range of about 1:499 to about 1:10000. In a particular embodiment, the radiolabeled compound is radiolabeled with ²²⁵Ac and the molar ratio of radiolabeled compound to unlabeled compound is about 1:499. In a particular embodiment, the radiolabeled compound is radiolabeled with ²²⁵Ac and the molar ratio of radiolabeled compound to unlabeled compound is about 1:999. In a particular embodiment, the radiolabeled compound is radiolabeled with ²²⁵Ac and the molar ratio of radiolabeled compound to unlabeled compound is about 1:9999.

The eluting through a chromatography column as recited in step (a) may entail any suitable chromatography column or technique. In some embodiments, step (a) comprises semi-preparative high performance liquid chromatography (HPLC).

The progress of the chromatographic separation of step (a) may be monitored, for example, to enable or confirm the separation from the desired radiolabeled compound of undesired materials such as quenched radionuclide and unlabeled compound, to determine the activity of the composition comprising the desired radiolabeled compound, and/or to determine the specific molar activity of the composition comprising the desired radiolabeled compound. Retention times for radiolabeled compounds may be measured directly or may be estimated based on larger-scale experiments with nonradioactive metal-labeled surrogate compounds. Accordingly, in some embodiments, the method further comprises monitoring the elution with a radiation detector. In an embodiment, the method further comprises monitoring the elution with a UV detector. In another embodiment, the unlabeled compound is detectable by the UV detector. In yet another embodiment, the radiolabeled compound in the eluate is present at a concentration below the limit of detection of the UV detector. In still another embodiment, the eluate containing the radiolabeled compound is identified by detection of radiation. In an embodiment, the radiolabeled compound exhibits a retention time within ±10% of the retention time of a nonradioactive metal-labeled surrogate compound

The stabilizing solution of the present disclosure comprises a radiolytic stabilizer and additional components which advantageously maintain the RCP and maximize the effective life (e.g., “shelf life) of the product. Accordingly, in some embodiments, the stabilizing solution comprises one or more components selected from the group consisting of: ethanol, L-methionine, selenomethionine, histidine, melatonin, a polysorbate, ammonium acetate, ascorbic acid or a pharmaceutically acceptable salt thereof, acetic acid or a pharmaceutically acceptable salt thereof, benzyl alcohol, p-aminobenzoic acid or a pharmaceutically acceptable salt thereof, cysteamine, 5-amino-2-hydroxybenzoic acid or a pharmaceutically acceptable salt thereof, nicotinic acid or a pharmaceutically acceptable salt thereof, nicotinamide, cysteine, monothioglycerol, sodium bisulfite, sodium metabisulfite, gentisic acid, and inositol.

In yet another embodiment, the stabilizing solution comprises one or more components selected from the group consisting of: L-methionine, a polysorbate, ascorbic acid or a pharmaceutically acceptable salt thereof, and acetic acid or a pharmaceutically acceptable salt thereof. In still another embodiment, the stabilizing solution comprises ascorbic acid or a pharmaceutically acceptable salt thereof, and a polysorbate.

In an embodiment, the ascorbic acid or a pharmaceutically acceptable salt thereof and the polysorbate are present in the stabilizing solution in amounts sufficient to provide an RCP of at least 90% for at least 168 hours at 2-8° C., optionally at least 95% for at least 168 hours at 2-8° C.

In an embodiment, the mass ratio of ascorbic acid or a pharmaceutically acceptable salt thereof to polysorbate in the stabilizing solution is from 50:1 to 150:1, optionally from 80:1 to 120:1, optionally about 100:1.

As shown in the Examples (e.g. Tables 14 and 18), it was surprisingly found that the presence of a polysorbate (e.g. Tween-20), even in a small amount, enables the amount of ascorbic acid or a pharmaceutically acceptable salt thereof (i.e. radiolytic stabilizer) to be reduced significantly without impacting the (high) RCP.

In an embodiment, the ascorbic acid or a pharmaceutically acceptable salt thereof is present in the stabilizing solution in an amount of ≤100 mg/mL, optionally ≤80 mg/mL, optionally from 40-80 mg/mL, optionally from 50-70 mg/mL, optionally about 64 mg/mL.

In another embodiment, the polysorbate is present in the stabilizing solution in an amount of ≤0.1% (w/v) (corresponding to ≤1 mg/mL), optionally 0.04-0.08% (w/v) (corresponding to 0.4-0.8 mg/mL), optionally 0.05-0.07% (w/v) (corresponding to 0.5-0.7 mg/mL), optionally about 0.06% (w/v) (corresponding to 0.6 mg/mL). In yet another embodiment, the polysorbate is polyoxyethylene (20) sorbitan monolaurate (Tween 20).

In an embodiment, the stabilizing solution further comprises L-methionine. In another embodiment, the stabilizing solution further comprises acetic acid or a pharmaceutically acceptable salt thereof. In yet another embodiment, the acetic acid or a pharmaceutically acceptable salt thereof is ammonium acetate.

In an embodiment, the stabilizing solution comprises L-methionine, a polyoxyethylene sorbitan monooleate, ascorbic acid or a pharmaceutically acceptable salt thereof, and acetic acid or a pharmaceutically acceptable salt thereof.

In another embodiment, the stabilizing solution comprises ammonium acetate, sodium ascorbate, L-methionine, and polyoxyethylene (20) sorbitan monolaurate.

In yet another embodiment, the pH of the stabilized composition is about 6. In still another embodiment, the pH of the pharmaceutical composition is about 6.

In an embodiment, the RCP of the stabilized composition as measured at T=0 hours (immediately after step (b)) is at least 94-97%. In an embodiment, the RCP is at least 94%. In an embodiment, the RCP is at least 95%. In an embodiment, the RCP is at least 96%. In an embodiment, the RCP is at least 97%. In an embodiment, the RCP is at least 98%. In an embodiment, the RCP is at least 99%. In any of the preceding embodiments, the RCP is measured by HPLC. Alternatively, in any of the preceding embodiments, the RCP is measured by iTLC.

In another embodiment, the RCP of the stabilized composition as measured at T=24 hours (24 hours after step (b)), optionally, at 2-8° C., is at least 94-97%. In an embodiment, the RCP is at least 94%. In an embodiment, the RCP is at least 95%. In an embodiment, the RCP is at least 96%. In an embodiment, the RCP is at least 97%. In an embodiment, the RCP is at least 98%. In an embodiment, the RCP is at least 99%. In any of the preceding embodiments, the RCP is measured by HPLC. Alternatively, in any of the preceding embodiments, the RCP is measured by iTLC.

In yet another embodiment, the RCP of the stabilized composition as measured at T=48 hours (48 hours after step (b)), optionally, at 2-8° C., is at least 90-95%. In an embodiment, the RCP is at least 90%. In an embodiment, the RCP is at least 91%. In an embodiment, the RCP is at least 92%. In an embodiment, the RCP is at least 93%. In an embodiment, the RCP is at least 94%. In an embodiment, the RCP is at least 95%. In any of the preceding embodiments, the RCP is measured by HPLC. Alternatively, in any of the preceding embodiments, the RCP is measured by iTLC.

In still another embodiment, the RCP of the stabilized composition as measured at T=72 hours (72 hours after step (b)), optionally, at 2-8° C., is at least 90-95%. In an embodiment, the RCP is at least 90%. In an embodiment, the RCP is at least 91%. In an embodiment, the RCP is at least 92%. In an embodiment, the RCP is at least 93%. In an embodiment, the RCP is at least 94%. In an embodiment, the RCP is at least 95%. In any of the preceding embodiments, the RCP is measured by HPLC. Alternatively, in any of the preceding embodiments, the RCP is measured by iTLC.

In an embodiment, the RCP of the stabilized composition as measured at T=96 hours (96 hours after step (b)), optionally, at 2-8° C., is at least 90-95%. In an embodiment, the RCP is at least 90%. In an embodiment, the RCP is at least 91%. In an embodiment, the RCP is at least 92%. In an embodiment, the RCP is at least 93%. In an embodiment, the RCP is at least 94%. In an embodiment, the RCP is at least 95%. In any of the preceding embodiments, the RCP is measured by HPLC. Alternatively, in any of the preceding embodiments, the RCP is measured by iTLC.

In another embodiment, the RCP of the stabilized composition as measured at T=120 hours (120 hours after step (b)), optionally, at 2-8° C., is at least 90-95%. In an embodiment, the RCP is at least 90%. In an embodiment, the RCP is at least 91%. In an embodiment, the RCP is at least 92%. In an embodiment, the RCP is at least 93%. In an embodiment, the RCP is at least 94%. In an embodiment, the RCP is at least 95%. In any of the preceding embodiments, the RCP is measured by HPLC. Alternatively, in any of the preceding embodiments, the RCP is measured by iTLC.

In yet another embodiment, the RCP of the stabilized composition as measured at T=144 hours (144 hours after step (b)), optionally, at 2-8° C., is at least 90-95%. In an embodiment, the RCP is at least 90%. In an embodiment, the RCP is at least 91%. In an embodiment, the RCP is at least 92%. In an embodiment, the RCP is at least 93%. In an embodiment, the RCP is at least 94%. In an embodiment, the RCP is at least 95%. In any of the preceding embodiments, the RCP is measured by HPLC. Alternatively, in any of the preceding embodiments, the RCP is measured by iTLC.

In still another embodiment, the RCP of the stabilized composition as measured at T=168 (168 hours after step (b)), optionally, at 2-8° C., is at least 90-95%. In an embodiment, the RCP is at least 90%. In an embodiment, the RCP is at least 91%. In an embodiment, the RCP is at least 92%. In an embodiment, the RCP is at least 93%. In an embodiment, the RCP is at least 94%. In an embodiment, the RCP is at least 95%. In any of the preceding embodiments, the RCP is measured by HPLC. Alternatively, in any of the preceding embodiments, the RCP is measured by iTLC.

In an embodiment, the RCP of the stabilized composition as measured at T=24 hours is at least 90%. In another embodiment, the RCP of the stabilized composition as measured at T=48 hours, optionally, at 2-8° C., is at least 90%. In yet another embodiment, the RCP of the stabilized composition as measured at T=72 hours, optionally, at 2-8° C., is at least 90%. In still another embodiment, the RCP of the stabilized composition as measured at T=96 hours, optionally, at 2-8° C., is at least 90%.

In an embodiment, the RCP of the stabilized composition as measured at T=120 hours, optionally, at 2-8° C., is at least 90%. In another embodiment, the RCP of the stabilized composition as measured at T=144 hours, optionally, at 2-8° C., is at least 90%. In yet another embodiment, the RCP of the stabilized composition as measured at T=168 hours, optionally, at 2-8° C., is at least 90%.

The radiolabeled compound comprises an organic molecule that is bound or coordinated to a radionuclide. The organic molecule may be a small molecule (e.g., ≤1000 Da), or a larger molecule. Accordingly, in some embodiments, the radiolabeled compound is a protein. In some embodiments, the radiolabeled compound is a radiolabeled peptide. In an embodiment, the radiolabeled peptide is a radiolabeled cyclic peptide. The cyclic peptide may comprise a disulfide bond. The cyclic peptide may comprise a thioacetal moiety. The cyclic peptide may comprise an olefin (i.e., alkene) moiety. The cyclic peptide may comprise a triazole moiety. In another embodiment, the radiolabeled compound has a molecular weight in the range of 1-10 KDa. The radiolabeled compound may have a molecular weight in the range of 1-5 KDa. The radiolabeled compound may have a molecular weight in the range of 2-3 KDa, The radiolabeled compound may have a molecular weight in the range of 3-5 KDa.

In some embodiments, the radiolabeled compound comprises: (i) a peptide comprising a chelator, and (ii) a radioisotope. In an embodiment, the radioisotope is chelated by the chelator. In an embodiment, the chelator is a non-cyclic chelator. In another embodiment, the chelator is a cyclic chelator. In an embodiment, the chelator is a bifunctional chelator. In an embodiment, the chelator is selected from DOTA, C-DOTA, PA-DOTA, DODASA, C4amino-DOTA, DTPA, DOTAGA, NOTA, NODASA, and NODAGA. In another embodiment, the chelator is DOTA.

In yet another embodiment, the radioisotope is selected from ¹¹¹In, ^{99m}Tc, ^{94m}Tc, ⁶⁶Ga, ⁶⁷Ga, ⁶⁸Ga, ¹³⁴Ce, ⁵²Fe, ¹⁶⁹Er, ⁷²As, ⁹⁷Ru, ²⁰³Pb, ⁶¹Cu, ⁶²Cu, ⁶⁴Cu, ⁶⁷Cu, ⁸⁹Sr, ¹⁸⁶Re, ¹⁸⁸Re, ⁸⁶Y, ⁹⁰Y, ⁸⁹Zr, ⁵¹Cr, ⁵²Mn, ⁵¹Mn, ¹⁷⁷Lu, ¹⁶⁹Yb, ¹⁷⁵Yb, ¹⁰⁵Rh, ¹⁶⁶Dy, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹²Bi, ²¹³Bi, ¹⁴²Pr, ¹⁴³Pr, ¹⁹⁸Au, ¹⁹⁹Au, ¹⁸F, ¹⁴⁹Tb, ¹⁵²Tb, ¹⁵⁵Tb, ¹⁶¹Tb, ⁴³Sc, ⁴⁴Sc, ⁴⁷Sc, ²¹²Pb, ²¹¹At, ²²³Ra, ²²⁷Th, ²²⁶Th, ⁸²Rb, ³²P, ⁷⁶As, ⁸⁹Zr, ¹¹¹Ag, ¹⁶⁵Er, ²²⁵Ac, and ²²⁷Ac.

In a particular embodiment, the radioisotope is ¹⁷⁷Lu. In a particular embodiment, the radioisotope is ¹¹¹In. In a particular embodiment, the radioisotope is ²²⁵Ac.

In an embodiment, the radiolabeled compound is a cyclic peptide comprising DOTA and ¹⁷⁷Lu. In an embodiment, the radiolabeled compound is a cyclic peptide comprising DOTA and ¹In. In an embodiment, the radiolabeled compound is a cyclic peptide comprising DOTA and ²²⁵Ac.

High specific activity (HSA) compositions of radiolabeled compounds are desirable for treatment of diseases (e.g., cancer) characterized by low-expressing targets (e.g., DLL3). Such compositions as described herein are also advantageous in that they are purified to reduce or remove undesired byproducts and/or unreacted radioisotope and compound substances. However, HSA radiolabeled compounds undergo radioactive decay faster than radiolabeled compounds with low specific activity (LSA). Thus, stabilized radiolabeled compounds and the corresponding methods used to make them are critical for effective treatment.

As used herein, “specific molar activity” or “specific activity” refers to the measured radioactivity of a composition comprising a radiolabeled compound per moles of the compound (i.e., total moles of the radiolabeled compound and moles of unlabeled compound, if present). A composition comprising only radiolabeled compound will be characterized as having the theoretical maximum specific molar activity. A composition comprising both radiolabeled and unlabeled compound will be characterized has having a specific molar activity that is less than the theoretical maximum, and the specific molar activity decreases as the ratio of unlabeled compound to radiolabeled compound increases. Compositions that are enriched in radiolabeled compound (e.g., using the methods disclosed herein) are referred to as “high specific activity.” Non-enriched compositions are referred to as “regular specific activity” or “low specific activity.”

For purposes of determining specific molar activity, radioactivity of the composition may be measured with a suitable radiodetector as described in the Examples herein. Moles (e.g., represented as millimoles or micromoles) of the compound may be calculated by known methods from measurements such as gravimetric or spectroscopic data. For example, spectroscopic data from a UV absorbance detector may be used to quantify the amount of a compound present in a chromatography eluant. From such measurements, the specific molar activity of the compound is calculated. In the case that the radioactivity of a compound may be quantified but the molar amount of the compound cannot be quantified (e.g., by data from a UV absorbance detector) because the compound is present in a quantity or at a concentration that is below the limit of quantification of the detector, the specific molar activity of the composition may be reported as a “greater than” value (e.g., >60 μCi/nmol), such value being the quotient of the measured radioactivity divided by the limit of quantification of the device (e.g., UV absorbance detector). The upper bound of such a “greater than” value will be the theoretical maximum specific molar activity. The theoretical maximum specific molar activity of a radiolabeled compound is a function of the activity of the radiolabel and the stoichiometric ratio of the radiolabel to the compound. For example, a mono-radiolabeled compound has a stoichiometric ratio of 1:1.

By way of example, the theoretical maximum specific molar activity of a mono-²²⁵Ac-labeled compound may be calculated as follows:
²²⁵Ac half-life=9.92 day=14284.8 min
Probability of 1 atom to decay every minute=Ln(2)/14284.8=0.693/14284.8=4.85E-5
Atom number in 1 nmol=6.022E23/E9=6.022E14
Decay number of 1 nmol every minute=6.022E14*4.85E-5=2.92E10 DPM (decay per minute)
1 mCi=2.22E9 DPM (unit conversion)
Theoretical specific activity=2.92E10 DPM/nmol=2.92E10/2.22E9 mCi/nmol=13.2 mCi/nmol.

As a further example, the theoretical maximum specific molar activity of a mono-²²⁵Ac-labeled compound may be calculated as follows:
¹⁷⁷Lu half-life=6.7 day=9648 min
Probability of 1 atom to decay every minute=Ln(2)/9648=0.693/9648=7.18E-5
Atom number in 1 nmol=6.022E23/E9=6.022E14
Decay number of 1 nmol every minute=6.022E14*7.18E-5=4.33E10 DPM (decay per minute)
1 mCi=2.22E9 DPM (unit conversion)
Theoretical specific activity=4.33E10 DPM/nmol=4.33E10/2.22E9 mCi/nmol=19.5 mCi/nmol.

Accordingly, in some embodiments, the stabilized composition has a specific molar activity in the range of 60-19500 μCi per nmol compound. In some embodiments, the stabilized composition has a specific molar activity in the range of 60-19000 μCi per nmol compound.

In some embodiments, the stabilized composition comprises a ¹⁷⁷Lu-labeled compound and has a specific molar activity in the range of 5-19.5 mCi per nmol compound. In some embodiments, the stabilized composition has a specific molar activity in the range of 15-19 mCi per nmol compound.

In some embodiments, the stabilized composition comprises a ²²⁵Ac-labeled compound and has a specific molar activity in the range of 60-13200 μCi per nmol compound. In an embodiment, the stabilized composition has a specific molar activity in the range of 60-13000 μCi per nmol compound.

The desired total activity of a pharmaceutical composition as disclosed herein may be determined based on factors including, for example, the energy emitted by the radioisotope, and the prevalence of the biological target of the radiolabeled compound. Accordingly, in some embodiments, the radioisotope is ¹⁷⁷Lu and the total activity of the pharmaceutical composition is in the range of 10-1000 mCi, for example, 10-100 mCi, 100-200 mCi, 200-300 mCi, 300-400 mCi, 400-500 mCi, 500-600 mCi, 600-700 mCi, 700-800 mCi, 800-900 mCi, or 900-1000 mCi.

In some embodiments, the radioisotope is ²²⁵Ac and the total activity of the pharmaceutical composition is in the range of 0.01-5 mCi, for example, 0.1-5 mCi, 1-5 mCi, 2-5 mCi, 3-5 mCi, 4-5 mCi, 0.01-4 mCi. 0.1-4 mCi, 1-4 mCi, 2-4 mCi, 3-4 mCi, 0.01-3 mCi. 0.1-3 mCi, 1-3 mCi, 2-3 mCi, 0.01-2 mCi. 0.1-2 mCi, 1-2 mCi, 2-3, 0.01-1 mCi, or 0.1-1 mCi.

Pharmaceutical compositions descried herein may be formulated to any appropriate volume. Accordingly, in some embodiments, the volume of the pharmaceutical composition is in the range of 1-100 mL. In an embodiment, the volume of the pharmaceutical composition is in the range of 1-10 mL. In another embodiment, the volume of the pharmaceutical composition is in the range of 11-20 mL. In yet another embodiment, the volume of the pharmaceutical composition is about 15 mL. In an embodiment, the volume of the pharmaceutical composition is in the range of 21-30 mL. In still another embodiment, the volume of the pharmaceutical composition is in the range of 31-40 mL. In an embodiment, the volume of the pharmaceutical composition is in the range of 41-50 mL. In another embodiment, the volume of the pharmaceutical composition is about 41 mL.

In yet another embodiment, the volume of the pharmaceutical composition is in the range of 51-60 mL. In still another embodiment, the volume of the pharmaceutical composition is in the range of 61-70 mL. In an embodiment, the volume of the pharmaceutical composition is in the range of 71-80 mL. In another embodiment, the volume of the pharmaceutical composition is in the range of 81-90 mL. In yet another embodiment, the volume of the pharmaceutical composition is in the range of 91-100 mL.

The mixture comprising the radiolabeled compound and unlabeled compound as recited in step (a) is representative of the product of a radiolabeling reaction in which a stoichiometric excess of compound is used in relation to radioisotope. Accordingly, in some embodiments, the mixture comprising the radiolabeled compound and unlabeled compound is the product of radiolabeling a peptide with a radioisotope. In another embodiment, the mixture comprising the radiolabeled compound and unlabeled compound is prepared by combining the radioisotope and a reagent solution comprising the peptide. In yet another embodiment, the method further comprises heating the radioisotope and the reagent solution comprising the peptide. In still another embodiment, the heating is at about 90° C.

In an embodiment, the amount of peptide used is in the range of 1-1500 nmol. In other embodiments, the amount of peptide used is in the range of 1-1000 nmol, 1-500 nmol, 1-200 nmol, or 1-100 nmol. In other embodiments, the amount of peptide used is in the range of 100-1400 nmol, 100-1200 nmol, or 100-1000 nmol. In some embodiments, the amount of peptide used is in the range of 2-80 nmol. In some embodiments, the amount of peptide used is in the range of 4-150 nmol. In some embodiments, the amount of peptide used is in the range of 120-1200 nmol. In another embodiment, the reagent solution comprises the peptide and metal free water.

In yet another embodiment, the activity of radioisotope prior to combining with the reagent solution comprising the peptide is in the range of 0.01-500 mCi. In an embodiment, the radioisotope is ¹⁷⁷Lu and the activity is in the range of 1-300 mCi. In an embodiment, the radioisotope is ¹¹¹In and the activity is in the range of 1-100 mCi. In an embodiment, the radioisotope is ²²⁵Ac and the activity is in the range of 0.01-5 mCi, 0.1-5 mCi, 0.1-2.5 mCi, or 1-2.5 mCi.

In still another embodiment, the specific molar activity of the combined peptide and radioisotope, prior to combining the radioisotope with the reagent solution comprising the peptide, is in the range of 0.01-30 μCi/nmol. In an embodiment, the specific molar activity is in the range of 0.1-20 μCi/nmol. In an embodiment, the radioisotope is ¹⁷⁷Lu and the specific molar activity is in the range of 0.1-2 μCi/nmol. In an embodiment, the radioisotope is ¹¹¹In and the specific molar activity is in the range of 0.1-4 μCi/nmol. In an embodiment, the radioisotope is ²²⁵Ac and the specific molar activity is in the range of 1-20 μCi/nmol.

In an embodiment, the reagent solution further comprises a reaction buffer. In another embodiment, the reaction buffer comprises a stabilizer. In yet another embodiment, the stabilizer comprises ascorbic acid or a pharmaceutically acceptable salt thereof. In still another embodiment, the reaction buffer has a pH in the range of 5-6. In an embodiment, the reaction buffer has a pH of about 5.5. In an embodiment, the reaction buffer has a pH of about 6.

In another embodiment, the reagent solution further comprises ethanol. In yet another embodiment, the reagent solution comprises a reaction buffer, ethanol, the peptide and metal free water. In still another embodiment, the reagent solution does not comprise a polysorbate.

In an embodiment, the method further comprises quenching the mixture comprising the radiolabeled compound and unlabeled compound with a quenching agent. In another embodiment, the quenching agent comprises a chelator and a buffer. In yet another embodiment, the chelator in the quenching agent is DTPA.

In still another embodiment, the buffer is ammonium acetate. In an embodiment, the buffer has a pH of about 5 to about 6, e.g., a pH of about 5.5, or a pH of about 6.

Provided herein are further methods of making enriched and stable radioligand therapy formulations. The methods disclosed herein allow isolation of formulations enriched in the desired radiolabeled peptide product, despite the similarity in structure and properties of the desired radiolabeled peptide and the undesired non-radiolabeled peptide starting material.

In an aspect, provided herein is a method of preparing a composition enriched in a radiolabeled peptide, wherein the method comprises:
(a) combining a peptide that is linked to a chelator, a stabilizer, a buffer, and a radionuclide to form a reaction mixture, wherein a radiolabeled peptide is formed;
(b) optionally adding a quencher to the reaction mixture;
(c) separating the radiolabeled peptide from non-radiolabeled peptide by chromatography; and
(d) isolating the composition enriched in the radiolabeled peptide.

In an embodiment, the method further comprises the steps of:
(e) passing the composition enriched in the radiolabeled peptide through a sterile filter; and
(f) assessing the RCP of the composition enriched in the radiolabeled peptide.

In another embodiment, the peptide is in molar excess of the radionuclide.

In another aspect, provided herein is a method of preparing a composition enriched in a radiolabeled peptide, wherein the method comprises:
(a) separating a mixture of radiolabeled peptide and non-radiolabeled peptide by chromatography; and
(b) isolating the composition enriched in the radiolabeled peptide.

In an embodiment, the method further comprises the steps of:
(c) passing the composition enriched in the radiolabeled peptide through a sterile filter;
(d) assessing the RCP of the composition enriched in the radiolabeled peptide.

In another embodiment, the method further comprises the step of:
dispensing the composition enriched in the radiolabeled peptide into a unit-dose or multi-dose container.

In an aspect, provided herein is a method of preparing an enriched radiolabeled peptide composition that is stable for at least about 24 hours as indicated by an RCP of greater than 90%, wherein the method comprises:
(a) combining a peptide that is linked to a chelator, a stabilizer, a buffer, and a radionuclide to form a reaction mixture, wherein a radiolabeled peptide is formed;
(b) optionally adding a quencher to the reaction mixture;
(c) separating the radiolabeled peptide from non-radiolabeled peptide by chromatography;
(d) isolating the radiolabeled peptide composition; and
(e) assessing the RCP of the radiolabeled peptide composition.

An additional discovery of the disclosed methods is that the addition of a surfactant to the radiolabeled peptide formulation improves the stability of the radiolabeled peptide over an extended period of time after isolation as measured by RCP. This result may be due to the tendency of RLTs to adhere to container surfaces, which could contribute to degradation of the product, affecting product shelf-life stability.

In an aspect, provided herein is a method of preparing a radiolabeled peptide composition that is stable for at least about 24 hours as indicated by an RCP of greater than 90%, wherein the method comprises:
(a) combining a peptide that is linked to a chelator, a stabilizer, a buffer, and a radionuclide to form a reaction mixture, wherein a radiolabeled peptide is formed;
(b) optionally adding a quencher to the reaction mixture;
(c) adding a surfactant to the reaction mixture; and
(d) isolating the composition enriched in the radiolabeled peptide.

In an embodiment, the method further comprises the steps of:
(e) passing the composition enriched in the radiolabeled peptide through a sterile filter; and
(f) assessing the RCP (RCP) of the composition enriched in the radiolabeled peptide.

In another embodiment, the peptide is in molar excess of the radionuclide.

In another embodiment, the method further comprises the step of:
dispensing the composition enriched in the radiolabeled peptide into a unit-dose or multi-dose container.

In an aspect, provided herein is a method of preparing a radiolabeled peptide composition that is stable for at least about 24 hours as indicated by an RCP of greater than 90%, wherein the method comprises:
(a) combining a peptide that is linked to a chelator, a stabilizer, a buffer, and a radionuclide to form a reaction mixture, wherein a radiolabeled peptide is formed;
(b) optionally adding a quencher to the reaction mixture;
(c) separating the radiolabeled peptide by chromatography;
(d) isolating the radiolabeled peptide composition;
(e) adding a surfactant to the radiolabeled peptide composition; and
(f) assessing the RCP of the radiolabeled peptide composition.

In an aspect, provided herein is a method of preparing a composition enriched in a radiolabeled peptide, wherein the method comprises:
(a) combining a peptide that is linked to a chelator, a stabilizer, a buffer, and a radionuclide to form a reaction mixture, wherein a radiolabeled peptide is formed;
(b) optionally adding a quencher to the reaction mixture;
(c) separating the radiolabeled peptide from non-radiolabeled peptide by chromatography; and
(d) isolating the composition enriched in the radiolabeled peptide.

In an embodiment, the method further comprises the steps of:
(e) optionally adding a surfactant to the composition enriched in the radiolabeled peptide;
(f) passing the composition enriched in the radiolabeled peptide through a sterile filter; and
(g) assessing the RCP of the composition enriched in the radiolabeled peptide.

In another embodiment, the peptide is in molar excess of the radionuclide.

In another aspect, provided herein is a method of preparing a composition enriched in a radiolabeled peptide, wherein the method comprises:
(a) separating a mixture of radiolabeled peptide and non-radiolabeled peptide by chromatography; and
(b) isolating the composition enriched in the radiolabeled peptide.

In an embodiment, the method further comprises the steps of:
(c) optionally adding a surfactant to the composition enriched in the radiolabeled peptide;
(d) passing the composition enriched in the radiolabeled peptide through a sterile filter;
(e) assessing the RCP of the composition enriched in the radiolabeled peptide.

In another embodiment, the method further comprises the step of:
dispensing the composition enriched in the radiolabeled peptide into a unit-dose or multi-dose container.

In an aspect, provided herein is a method of preparing a radiolabeled peptide composition that is stable for at least about 24 hours as indicated by an RCP of greater than 90%, wherein the method comprises:
(a) combining a peptide that is linked to a chelator, a stabilizer, a buffer, and a radionuclide to form a reaction mixture, wherein a radiolabeled peptide is formed;
(b) optionally adding a quencher to the reaction mixture;
(c) separating the radiolabeled peptide from non-radiolabeled peptide by chromatography;
(d) isolating the radiolabeled peptide composition;
(e) adding a surfactant to the radiolabeled peptide composition; and
(f) assessing the RCP of the radiolabeled peptide composition.

In still another embodiment, the stabilizer is one or more of gentisic acid, ascorbic acid, acetic acid, methionine, selenomethionine, histidine, melatonin, ethanol, and or a pharmaceutically acceptable salt thereof.

In an embodiment, the stabilizer is one or more of gentisic acid, ascorbic acid, sodium ascorbate, methionine, ethanol, and ammonium acetate.

In another embodiment, the stabilizer is a combination of:
(i) sodium ascorbate;
(ii) gentisic acid;
(iii) methionine; and
(iv) ethanol.

In another embodiment, the stabilizer is:
(i) 10-700 mg/mL sodium ascorbate;
(ii) 0-100 mg/mL gentisic acid;
(iii) 0-100 mg/mL methionine; and
(iv) 5-45% v/v ethanol, each expressed as a concentration or a volume percentage of the composition.

In yet another embodiment, the stabilizer is:
(i) 25-600 mg/mL sodium ascorbate;
(ii) 0-50 mg/mL gentisic acid;
(iii) 0-30 mg/mL methionine; and
(iv) 5-15% v/v ethanol, each expressed as a concentration or a volume percentage of the composition.

In another embodiment, the stabilizer is a combination of:
(i) sodium ascorbate;
(ii) methionine; and
(iii) ethanol.

In still another embodiment, the stabilizer is:
(i) 200-300 mg/mL sodium ascorbate;
(ii) 22-28 mg/mL methionine; and
(iii) 8-13% v/v ethanol, each expressed as a concentration or a volume percentage of the composition.

In an embodiment, the stabilizer is a combination of:
(i) sodium ascorbate;
(ii) gentisic acid; and
(iii) ethanol.

In an embodiment, the stabilizer is:
(i) 200-550 mg/mL sodium ascorbate;
(ii) 5-45 mg/mL gentisic acid; and
(iii) 8-13% v/v ethanol, each expressed as a concentration or a volume percentage of the composition.

In another embodiment, the buffer is selected from ammonium acetate, sodium acetate, acetic acid, sodium chloride, sodium phosphate, ascorbic acid, sodium ascorbate, or a combination thereof.

In yet another embodiment, the radionuclide is selected from the group consisting of ¹⁷⁷Lu, ⁶⁸Ga, ¹⁸F, ^{99m}Tc, ²¹¹At, ¹⁶⁶Ho, ²²⁵Ac, ¹¹¹In, ¹²³I, ¹³¹I, ⁸⁹Zr, ⁹⁰Y, ²¹²Pb, ¹⁶¹Tb, ¹⁸⁸Re, ⁶⁴Cu, and ⁶⁷Cu.

In still another embodiment, the radiolabeled peptide is radiolabeled with a radionuclide selected from the group consisting of ¹⁷⁷Lu, ⁶⁸Ga, ¹⁸F, ^{99m}Tc, ²¹¹At, ¹⁶⁶Ho, ²²⁵Ac, ¹¹¹In, ¹²³I, ¹³¹I, ⁸⁹Zr, ⁹⁰Y, ²¹²Pb, ¹⁶¹Tb, ¹⁸⁸Re, ⁶⁴Cu, and ⁶⁷Cu.

In an embodiment, the quencher is diethylenetriaminepentaacetic acid.

In another embodiment, the surfactant is selected from the group consisting of polyoxyethylene (20) sorbitan monolaurate, polyoxyethylene (20) sorbitan monopalmitate, polyoxyethylene (20) sorbitan monostearate, polyoxyethylene (20) sorbitan monooleate, monooleate polyoxyethylene (20) cetyl ether, and 2,3-dihydroxypropyl octadecanoate.

In still another embodiment, the surfactant is selected from the group consisting of polyoxyethylene (20) sorbitan monolaurate, polyoxyethylene (20) sorbitan monopalmitate, polyoxyethylene (20) sorbitan monostearate, and polyoxyethylene (20) sorbitan monooleate.

In an embodiment, the composition is isolated with high-performance liquid chromatography (HPLC).

In another embodiment, the composition is isolated with ultra-high performance liquid chromatography (UHPLC).

In yet another embodiment, the composition is isolated with an isocratic HPLC mobile phase.

In still another embodiment, the composition is isolated with a gradient HPLC mobile phase.

In an embodiment, the mobile phase is selected from ammonium acetate and ethanol, or a combination thereof.

In another embodiment, the RCP of the composition is at least about 90%. In yet another embodiment, the RCP of the composition is at least about 92%. In still another embodiment, the RCP of the composition is at least about 94%. In an embodiment, the RCP of the composition is at least about 96%. In another embodiment, the RCP of the composition is at least about 98%.

In yet another embodiment, prior to assessing the RCP, the composition is stored at −22° C. to 27° C. In still another embodiment, the composition is stable at −22° C. to 27° C. for at least about 24 hours as indicated by an RCP of greater than 90%. In an embodiment, the composition is stable at −22° C. to 27° C. for at least about 48 hours as indicated by an RCP of greater than 90%. In another embodiment, the composition is stable at −22° C. to 27° C. for at least about 72 hours as indicated by an RCP of greater than 90%. In yet another embodiment, the composition is stable at −22° C. to 27° C. for at least about 96 hours as indicated by an RCP of greater than 90%. In still another embodiment, the composition is stable at −22° C. to 27° C. for at least about 120 hours as indicated by an RCP of greater than 90%. In an embodiment, the composition is stable at −22° C. to 27° C. for at least about 220 hours as indicated by an RCP of greater than 90%.

In another embodiment, the composition is stable at −22° C. to −18° C. In yet another embodiment, the composition is stable at 2° C. to 8° C. In still another embodiment, the composition is stable at 23° C. to 27° C.

In an embodiment, provided herein is the composition formed by the any of the methods disclosed herein.

### Pharmaceutical Compositions Comprising Stabilized Compositions of Radiolabeled Compounds

Provided herein are stabilized compositions of radiolabeled compounds. The pharmaceutical compositions and formulations disclosed herein are enriched in the desired radiolabeled peptide product, despite the similarity in structure and properties of the desired radiolabeled peptide and the undesired non-radiolabeled peptide starting material. The stabilized compositions disclosed herein also have a high specific molar activity (HSA), thus they are useful in both the diagnostic imaging of and the treatment of diseases (e.g., cancer) characterized by low-expressing targets (e.g., DLL3).

The enriched and stable radioligand formulations of the present disclosure can also be used in theranostic methods. “Theranostics,” a term derived from a combination of therapeutics and diagnostics, is an emerging field of medicine where specific disease-targeting agents, e.g., radiopharmaceuticals, may be used to simultaneously or sequentially diagnose and treat medical conditions. Theranostics has become an important field of research and development in medical physics, where varying the isotope of the radionuclide present in a given disease-targeting agent, e.g., a radioligand therapy, can change the disease-targeting agent from an imaging probe (by, e.g., using β+ or γ emitting isotopes to facilitate positron emission tomography (PET) or single photon emission computed tomography (CT) imaging, respectively), to a therapy probe (by, e.g., using α or β-particle or Auger electron emitting isotopes to facilitate targeted radiotherapy).

Thus, in an aspect, provided herein is a pharmaceutical composition comprising a radiolabeled compound and a stabilizing solution.

In an embodiment, the pharmaceutical composition (which comprises the stabilizing solution) comprises one or more components selected from the group consisting of: ethanol, L-methionine, selenomethionine, histidine, melatonin, a polysorbate, ammonium acetate, ascorbic acid or a pharmaceutically acceptable salt thereof, acetic acid or a pharmaceutically acceptable salt thereof, benzyl alcohol, p-aminobenzoic acid or a pharmaceutically acceptable salt thereof, cysteamine, 5-amino-2-hydroxybenzoic acid or a pharmaceutically acceptable salt thereof, nicotinic acid or a pharmaceutically acceptable salt thereof, nicotinamide, cysteine, monothioglycerol, sodium bisulfite, sodium metabisulfite, gentisic acid, and inositol.

In another embodiment, the pharmaceutical composition comprises one or more components selected from the group consisting of: ethanol, L-methionine, a polysorbate, ascorbic acid or a pharmaceutically acceptable salt thereof, and acetic acid or a pharmaceutically acceptable salt thereof.

In yet another embodiment, the pharmaceutical composition comprises ascorbic acid or a pharmaceutically acceptable salt thereof, and a polysorbate. In an embodiment, the ascorbic acid or a pharmaceutically acceptable salt thereof and the polysorbate are present in the pharmaceutical composition amounts sufficient to provide an RCP of at least 90% for at least 168 hours at 2-8° C., optionally at least 95% for at least 168 hours at 2-8° C. In an embodiment, the mass ratio of ascorbic acid or a pharmaceutically acceptable salt thereof to polysorbate in the pharmaceutical composition is from 50:1 to 150:1, optionally from 80:1 to 120:1, optionally about 100:1. As shown in the Examples (e.g. Tables 14 and 18), it has surprisingly been found that the presence of a polysorbate (e.g. Tween 20), even in a small amount, enables the amount of ascorbic acid or a pharmaceutically acceptable salt thereof (i.e. radiolytic stabilizer) to be reduced significantly without impacting the (high) RCP. In still another embodiment, the ascorbic acid or a pharmaceutically acceptable salt thereof is present in an amount of ≤100 mg/mL, optionally ≤80 mg/mL, optionally from 30-70 mg/mL, optionally from 40-60 mg/mL, optionally about 50 mg/mL. In an embodiment, the polysorbate is present in the pharmaceutical composition in an amount of ≤0.1% (w/v) (corresponding to ≤1 mg/mL), optionally 0.03-0.07% (w/v) (corresponding to 0.3-0.7 mg/mL), optionally 0.04-0.06% (w/v) (corresponding to 0.4-0.6 mg/mL), optionally about 0.05% (w/v) (corresponding to 0.5 mg/mL). In another embodiment, the polysorbate is polyoxyethylene (20) sorbitan monolaurate (Tween 20), optionally wherein the polyoxyethylene (20) sorbitan monolaurate is present in the pharmaceutical composition in an amount of about 0.1 mg/mL to about 1 mg/mL.

In yet another embodiment, the pharmaceutical composition further comprises L-methionine, optionally wherein the L-methionine is present in the pharmaceutical composition at a concentration of about 10 mg/mL to about 30 mg/mL.

In still another embodiment, the pharmaceutical composition further comprises acetic acid or a pharmaceutically acceptable salt thereof, optionally wherein the acetic acid or pharmaceutically acceptable salt thereof is present in the pharmaceutical composition at a concentration of about 0.01 M to about 0.15 M. In an embodiment, the acetic acid or a pharmaceutically acceptable salt thereof is ammonium acetate. In another embodiment, the pharmaceutical composition further comprises ethanol, optionally wherein the ethanol is present in the pharmaceutical composition at about 1% v/v to about 15% v/v, optionally wherein the ethanol is present at about 5% v/v to about 10% v/v.

In yet another embodiment, the pharmaceutical composition comprises ethanol, L-methionine, a polyoxyethylene sorbitan monooleate, ascorbic acid or a pharmaceutically acceptable salt thereof, and acetic acid or a pharmaceutically acceptable salt thereof. In still another embodiment, the pharmaceutical composition comprises ethanol, ammonium acetate, sodium ascorbate, L-methionine, and polyoxyethylene (20) sorbitan monolaurate.

In an embodiment, the pH of the stabilized solution is about 6 and/or the pH of the pharmaceutical composition is about 6.

In another embodiment, the radiolabeled compound comprises a radioisotope selected from ¹¹¹In, ^{99m}Tc, ^{94m}Tc, ⁶⁶Ga, ⁶⁷Ga, ⁶⁸Ga, ¹³⁴Ce, ⁵²Fe, ¹⁶⁹Er, ⁷²As, ⁹⁷Ru, ²⁰³Pb, ⁶¹Cu, ⁶²Cu, ⁶⁴Cu, ⁶⁷Cu, ⁸⁹Sr, ¹⁸⁶Re, ¹⁸⁸Re, ⁸⁶Y, ⁹⁰Y, ⁸⁹Zr, ⁵¹Cr, ⁵²Mn, ⁵¹Mn, ¹⁷⁷Lu, ¹⁶⁹Yb, ¹⁷⁵Yb, ¹⁰⁵Rh, ¹⁶⁶Dy, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹²Bi, ²¹³Bi, ¹⁴²Pr, ¹⁴³Pr, ¹⁹⁸Au, ¹⁹⁹Au, ¹⁸F (e.g., [¹⁸F]AlF), ¹⁴⁹Tb, ¹⁵²Tb, ¹⁵⁵Tb, ¹⁶¹Tb, ⁴³Sc, ⁴⁴Sc, ⁴⁷Sc, ²¹²Pb, ²¹¹At, ²²³Ra, ²²⁷Th, ²²⁶Th, ⁸²Rb, ³²P, ⁷⁶As, ⁸⁹Zr, ¹¹¹Ag, ¹⁶⁵Er, ²²⁵Ac, and ²²⁷Ac. In still another embodiment, the radiolabeled compound comprises ¹¹¹In (i.e., the radioisotope is ¹¹¹In). In an embodiment, the radiolabeled compound comprises ¹⁷⁷Lu (i.e., the radioisotope is ¹⁷⁷Lu). In another embodiment, the radiolabeled compound comprises ²²⁵Ac (i.e., the radioisotope is ²²⁵Ac).

In yet another embodiment, the radiolabeled compound is a small molecule, a protein, or a peptide. In still another embodiment, the radiolabeled compound comprises: (i) a peptide comprising a chelator, and (ii) a radioisotope. In an embodiment, the radioisotope is chelated by the chelator. In another embodiment, the peptide is cyclic peptide. In yet another embodiment, the cyclic peptide comprises a disulfide bond. In yet another embodiment, the cyclic peptide does not comprise a disulfide bond. In still another embodiment, the peptide comprises one or more non-natural amino acid residues. In an embodiment, the peptide has a molecular weight in the range of 1-10 KDa.

In some embodiments, the radiolabeled compound has an affinity for a cell surface protein. In another embodiment, the affinity in units K_{D} of ≤1.0 nM. In other embodiments, the affinity is characterized as: 1.0 nM<K_{D}≤10 nM, 10 nM<K_{D}≤100 nM, or 100 nM<K_{D}≤300 nM.

In some embodiments, the cell surface protein is characterized by an expression of about 1000-100,000 copies per cell. In some embodiments, the cell surface protein is characterized by an expression of about 1000-5000 copies per cell. In some embodiments, the cell surface protein is characterized by an expression of about 3000-5000 copies per cell. In some embodiments, the cell surface protein is characterized by an expression of about 5000-10,000 copies per cell. In some embodiments, the cell surface protein is characterized by an expression of about 10,000-30,000 copies per cell. In some embodiments, the cell surface protein is characterized by an expression of about 20,000-30,000 copies per cell. In some embodiments, the cell surface receptor is DLL3. In some embodiments, the cell surface receptor is B7-H3. In still another embodiment, the cell is a cancerous cell.

In an embodiment, the pharmaceutical composition has a total activity in the range of 0.01-1000 mCi. In some embodiments, the radioisotope is ¹⁷⁷Lu and the total activity of the pharmaceutical composition is in the range of 10-1000 mCi, for example, 10-100 mCi, 100-200 mCi, 200-300 mCi, 300-400 mCi, 400-500 mCi, 500-600 mCi, 600-700 mCi, 700-800 mCi, 800-900 mCi, or 900-1000 mCi. In some embodiments, the radioisotope is ²²⁵Ac and the total activity of the pharmaceutical composition is in the range of 0.01-5 mCi, for example, 0.1-5 mCi, 1-5 mCi, 2-5 mCi, 3-5 mCi, 4-5 mCi, 0.01-4 mCi. 0.1-4 mCi, 1-4 mCi, 2-4 mCi, 3-4 mCi, 0.01-3 mCi. 0.1-3 mCi, 1-3 mCi, 2-3 mCi, 0.01-2 mCi. 0.1-2 mCi, 1-2 mCi, 2-3, 0.01-1 mCi, or 0.1-1 mCi.

In another embodiment, the pharmaceutical composition has a volume of about 1-100 mL. In an embodiment, the pharmaceutical composition has a volume of about 1-10 mL. In another embodiment, the pharmaceutical composition has a volume of about 11-20 mL. In yet another embodiment, the pharmaceutical composition has a volume of about 15 mL. In an embodiment, the pharmaceutical composition has a volume of about 21-30 mL. In still another embodiment, the pharmaceutical composition has a volume of about 31-40 mL. In an embodiment, the pharmaceutical composition has a volume of about 41-50 mL. In another embodiment, the pharmaceutical composition has a volume of about 41 mL.

In yet another embodiment, the pharmaceutical composition has a volume of about 51-60 mL. In still another embodiment, the pharmaceutical composition has a volume of about 61-70 mL. In an embodiment, the pharmaceutical composition has a volume of about 71-80 mL. In another embodiment, the pharmaceutical composition has a volume of about 81-90 mL. In yet another embodiment, the pharmaceutical composition has a volume of about 91-100 mL.

In still another embodiment, the pharmaceutical composition comprises sodium ascorbate in a concentration of about 40 mg/mL to about 60 mg/mL, L-methionine in a concentration of about 10 mg/mL to about 30 mg/mL, polyoxyethylene (20) sorbitan monolaurate in an amount of from about 0.01% w/v to about 0.1% w/v, ethanol in an amount of from about 1% v/v to about 10% v/v, and acetate in a concentration of about 0.01 M to about 0.1 M.

In an embodiment, the RCP as measured at T=0 hours (immediately after manufacture of the pharmaceutical composition) is at least 94-97%. In an embodiment, the RCP is at least 94%. In an embodiment, the RCP is at least 95%. In an embodiment, the RCP is at least 96%. In an embodiment, the RCP is at least 97%. In an embodiment, the RCP is at least 98%. In an embodiment, the RCP is at least 99%. In any of the preceding embodiments, the RCP is measured by HPLC. Alternatively, in any of the preceding embodiments, the RCP is measured by iTLC.

In another embodiment, the RCP as measured at T=24 hours (24 hours after manufacture of the pharmaceutical composition), optionally, at 2-8° C., is at least 94-97%. In an embodiment, the RCP is at least 94%. In an embodiment, the RCP is at least 95%. In an embodiment, the RCP is at least 96%. In an embodiment, the RCP is at least 97%. In an embodiment, the RCP is at least 98%. In an embodiment, the RCP is at least 99%. In any of the preceding embodiments, the RCP is measured by HPLC. Alternatively, in any of the preceding embodiments, the RCP is measured by iTLC.

In yet another embodiment, the RCP as measured at T=48 hours (48 hours after manufacture of the pharmaceutical composition), optionally, at 2-8° C., is at least 90-95%. In an embodiment, the RCP is at least 90%. In an embodiment, the RCP is at least 91%. In an embodiment, the RCP is at least 92%. In an embodiment, the RCP is at least 93%. In an embodiment, the RCP is at least 94%. In an embodiment, the RCP is at least 95%. In any of the preceding embodiments, the RCP is measured by HPLC. Alternatively, in any of the preceding embodiments, the RCP is measured by iTLC.

In still another embodiment, the RCP as measured at T=72 hours (72 hours after manufacture of the pharmaceutical composition), optionally, at 2-8° C., is at least 90-95%. In an embodiment, the RCP is at least 90%. In an embodiment, the RCP is at least 91%. In an embodiment, the RCP is at least 92%. In an embodiment, the RCP is at least 93%. In an embodiment, the RCP is at least 94%. In an embodiment, the RCP is at least 95%. In any of the preceding embodiments, the RCP is measured by HPLC. Alternatively, in any of the preceding embodiments, the RCP is measured by iTLC.

In an embodiment, the RCP as measured at T=96 hours (96 hours after manufacture of the pharmaceutical composition), optionally, at 2-8° C., is 90-95%. In an embodiment, the RCP is at least 90%. In an embodiment, the RCP is at least 91%. In an embodiment, the RCP is at least 92%. In an embodiment, the RCP is at least 93%. In an embodiment, the RCP is at least 94%. In an embodiment, the RCP is at least 95%. In any of the preceding embodiments, the RCP is measured by HPLC. Alternatively, in any of the preceding embodiments, the RCP is measured by iTLC.

In another embodiment, the RCP as measured at T=120 hours (120 hours after manufacture of the pharmaceutical composition), optionally, at 2-8° C., is 90-95%. In an embodiment, the RCP is at least 90%. In an embodiment, the RCP is at least 91%. In an embodiment, the RCP is at least 92%. In an embodiment, the RCP is at least 93%. In an embodiment, the RCP is at least 94%. In an embodiment, the RCP is at least 95%. In any of the preceding embodiments, the RCP is measured by HPLC. Alternatively, in any of the preceding embodiments, the RCP is measured by iTLC.

In yet another embodiment, the RCP as measured at T=144 hours (144 hours after manufacture of the pharmaceutical composition), optionally, at 2-8° C., is 90-95%. In an embodiment, the RCP is at least 90%. In an embodiment, the RCP is at least 91%. In an embodiment, the RCP is at least 92%. In an embodiment, the RCP is at least 93%. In an embodiment, the RCP is at least 94%. In an embodiment, the RCP is at least 95%. In any of the preceding embodiments, the RCP is measured by HPLC. Alternatively, in any of the preceding embodiments, the RCP is measured by iTLC.

In still another embodiment, the RCP as measured at T=168 hours (168 hours after manufacture of the pharmaceutical composition), optionally, at 2-8° C., is 90-95%. In an embodiment, the RCP is at least 90%. In an embodiment, the RCP is at least 91%. In an embodiment, the RCP is at least 92%. In an embodiment, the RCP is at least 93%. In an embodiment, the RCP is at least 94%. In an embodiment, the RCP is at least 95%. In any of the preceding embodiments, the RCP is measured by HPLC. Alternatively, in any of the preceding embodiments, the RCP is measured by iTLC.

In an embodiment, the RCP of the stabilized composition as measured at T=24 is at least 90%. In another embodiment, the RCP of the stabilized composition as measured at T=48 hours, optionally, at 2-8° C., is at least 90%. In yet another embodiment, the RCP of the stabilized composition as measured at T=72 hours, optionally, at 2-8° C., is at least 90%. In still another embodiment, the RCP of the stabilized composition as measured at T=96 hours, optionally, at 2-8° C., is at least 90%. In an embodiment, the RCP of the stabilized composition as measured at T=120 hours, optionally, at 2-8° C., is at least 90%. In another embodiment, the RCP of the stabilized composition as measured at T=144 hours, optionally, at 2-8° C., is at least 90%. In yet another embodiment, the RCP of the stabilized composition as measured at T=168 hours, optionally, at 2-8° C., is at least 90%.

In still another embodiment, the pharmaceutical composition comprises a radiolabeled compound having a specific molar activity in the range of 60-19500 μCi per nmol. In an embodiment, the radioisotope is ¹⁷⁷Lu and the radiolabeled compound has a specific molar activity in the range of 5-19.5 mCi per nmol. In an embodiment, the radioisotope is ¹⁷⁷Lu and the radiolabeled compound has a specific molar activity in the range of 15-19 mCi per nmol. In yet another embodiment, the radioisotope is ²²⁵Ac and the radiolabeled compound has a specific molar activity in the range of 60-13200 μCi per nmol. In yet another embodiment, the radioisotope is ²²⁵Ac and the radiolabeled compound has a specific molar activity in the range of 60-13000 μCi per nmol.

In still another embodiment, provided herein is a pharmaceutical composition prepared using any of the methods disclosed herein.

In an aspect, provided herein is a pharmaceutical composition comprising:
a radiolabeled compound comprising (i) a peptide comprising a chelator, which is optionally DOTA, and (ii) a radioisotope which is ²²⁵Ac, wherein the ²²⁵Ac is chelated by the chelator;
ascorbic acid or a pharmaceutically acceptable salt thereof; and a polysorbate;
wherein the pharmaceutical composition has a RCP (RCP) of >90% after 72 hours at 2-8° C.

In an embodiment, the ascorbic acid or a pharmaceutically acceptable salt thereof and the polysorbate are present in the pharmaceutical composition in amounts sufficient to provide an RCP of at least 90% for at least 168 hours at 2-8° C. In an embodiment, the mass ratio of ascorbic acid or a pharmaceutically acceptable salt thereof to polysorbate in the stabilizing solution is from 50:1 to 150:1, optionally from 80:1 to 120:1, optionally about 100:1.

In an embodiment, the ascorbic acid or a pharmaceutically acceptable salt thereof is sodium ascorbate. In another embodiment, the ascorbic acid or a pharmaceutically acceptable salt thereof is present in the pharmaceutical composition in an amount of ≤100 mg/mL, optionally ≤80 mg/mL, optionally from 30-70 mg/mL, optionally from 40-60 mg/mL, optionally about 50 mg/mL.

In yet another embodiment, the polysorbate is polyoxyethylene (20) sorbitan monolaurate (Tween 20). In still another embodiment, the polysorbate is present in the pharmaceutical composition in an amount of ≤1 mg/mL, optionally 0.3-0.7 mg/mL, optionally 0.4-0.6 mg/mL, optionally about 0.5 mg/mL.

In another embodiment, the pharmaceutical composition further comprises L-methionine, optionally wherein the L-methionine is present in the pharmaceutical composition at a concentration of about 10 mg/mL to about 30 mg/mL. In yet another embodiment, the pharmaceutical composition further comprises ammonium acetate optionally wherein the ammonium acetate is present in the pharmaceutical composition at a concentration of about 0.01 M to about 0.15 M. In still another embodiment, the pharmaceutical composition also comprises ethanol, optionally wherein the ethanol is present in the pharmaceutical composition at about 1% v/v to about 15% v/v, optionally wherein the ethanol is present in the pharmaceutical composition at about 5% v/v to about 10% v/v.

In an embodiment, the pharmaceutical composition has an RCP>90% after 168 hours at 2-8° C.

In an embodiment, the radiolabeled compound has a specific molar activity in the range of 60-13000 μCi per nmol.

In another embodiment, the pharmaceutical composition comprises:
a radiolabeled compound comprising (i) a peptide comprising a DOTA chelator and (ii) a radioisotope which is ²²⁵Ac, wherein the ²²⁵Ac is chelated by the DOTA chelator;
sodium ascorbate, which is present in an amount of ≤100 mg/mL; and
polyoxyethylene (20) sorbitan monolaurate, which is present in an amount of ≤0.1% (w/v);
wherein the pharmaceutical composition has an RCP>90% after 72 hours.

In another aspect, provided herein is a pharmaceutical composition comprising:
a radiolabeled compound comprising (i) a peptide comprising a chelator, which is optionally DOTA, and (ii) a radioisotope which is ¹⁷⁷Lu, wherein the ¹⁷⁷Lu is chelated by the chelator;
ascorbic acid or a pharmaceutically acceptable salt thereof; and a polysorbate;
wherein the pharmaceutical composition has a RCP of >90% after 72 hours at 2-8° C.

In an embodiment, the ascorbic acid or a pharmaceutically acceptable salt thereof and the polysorbate are present in the pharmaceutical composition in amounts sufficient to provide an RCP of at least 90% for at least 168 hours at 2-8° C. In an embodiment, the mass ratio of ascorbic acid or a pharmaceutically acceptable salt thereof to polysorbate in the stabilizing solution is from 50:1 to 150:1, optionally from 80:1 to 120:1, optionally about 100:1.

In an embodiment, the ascorbic acid or a pharmaceutically acceptable salt thereof is sodium ascorbate. In another embodiment, the ascorbic acid or a pharmaceutically acceptable salt thereof is present in the pharmaceutical composition in an amount of ≤100 mg/mL, optionally ≤80 mg/mL, optionally from 30-70 mg/mL, optionally from 40-60 mg/mL, optionally about 50 mg/mL.

In yet another embodiment, the polysorbate is polyoxyethylene (20) sorbitan monolaurate (Tween 20). In still another embodiment, the polysorbate is present in the pharmaceutical composition in an amount of ≤1 mg/mL, optionally 0.3-0.7 mg/mL, optionally 0.4-0.6 mg/mL, optionally about 0.5 mg/mL.

In an embodiment, the pharmaceutical composition has an RCP>90% after 168 hours at 2-8° C.

In another embodiment, the pharmaceutical composition further comprises L-methionine, optionally wherein the L-methionine is present present in the pharmaceutical composition at a concentration of about 10 mg/mL to about 30 mg/mL.

In yet another embodiment, the pharmaceutical composition further comprises ammonium acetate optionally wherein the ammonium acetate is present present in the pharmaceutical composition at a concentration of about 0.01 M to about 0.15 M.

In an embodiment, the pharmaceutical composition also comprises ethanol, optionally wherein the ethanol is present present in the pharmaceutical composition at about 1% v/v to about 15% v/v, optionally wherein the ethanol is present present in the pharmaceutical composition at about 5% v/v to about 10% v/v.

In another embodiment, the radiolabeled compound has a specific molar activity in the range of 5-19.5 mCi per nmol.

In yet another embodiment, the pharmaceutical composition comprises:
a radiolabeled compound comprising (i) a peptide comprising a DOTA chelator and (ii) a radioisotope which is ¹⁷⁷Lu, wherein the ¹⁷⁷Lu is chelated by the DOTA chelator; sodium ascorbate, which is present in the pharmaceutical composition in an amount of ≤100 mg/mL; and
polyoxyethylene (20) sorbitan monolaurate, which is present in the pharmaceutical composition in an amount of ≤0.1% (mg/mL);
wherein the pharmaceutical composition has an RCP>90% after 72 hours.

In yet another aspect, provided herein is a pharmaceutical composition comprising:
(a) a radiolabeled peptide;
(b) a stabilizer; and
(c) a buffer,
wherein the composition is enriched in the radiolabeled peptide.

In an embodiment, the stabilizer is one or more of gentisic acid, ascorbic acid, acetic acid, methionine, selenomethionine, histidine, melatonin, ethanol, and or a pharmaceutically acceptable salt thereof. In another embodiment, the stabilizer is one or more of gentisic acid, ascorbic acid, sodium ascorbate, methionine, ethanol, and ammonium acetate.

In yet another embodiment, the stabilizer is a combination of:
(i) sodium ascorbate;
(ii) gentisic acid;
(iii) methionine; and
(iv) ethanol.

In still another embodiment, the stabilizer is:
(i) 10-700 mg/mL sodium ascorbate;
(ii) 0-100 mg/mL gentisic acid;
(iii) 0-100 mg/mL methionine; and
(iv) 5-45% v/v ethanol, each expressed as a concentration or a volume percentage of the composition.

In an embodiment, the stabilizer is:
(i) 25-600 mg/mL sodium ascorbate;
(ii) 0-50 mg/mL gentisic acid;
(iii) 0-30 mg/mL methionine; and
(iv) 5-15% v/v ethanol, each expressed as a concentration or a volume percentage of the composition.

In another embodiment, the stabilizer is a combination of:
(i) sodium ascorbate;
(ii) gentisic acid; and
(iii) ethanol.

In yet another embodiment, the stabilizer is:
(i) 200-300 mg/mL sodium ascorbate;
(ii) 22-28 mg/mL methionine; and
(iii) 8-13% v/v ethanol, each expressed as a concentration or a volume percentage of the composition.

In still another embodiment, the stabilizer is:
(i) 200-550 mg/mL sodium ascorbate;
(ii) 5-45 mg/mL gentisic acid; and
(iii) 8-13% v/v ethanol, each expressed as a concentration or a volume percentage of the composition.

In an embodiment, the concentration of buffer in the composition is 0.01-1.5 molar. In another embodiment, the buffer is selected from ammonium acetate, sodium acetate, acetic acid, sodium chloride, sodium phosphate, ascorbic acid, and sodium ascorbate, or a combination thereof.

In yet another embodiment, the radiolabeled peptide is labeled with a radionuclide selected from the group consisting of ¹⁷⁷Lu, ⁶⁸Ga, ¹⁸F, ^{99m}Tc, ²¹¹At, ¹⁶⁶Ho, ²²⁵Ac, ¹¹¹In, ¹²³I, ¹³¹I, ⁸⁹Zr, ⁹⁰Y, ²¹²Pb, ¹⁶¹Tb, ¹⁸⁸Re, ⁶⁴Cu, and ⁶⁷Cu.

In still another embodiment, the radiolabeled peptide is labeled with ²²⁵Ac.

In an embodiment, the pharmaceutical composition comprises:
(a) a radiolabeled peptide;
(b) ethanol, methionine, and sodium ascorbate; and
(c) an ammonium acetate buffer.

In another embodiment, the pharmaceutical composition has an RCP of at least about 90%. In yet another embodiment, the pharmaceutical composition has an RCP of at least about 93%. In still another embodiment, the pharmaceutical composition has an RCP of at least about 95%.

In an embodiment, the pharmaceutical composition has an RCP of at least about 96%. In another embodiment, the pharmaceutical composition has an RCP of at least about 97%. In yet another embodiment, the pharmaceutical composition has an RCP of at least about 98%.

An additional discovery of the disclosed pharmaceutical compositions is that the addition of a surfactant improves the stability of the resulting RLT composition over an extended period of time after isolation as measured by RCP. This result may be due to the tendency of RLTs to adhere to container surfaces, which could contribute to degradation of the product, affecting product shelf-life stability.

In an aspect, provided herein is a pharmaceutical composition comprising:
(a) a radiolabeled peptide;
(b) a stabilizer;
(c) a surfactant; and
(d) a buffer.

In an embodiment, the stabilizer is one or more of gentisic acid, ascorbic acid, acetic acid, methionine, selenomethionine, histidine, melatonin, ethanol, and or a pharmaceutically acceptable salt thereof. In another embodiment, the stabilizer is one or more of gentisic acid, ascorbic acid, sodium ascorbate, methionine, ethanol, and ammonium acetate.

In another embodiment, the stabilizer is a combination of:
(i) sodium ascorbate;
(ii) gentisic acid;
(iii) methionine; and
(iv) ethanol.

In still another embodiment, the stabilizer is:
(i) 10-700 mg/mL sodium ascorbate;
(ii) 0-100 mg/mL gentisic acid;
(iii) 0-100 mg/mL methionine; and
(iv) 5-45% v/v ethanol, each expressed as a concentration or a volume percentage of the composition.

In yet another embodiment, the stabilizer is:
(i) 25-600 mg/mL sodium ascorbate;
(ii) 0-50 mg/mL gentisic acid;
(iii) 0-30 mg/mL methionine; and
(iv) 5-15% v/v ethanol, each expressed as a concentration or a volume percentage of the composition.

In still another embodiment, the stabilizer is a combination of:
(i) sodium ascorbate;
(ii) gentisic acid; and
(iii) ethanol.

In an embodiment, the stabilizer is:
(i) 200-300 mg/mL sodium ascorbate;
(ii) 22-28 mg/mL methionine; and
(iii) 8-13% v/v ethanol, each expressed as a concentration or a volume percentage of the composition.

In another embodiment, the stabilizer is:
(i) 200-550 mg/mL sodium ascorbate;
(ii) 5-45 mg/mL gentisic acid; and
(iii) 8-13% v/v ethanol, each expressed as a concentration or a volume percentage of the composition.

In yet another embodiment, the surfactant is present in 1-5 volume percent (% v/v) of the composition.

In still another embodiment, the concentration of buffer in the composition is 0.01-1.5 molar. In another embodiment, the buffer is selected from ammonium acetate, sodium acetate, acetic acid, sodium chloride, sodium phosphate, ascorbic acid, and sodium ascorbate, or a combination thereof.

In yet another embodiment, the radiolabeled peptide is labeled with a radionuclide selected from the group consisting of ¹⁷⁷Lu, ⁶⁸Ga, ¹⁸F, ^{99m}Tc, ²¹¹At, ¹⁶⁶Ho, ²²⁵Ac, ¹¹¹In, ¹²³I, ¹³¹I, ⁸⁹Zr, ⁹⁰Y, ²¹²Pb, ¹⁶¹Tb, ¹⁸⁸Re, ⁶⁴Cu, and ⁶⁷Cu.

In still another embodiment, the surfactant is selected from the group consisting of polyoxyethylene (20) sorbitan monolaurate, polyoxyethylene (20) sorbitan monopalmitate, polyoxyethylene (20) sorbitan monostearate, polyoxyethylene (20) sorbitan monooleate, monooleate polyoxyethylene (20) cetyl ether, and 2,3-dihydroxypropyl octadecanoate.

In an embodiment, the surfactant is selected from the group consisting of polyoxyethylene (20) sorbitan monolaurate, polyoxyethylene (20) sorbitan monopalmitate, polyoxyethylene (20) sorbitan monostearate, and polyoxyethylene (20) sorbitan monooleate.

In another embodiment, the pharmaceutical composition comprises:
(a) a radiolabeled peptide;
(b) ethanol, methionine, and sodium ascorbate;
(c) polyoxyethylene (20) sorbitan monolaurate; and
(d) an ammonium acetate buffer.

In another embodiment, the pharmaceutical composition has an RCP of at least about 90%. In yet another embodiment, the pharmaceutical composition has an RCP of at least about 93%. In still another embodiment, the pharmaceutical composition has an RCP of at least about 95%.

In an embodiment, the pharmaceutical composition has an RCP of at least about 96%. In another embodiment, the pharmaceutical composition has an RCP of at least about 97%. In yet another embodiment, the pharmaceutical composition has an RCP of at least about 98%.

In still another embodiment, the pharmaceutical composition is stable at −22° C. to 27° C. for at least about 24 hours as indicated by an RCP of greater than 90%. In an embodiment, the pharmaceutical composition is stable at −22° C. to 27° C. for at least about 48 hours as indicated by an RCP of greater than 90%. In another embodiment, the pharmaceutical composition is stable at −22° C. to 27° C. for at least about 72 hours as indicated by an RCP of greater than 90%. In yet another embodiment, the pharmaceutical composition is stable at −22° C. to 27° C. for at least about 96 hours as indicated by an RCP of greater than 90%. In still another embodiment, the pharmaceutical composition is stable at −22° C. to 27° C. for at least about 120 hours as indicated by an RCP of greater than 90%.

In an embodiment, the pharmaceutical composition is stable at −22° C. to 27° C. for at least about 220 hours as indicated by an RCP of greater than 90%.

In another embodiment, the pharmaceutical composition is stable at −22° C. to −18° C. In yet another embodiment, the pharmaceutical composition is stable at 2° C. to 8° C. In still another embodiment, the pharmaceutical composition is stable at 23° C. to 27° C.

In an aspect, provided herein is a pharmaceutical composition comprising:
(a) a radiolabeled peptide;
(b) a stabilizer;
(c) a surfactant; and
(d) a buffer,
wherein the composition is enriched in the radiolabeled peptide.

In an embodiment, the stabilizer is one or more of gentisic acid, ascorbic acid, acetic acid, methionine, selenomethionine, histidine, melatonin, ethanol, and or a pharmaceutically acceptable salt thereof. In another embodiment, the stabilizer is one or more of gentisic acid, ascorbic acid, sodium ascorbate, methionine, ethanol, and ammonium acetate.

In yet another embodiment, the stabilizer is a combination of:
(i) sodium ascorbate;
(ii) gentisic acid;
(iii) methionine; and
(iv) ethanol.

In still another embodiment, the stabilizer is:
(i) 10-700 mg/mL sodium ascorbate;
(ii) 0-100 mg/mL gentisic acid;
(iii) 0-100 mg/mL methionine; and
(iv) 5-45% v/v ethanol, each expressed as a concentration or a volume percentage of the composition.

In still another embodiment, the stabilizer is:
(i) 25-600 mg/mL sodium ascorbate;
(ii) 0-50 mg/mL gentisic acid;
(iii) 0-30 mg/mL methionine; and
(iv) 5-15% v/v ethanol, each expressed as a concentration or a volume percentage of the composition.

In an embodiment, the stabilizer is a combination of:
(i) sodium ascorbate;
(ii) gentisic acid; and
(iii) ethanol.

In another embodiment, the stabilizer is:
(i) 200-300 mg/mL sodium ascorbate;
(ii) 22-28 mg/mL methionine; and
(iii) 8-13% v/v ethanol, each expressed as a concentration or a volume percentage of the composition.

In yet another embodiment, the stabilizer is:
(i) 200-550 mg/mL sodium ascorbate;
(ii) 5-45 mg/mL gentisic acid; and
(iii) 8-13% v/v ethanol, each expressed as a concentration or a volume percentage of the composition.

In still another embodiment, the surfactant is present in 1-5 volume percent (% v/v) of the composition.

In an embodiment, the concentration of buffer in the composition is 0.01-1.5 molar. In another embodiment, the buffer is selected from ammonium acetate, sodium acetate, acetic acid, sodium chloride, sodium phosphate, ascorbic acid, and sodium ascorbate, or a combination thereof.

In yet another embodiment, the radiolabeled peptide is labeled with a radionuclide selected from the group consisting of ¹⁷⁷Lu, ⁶⁸Ga, ¹⁸F, ^{99m}Tc, ²¹¹At, ¹⁶⁶Ho, ²²⁵Ac, ¹¹¹In, ¹²³I, ¹³¹I, ⁸⁹Zr, ⁹⁰Y, ²¹²Pb, ¹⁶¹Tb, ¹⁸⁸Re, ⁶⁴Cu, and ⁶⁷Cu.

In still another embodiment, the surfactant is selected from the group consisting of polyoxyethylene (20) sorbitan monolaurate, polyoxyethylene (20) sorbitan monopalmitate, polyoxyethylene (20) sorbitan monostearate, polyoxyethylene (20) sorbitan monooleate, monooleate polyoxyethylene (20) cetyl ether, and 2,3-dihydroxypropyl octadecanoate.

In an embodiment, the surfactant is selected from the group consisting of polyoxyethylene (20) sorbitan monolaurate, polyoxyethylene (20) sorbitan monopalmitate, polyoxyethylene (20) sorbitan monostearate, and polyoxyethylene (20) sorbitan monooleate.

In another embodiment, the pharmaceutical composition comprises:
(a) a radiolabeled peptide;
(b) ethanol, methionine, and sodium ascorbate;
(c) polyoxyethylene (20) sorbitan monolaurate; and
(d) an ammonium acetate buffer.

In another embodiment, the pharmaceutical composition has an RCP of at least about 90%. In yet another embodiment, the pharmaceutical composition has an RCP of at least about 93%. In still another embodiment, the pharmaceutical composition has an RCP of at least about 95%.

In an embodiment, the pharmaceutical composition has an RCP of at least about 96%. In another embodiment, the pharmaceutical composition has an RCP of at least about 97%. In yet another embodiment, the pharmaceutical composition has an RCP of at least about 98%.

In still another embodiment, the pharmaceutical composition is stable at −22° C. to 27° C. for at least about 24 hours as indicated by an RCP of greater than 90%. In an embodiment, the pharmaceutical composition is stable at −22° C. to 27° C. for at least about 48 hours as indicated by an RCP of greater than 90%. In another embodiment, the pharmaceutical composition is stable at −22° C. to 27° C. for at least about 72 hours as indicated by an RCP of greater than 90%. In yet another embodiment, the pharmaceutical composition is stable at −22° C. to 27° C. for at least about 96 hours as indicated by an RCP of greater than 90%. In still another embodiment, the pharmaceutical composition is stable at −22° C. to 27° C. for at least about 120 hours as indicated by an RCP of greater than 90%.

In an embodiment, the pharmaceutical composition is stable at −22° C. to 27° C. for at least about 220 hours as indicated by an RCP of greater than 90%.

In another embodiment, the pharmaceutical composition is stable at −22° C. to −18° C. In yet another embodiment, the pharmaceutical composition is stable at 2° C. to 8° C. In still another embodiment, the pharmaceutical composition is stable at 23° C. to 27° C.

The pharmaceutical compositions of the disclosure can also comprise additional excipients, including surfactants, polymers, and antioxidants. Surfactants suitable for use in the formulations of the disclosure include surfactants commonly used in the formulation of pharmaceuticals. Examples of surfactants include, but are not limited to, ionic- and nonionic surfactants or wetting agents commonly used in the formulation of pharmaceuticals, such as ethoxylated castor oil, polyglycolyzed glycerides, acetylated monoglycerides, sorbitan fatty acid esters, poloxamers, polyoxyethylene sorbitan fatty acid esters, polyoxyethylene derivatives, monoglycerides or ethoxylated derivatives thereof, diglycerides or polyoxyethylene derivatives thereof, sodium docusate, sodium laurylsulfate, cholic acid or derivatives thereof, lecithins, phospholipids, combinations thereof, and the like. Additional surfactants include but are not limited to fatty alcohols such as polyethylene glycols (PEGs) and cetyl alcohol.

Antioxidants suitable for use in the formulations of the disclosure include butylated hydroxyanisole (BHA), butylated hydroxytoluene (BHT), propyl gallate (PG), sodium metabisulfite, ascorbyl palmitate, potassium metabisulfite, tartaric acid, citric acid, citric acid monohydrate, and sodium sulfite.

### Peptides

Provided herein are peptide precursors that are useful in the preparation of the radiolabeled peptides of the disclosure and the formulations and pharmaceutical compositions comprising the same. Peptides of Formula (I) (II), and (III) are synthesized and characterized according to the procedures disclosed in US Patent Application Serial Nos. 63/508,191, 63/508,202, and 63/508,208, each of which is incorporated by reference in its entirety. The peptides disclosed herein have high-specific activity for proteins involved in cancer such as Delta Like Canonical Notch Ligand 3 (DLL3) and B7-H3.

Delta Like Canonical Notch Ligand 3 (also known as Delta-like protein 3, Drosophila, or DLL3) is a member of the delta protein ligand family. It is encoded by the DLL3 gene. DLL3 inhibits primary neurogenesis and is involved in diverting neurons along a specific differentiation pathway. It also plays a role in the formation of somite boundaries during segmentation of the paraxial mesoderm. Mutations in the DLL3 gene cause the autosomal recessive genetic disorder Jarcho-Levin syndrome. Expression of the DLL3 gene occurs in neuroendocrine tumors. DLL3 can be a potential target for treating tumors such as lung cancer.

In some embodiments, targeting constructs include targeting moieties specific for one or more DLL3 domains.

DLL3 is considered a low-expressing cancer target because it is generally expressed in low levels if at all (The Oncologist 2022, 27, 940). Low expression levels can prevent effectiveness of the diagnostic or therapeutic.

B7-H3, also known as CD276, is a transmembrane protein composed of either one or two pair(s) of IgV-like and IgC-like extracellular immunoglobulin domains, a transmembrane region, and a cytoplasmic tail. B7-H3 is an immune checkpoint molecule that produces a coinhibitory signal that decreases the activity of the Major Histocompatibility Complex and T cell receptor (MHC-TCR) signal between an antigen presenting cell (APC) and a T cell. By reducing the activity of the MHC-TCR signal, B7-H3 attenuates immune responses, thus preventing T cell proliferation and cytokine production while promoting interleukin 10 (IL-10) and transforming growth factor beta-1 (TGF-β1) production.

B7-H3 can be found in several different cellular compartments, but its expression is generally low in healthy cells. However, B7-H3 is highly expressed in tumor cell types, especially metastatic tumor cells, as well as activated immune cells in the tumor microenvironment. As the expression and role of B7-H3 in cancer progression is ubiquitous, it has led to the correlation between B7-H3 expression and poor overall survival in cancer patients.

Due to the significance of B7-H3 and/or DLL3 expression in cancer, stabilized RLTs and methods used to produce them are important for the treatment of cancer characterized by B7-H3 and/or DLL3 expression.

DOTA-TATE is an eight amino acid long peptide with a covalently bonded DOTA bifunctional chelator (Nockel P., et al., Thyroid. 2016, 26 (6): 831-5). DOTA-TATE can be reacted with radionuclides such as actinium-225, gallium-68, lutetium-177, and copper-64 to form radiopharmaceuticals for positron emission tomography (PET) imaging or radionuclide therapy. Radionuclide therapy with DOTA-TATE targets somatostatin receptors (SSR) (Aktolun, C., et al., Nuclear Medicine Therapy: Principles and Clinical Applications. 2012, Springer. p. 364). Somatostatin (SST) is a small peptide that exerts inhibitory effects on a wide range of neuroendocrine cells. Because somatostatin regulates cell growth and hormone secretion, somatostatin receptors (SSTRs) have become valuable targets for the treatment of different types of neuroendocrine tumors (NETs).

In a non-limiting embodiment, the methods provided herein are used to prepare stabilized compositions of radiolabeled compounds from a mixture comprising the radiolabeled compound and a corresponding unlabeled compound. In an embodiment, the radiolabeled compound is a radiolabeled peptide of Formula I, Formula II, Formula III, or any subgeneric formula thereof. In another embodiment, the radiolabeled compound is a radiolabeled peptide that is DOTA-TATE.

In a non-limiting embodiment, the radiolabeled compounds are characterized by a high specific molar activity (e.g., 60-19500 μCi per nmol of compound).

### Peptides of Formula (I)

In an embodiment, the peptide comprises the amino acid sequence:

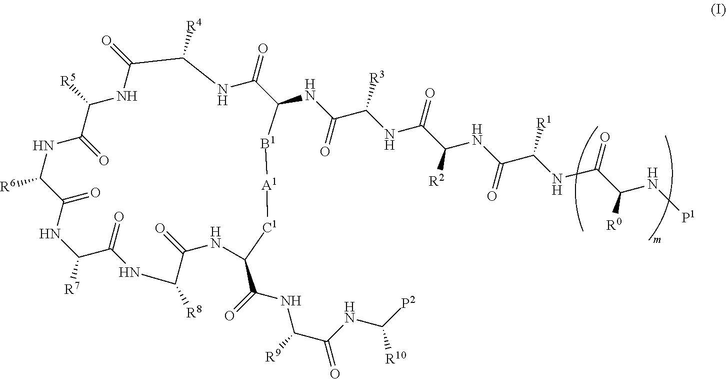

or a pharmaceutically acceptable salt thereof,
   wherein:
P¹ is selected from: -L¹-chelator,

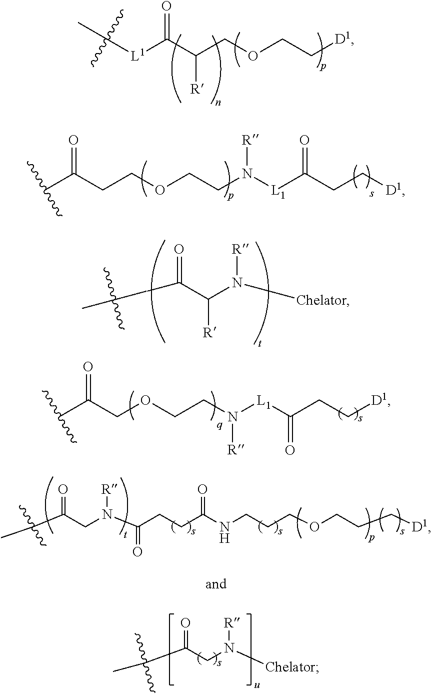

D¹ is —NR″-chelator;
L¹ is absent or selected from

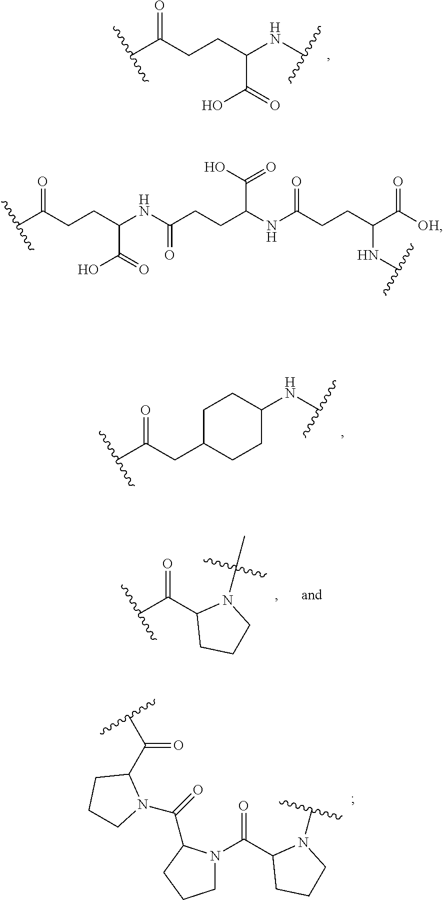 [SMILES: *NC(CCC(*)=O)C(=O)O ; *NC(CCC(=O)NC(CCC(=O)NC(CCC(*)=O)C(=O)O)C(=O)O)C(=O)O ; *NC1CCC(CC(*)=O)CC1 ; *C(=O)C1CCCN1* ; *C(=O)C1CCCN1C(=O)C1CCCN1C(=O)C1CCCN1*]

wherein the amino group of L¹ connects to the carbonyl group of P¹ or chelator to form an amide bond;
P² is selected from C(O)NH₂, C(O)OH,

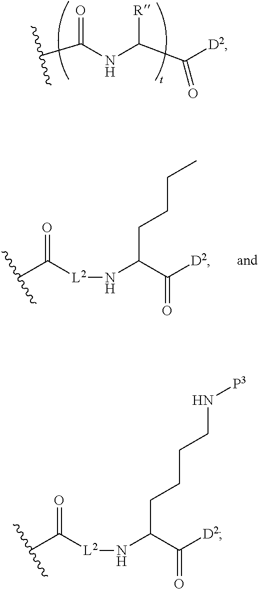

D² is OH or NH₂;
L² is absent or selected from:

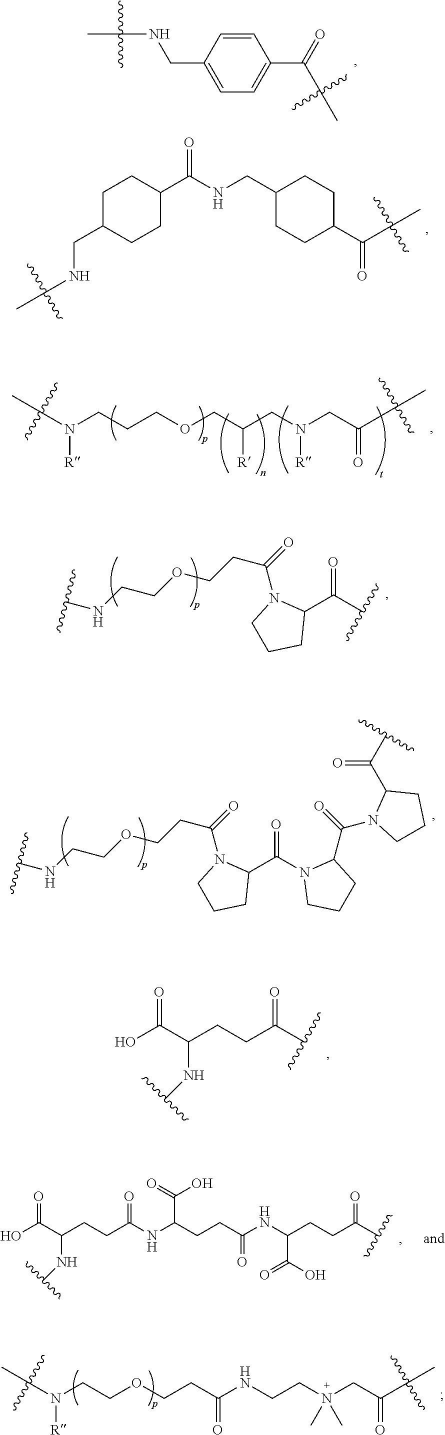

wherein the amino group of L² connects to the carbonyl group of P² to form an amide bond;
P³ is selected from H,

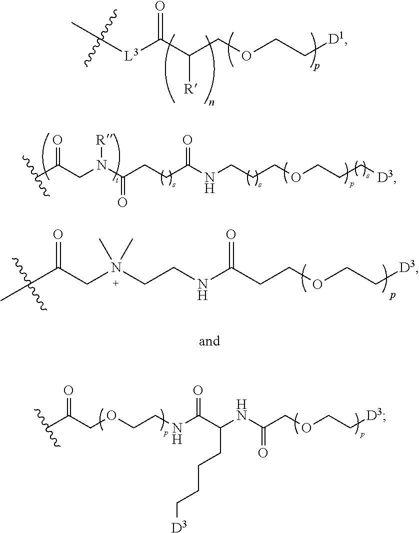

L³ is absent or independently selected from

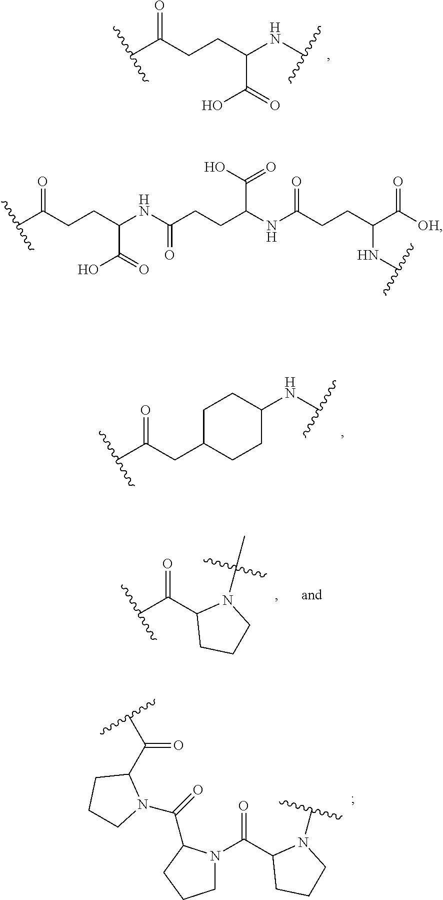 [SMILES: *NC(CCC(*)=O)C(=O)O ; *NC(CCC(=O)NC(CCC(=O)NC(CCC(*)=O)C(=O)O)C(=O)O)C(=O)O ; *NC1CCC(CC(*)=O)CC1 ; *C(=O)C1CCCN1* ; *C(=O)C1CCCN1C(=O)C1CCCN1C(=O)C1CCCN1*]

wherein the carbonyl group of L³ connects to an amine group of P² to form an amide bond;
D³ is independently selected from: CH₃, C(O)OH, and

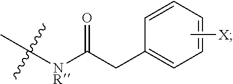

X is halogen;
R⁰ is an amino acid side chain of a natural amino acid or an amino acid side chain of an unnatural amino acid;
R¹ is an amino acid side chain of a natural amino acid or an amino acid side chain of an unnatural amino acid;
R² is an amino acid side chain of a natural amino acid or an amino acid side chain of an unnatural amino acid;
R³ is an amino acid side chain of a natural amino acid or an amino acid side chain of an unnatural amino acid;
B¹ is C₁₋₆ alkylene;
C¹ is C₁₋₆ alkylene;
A¹ is selected from:

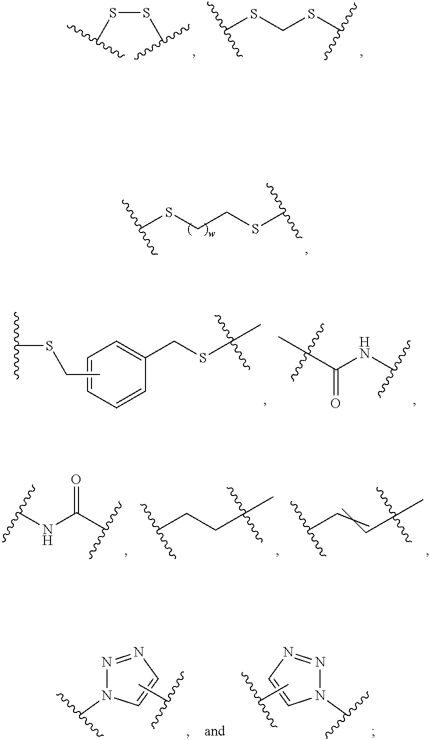

wherein w is selected from 1, 2, or 3;
R⁴ is an amino acid side chain of a natural amino acid or an amino acid side chain of an unnatural amino acid, both of which are optionally substituted with CH₂C(O)OH or C(O)(CH₂CH₂O)ₚ(CH₂)₂N(CH₃)₃⁺;
R⁵ is an amino acid side chain of a natural amino acid or an amino acid side chain of an unnatural amino acid;
R⁶ is an amino acid side chain of a natural amino acid or an amino acid side chain of an unnatural amino acid;
R⁷ is selected from:
(i) an amino acid side chain of a natural amino acid,
(ii) an amino acid side chain of an unnatural amino acid, or
(iii) selected from the group consisting of:

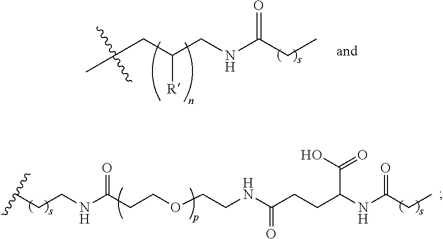

R⁸ is an amino acid side chain of a natural amino acid or an amino acid side chain of an unnatural amino acid;
R⁹ is an amino acid side chain of a natural amino acid or an amino acid side chain of an unnatural amino acid;
R¹⁰ is an amino acid side chain of a natural amino acid or an amino acid side chain of an unnatural amino acid;
m is 0 or 1;
each n, q, and u are independently an integer from 0 to 16;
each p is independently an integer from 0 to 24;
each s is independently an integer from 0 to 16;
each t is independently 1, 2, 3, 4, 5, or 6;
R′ is independently selected from H, C(O)OH, (CH₂)OH and NHAc; and
R″ is independently selected from H and CH₃.

In an embodiment, the peptide of Formula I is a peptide of Formula Ia comprising the amino acid sequence:

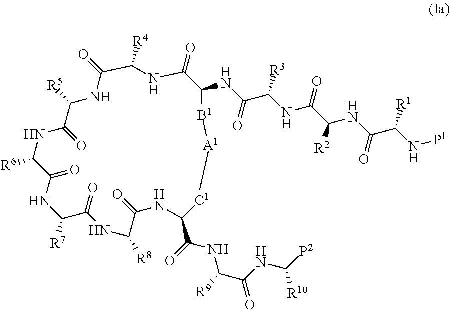

or a pharmaceutically acceptable salt thereof.

In another embodiment, the peptide of Formula I is a peptide of Formula Ib comprising the amino acid sequence:

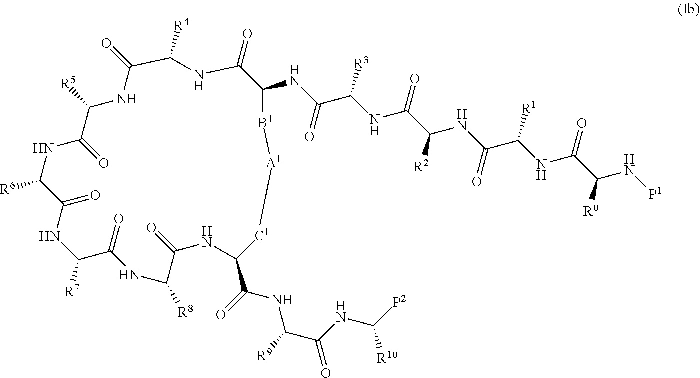

or a pharmaceutically acceptable salt thereof.

In yet another embodiment,
P¹ is selected from

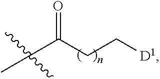

L¹-chelator, and

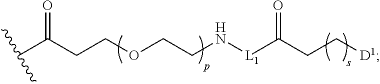

wherein:
D¹ is NR″-chelator;
L¹ is absent or

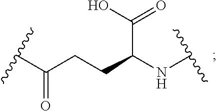 [SMILES: *N[C@@H](CCC(*)=O)C(=O)O]

n and s are independently an integer from 2 to 15; and
p is 8, 9, 10, 11, or 12.

In still another embodiment, P¹ is -L¹-chelator.

In an embodiment,
P¹ is selected from DOTA,

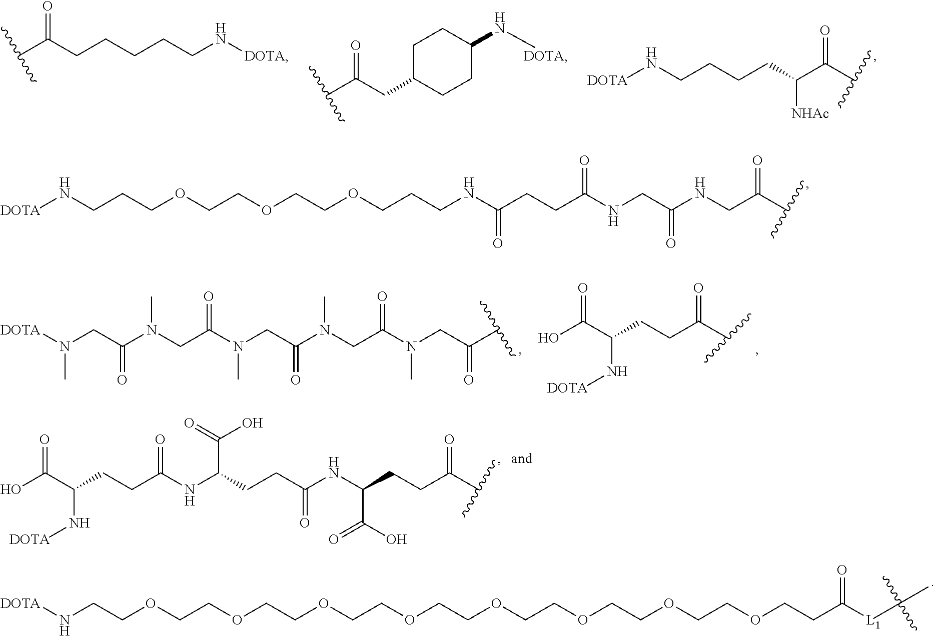

In another embodiment, P² is selected from C(O)NH₂ and C(O)OH.

In yet another embodiment, P² is selected from C(O)NH₂, C(O)OH,

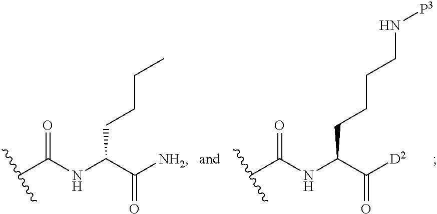

P³ is selected from Ac and

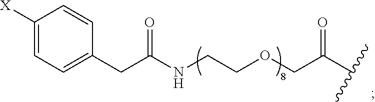

X is halo.

In still another embodiment, the peptide of Formula I is a peptide of Formula II-I comprising the amino acid sequence:

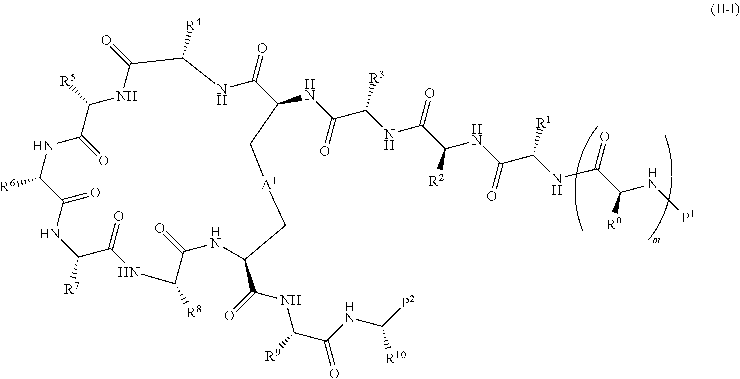

or a pharmaceutically acceptable salt thereof,
wherein P² is C(O)NH₂ or C(O)OH.

In yet another embodiment,
m is 0;
P¹ is selected from DOTA,

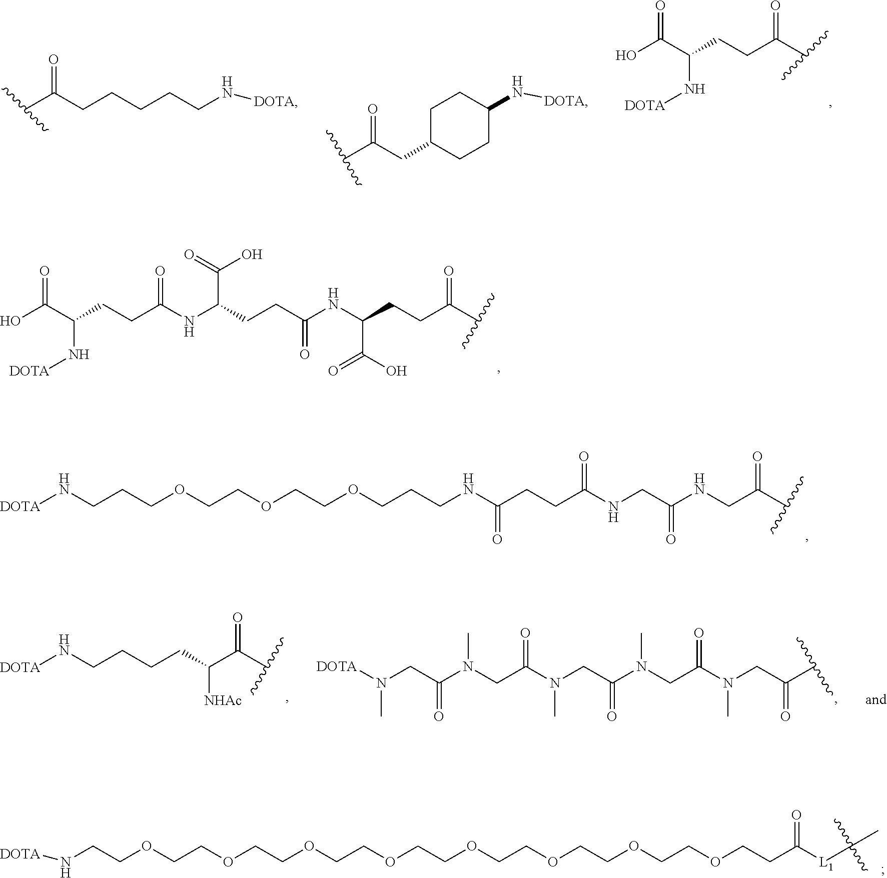

P² is selected from C(O)NH₂, C(O)OH,

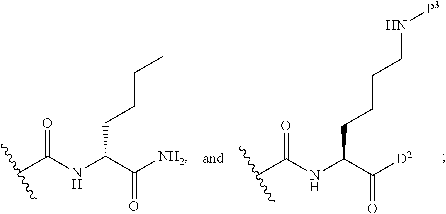

P³ is Ac or

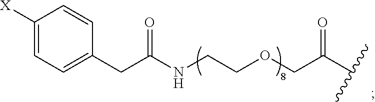

X is halo;
R¹ is an amino acid side chain selected from the group consisting of Trp, 2Nal, 1 Nal, 4CF₃-Phe, 7Aza-Trp, 1Me-Trp, 5OH-Trp, BIP, 5OMe-Trp, 4F-Phe, 3Pya, 4Pya, PAF, MAF, OAF, 5Qui, 7MeO-Trp, 7Me-Trp, 5F-Trp, 7Cl-Trp, D-Ala, Ala, alpha-Me-Trp, and NMe-Trp;
R² is an amino acid side chain selected from the group consisting of Thr, D-Ala, Ala, alpha-Me-Thr, Lys, and NMe-Thr;
R³ an amino acid side chain selected from the group consisting of Ile, Env, CHA, CBA, Nle, Tbg, THPG, Chg, 2Nal, 1Nal, 2CF₃-Phe, 2PhEt-Ala, D-Ala, Ala, Leu, t-Bu-Ala, NMe-Nle, α-tert-amylGly, Allo-Ile, Lys(C12), Lys(C14), Lys(C16), alpha-Me-Ile, and NMe-tBuAla;
A¹ is selected from the group consisting of:

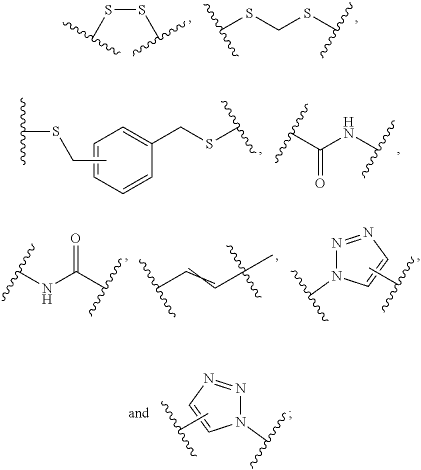

R⁴ is an amino acid side chain selected from the group consisting of Asn, D-Ala, Ala, DAB-4-NHCOC₅H₁₁, DAB-4-NHCOC₇H₁₅, Asp, Ser, Lys, 3-(4-piperidinyl)-Ala, 3-(1-morpholinyl)-Ala, 3Pya, 4Pya, Glu, NMe-Asn, Pip(CH₂CO₂H)Ala, and Pip(PegNMe₃)Ala;
R⁵ is an amino acid side chain selected from the group consisting of Asn, Ala, D-Ala, Trp, Asp, Lys, 3Pya, 4Pya, 3-(4-piperidinyl)-Ala, 3-(1-morpholinyl)-Ala, Glu, NMe-Asn, and Ser;
R⁶ is an amino acid side chain selected from the group consisting of Trp, 4CF₃-Phe, 1Me-Trp, 7Aza-Trp, BIP, 2Nal, 1Nal, alpha-Me-Trp, D-Ala, Ala, 4F-Phe, 1Nal, 2Nal, 5F-Trp, 5OMe-Trp, Asn, 50H-Trp, 7Me-Trp, 7MeO-Trp, 7Cl-Trp, and NMe-Trp;
R⁷ is:
(i) an amino acid side chain selected from the group consisting of 3Pya, 4Pya, Lys(Me)₃, His, Ala, D-Ala, Gln, Lys, Glu, Arg, Orn, NMe-His, and Ser; or
(ii) selected from the group consisting of:

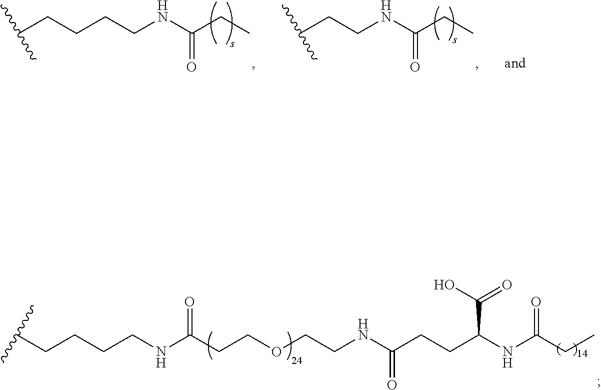

each s is independently 3, 5, 10, 12, or 14;
R⁸ is an amino acid side chain selected from the group consisting of Asp, D-Ala, Ala, Asn, Thr, NMe-Asp, and alpha-Me-Asp;
R⁹ is an amino acid side chain selected from the group consisting of Trp, 7Aza-Trp, 1Me-Trp, D-Ala, Ala, 4F-Phe, 1Nal, 2Nal, 5F-Trp, 5OMe-Trp, alpha-Me-Trp, 7Cl-Trp, 50H-Trp, 7Me-Trp, 7MeO-Trp, and NMe-Trp; and
R¹⁰ is an amino acid side chain selected from the group consisting of Pro, D-Ala, Ala, alpha-Me-Pro, trans4Fluoro-Pro, cis4Fluoro-Pro, trans40H-Pro, cis40H-Pro, Pip, 5,5-diMe-Pro, NMe-Ser, trans4NH2-Pro, cis4NH2-Pro, Sar, Aze, NMe-Ala, NMe-Leu, R-3Me-Aze, alpha-Me-Aze, ACl, and 3Me2-Aze.

In another embodiment,
m is 1;
P¹ is selected from DOTA,

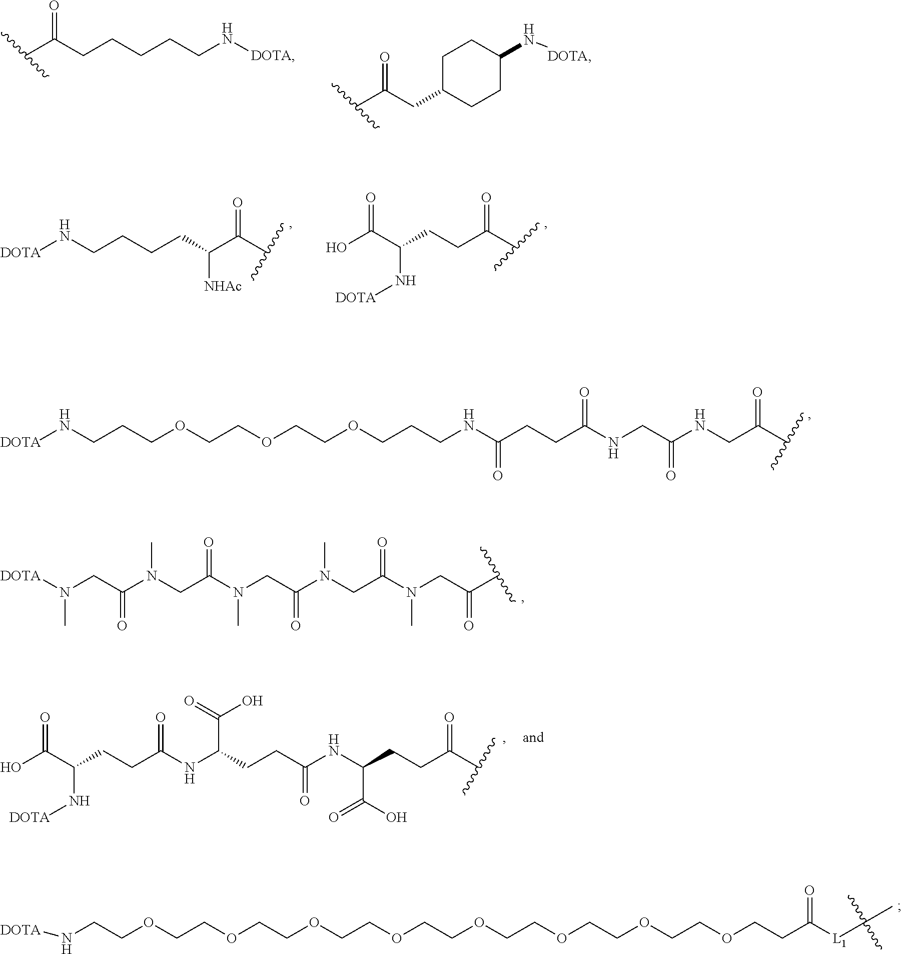

P² is selected from C(O)NH₂, C(O)OH,

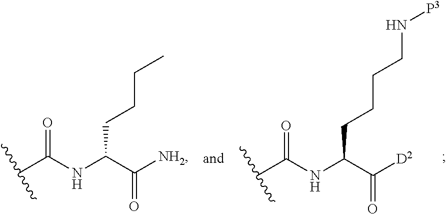

P³ is selected from Ac and

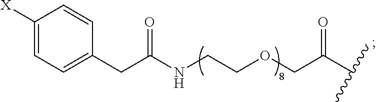

X is halo;
R⁰ is an amino acid side chain selected from the group consisting of Gly, Met, D-Ala, Ala, Nle, and Nva;
R¹ is an amino acid side chain selected from the group consisting of Trp, 2Nal, 1 Nal, 4CF₃-Phe, 7Aza-Trp, 1Me-Trp, 50H-Trp, BIP, 5OMe-Trp, 4F-Phe, 3Pya, 4Pya, PAF, MAF, OAF, 5Qui, 7MeO-Trp, 7Me-Trp, 5F-Trp, 7Cl-Trp, D-Ala, Ala, alpha-Me-Trp, and NMe-Trp;
R² is an amino acid side chain selected from the group consisting of Thr, D-Ala, Ala, alpha-Me-Thr, Lys, and NMe-Thr;
R³ is an amino acid side chain selected from the group consisting of Ile, Env, CHA, CBA, Nle, Tbg, THPG, Chg, 2Nal, 1Nal, 2CF₃-Phe, 2PhEt-Ala, D-Ala, Ala, Leu, t-Bu-Ala, NMe-Nle, α-tert-amylGly, Allo-Ile, Lys(C12), Lys(C14), Lys(C16), alpha-Me-Ile, and NMe-tBuAla;
A¹ is selected from the group consisting of

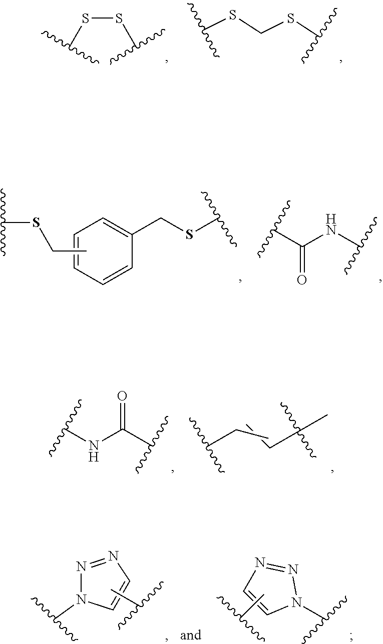

R⁴ is an amino acid side chain selected from the group consisting of Asn, D-Ala, Ala, DAB-4-NHCOC₅H₁₁, DAB-4-NHCOC₇H₁₅, Asp, Ser, Lys, 3-(4-piperidinyl)-Ala, 3-(1-morpholinyl)-Ala, 3Pya, 4Pya, Glu, and NMe-Asn;
R⁵ is an amino acid side chain selected from the group consisting of Asn, Ala, D-Ala, Trp, Asp, Lys, 3Pya, 4Pya, 3-(4-piperidinyl)-Ala, 3-(1-morpholinyl)-Ala, Glu, NMe-Asn, and Ser;
R⁶ is an amino acid side chain selected from the group consisting of Trp, 4CF₃-Phe, 1Me-Trp, 7Aza-Trp, BIP, 2Nal, 1Nal, alpha-Me-Trp, D-Ala, Ala, 4F-Phe, 1Nal, 2Nal, 5F-Trp, 5OMe-Trp, Asn, 50H-Trp, 7Me-Trp, 7MeO-Trp, 7Cl-Trp, and NMe-Trp;
R⁷ is an amino acid side chain selected from the group consisting of 3Pya, 4Pya, Lys(Me)3, His, Ala, D-Ala, Gln, Lys, Glu, Arg, Orn, NMe-His, and Ser; or
R⁷ is selected from the group consisting of

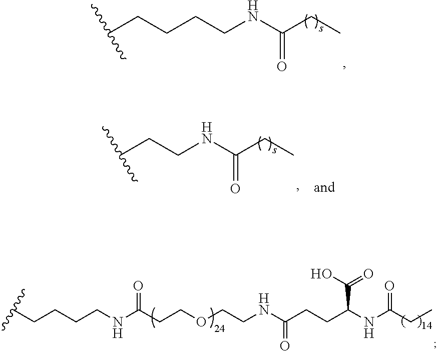

each s is independently 3, 5, 10, 12, or 14;
R⁸ is an amino acid side chain selected from the group consisting of Asp, D-Ala, Ala, Asn, Thr, NMe-Asp, and alpha-Me-Asp;
R⁹ is an amino acid side chain selected from the group consisting of Trp, 7Aza-Trp, 1Me-Trp, D-Ala, Ala, 4F-Phe, 1Nal, 2Nal, 5F-Trp, 5OMe-Trp, alpha-Me-Trp, 7Cl-Trp, 50H-Trp, 7Me-Trp, 7MeO-Trp, and NMe-Trp; and
R¹⁰ is an amino acid side chain selected from the group consisting of Pro, D-Ala, Ala, alpha-Me-Pro, trans4Fluoro-Pro, cis4Fluoro-Pro, trans40H-Pro, cis40H-Pro, Pip, 5,5-diMe-Pro, NMe-Ser, trans4NH2-Pro, cis4NH2-Pro, Sar, Aze, NMe-Ala, NMe-Leu, R-3Me-Aze, alpha-Me-Aze, ACl, and 3Me2-Aze.

In yet another embodiment,
B¹ is CH₂ or C(CH₃)₂; and
C¹ is CH₂ or C(CH₃)₂.

In still another embodiment,
B¹ is CH₂; and
C¹ is CH₂.

In an embodiment, chelator is independently selected from the group consisting of ethylenediamine tetraacetic acid (EDTA), diethylenetriamine pentaacetic acid (DTPA), 1,4,7,10-tetra-azacylcododecane-N,N′,N″,N′″-tetraacetic acid (DOTA), 6-((16-((6-Carboxypyridin-2-yl)methyl)-1,4,10,13-tetraoxa-7,16-diazacyclooctadecan-7-yl)methyl)-4-isothiocyanatopicolinic acid (Macropa), Macrodipa, 2,2′,2″,2′″-(1,10-dioxa-4,7,13,16-tetraazacyclooctadecane-4,7,13,16-tetrayl)tetraacetic acid) (Crown), 1,4,7,10-Tetraazacyclododecane-1,4,7,10-tetraacetic acid, α-(2-carboxyethyl) (DOTAGA), 1,4,7-Triazacyclononane-N,N′,N″-triacetic acid (NOTA), 1,4,7,10-tetraazacyclododecane-N,N′,N″,N′″-tetraacetic acid (TETA), 1,4,7,10,13-pentaazacyclopentadecane-N,N′,N″,N′″,N″″-pentaacetic acid (PEPA), and 1,4,7,10,13,16-hexaazacyclohexadecane-N,N′,N″,N′″,N″″,N′″″-hexaacetic acid (HEHA).

In another embodiment, chelator is DOTA. In yet another embodiment, chelator is DOTAGA. In still another embodiment, chelator is macrodipa. In an embodiment, chelator is macropa.

In another embodiment, Formula I is substituted by at least one chelator. In yet another embodiment, Formula I is substituted by one chelator. In still another embodiment, Formula I is substituted by at two chelators.

In an embodiment, the peptide comprises the amino acid sequence:

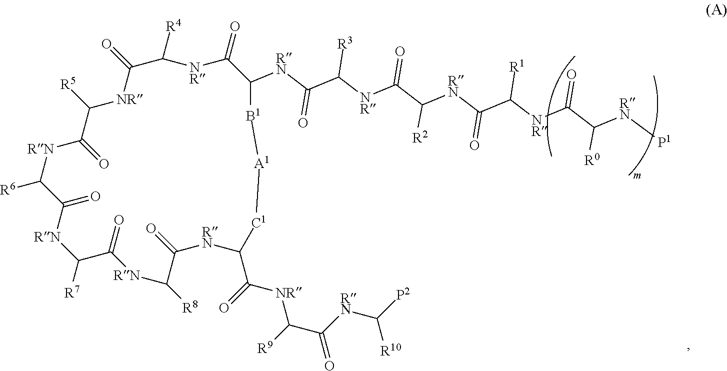

or a pharmaceutically acceptable salt thereof,
wherein the variable definitions are the same as those defined above for Formula I;
wherein:
each alpha-carbon atom on the peptide backbone is optionally substituted with methyl;
when a variable group R⁰, R¹, R², R³, R⁴, R⁵, R⁶, R⁷, R⁸, R⁹, or R¹⁰ is the side chain of a cyclic amino acid, the corresponding amino acid nitrogen of the peptide backbone forms part of the cyclic group; and
   wherein the cyclic peptide optionally comprises a radionuclide. In an embodiment, the peptide of Formula I is selected from a peptide in Table 2. In another embodiment, the peptide of Formula A is selected from a peptide in Table 2.

TABLE 2

| Cpd# | Sequence |
|---|---|
| 17 | DOTA-W-T-I-C(3)-N-N-W-H-D-C(3)-W-P-CONH2 |
| 18 | DOTA-Ahx-W-T-I-C(3)-N-N-W-H-D-C(3)-W-P-CONH2 |
| 19 | DOTA-CyHex-W-T-I-C(3)-N-N-W-H-D-C(3)-W-P-CONH2 |
| 20 | DOTA-PEG8-W-T-I-C(3)-N-N-W-H-D-C(3)-W-P-CONH2 |
| 22 | DOTA-(Sar)5-W-T-I-C(3)-N-N-W-H-D-C(3)-W-P-CONH2 |
| 54 | DOTA-Ahx-W-T-I-C(3)-N-N-W-H-D-C(3)-(1Nal)-P-CONH2 |
| 55 | DOTA-Ahx-W-T-I-C(3)-N-N-W-H-D-C(3)-(2Nal)-P-CONH2 |
| 56 | DOTA-Ahx-W-T-I-C(3)-N-N-(1Nal)-H-D-C(3)-W-P-CONH2 |
| 57 | DOTA-Ahx-W-T-I-C(3)-N-N-(2Nal)-H-D-C(3)-W-P-CONH2 |
| 58 | DOTA-Ahx-(5Fluoro-W)-T-I-C(3)-N-N-W-H-D-C(3)-W-P-CONH2 |
| 59 | DOTA-Ahx-W-T-I-C(3)-N-N-(5Fluoro-W)-H-D-C(3)-W-P-CONH2 |
| 60 | DOTA-Ahx-W-T-I-C(3)-N-N-W-H-D-C(3)-(5Fluoro-W)-P-CONH2 |
| 61 | DOTA-Ahx-W-T-I-C(3)-N-N-W-H-D-C(3)-W-(aMe-P)-CONH2 |
| 62 | DOTA-Ahx-W-T-(1Nal)-C(3)-N-N-W-H-D-C(3)-W-P-CONH2 |
| 63 | DOTA-Ahx-W-T-(2Nal)-C(3)-N-N-W-H-D-C(3)-W-P-CONH2 |
| 64 | DOTA-Ahx-W-T-(2PhEt-A)-C(3)-N-N-W-H-D-C(3)-W-P-CONH2 |
| 65 | DOTA-Ahx-W-T-(2CF3-F)-C(3)-N-N-W-H-D-C(3)-W-P-CONH2 |
| 66 | DOTA-Ahx-W-T-I-C(3)-N-N-W-H-D-C(3)-W-(trans4Fluoro-P)-CONH2 |
| 67 | DOTA-Ahx-W-T-I-C(3)-N-N-W-H-D-C(3)-W-(Cis4Fluoro-P)-CONH2 |
| 68 | DOTA-Ahx-W-T-I-C(3)-N-N-W-H-D-C(3)-W-(trans4OH-P)-CONH2 |
| 69 | DOTA-Ahx-W-T-I-C(3)-N-N-W-H-D-C(3)-W-(cis4OH-P)-CONH2 |
| 70 | DOTA-Ahx-W-T-I-C(3)-N-N-W-H-D-C(3)-W-(Pip)-CONH2 |
| 73 | Ac-D-Lys(DOTA)-W-T-I-C(3)-N-N-W-H-D-C(3)-W-P-CONH2 |
| 78 | DOTA-PEG8-CyHex-W-T-I-C(3)-N-N-W-H-D-C(3)-W-P-CONH2 |
| 79 | DOTA-PEG8-(dPro)-W-T-I-C(3)-N-N-W-H-D-C(3)-W-P-CONH2 |
| 80 | DOTA-PEG8-(dPro)3--W-T-I-C(3)-N-N-W-H-D-C(3)-W-P-CONH2 |
| 81 | DOTA-(gE)-W-T-I-C(3)-N-N-W-H-D-C(3)-W-P-CONH2 |
| 82 | DOTA-(gE)3-W-T-I-C(3)-N-N-W-H-D-C(3)-W-P-CONH2 |
| 83 | DOTA-PEG8-(gE)-W-T-I-C(3)-N-N-W-H-D-C(3)-W-P-CONH2 |
| 84 | DOTA-PEG8-(gE)3--W-T-I-C(3)-N-N-W-H-D-C(3)-W-P-CONH2 |
| 96 | DOTA-Ahx-W-(aMe-T)-I-C(3)-N-N-W-H-D-C(3)-W-P-CONH2 |
| 97 | DOTA-Ahx-W-T-I-C(3)-N-N-(aMe-W)-H-D-C(3)-W-P-CONH2 |
| 98 | DOTA-Ahx-W-T-I-C(3)-N-N-W-H-D-C(3)-W-(5,5-diMe-P)-CONH2 |
| 163 | DOTA-Ahx-W-T-I-C(3)-N-N-W-H-D-C(3)-W-P-(D-Nle)-NH2 |
| 219 | DOTA-Ahx-W-T-I-C(1)-N-N-W-H-D-C(1)-W-P-CONH2 |
| 220 | DOTA-(D-gE)-W-T-I-C(3)-N-N-W-H-D-C(3)-W-P-CONH2 |
| 221 | DOTA-(D-gE)3-W-T-I-C(3)-N-N-W-H-D-C(3)-W-P-CONH2 |
| 222 | DOTA-PEG8-(D-gE)-W-T-I-C(3)-N-N-W-H-D-C(3)-W-P-CONH2 |
| 223 | DOTA-PEG8-(D-gE)3--W-T-I-C(3)-N-N-W-H-D-C(3)-W-P-CONH2 |
| 224 | DOTA-Ahx-W-T-I-C(3)-N-N-W-H-D-C(3)-W-(trans4NH2-P)-CONH2 |
| 225 | DOTA-Ahx-W-T-I-C(3)-N-N-W-H-D-C(3)-W-(cis4NH2-P)-CONH2 |

or a pharmaceutically acceptable salt thereof.

### Peptides of Formula (II)

In another embodiment, the peptide comprises the amino acid sequence:

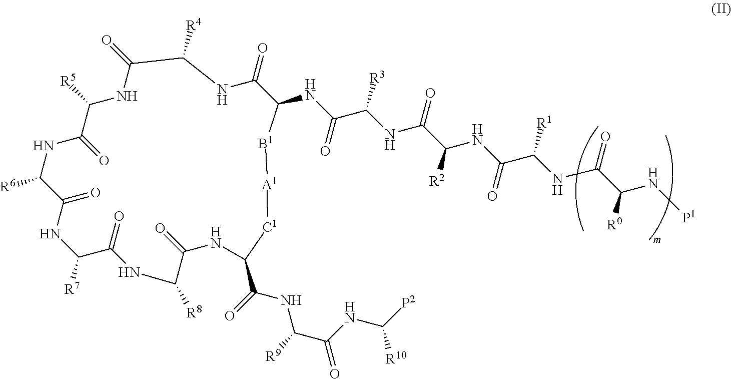

or a pharmaceutically acceptable salt thereof,
   wherein:
P¹ is selected from: H,

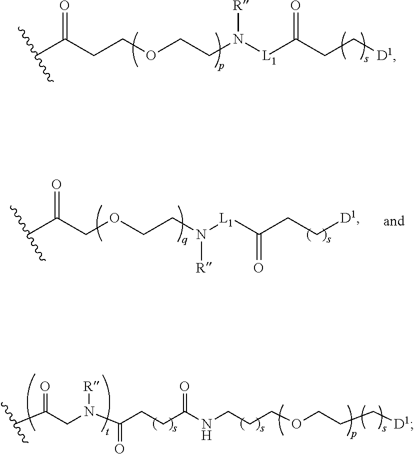

D¹ is selected from H, CH₃, and C(O)OH;
L¹ is absent or selected from

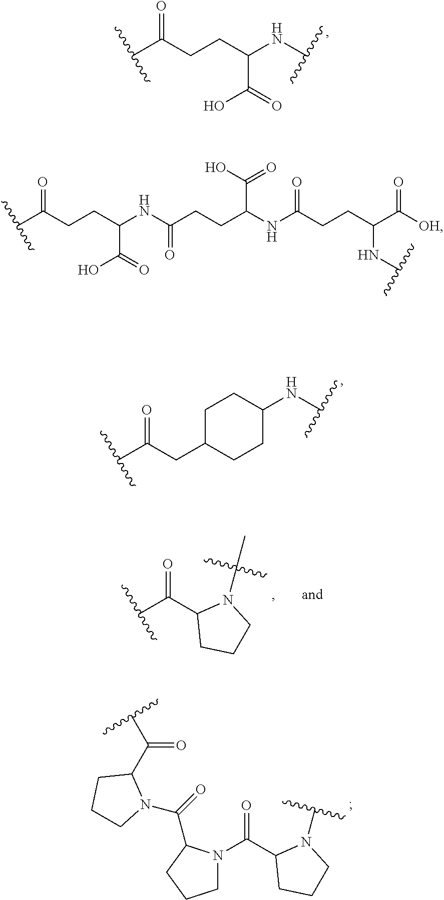 [SMILES: *NC(CCC(*)=O)C(=O)O ; *NC(CCC(=O)NC(CCC(=O)NC(CCC(*)=O)C(=O)O)C(=O)O)C(=O)O ; *NC1CCC(CC(*)=O)CC1 ; *C(=O)C1CCCN1* ; *C(=O)C1CCCN1C(=O)C1CCCN1C(=O)C1CCCN1*]

wherein the amino group of L¹ connects to the carbonyl group of P¹ to form an amide bond;
P² is -L²-chelator or

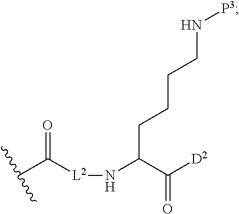

D² is OH or NH₂;
L² is absent or selected from:

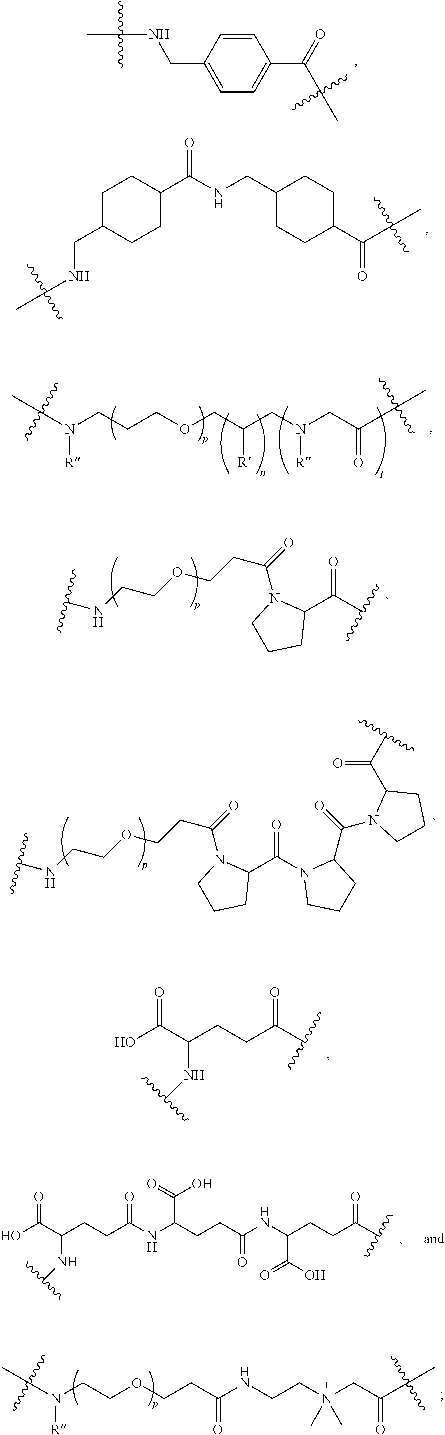

wherein the amino group of L² connects to the carbonyl group of P² or chelator to form an amide bond;
P³ is selected from -L³-chelator,

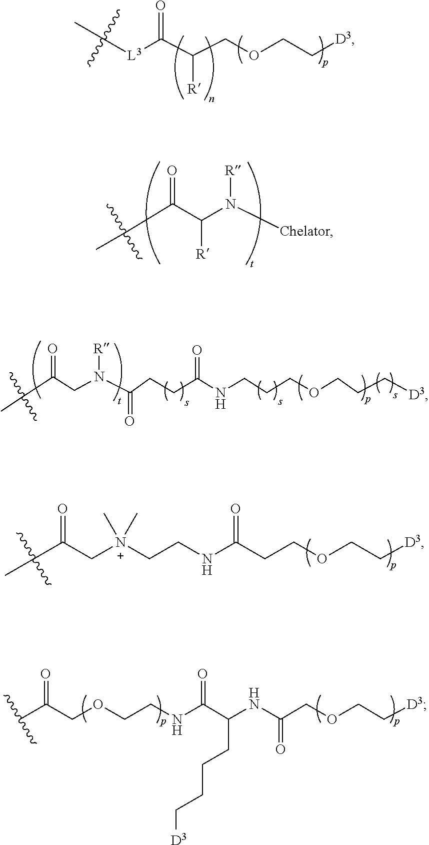

L³ is absent or independently selected from

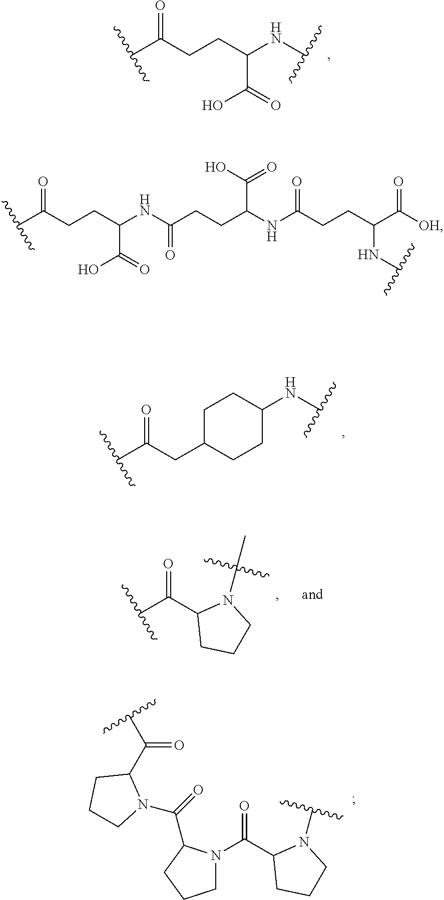 [SMILES: *NC(CCC(*)=O)C(=O)O ; *NC(CCC(=O)NC(CCC(=O)NC(CCC(*)=O)C(=O)O)C(=O)O)C(=O)O ; *NC1CCC(CC(*)=O)CC1 ; *C(=O)C1CCCN1* ; *C(=O)C1CCCN1C(=O)C1CCCN1C(=O)C1CCCN1*]

wherein the carbonyl group of L³ connects to an amine group of P² to form an amide bond;
D³ is —NR″-chelator;
R⁰ is an amino acid side chain of a natural amino acid or an amino acid side chain of an unnatural amino acid;
R¹ is an amino acid side chain of a natural amino acid or an amino acid side chain of an unnatural amino acid;
R² is an amino acid side chain of a natural amino acid or an amino acid side chain of an unnatural amino acid;
R³ is an amino acid side chain of a natural amino acid or an amino acid side chain of an unnatural amino acid;
B¹ is C₁₋₆ alkylene;
C¹ is C₁₋₆ alkylene;
A¹ is selected from:

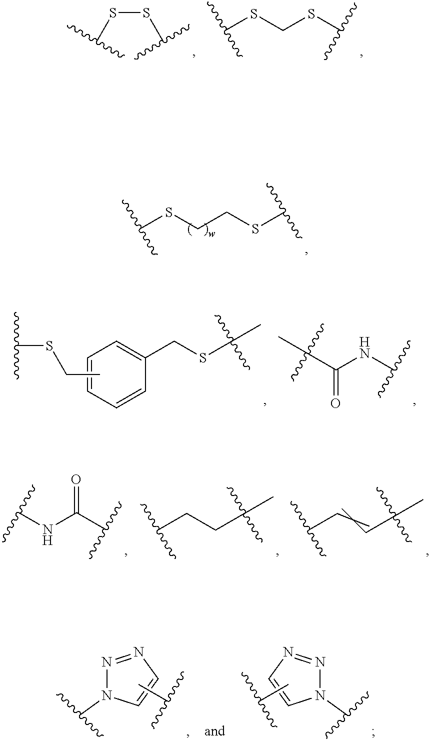

wherein w is selected from 1, 2, or 3;
R⁴ is an amino acid side chain of a natural amino acid or an amino acid side chain of an unnatural amino acid, both of which are optionally substituted with CH₂C(O)OH or C(O)(CH₂CH₂O)ₚ(CH₂)₂N(CH₃)₃⁺;
R⁵ is an amino acid side chain of a natural amino acid or an amino acid side chain of an unnatural amino acid;
R⁶ is an amino acid side chain of a natural amino acid or an amino acid side chain of an unnatural amino acid;
R⁷ is selected from:
(i) an amino acid side chain of a natural amino acid,
(ii) an amino acid side chain of an unnatural amino acid, or
(iii) selected from the group consisting of:

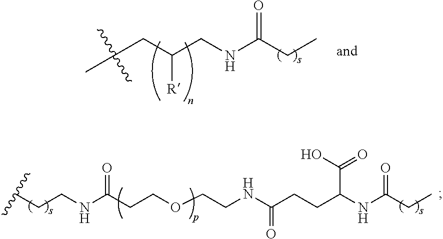

R⁸ is an amino acid side chain of a natural amino acid or an amino acid side chain of an unnatural amino acid;
R⁹ is an amino acid side chain of a natural amino acid or an amino acid side chain of an unnatural amino acid;
R¹⁰ is an amino acid side chain of a natural amino acid or an amino acid side chain of an unnatural amino acid;
m is 0 or 1;
each n, q, and u are independently an integer from 0 to 16;
each p is independently an integer from 0 to 24;
each s is independently an integer from 0 to 16;
each t is independently 1, 2, 3, 4, 5, or 6;
R′ is independently selected from H, C(O)OH, (CH₂)OH and NHAc; and
R″ is independently selected from H and CH₃.

In an embodiment, the peptide of Formula II is a peptide of Formula II-Ia comprising the amino acid sequence:

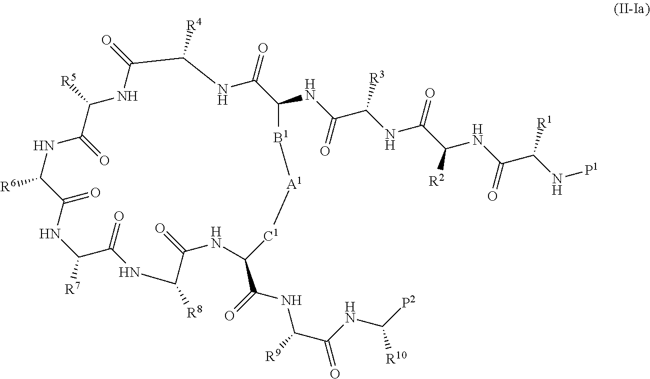

or a pharmaceutically acceptable salt thereof.

In another embodiment, the peptide of Formula II is a peptide of Formula II-Ib comprising the amino acid sequence:

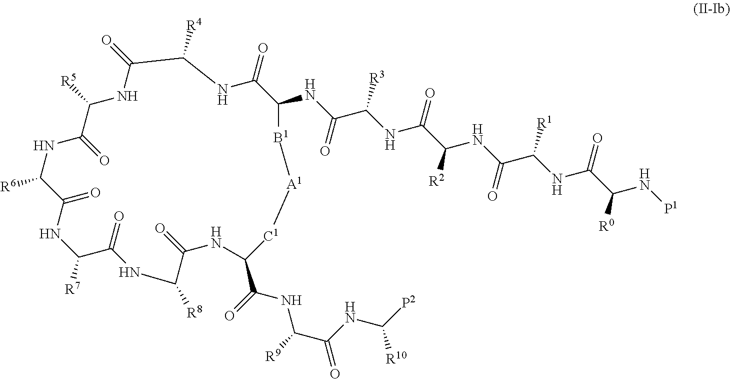

or a pharmaceutically acceptable salt thereof.

In yet another embodiment,
P¹ is selected from Ac,

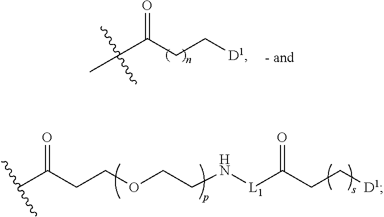

wherein:
D¹ is CH₃ or C(O)OH;
L¹ is absent or

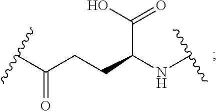 [SMILES: *N[C@@H](CCC(*)=O)C(=O)O]

n and s are independently an integer from 2 to 15; and
p is 8, 9, 10, 11, or 12.

In still another embodiment, P¹ is Ac or CH₃(OCH₂CH₂)₈₋₁₆C(O).

In an embodiment,
P¹ is selected from H, Ac,

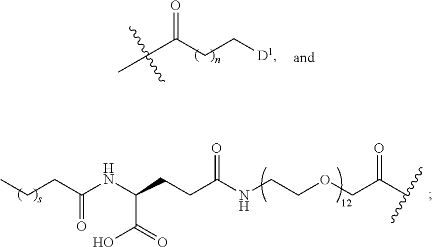

wherein:
n and s are independently 9, 10, 11, 12, or 13; and
D¹ is CH₃ or C(O)OH.

In another embodiment, P² is -L²-chelator.

In yet another embodiment, P² is

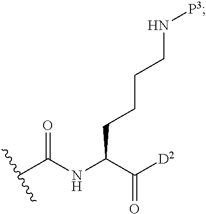

P³ is selected from DOTA,

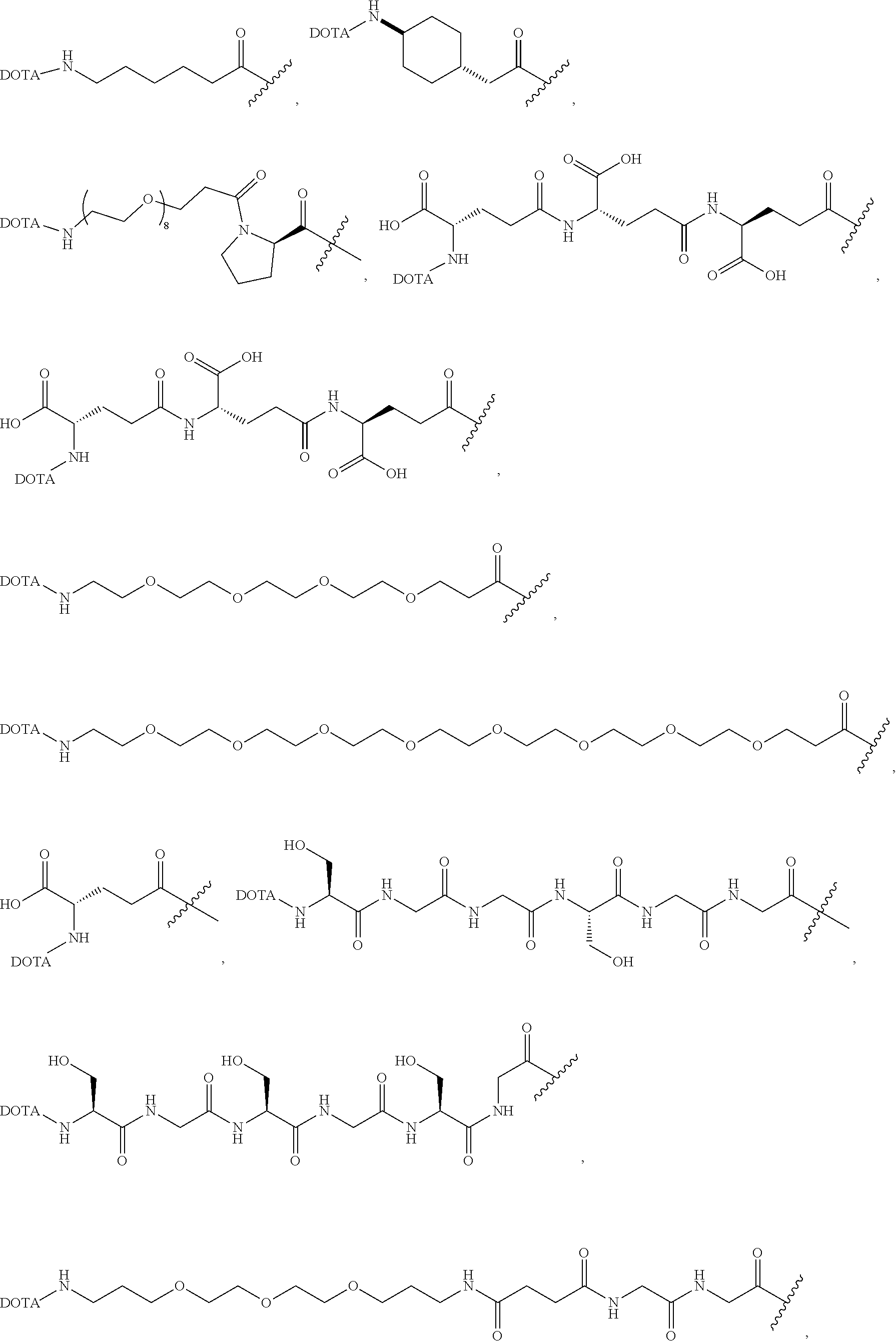

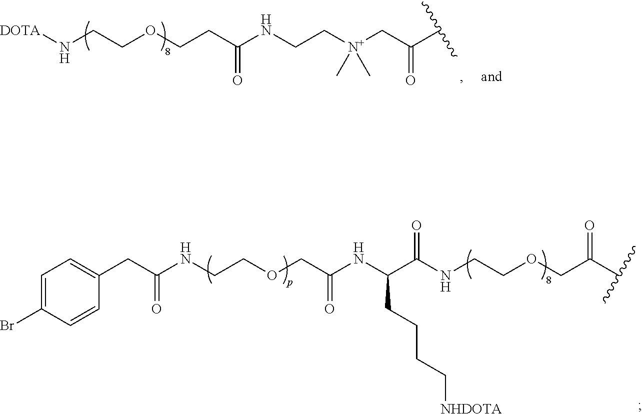

and
p is 8, 12, or 24.

In still another embodiment, P³ is -L³-chelator.

In an embodiment P³ is

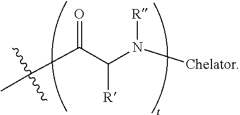

In another embodiment, D³ is-NR″-chelator.

In still another embodiment, the peptide of Formula II is a peptide of Formula II-II comprising the amino acid sequence:

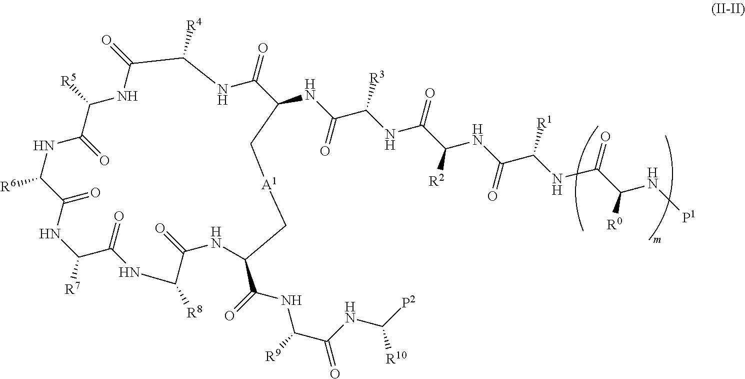

or a pharmaceutically acceptable salt thereof.

In an embodiment, the peptide of Formula II is a peptide of Formula II-III comprising the amino acid sequence:

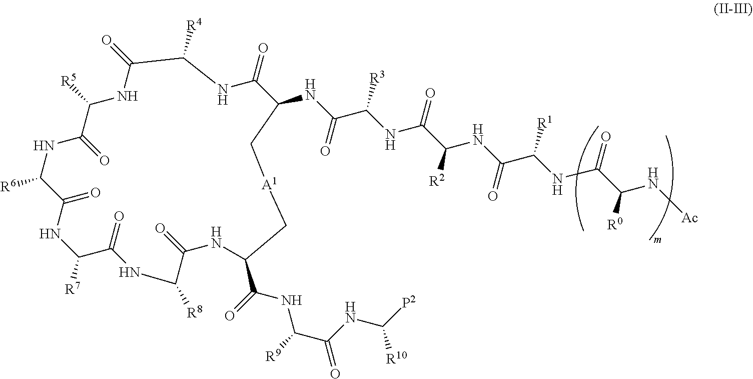

or a pharmaceutically acceptable salt thereof.

In yet another embodiment,
m is 0;
P¹ is selected from H,

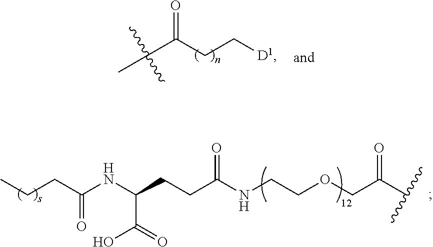

wherein:
n and s are each independently 9, 10, 11, 12, or 13;
P² is

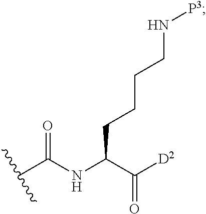

P³ is selected from DOTA,

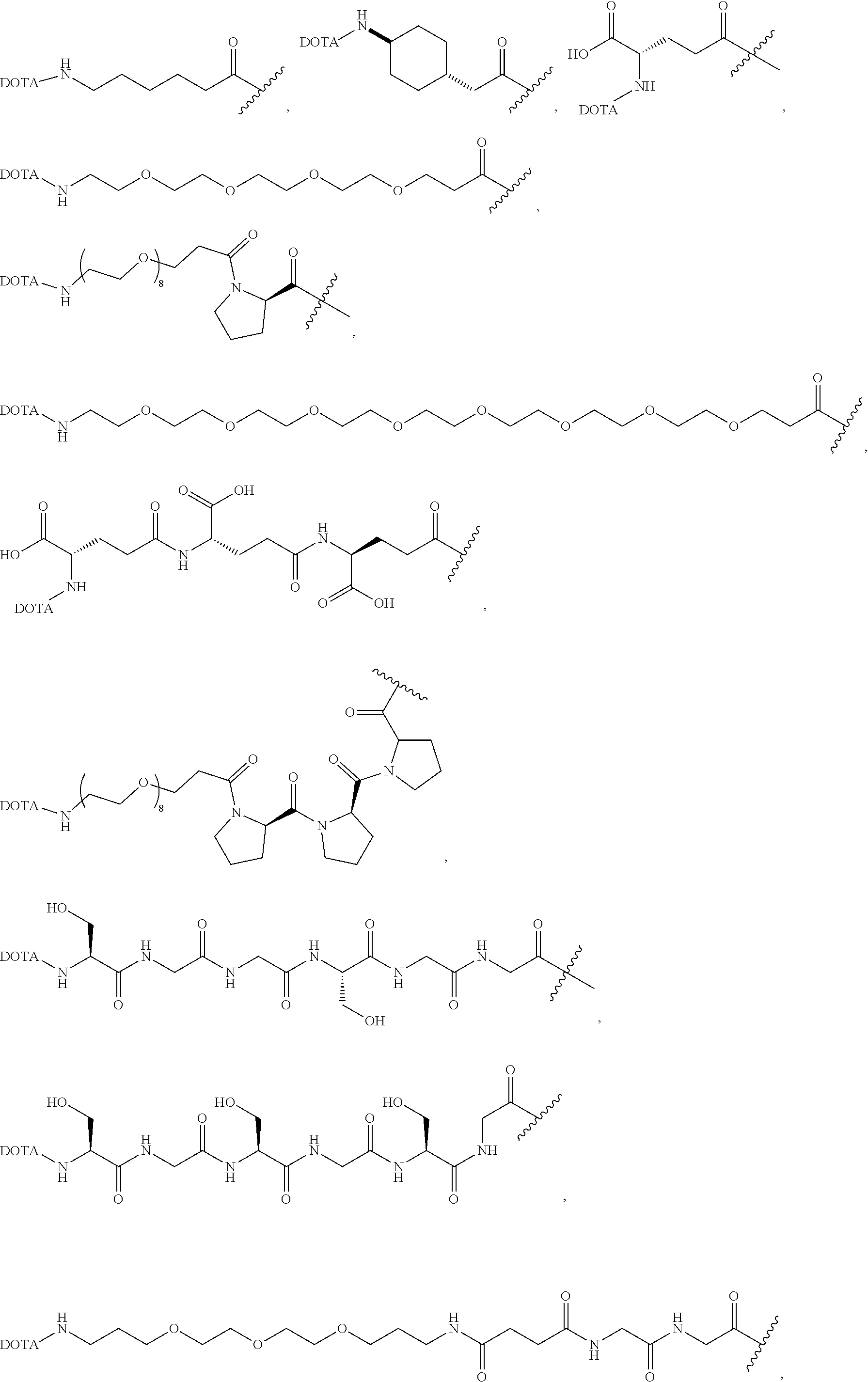

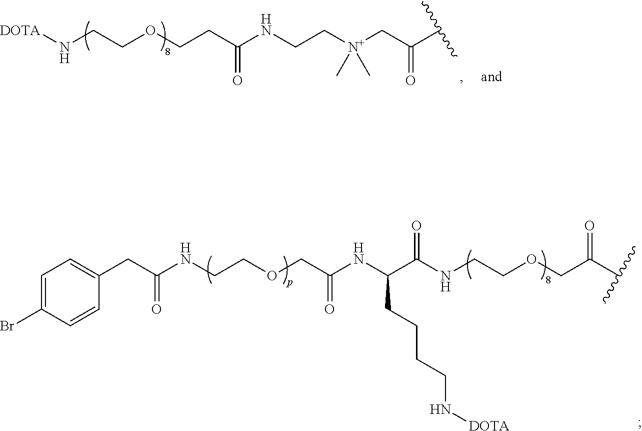

p is 8, 12, or 24;
D¹ is CH₃ or C(O)OH;
R¹ is an amino acid side chain selected from the group consisting of Trp, 2Nal, 1 Nal, 4CF₃-Phe, 7Aza-Trp, 1Me-Trp, 50H-Trp, BIP, 5OMe-Trp, 4F-Phe, 3Pya, 4Pya, PAF, MAF, OAF, 5Qui, 7MeO-Trp, 7Me-Trp, 5F-Trp, 7Cl-Trp, D-Ala, Ala, alpha-Me-Trp, and NMe-Trp;
R² is an amino acid side chain selected from the group consisting of Thr, D-Ala, Ala, alpha-Me-Thr, Lys, and NMe-Thr;
R³ an amino acid side chain selected from the group consisting of Ile, Env, CHA, CBA, Nle, Tbg, THPG, Chg, 2Nal, 1 Nal, 2CF₃-Phe, 2PhEt-Ala, D-Ala, Ala, Leu, t-Bu-Ala, NMe-Nle, α-tert-amylGly, Allo-Ile, Lys(C12), Lys(C14), Lys(C16), alpha-Me-Ile, and NMe-tBuAla;
A¹ is selected from the group consisting of:

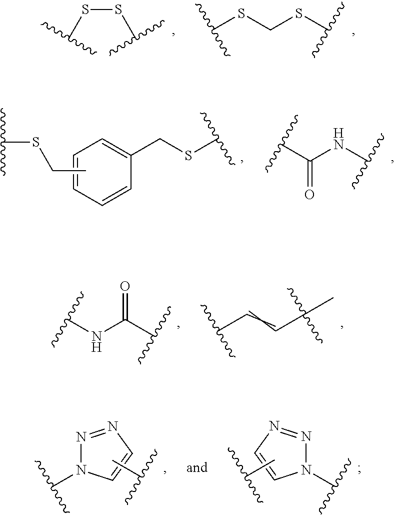

R⁴ is an amino acid side chain selected from the group consisting of Asn, D-Ala, Ala, DAB-4-NHCOC₅H₁₁, DAB-4-NHCOC₇H₁₅, Asp, Ser, Lys, 3-(4-piperidinyl)-Ala, 3-(1-morpholinyl)-Ala, 3Pya, 4Pya, Glu, NMe-Asn, Pip(CH₂CO₂H)Ala, and Pip(PegNMe₃)Ala;
R⁵ is an amino acid side chain selected from the group consisting of Asn, Ala, D-Ala, Trp, Asp, Lys, 3Pya, 4Pya, 3-(4-piperidinyl)-Ala, 3-(1-morpholinyl)-Ala, Glu, NMe-Asn, and Ser;
R⁶ is an amino acid side chain selected from the group consisting of Trp, 4CF₃-Phe, 1Me-Trp, 7Aza-Trp, BIP, 2Nal, 1Nal, alpha-Me-Trp, D-Ala, Ala, 4F-Phe, 1Nal, 2Nal, 5F-Trp, 5OMe-Trp, Asn, 50H-Trp, 7Me-Trp, 7MeO-Trp, 7Cl-Trp, and NMe-Trp;
R⁷ is:
(i) an amino acid side chain selected from the group consisting of 3Pya, 4Pya, Lys(Me)3, His, Ala, D-Ala, Gln, Lys, Glu, Arg, Orn, NMe-His, and Ser; or
(ii) selected from the group consisting of:

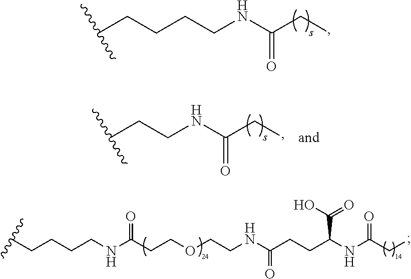

each s is independently 3, 5, 10, 12, or 14;
R⁸ is an amino acid side chain selected from the group consisting of Asp, D-Ala, Ala, Asn, Thr, NMe-Asp, and alpha-Me-Asp;
R⁹ is an amino acid side chain selected from the group consisting of Trp, 7Aza-Trp, 1Me-Trp, D-Ala, Ala, 4F-Phe, 1Nal, 2Nal, 5F-Trp, 5OMe-Trp, alpha-Me-Trp, 7Cl-Trp, 50H-Trp, 7Me-Trp, 7MeO-Trp, and NMe-Trp; and
R¹⁰ is an amino acid side chain selected from the group consisting of Pro, D-Ala, Ala, alpha-Me-Pro, trans4Fluoro-Pro, cis4Fluoro-Pro, trans40H-Pro, cis40H-Pro, Pip, 5,5-diMe-Pro, NMe-Ser, trans4NH2-Pro, cis4NH2-Pro, Sar, Aze, NMe-Ala, NMe-Leu, R-3Me-Aze, alpha-Me-Aze, ACl, and 3Me2-Aze.

In another embodiment,
m is 1;
P¹ is selected from Ac,

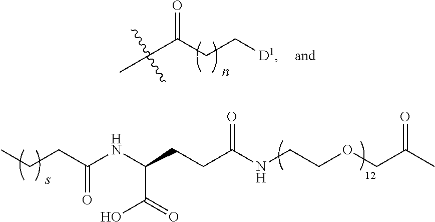

wherein:
n and s are each independently 9, 10, 11, 12, or 13;
P² is

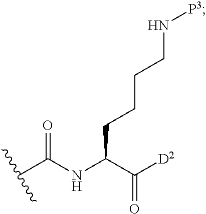

P³ is selected from DOTA,

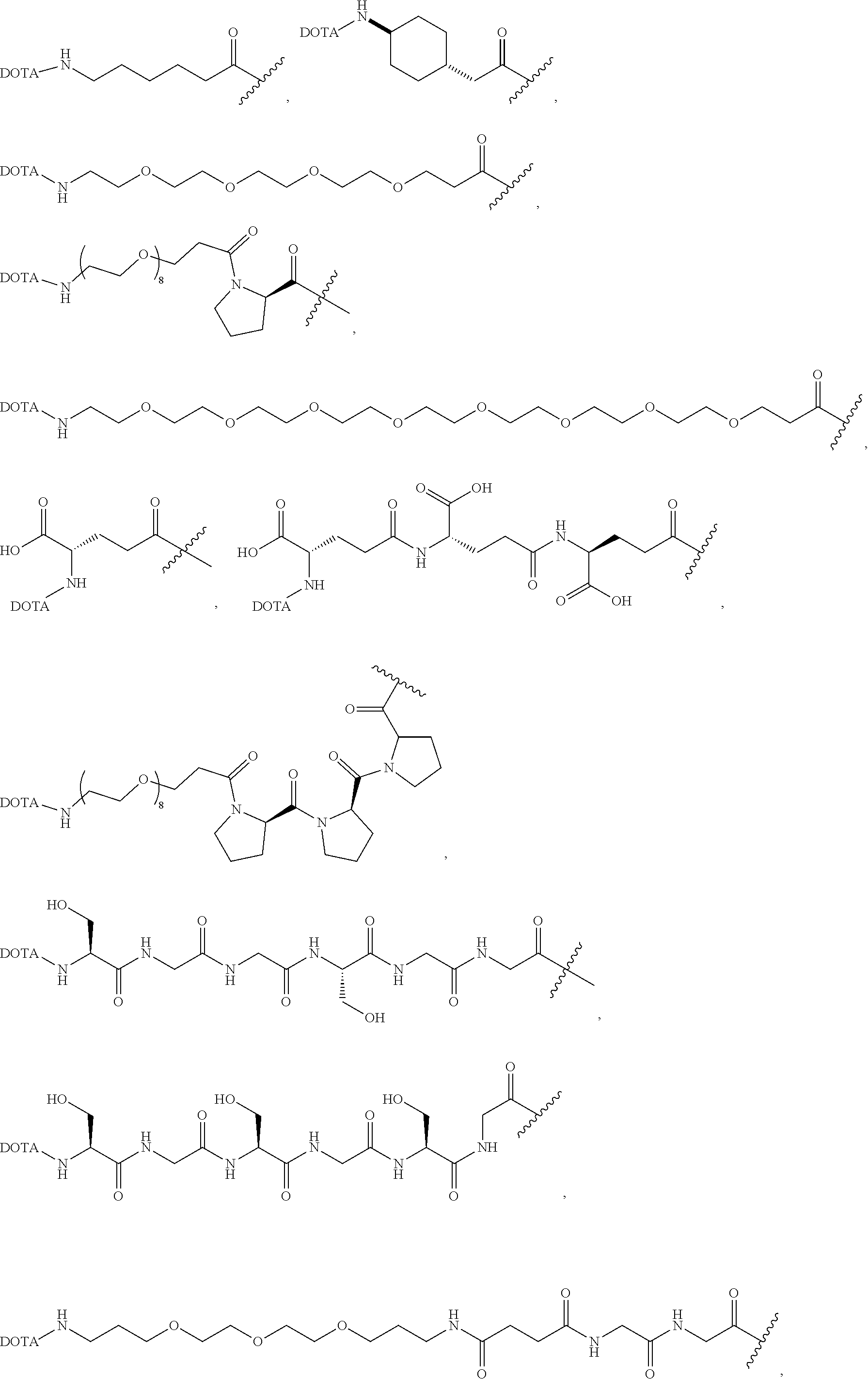

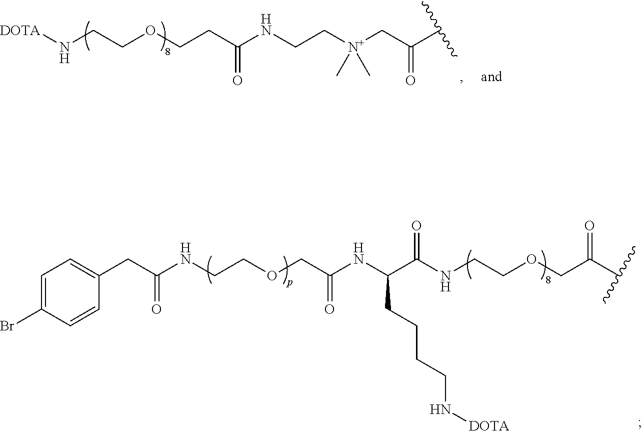

wherein
p is 8, 12, or 24;
D¹ is CH₃ or C(O)OH;
R⁰ is an amino acid side chain selected from the group consisting of Gly, Met, D-Ala, Ala, Nle, and Nva;
R¹ is an amino acid side chain selected from the group consisting of Trp, 2Nal, 1 Nal, 4CF₃-Phe, 7Aza-Trp, 1Me-Trp, 50H-Trp, BIP, 5OMe-Trp, 4F-Phe, 3Pya, 4Pya, PAF, MAF, OAF, 5Qui, 7MeO-Trp, 7Me-Trp, 5F-Trp, 7Cl-Trp, D-Ala, Ala, alpha-Me-Trp, and NMe-Trp;
R² is an amino acid side chain selected from the group consisting of Thr, D-Ala, Ala, alpha-Me-Thr, Lys, and NMe-Thr;
R³ is an amino acid side chain selected from the group consisting of Ile, Env, CHA, CBA, Nle, Tbg, THPG, Chg, 2Nal, 1Nal, 2CF₃-Phe, 2PhEt-Ala, D-Ala, Ala, Leu, t-Bu-Ala, NMe-Nle, α-tert-amylGly, Allo-Ile, Lys(C12), Lys(C14), Lys(C16), alpha-Me-Ile, and NMe-tBuAla;
A¹ is selected from the group consisting of

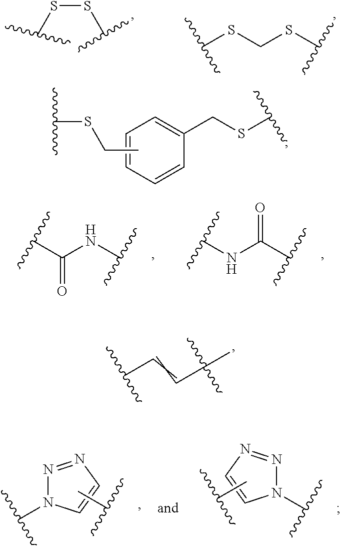

R⁴ is an amino acid side chain selected from the group consisting of Asn, D-Ala, Ala, DAB-4-NHCOC₅H₁₁, DAB-4-NHCOC₇H₁₅, Asp, SerLys, 3-(4-piperidinyl)-Ala, 3-(1-morpholinyl)-Ala, 3Pya, 4Pya, Glu, and NMe-Asn;
R⁵ is an amino acid side chain selected from the group consisting of Asn, Ala, D-Ala, Trp, Asp, Lys, 3Pya, 4Pya, 3-(4-piperidinyl)-Ala, 3-(1-morpholinyl)-Ala, Glu, NMe-Asn, and Ser;
R⁶ is an amino acid side chain selected from the group consisting of Trp, 4CF₃-Phe, 1Me-Trp, 7Aza-Trp, BIP, 2Nal, 1Nal, alpha-Me-Trp, D-Ala, Ala, 4F-Phe, 1Nal, 2Nal, 5F-Trp, 5OMe-Trp, Asn, 50H-Trp, 7Me-Trp, 7MeO-Trp, 7Cl-Trp, and NMe-Trp;
R⁷ is an amino acid side chain selected from the group consisting of 3Pya, 4Pya, Lys(Me)3, His, Ala, D-Ala, Gln, Lys, Glu, Arg, Orn, NMe-His, and Ser; or
R⁷ is selected from the group consisting of

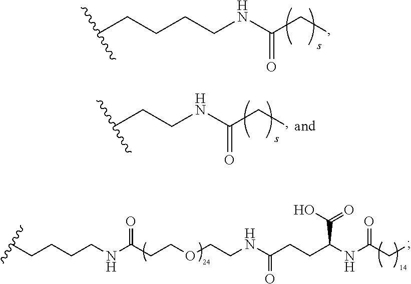

each s is independently 3, 5, 10, 12, or 14;
R⁸ is an amino acid side chain selected from the group consisting of Asp, D-Ala, Ala, Asn, Thr, NMe-Asp, and alpha-Me-Asp;
R⁹ is an amino acid side chain selected from the group consisting of Trp, 7Aza-Trp, 1Me-Trp, D-Ala, Ala, 4F-Phe, 1Nal, 2Nal, 5F-Trp, 5OMe-Trp, alpha-Me-Trp, 7Cl-Trp, 50H-Trp, 7Me-Trp, 7MeO-Trp, and NMe-Trp; and
R¹⁰ is an amino acid side chain selected from the group consisting of Pro, D-Ala, Ala, alpha-Me-Pro, trans4Fluoro-Pro, cis4Fluoro-Pro, trans40H-Pro, cis40H-Pro, Pip, 5,5-diMe-Pro, NMe-Ser, trans4NH2-Pro, cis4NH2-Pro, Sar, Aze, NMe-Ala, NMe-Leu, R-3Me-Aze, alpha-Me-Aze, ACl, and 3Me2-Aze.

In yet another embodiment,
B¹ is CH₂ or C(CH₃)₂; and
C¹ is CH₂ or C(CH₃)₂.

In still another embodiment,
B¹ is CH₂; and
C¹ is CH₂.

In an embodiment, chelator is independently selected from the group consisting of ethylenediamine tetraacetic acid (EDTA), diethylenetriamine pentaacetic acid (DTPA), 1,4,7,10-tetra-azacylcododecane-N,N′,N″,N′″-tetraacetic acid (DOTA), 6-((16-((6-Carboxypyridin-2-yl)methyl)-1,4,10,13-tetraoxa-7,16-diazacyclooctadecan-7-yl)methyl)-4-isothiocyanatopicolinic acid (Macropa), Macrodipa, 2,2′,2″,2′″-(1,10-dioxa-4,7,13,16-tetraazacyclooctadecane-4,7,13,16-tetrayl)tetraacetic acid) (Crown), 1,4,7,10-Tetraazacyclododecane-1,4,7,10-tetraacetic acid, α-(2-carboxyethyl) (DOTAGA), 1,4,7-Triazacyclononane-N,N′,N″-triacetic acid (NOTA), 1,4,7,10-tetraazacyclododecane-N,N′,N″,N′″-tetraacetic acid (TETA), 1,4,7,10,13-pentaazacyclopentadecane-N,N′,N″,N′″,N″″-pentaacetic acid (PEPA), and 1,4,7,10,13,16-hexaazacyclohexadecane-N,N′,N″,N′″,N″″,N′″″-hexaacetic acid (HEHA).

In another embodiment, chelator is DOTA. In yet another embodiment, chelator is DOTAGA. In still another embodiment, chelator is macrodipa. In an embodiment, chelator is macropa.

In another embodiment, Formula II is substituted by at least one chelator. In yet another embodiment, Formula I is substituted by one chelator. In still another embodiment, Formula II is substituted by at two chelators.

In an embodiment, the peptide comprises the amino acid sequence:

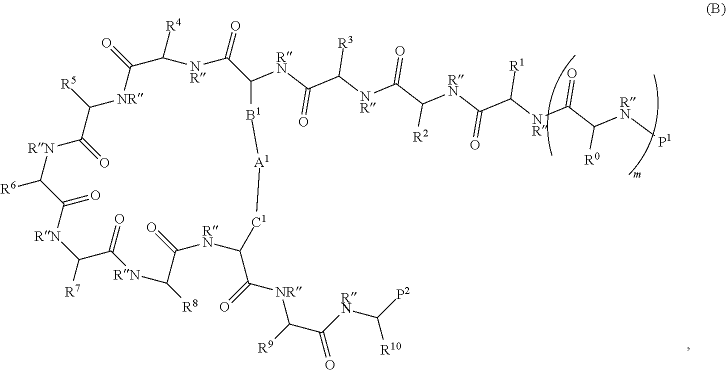

or a pharmaceutically acceptable salt thereof,
wherein the variable definitions are the same as those defined above for Formula II;
wherein:
each alpha-carbon atom on the peptide backbone is optionally substituted with methyl;
when a variable group R⁰, R¹, R², R³, R⁴, R⁵, R⁶, R⁷, R⁸, R⁹, or R¹⁰ is the side chain of a cyclic amino acid, the corresponding amino acid nitrogen of the peptide backbone forms part of the cyclic group; and
wherein the cyclic peptide optionally comprises a radionuclide.

In an embodiment, the peptide of Formula II is selected from a peptide in Table 3. In an embodiment, the peptide of Formula B is selected from a peptide in Table 3.

TABLE 3

| Cpd# | Sequence |
|---|---|
| 23 | Ac-W-T-I-C(3)-N-N-W-H-D-C(3)-W-P-[D-Lys(DOTA)]-NH2 |
| 47 | Ac-W-T-I-C(3)-N-N-W-H-D-C(3)-W-P-(D-Lys)-Ahx-DOTA-NH2 |
| 48 | Ac-W-T-I-C(3)-N-N-W-H-D-C(3)-W-P-(D-Lys)-CyHex-DOTA-NH2 |
| 74 | Ac-W-T-I-C(3)-N-N-(1Nal)-H-D-C(3)-W-P-[D-Lys(DOTA)]-NH2 |
| 75 | Ac-W-T-I-C(3)-N-N-(2Nal)-H-D-C(3)-W-P-[D-Lys(DOTA)]-NH2 |
| 76 | Ac-W-T-I-C(3)-N-N-W-H-D-C(3)-(1Nal)-P-[D-Lys(DOTA)]-NH2 |
| 77 | Ac-W-T-I-C(3)-N-N-W-H-D-C(3)-(2Nal)-P-[D-Lys(DOTA)]-NH2 |
| 85 | Ac-W-T-I-C(3)-N-N-(7Aza-W)-H-D-C(3)-W-P-[D-Lys(DOTA)]-NH2 |
| 86 | Ac-W-T-I-C(3)-N-N-(5OMe-W)-H-D-C(3)-W-P-[D-Lys(DOTA)]-NH2 |
| 87 | Ac-W-T-I-C(3)-N-N-W-H-D-C(3)-(7Aza-W)-P-[D-Lys(DOTA)]-NH2 |
| 88 | Ac-W-T-I-C(3)-N-N-W-H-D-C(3)-(5OMe-W)-P-[D-Lys(DOTA)]-NH2 |
| 89 | Ac-W-T-I-C(3)-N-N-W-H-D-C(3)-W-P-(D-Lys)-PEG8-DOTA-NH2 |
| 90 | Ac-W-T-I-C(3)-(DAB-4-NHCOC5H11)-N-W-H-D-C(3)-W-P-[D-Lys(DOTA)]-NH2 |
| 91 | Ac-W-T-I-C(3)-(DAB-4-NHCOC7H15)-N-W-H-D-C(3)-W-P-[D-Lys(DOTA)]-NH2 |
| 92 | Ac-W-T-I-C(3)-N-N-W-(DAB-4-NHCOC5H11)-D-C(3)-W-P-[D-Lys(DOTA)]-NH2 |
| 93 | Ac-W-T-I-C(3)-N-N-W-(DAB-4-NHCOC7H15)-D-C(3)-W-P-[D-Lys(DOTA)]-NH2 |
| 100 | Ac-(5Fluoro-W)-T-I-C(3)-N-N-W-H-D-C(3)-W-P-[D-Lys(DOTA)]-NH2 |
| 101 | Ac-W-T-I-C(3)-N-N-(5Fluoro-W)-H-D-C(3)-W-P-[D-Lys(DOTA)]-NH2 |
| 105 | Ac-W-T-I-C(3)-N-N-W-H-D-C(3)-W-(aMe-P)-[D-Lys(DOTA)]-NH2 |
| 106 | Ac-W-T-(Chg)-C(3)-N-N-W-H-D-C(3)-W-P-[D-Lys(DOTA)]-NH2 |
| 107 | Ac-W-T-(1Nal)-C(3)-N-N-W-H-D-C(3)-W-P-[D-Lys(DOTA)]-NH2 |
| 108 | Ac-W-T-I-C(3)-N-N-W-H-D-C(3)-W-(Pip)-[D-Lys(DOTA)]-NH2 |
| 109 | Ac-W-T-I-C(3)-N-N-W-H-D-C(3)-W-(5,5-diMe-P)-[D-Lys(DOTA)]-NH2 |
| 110 | Ac-W-T-I-C(3)-N-N-W-H-D-C(3)-W-(cis4OH-P)-[D-Lys(DOTA)]-NH2 |
| 111 | Ac-W-T-I-C(3)-N-N-W-H-D-C(3)-W-(trans4OH-P)-[D-Lys(DOTA)]-NH2 |
| 112 | Ac-W-T-I-C(3)-N-N-W-H-D-C(3)-W-(Cis4Fluoro-P)-[D-Lys(DOTA)]-NH2 |
| 113 | Ac-W-T-I-C(3)-N-N-W-H-D-C(3)-W-(trans4Fluoro-P)-[D-Lys(DOTA)]-NH2 |
| 114 | Ac-W-T-(2CF3-F)-C(3)-N-N-W-H-D-C(3)-W-P-[D-Lys(DOTA)]-NH2 |
| 115 | Ac-W-T-(2PhEt-A)-C(3)-N-N-W-H-D-C(3)-W-P-[D-Lys(DOTA)]-NH2 |
| 116 | Ac-W-T-(2Nal)-C(3)-N-N-W-H-D-C(3)-W-P-[D-Lys(DOTA)]-NH2 |
| 117 | Ac-W-T-I-C(3)-N-N-W-H-D-C(3)-(aMe-W)-P-[D-Lys(DOTA)]-NH2 |
| 118 | Ac-W-T-I-C(3)-N-N-(aMe-W)-H-D-C(3)-W-P-[D-Lys(DOTA)]-NH2 |
| 119 | C15-(L-gE)-PEG12-W-T-I-C(3)-N-N-W-H-D-C(3)-W-P-[D-Lys(DOTA)]-NH2 |
| 121 | Ac-(5Fluoro-W)-T-(Tbg)-C(3)-N-N-(4Fluoro-F)-H-D-C(3)-(7Aza-W)-(Aze)-[D- |
| | Lys(DOTA)]-NH2 |
| 122 | Ac-(5Fluoro-W)-T-(Env)-C(3)-N-N-(4Fluoro-F)-H-D-C(3)-(7Aza-W)-(Aze)-[D- |
| | Lys(DOTA)]-NH2 |
| 123 | Ac-(5Fluoro-W)-T-(THPG)-C(3)-N-N-(4Fluoro-F)-H-D-C(3)-(7Aza-W)-(Aze)-[D- |
| | Lys(DOTA)]-NH2 |
| 124 | Ac-(5Fluoro-W)-T-(Nle)-C(3)-N-N-(4Fluoro-F)-H-D-C(3)-(7Aza-W)-(Aze)-[D- |
| | Lys(DOTA)]-NH2 |
| 125 | Ac-(5Fluoro-W)-T-(CHA)-C(3)-N-N-(4Fluoro-F)-H-D-C(3)-(7Aza-W)-(Aze)-[D- |
| | Lys(DOTA)]-NH2 |
| 126 | Ac-(5Fluoro-W)-T-(CBA)-C(3)-N-N-(4Fluoro-F)-H-D-C(3)-(7Aza-W)-(Aze)-[D- |
| | Lys(DOTA)]-NH2 |
| 127 | Ac-(5Fluoro-W)-T-(Chg)-C(3)-N-N-(4Fluoro-F)-H-D-C(3)-(7Aza-W)-(Aze)-[D- |
| | Lys(DOTA)]-NH2 |
| 128 | Ac-(5Fluoro-W)-T-(2CF3-F)-C(3)-N-N-(4Fluoro-F)-H-D-C(3)-(7Aza-W)-(Aze)-[D- |
| | Lys(DOTA)]-NH2 |
| 129 | Ac-(5Fluoro-W)-T-(Tbg)-C(3)-N-N-(4Fluoro-F)-H-D-C(3)-(7Aza-W)-(trans4Fluoro- |
| | P)-[D-Lys(DOTA)]-NH2 |
| 130 | Ac-(5Fluoro-W)-T-(Env)-C(3)-N-N-(4Fluoro-F)-H-D-C(3)-(7Aza-W)-(trans4Fluoro- |
| | P)-[D-Lys(DOTA)]-NH2 |
| 131 | Ac-(5Fluoro-W)-T-(THPG)-C(3)-N-N-(4Fluoro-F)-H-D-C(3)-(7Aza-W)- |
| | (trans4Fluoro-P)-[D-Lys(DOTA)]-NH2 |
| 132 | Ac-(5Fluoro-W)-T-(Nle)-C(3)-N-N-(4Fluoro-F)-H-D-C(3)-(7Aza-W)-(trans4Fluoro- |
| | P)-[D-Lys(DOTA)]-NH2 |
| 133 | Ac-(5Fluoro-W)-T-(CHA)-C(3)-N-N-(4Fluoro-F)-H-D-C(3)-(7Aza-W)-(trans4Fluoro- |
| | P)-[D-Lys(DOTA)]-NH2 |
| 134 | Ac-(5Fluoro-W)-T-(CBA)-C(3)-N-N-(4Fluoro-F)-H-D-C(3)-(7Aza-W)-(trans4Fluoro- |
| | P)-[D-Lys(DOTA)]-NH2 |
| 135 | Ac-(5Fluoro-W)-T-(Chg)-C(3)-N-N-(4Fluoro-F)-H-D-C(3)-(7Aza-W)-(trans4Fluoro- |
| | P)-[D-Lys(DOTA)]-NH2 |
| 136 | Ac-(5Fluoro-W)-T-(2CF3-F)-C(3)-N-N-(4Fluoro-F)-H-D-C(3)-(7Aza-W)- |
| | (trans4Fluoro-P)-[D-Lys(DOTA)]-NH2 |
| 137 | Ac-(5Fluoro-W)-T-(Tbg)-C(3)-N-N-(4CF3-F)-H-D-C(3)-(7Aza-W)-(Aze)-[D- |
| | Lys(DOTA)]-NH2 |
| 138 | Ac-(5Fluoro-W)-T-(Env)-C(3)-N-N-(4CF3-F)-H-D-C(3)-(7Aza-W)-(Aze)-[D- |
| | Lys(DOTA)]-NH2 |
| 139 | Ac-(5Fluoro-W)-T-(THPG)-C(3)-N-N-(4CF3-F)-H-D-C(3)-(7Aza-W)-(Aze)-[D- |
| | Lys(DOTA)]-NH2 |
| 140 | Ac-(5Fluoro-W)-T-(Nle)-C(3)-N-N-(4CF3-F)-H-D-C(3)-(7Aza-W)-(Aze)-[D- |
| | Lys(DOTA)]-NH2 |
| 141 | Ac-(5Fluoro-W)-T-(CHA)-C(3)-N-N-(4CF3-F)-H-D-C(3)-(7Aza-W)-(Aze)-[D- |
| | Lys(DOTA)]-NH2 |
| 142 | Ac-(5Fluoro-W)-T-(CBA)-C(3)-N-N-(4CF3-F)-H-D-C(3)-(7Aza-W)-(Aze)-[D- |
| | Lys(DOTA)]-NH2 |
| 143 | Ac-(5Fluoro-W)-T-(Chg)-C(3)-N-N-(4CF3-F)-H-D-C(3)-(7Aza-W)-(Aze)-[D- |
| | Lys(DOTA)]-NH2 |
| 144 | Ac-(5Fluoro-W)-T-(2CF3-F)-C(3)-N-N-(4CF3-F)-H-D-C(3)-(7Aza-W)-(Aze)-[D- |
| | Lys(DOTA)]-NH2 |
| 145 | Ac-(5Fluoro-W)-T-(Tbg)-C(3)-N-N-(4CF3-F)-H-D-C(3)-(7Aza-W)-(trans4Fluoro-P)- |
| | [D-Lys(DOTA)]-NH2 |
| 146 | Ac-(5Fluoro-W)-T-(Env)-C(3)-N-N-(4CF3-F)-H-D-C(3)-(7Aza-W)-(trans4Fluoro-P)- |
| | [D-Lys(DOTA)]-NH2 |
| 147 | Ac-(5Fluoro-W)-T-(THPG)-C(3)-N-N-(4CF3-F)-H-D-C(3)-(7Aza-W)-(trans4Fluoro- |
| | P)-[D-Lys(DOTA)]-NH2 |
| 148 | Ac-(5Fluoro-W)-T-(Nle)-C(3)-N-N-(4CF3-F)-H-D-C(3)-(7Aza-W)-(trans4Fluoro-P)- |
| | [D-Lys(DOTA)]-NH2 |
| 149 | Ac-(5Fluoro-W)-T-(CHA)-C(3)-N-N-(4CF3-F)-H-D-C(3)-(7Aza-W)-(trans4Fluoro-P)- |
| | [D-Lys(DOTA)]-NH2 |
| 150 | Ac-(5Fluoro-W)-T-(CBA)-C(3)-N-N-(4CF3-F)-H-D-C(3)-(7Aza-W)-(trans4Fluoro-P)- |
| | [D-Lys(DOTA)]-NH2 |
| 151 | Ac-(5Fluoro-W)-T-(Chg)-C(3)-N-N-(4CF3-F)-H-D-C(3)-(7Aza-W)-(trans4Fluoro-P)- |
| | [D-Lys(DOTA)]-NH2 |
| 152 | Ac-(5Fluoro-W)-T-(2CF3-F)-C(3)-N-N-(4CF3-F)-H-D-C(3)-(7Aza-W)-(trans4Fluoro- |
| | P)-[D-Lys(DOTA)]-NH2 |
| 156 | Ac-(7-Me-W)-T-I-C(3)-N-N-W-H-D-C(3)-W-P-[D-Lys(DOTA)]-NH2 |
| 157 | Ac-W-T-I-C(3)-N-N-W-H-D-C(3)-W-NMeS-[D-Lys(DOTA)]-NH2 |
| 158 | Ac-W-T-I-C(3)-N-N-W-K(C12)-D-C(3)-W-P-[D-Lys(DOTA)]-NH2 |
| 159 | Ac-W-T-I-C(3)-N-N-W-K(C14)-D-C(3)-W-P-[D-Lys(DOTA)]-NH2 |
| 160 | Ac-W-T-I-C(3)-N-N-W-K(C16)-D-C(3)-W-P-[D-Lys(DOTA)]-NH2 |
| 161 | C14-W-T-I-C(3)-N-N-W-H-D-C(3)-W-P-[D-Lys(DOTA)]-NH2 |
| 162 | C16-W-T-I-C(3)-N-N-W-H-D-C(3)-W-P-[D-Lys(DOTA)]-NH2 |
| 164 | Ac-WTI-C(1)-NNWHD-C(1)-WP-[D-Lys(DOTA)]-NH2 |
| 165 | Ac-WTI-C(1)-NN(2Nal)HD-C(1)-WP-[D-Lys(DOTA)]-NH2 |
| 166 | Ac-W-T-I-C(3)-N-N-(2Nal)-H-D-C(3)-W-P-[L-Lys(DOTA)]-NH2 |
| 167 | Ac-W-T-I-C(3)-N-N-(2Nal)-H-D-C(3)-W-P-G-G-TTDS-[D-Lys(DOTA)]-NH2 |
| 168 | Ac-(5Fluoro-W)-T-(Tbg)-C(3)-N-N-(1Nal)-H-D-C(3)-(7Aza-W)-(Aze)-[D- |
| | Lys(DOTA)]-NH2 |
| 169 | Ac-(5Fluoro-W)-T-(THPG)-C(3)-N-N-(1Nal)-H-D-C(3)-(7Aza-W)-(Aze)-[D- |
| | Lys(DOTA)]-NH2 |
| 170 | Ac-(5Fluoro-W)-T-(Nle)-C(3)-N-N-(1Nal)-H-D-C(3)-(7Aza-W)-(Aze)-[D- |
| | Lys(DOTA)]-NH2 |
| 171 | Ac-(5Fluoro-W)-T-(CHA)-C(3)-N-N-(1Nal)-H-D-C(3)-(7Aza-W)-(Aze)-[D- |
| | Lys(DOTA)]-NH2 |
| 172 | Ac-(5Fluoro-W)-T-(CBA)-C(3)-N-N-(1Nal)-H-D-C(3)-(7Aza-W)-(Aze)-[D- |
| | Lys(DOTA)]-NH2 |
| 173 | Ac-(5Fluoro-W)-T-(Chg)-C(3)-N-N-(1Nal)-H-D-C(3)-(7Aza-W)-(trans4Fluoro-P)-[D- |
| | Lys(DOTA)]-NH2 |
| 174 | Ac-(5Fluoro-W)-T-(2CF3-F)-C(3)-N-N-(1Nal)-H-D-C(3)-(7Aza-W)-(trans4Fluoro-P)- |
| | [D-Lys(DOTA)]-NH2 |
| 175 | Ac-(5Fluoro-W)-T-(Chg)-C(3)-N-N-(1Nal)-H-D-C(3)-(7Aza-W)-(Aze)-[D- |
| | Lys(DOTA)]-NH2 |
| 176 | Ac-(5Fluoro-W)-T-(2CF3-F)-C(3)-N-N-(1Nal)-H-D-C(3)-(7Aza-W)-(Aze)-[D- |
| | Lys(DOTA)]-NH2 |
| 177 | Ac-(5Fluoro-W)-T-(Tbg)-C(3)-N-N-(1Nal)-H-D-C(3)-(7Aza-W)-(trans4Fluoro-P)-[D- |
| | Lys(DOTA)]-NH2 |
| 178 | Ac-(5Fluoro-W)-T-(Env)-C(3)-N-N-(1Nal)-H-D-C(3)-(7Aza-W)-(trans4Fluoro-P)-[D- |
| | Lys(DOTA)]-NH2 |
| 179 | Ac-(5Fluoro-W)-T-(THPG)-C(3)-N-N-(1Nal)-H-D-C(3)-(7Aza-W)-(trans4Fluoro-P)- |
| | [D-Lys(DOTA)]-NH2 |
| 180 | Ac-(5Fluoro-W)-T-(Nle)-C(3)-N-N-(1Nal)-H-D-C(3)-(7Aza-W)-(trans4Fluoro-P)-[D- |
| | Lys(DOTA)]-NH2 |
| 181 | Ac-(5Fluoro-W)-T-(CHA)-C(3)-N-N-(1Nal)-H-D-C(3)-(7Aza-W)-(trans4Fluoro-P)-[D- |
| | Lys(DOTA)]-NH2 |
| 182 | Ac-(5Fluoro-W)-T-(CBA)-C(3)-N-N-(1Nal)-H-D-C(3)-(7Aza-W)-(trans4Fluoro-P)-[D- |
| | Lys(DOTA)]-NH2 |
| 183 | Ac-(5Fluoro-W)-T-(Tbg)-C(3)-N-N-(2Nal)-H-D-C(3)-(7Aza-W)-(Aze)-[D- |
| | Lys(DOTA)]-NH2 |
| 184 | Ac-(5Fluoro-W)-T-(Nle)-C(3)-N-N-(2Nal)-H-D-C(3)-(7Aza-W)-(Aze)-[D- |
| | Lys(DOTA)]-NH2 |
| 185 | Ac-(5Fluoro-W)-T-(CBA)-C(3)-N-N-(2Nal)-H-D-C(3)-(7Aza-W)-(Aze)-[D- |
| | Lys(DOTA)]-NH2 |
| 186 | Ac-(5Fluoro-W)-T-(Env)-C(3)-N-N-(2Nal)-H-D-C(3)-(7Aza-W)-(Aze)-[D- |
| | Lys(DOTA)]-NH2 |
| 187 | Ac-(5Fluoro-W)-T-(Chg)-C(3)-N-N-(2Nal)-H-D-C(3)-(7Aza-W)-(Aze)-[D- |
| | Lys(DOTA)]-NH2 |
| 188 | Ac-(5Fluoro-W)-T-(THPG)-C(3)-N-N-(2Nal)-H-D-C(3)-(7Aza-W)-(Aze)-[D- |
| | Lys(DOTA)]-NH2 |
| 189 | Ac-(5Fluoro-W)-T-(CHA)-C(3)-N-N-(2Nal)-H-D-C(3)-(7Aza-W)-(Aze)-[D- |
| | Lys(DOTA)]-NH2 |
| 190 | Ac-(5Fluoro-W)-T-(2CF3-F)-C(3)-N-N-(2Nal)-H-D-C(3)-(7Aza-W)-(Aze)-[D- |
| | Lys(DOTA)]-NH2 |
| 193 | Ac-W-T-(Env)-C(3)-N-N-(2Nal)-H-D-C(3)-(7-Aza-W)-P-[D-Lys(DOTA)]-NH2 |
| 194 | Ac-W-T-(Chg)-C(3)-A-N-(2Nal)-H-D-C(3)-(7-Aza-W)-P-[D-Lys(DOTA)]-NH2 |
| 195 | Ac-W-T-(CHA)-C(3)-A-N-(2Nal)-H-D-C(3)-(7-Aza-W)-P-[D-Lys(DOTA)]-NH2 |
| 196 | Ac-W-T-(Env)-C(3)-A-N-(2Nal)-H-D-C(3)-(7-Aza-W)-P-[D-Lys(DOTA)]-NH2 |
| 197 | Ac-W-T-(Chg)-C(3)-N-A-(2Nal)-H-D-C(3)-(7-Aza-W)-P-[D-Lys(DOTA)]-NH2 |
| 198 | Ac-W-T-(CHA)-C(3)-N-A-(2Nal)-H-D-C(3)-(7-Aza-W)-P-[D-Lys(DOTA)]-NH2 |
| 199 | Ac-W-T-(Env)-C(3)-N-A-(2Nal)-H-D-C(3)-(7-Aza-W)-P-[D-Lys(DOTA)]-NH2 |
| 201 | Ac-M-W-T-L-C(3)-D-W-N-S-D-C(3)-W-P-[D-Lys(DOTA)]-NH2 |
| 202 | Ac-W-T-(Env)-C(3)-N-N-(2Nal)-H-D-C(3)-(7-Aza-W)-P-[D-Lys(Chx-DOTA)]-NH2 |
| 203 | Ac-W-T-(Chg)-C(3)-N-N-(2Nal)-H-D-C(3)-(7-Aza-W)-P-[D-Lys(Chx-DOTA)]-NH2 |
| 204 | Ac-W-T-(Env)-C(3)-N-N-(2Nal)-H-D-C(3)-(7-Aza-W)-P-[D-Lys(G-G-TTDS-DOTA)]- |
| | NH2 |
| 205 | Ac-W-(Lys)-I-C(3)-N-N-(2Nal)-H-D-C(3)-W-P-[D-Lys(DOTA)]-NH2 |
| 206 | Ac-(7-Me-W)-T-I-C(3)-N-N-(2Nal)-H-D-C(3)-W-P-[D-Lys(DOTA)]-NH2 |
| 207 | Ac-(7-Me-W)-T-I-C(3)-N-N-(2Nal)-H-D-C(3)-W-P-[D-Lys(gE-gE-gE-DOTA)]-NH2 |
| 208 | Ac-W-T-I-C(3)-(Ser)-N-(2Nal)-H-D-C(3)-W-P-[D-Lys(DOTA)]-NH2 |
| 209 | Ac-W-T-I-C(3)-N-(Asp)-(2Nal)-H-D-C(3)-W-P-[D-Lys(DOTA)]-NH2 |
| 210 | Ac-W-T-I-C(3)-N-N-(2Nal)-(Gln)-D-C(3)-W-P-[D-Lys(DOTA)]-NH2 |
| 211 | Ac-W-T-I-C(3)-N-N-(2Nal)-H-(Asn)-C(3)-W-P-[D-Lys(DOTA)]-NH2 |
| 212 | Ac-W-T-I-C(3)-(Ser)-(Asp)-(2Nal)-(Gln)-(Asn)-C(3)-W-P-[D-Lys(DOTA)]-NH2 |
| 213 | Ac-W-T-I-C(3)-N-(Lys)-(2Nal)-H-D-C(3)-W-P-[D-Lys(DOTA)]-NH2 |
| 226 | Ac-(7-CI-W)-T-I-C(3)-N-N-W-H-D-C(3)-W-P-[D-Lys(DOTA)]-NH2 |
| 227 | Ac-W-T-I-C(3)-N-N-W-H-D-C(3)-W-Sar-[D-Lys(DOTA)]-NH2 |
| 228 | Ac-W-T-I-C(3)-N-N-W-H-D-C(3)-W-Aze-[D-Lys(DOTA)]-NH2 |
| 229 | Ac-W-T-I-C(3)-N-N-W-H-D-C(3)-W-NMeA-[D-Lys(DOTA)]-NH2 |
| 230 | Ac-W-T-I-C(3)-N-N-(2Nal)-H-D-C(3)-W-P-[D-Lys(G-G-TTDS-DOTA)]-NH2 |
| 231 | Ac-W-T-I-C(3)-N-N-(2Nal)-H-D-C(3)-W-P-G-G-S-G-G-S-[D-Lys(DOTA)]-NH2 |
| 232 | Ac-W-T-I-C(3)-N-N-(2Nal)-H-D-C(3)-W-P-G-S-G-S-G-S-[D-Lys(DOTA)]-NH2 |
| 233 | Ac-W-T-I-C(3)-N-N-(2Nal)-H-D-C(3)-W-P-gE-[D-Lys(DOTA)]-NH2 |
| 234 | Ac-W-T-I-C(3)-N-N-(2Nal)-H-D-C(3)-W-P-gE-gE-gE-[D-Lys(DOTA)]-NH2 |
| 235 | Ac-W-T-I-C(3)-N-N-(2Nal)-H-D-C(3)-W-P-(D-Pro)-PEG8-[D-Lys(DOTA)]-NH2 |
| 237 | Ac-W-T-I-C(3)-N-N-(2Nal)-K-D-C(3)-W-P-[D-Lys(DOTA)]-NH2 |
| 238 | Ac-W-T-I-C(3)-N-N-(2Nal)-(K(Me)3)-D-C(3)-W-P-[D-Lys(DOTA)]-NH2 |
| 241 | Ac-(5Fluoro-W)-T-(Env)-C(3)-N-N-(1Nal)-H-D-C(3)-(7Aza-W)-(Aze)-[D- |
| | Lys(DOTA)]-NH2 |
| 242 | Ac-(5Fluoro-W)-T-(Env)-C(3)-N-N-(2Nal)-H-D-C(3)-(7Aza-W)-(trans4Fluoro-P)-[D- |
| | Lys(DOTA)]-NH2 |
| 243 | Ac-(5Fluoro-W)-T-(THPG)-C(3)-N-N-(2Nal)-H-D-C(3)-(7Aza-W)-(trans4Fluoro-P)- |
| | [D-Lys(DOTA)]-NH2 |
| 244 | Ac-(5Fluoro-W)-T-(Nle)-C(3)-N-N-(2Nal)-H-D-C(3)-(7Aza-W)-(trans4Fluoro-P)-[D- |
| | Lys(DOTA)]-NH2 |
| 245 | Ac-(5Fluoro-W)-T-(CHA)-C(3)-N-N-(2Nal)-H-D-C(3)-(7Aza-W)-(trans4Fluoro-P)-[D- |
| | Lys(DOTA)]-NH2 |
| 246 | Ac-(5Fluoro-W)-T-(2CF3-F)-C(3)-N-N-(2Nal)-H-D-C(3)-(7Aza-W)-(trans4Fluoro-P)- |
| | [D-Lys(DOTA)]-NH2 |
| 251 | Ac-W-T-I-C(3)-N-N-(2Nal)-H-D-C(3)-W-P-{D-Lys[PEG8-D-Lys(DOTA)-PEG8- |
| | COCH2Ph-4Br]}-NH2 |
| 252 | Ac-W-T-I-C(3)-N-N-(2Nal)-H-D-C(3)-W-P-{D-Lys[PEG8-D-Lys(DOTA)-PEG12- |
| | COCH2Ph-4Br]}-NH2 |
| 255 | Ac-W-T-I-C(3)-N-N-(2Nal)-H-D-C(3)-W-P-{D-Lys[PEG8-D-Lys(DOTA)-PEG24- |
| | COCH2Ph-4Br]}-NH2 |
| 256 | Ac-W-T-I-C(3)(pDBX)-N-N-(2Nal)-H-D-C(2)-W-P-[D-Lys(DOTA)]-NH2 |
| 257 | Ac-(5Fluoro-W)-T-(Tbg)-C(3)-N-N-(2Nal)-H-D-C(3)-(7Aza-W)-(trans4Fluoro-P)-[D- |
| | Lys(DOTA)]-NH2 |
| 258 | Ac-(5Fluoro-W)-T-(Chg)-C(3)-N-N-(2Nal)-H-D-C(3)-(7Aza-W)-(trans4Fluoro-P)-[D- |
| | Lys(DOTA)]-NH2 |
| 260 | Ac-W-T-I-C(3)-[3-(4-piperidinyl)-Ala]-N-(2Nal)-H-D-C(3)-W-P-[D-Lys(DOTA)]-NH2 |
| 261 | Ac-W-T-I-C(3)-[3-(1-morpholinyl)-Ala]-N-(2Nal)-H-D-C(3)-W-P-[D-Lys(DOTA)]-NH2 |
| 262 | Ac-W-T-I-C(3)-N-N-(2Nal)-H-D-C(3)-W-P-[D-Lys(DOTA)]-OH |
| 263 | Ac-W-T-I-C(3)-(3Pya)-N-(2Nal)-H-D-C(3)-W-P-[D-Lys(DOTA)]-NH2 |
| 264 | Ac-W-T-I-C(3)-(4Pya)-N-(2Nal)-H-D-C(3)-W-P-[D-Lys(DOTA)]-NH2 |
| 265 | Ac-W-T-I-C(3)-N-(3Pya)-(2Nal)-H-D-C(3)-W-P-[D-Lys(DOTA)]-NH2 |
| 266 | Ac-W-T-I-C(3)-N-(4Pya)-(2Nal)-H-D-C(3)-W-P-[D-Lys(DOTA)]-NH2 |
| 267 | Ac-W-T-I-C(3)-N-[3-(4-piperidinyl)-Ala]-(2Nal)-H-D-C(3)-W-P-[D-Lys(DOTA)]-NH2 |
| 268 | Ac-W-T-I-C(3)-N-[3-(1-morpholinyl)-Ala]-(2Nal)-H-D-C(3)-W-P-[D-Lys(DOTA)]-NH2 |
| 269 | Ac-(5Fluoro-W)-T-(CBA)-C(3)-N-N-(2Nal)-H-D-C(3)-(7Aza-W)-(trans4Fluoro-P)-[D- |
| | Lys(DOTA)]-NH2 |
| 270 | Ac-W-T-I-C(3)-N-N-(2Nal)-H-D-C(3)-W-P-PEG4-[D-Lys(DOTA)]-NH2 |
| 271 | Ac-W-T-I-C(3)-N-N-(2Nal)-H-D-C(3)-W-P-[D-Lys(PEG4-DOTA)]-NH2 |
| 272 | Ac-W-T-I-C(3)-N-N-W-H-D-C(3)-(7-CI-W)-P-[D-Lys(DOTA)]-NH2 |
| 273 | Ac-W-T-I-C(3)-Glu-N-W-Glu-D-C(3)-W-P-[D-Lys(DOTA)]-NH2 |
| 274 | Ac-W-T-I-C(3)-N-N-W-Glu-D-C(3)-W-P-[D-Lys(DOTA)]-NH2 |
| 275 | Ac-W-T-I-C(3)-Glu-Glu-W-Glu-D-C(3)-W-P-[D-Lys(DOTA)]-NH2 |
| 276 | Ac-W-T-I-C(3)-N-Glu-W-Glu-D-C(3)-W-P-[D-Lys(DOTA)]-NH2 |
| 277 | Ac-W-T-I-C(3)-Glu-N-W-H-D-C(3)-W-P-[D-Lys(DOTA)]-NH2 |
| 278 | Ac-W-T-I-C(3)-Glu-Glu-W-H-D-C(3)-W-P-[D-Lys(DOTA)]-NH2 |
| 279 | Ac-W-T-I-C(3)-N-Glu-W-H-D-C(3)-W-P-[D-Lys(DOTA)]-NH2 |
| 280 | Ac-W-T-(Chg)-C(3)-[3-(4-piperidinyl)-Ala]-N-(2Nal)-H-D-C(3)-W-P-[D-Lys(DOTA)]- |
| | NH2 |
| 281 | Ac-W-T-(Tbg)-C(3)-[3-(4-piperidinyl)-Ala]-N-(2Nal)-H-D-C(3)-W-P-[D-Lys(DOTA)]- |
| | NH2 |
| 282 | Ac-W-T-(t-Bu-Ala)-C(3)-[3-(4-piperidinyl)-Ala]-N-(2Nal)-H-D-C(3)-W-P-[D- |
| | Lys(DOTA)]-NH2 |
| 283 | Ac-W-T-(Chg)-C(3)-[3-(4-piperidinyl)-Ala]-N-(2Nal)-H-(Asn)-C(3)-W-P-[D- |
| | Lys(DOTA)]-NH2 |
| 284 | Ac-W-T-(Chg)-C(3)-[3-(4-piperidinyl)-Ala]-N-(2Nal)-H-(Ala)-C(3)-W-P-[D- |
| | Lys(DOTA)]-NH2 |
| 285 | Ac-W-T-(Chg)-C(3)-[3-(4-piperidinyl)-Ala]-N-(2Nal)-H-(Thr)-C(3)-W-P-[D- |
| | Lys(DOTA)]-NH2 |
| 286 | Ac-W-T-(Chg)-C(3)-[3-(4-piperidinyl)-Ala]-N-(2Nal)-H-D-C(3)-W-P-[D-Lys(Chx- |
| | DOTA)]-NH2 |
| 287 | Ac-W-T-(Chg)-C(3)-(3Pya)-N-(2Nal)-H-D-C(3)-W-P-[D-Lys(DOTA)]-NH2 |
| 288 | Ac-W-T-(Tbg)-C(3)-(3Pya)-N-(2Nal)-H-D-C(3)-W-P-[D-Lys(DOTA)]-NH2 |
| 289 | Ac-W-T-(t-Bu-Ala)-C(3)-(3Pya)-N-(2Nal)-H-D-C(3)-W-P-[D-Lys(DOTA)]-NH2 |
| 290 | Ac-W-T-(Chg)-C(3)-(4Pya)-N-(2Nal)-H-D-C(3)-W-P-[D-Lys(DOTA)]-NH2 |
| 291 | Ac-W-T-(Tbg)-C(3)-(4Pya)-N-(2Nal)-H-D-C(3)-W-P-[D-Lys(DOTA)]-NH2 |
| 292 | Ac-W-T-I-C(3)-N-N-(5OH-W)-H-D-C(3)-W-P-[D-Lys(DOTA)]-NH2 |
| 293 | Ac-W-T-I-C(3)-N-N-W-H-D-C(3)-(5OH-W)-P-[D-Lys(DOTA)]-NH2 |
| 294 | Ac-(1Me-W)-T-I-C(3)-N-N-W-H-D-C(3)-W-P-[D-Lys(DOTA)]-NH2 |
| 295 | Ac-W-T-I-C(3)-N-N-(7-Me-W)-H-D-C(3)-W-P-[D-Lys(DOTA)]-NH2 |
| 296 | Ac-W-T-I-C(3)-N-N-(7-MeO-W)-H-D-C(3)-W-P-[D-Lys(DOTA)]-NH2 |
| 297 | Ac-W-T-I-C(3)-N-N-(7-CI-W)-H-D-C(3)-W-P-[D-Lys(DOTA)]-NH2 |
| 298 | Ac-W-T-I-C(3)-N-N-W-H-D-C(3)-(7-Me-W)-P-[D-Lys(DOTA)]-NH2 |
| 299 | Ac-W-T-I-C(3)-N-N-W-H-D-C(3)-(7-MeO-W)-P-[D-Lys(DOTA)]-NH2 |
| 326 | Ac-W-T-I-C(3)-[3-(4-piperidinyl)-Ala]-N-(2Nal)-K(C12)-D-C(3)-W-P-[D-Lys(DOTA)]- |
| | NH2 |
| 329 | Ac-W-T-I-C(1)-N-N-(2Nal)-H-D-C(1)-W-P-[L-Lys(DOTA)]-NH2 |
| 330 | Ac-7MeW-T-I-C(1)-N-N-(2Nal)-H-D-C(1)-W-P-[D-Lys(DOTA)]-NH2 |
| 344 | Ac-(7MeO-Trp)-T-I-C(3)-N-N-W-H-D-C(3)-W-P-[D-Lys(DOTA)]-CONH2 |
| 345 | Ac-W-T-I-C(3)-N-N-W-H-D-C(3)-W-NMeL-[D-Lys(DOTA)]-NH2 |
| 346 | Ac-W-T-K(C12)-C(3)-N-N-W-H-D-C(3)-W-P-[D-Lys(DOTA)]-NH2 |
| 347 | Ac-W-T-K(C14)-C(3)-N-N-W-H-D-C(3)-W-P-[D-Lys(DOTA)]-NH2 |
| 348 | Ac-W-T-K(C16)-C(3)-N-N-W-H-D-C(3)-W-P-[D-Lys(DOTA)]-NH2 |
| 349 | C12OH-W-T-I-C(3)-N-N-W-H-D-C(3)-W-P-[D-Lys(DOTA)]-NH2 |
| 352 | Ac-W-T-I-C(3)-N-N-(2Nal)-H-D-C(3)-W-[NMeK(DOTA)]-NH2 |
| 353 | Ac-W-T-I-C(3)-N-N-(2Nal)-H-D-C(3)-W-P-[D-Lys(G-S-G-S-G-S-DOTA)]-NH2 |
| 354 | Ac-W-T-I-C(3)-N-N-(2Nal)-H-D-C(3)-W-P-[D-Lys(gE-DOTA)]-NH2 |
| 355 | Ac-W-T-I-C(3)-N-N-(2Nal)-H-D-C(3)-W-P-[D-Lys(gE-gE-gE-DOTA)]-NH2 |
| 356 | Ac-W-T-I-C(3)-N-N-(2Nal)-H-D-C(3)-W-P-[D-Lys((D-Pro)-PEG8-DOTA)]-NH2 |
| 357 | Ac-W-T-I-C(3)-N-N-(2Nal)-H-D-C(3)-W-P-(D-Pro)3-PEG8-[D-Lys(DOTA)]-NH2 |
| 358 | Ac-W-T-I-C(3)-N-N-(2Nal)-H-D-C(3)-W-P-[D-Lys((D-Pro)3-PEG8-DOTA)]-NH2 |
| 359 | Ac-W-T-I-C(3)-N-N-(2Nal)-H-D-C(3)-W-R-3Me-Aze-[D-Lys(DOTA)]-NH2 |
| 360 | Ac-W-T-I-C(3)-N-N-(2Nal)-H-D-C(3)-W-aMe-Aze-[D-Lys(DOTA)]-NH2 |
| 361 | Ac-W-T-I-C(3)-N-N-(2Nal)-H-D-C(3)-W-ACI-[D-Lys(DOTA)]-NH2 |
| 362 | Ac-W-T-I-C(3)-N-N-(2Nal)-H-D-C(3)-W-3Me2-Aze-[D-Lys(DOTA)]-NH2 |
| 363 | Ac-W-T-I-C(3)-N-N-(2Nal)-H-D-C(3)-W-P-[D-Lys(SP6-PEG8-DOTA)]-NH2 |
| 364 | Ac-W-T-I-C(3)-N-N-(2Nal)-H-D-C(3)-W-P-SP6-PEG8-[D-Lys(DOTA)]-NH2 |
| 365 | Ac-W-T-I-C(3)-N-N-(2Nal)-H-D-C(3)-W-P-[D-Lys(G-G-S-G-G-S-DOTA)]-NH2 |
| 366 | Ac-W-T-I-C(3)-N-N-(2Nal)-(3Pya)-D-C(3)-W-P-[D-Lys(DOTA)]-NH2 |
| 367 | Ac-W-T-I-C(3)-N-N-(2Nal)-(4Pya)-D-C(3)-W-P-[D-Lys(DOTA)]-NH2 |
| 368 | Ac-W-T-I-C(3)-N-N-(2Nal)-K(C12)-D-C(3)-W-P-[D-Lys(gE-gE-gE-DOTA)]-NH2 |
| 369 | Ac-(7Me-W)-T-I-C(3)-N-N-(2Nal)-H-D-C(3)-W-P-[D-Lys(DOTA)]-NH2 |
| 371 | Ac-W-T-I-C(3)-N-N-(2Nal)-H-D-C(3)-W-P-(Sar)5-[D-Lys(DOTA)]-NH2 |
| 372 | Ac-W-T-I-C(3)-N-N-(2Nal)-H-D-C(3)-W-P-(Ahx)2-[D-Lys(DOTA)]-NH2 |
| 373 | Ac-W-T-I-C(3)-N-N-(2Nal)-H-D-C(3)-W-P-(Cyx)2-[D-Lys(DOTA)]-NH2 |
| 374 | Ac-W-T-I-C(3)-N-N-(2Nal)-H-D-C(3)-W-P-(AMBX)-[D-Lys(DOTA)]-NH2 |
| 375 | Ac-W-T-I-C(3)-N-N-(2Nal)-Arg-D-C(3)-W-P-[D-Lys(DOTA)]-NH2 |
| 376 | Ac-W-T-I-C(3)-N-N-(2Nal)-Orn-D-C(3)-W-P-[D-Lys(DOTA)]-NH2 |
| 409 | Ac-W-T-(aMe-I)-C(3)-N-N-W-H-D-C(3)-W-P-[D-Lys(DOTA)]-NH2 |
| 410 | Ac-W-T-I-C(3)-N-N-W-H-(aMe-D)-C(3)-W-P-[D-Lys(DOTA)]-NH2 |
| 411 | Ac-W-T-I-C(3)-Ser-N-W-Ser-D-C(3)-W-P-[D-Lys(DOTA)]-NH2 |
| 412 | Ac-W-T-I-C(3)-Ser-Ser-W-Ser-D-C(3)-W-P-[D-Lys(DOTA)]-NH2 |
| 413 | Ac-W-T-I-C(3)-N-Ser-W-Ser-D-C(3)-W-P-[D-Lys(DOTA)]-NH2 |
| 414 | Ac-W-T-I-C(3)-Ser-N-W-H-D-C(3)-W-P-[D-Lys(DOTA)]-NH2 |
| 415 | Ac-W-T-I-C(3)-Ser-Ser-W-H-D-C(3)-W-P-[D-Lys(DOTA)]-NH2 |
| 426 | Ac-WT-(Chg)-C(3)-[3-(4-piperidinyl)-Ala]-N-(2Nal)-Lys(PEG24-[gE(C16)]-OH)-D- |
| | C(3)-W-P-[D-Lys(DOTA)]-NH2 |
| 427 | Ac-WT-(NMe-Nle)-C(3)-[3-(4-piperidinyl)-Ala]-N-(2Nal)-Lys(PEG24-[gE(C16)]-OH)- |
| | D-C(3)-W-P-[D-Lys(DOTA)]-NH2 |

or a pharmaceutically acceptable salt thereof.

### Peptides of Formula (III)

In yet another embodiment, the peptide comprises the amino acid sequence:

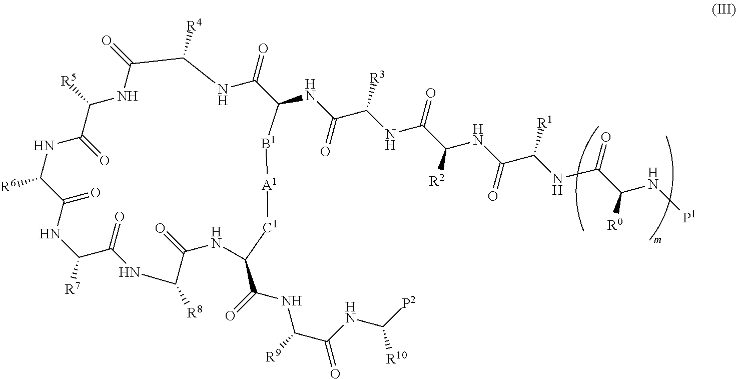

or a pharmaceutically acceptable salt thereof,
   wherein:
P¹ is selected from: H, -L¹-chelator,

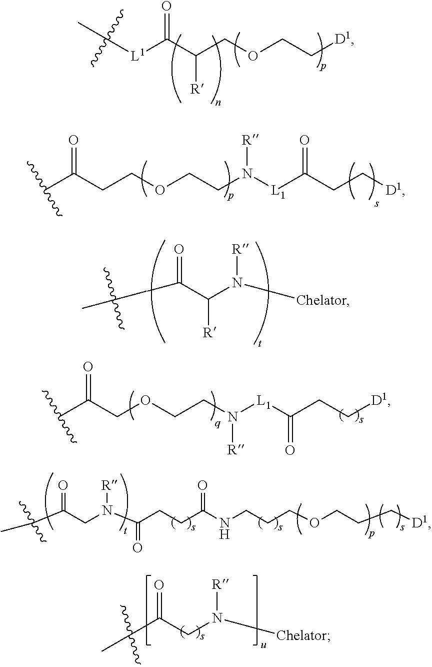

D¹ is selected from H, CH₃, C(O)OH, and —NR″-chelator;
L¹ is absent or selected from

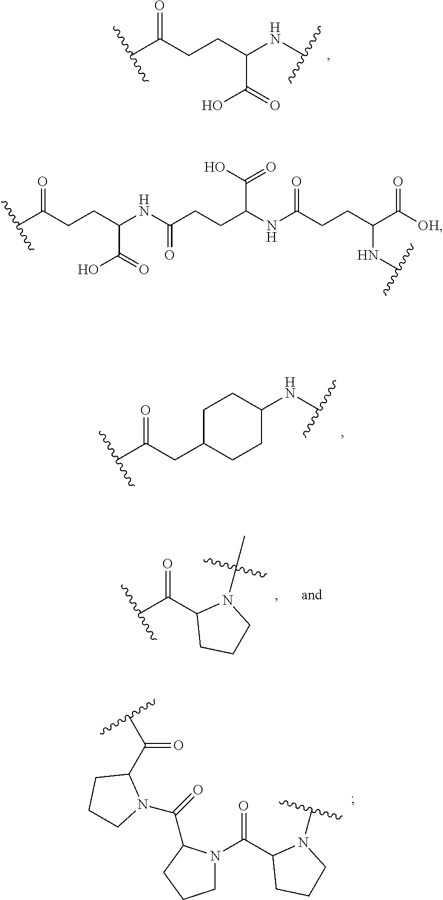 [SMILES: *NC(CCC(*)=O)C(=O)O ; *NC(CCC(=O)NC(CCC(=O)NC(CCC(*)=O)C(=O)O)C(=O)O)C(=O)O ; *NC1CCC(CC(*)=O)CC1 ; *C(=O)C1CCCN1* ; *C(=O)C1CCCN1C(=O)C1CCCN1C(=O)C1CCCN1*]

wherein the amino group of L¹ connects to the carbonyl group of P¹ or chelator to form an amide bond;
P² is selected from C(O)NH₂, C(O)OH, -L²-chelator,

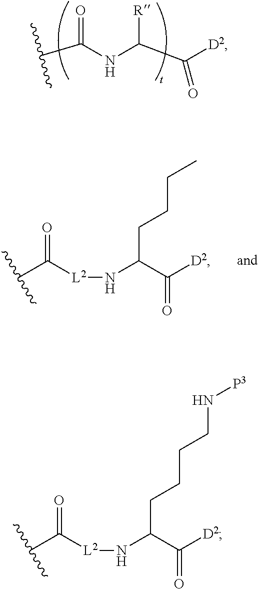

D² is OH or NH₂;
L² is absent or selected from:

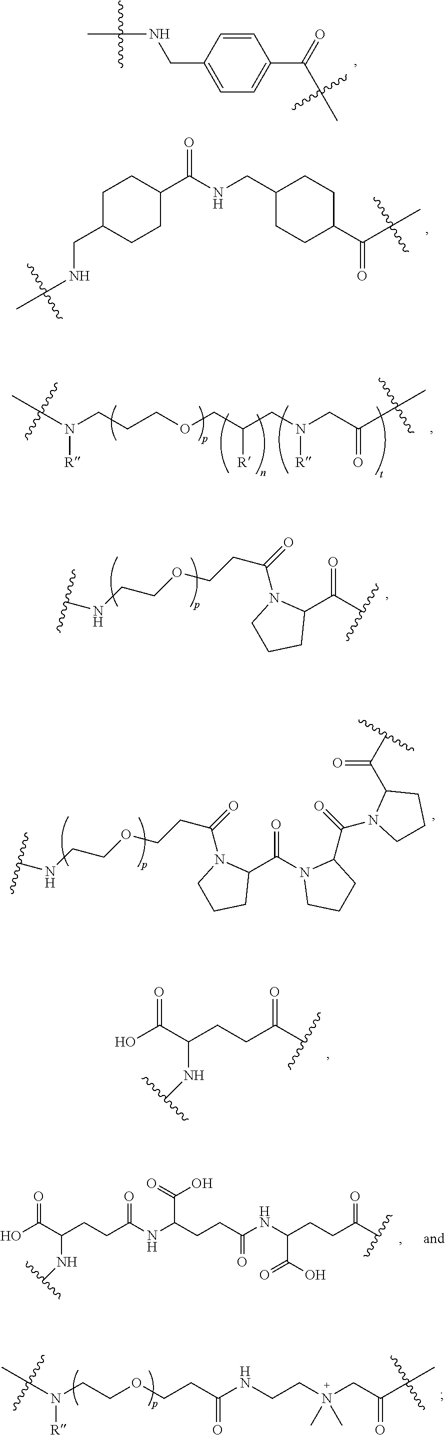

wherein the amino group of L² connects to the carbonyl group of P² or chelator to form an amide bond;
P³ is selected from H, Ac, -L³-chelator,

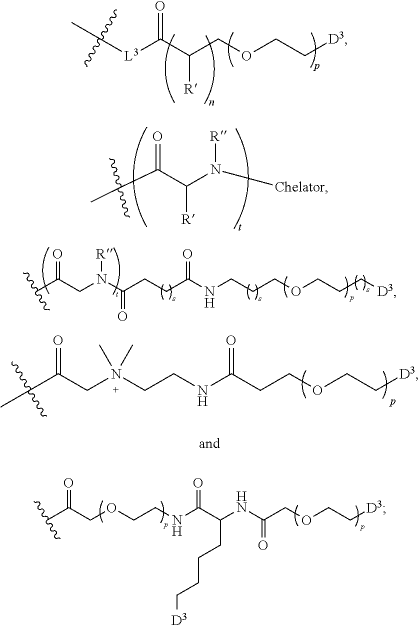

L³ is absent or independently selected from

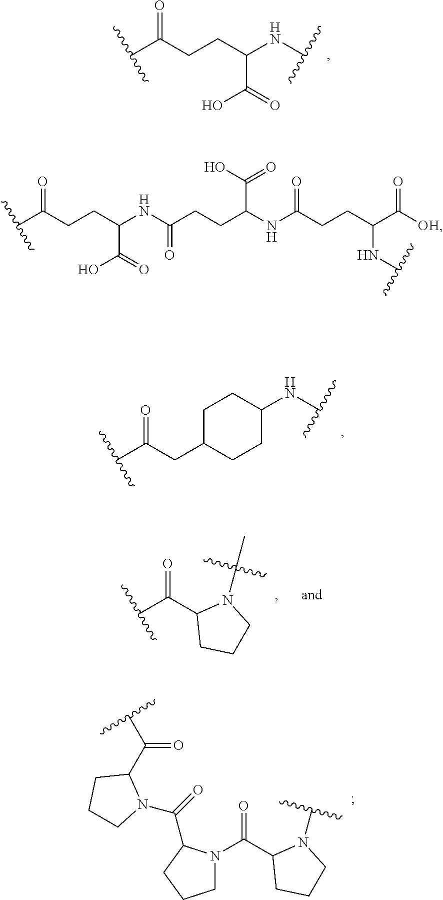 [SMILES: *NC(CCC(*)=O)C(=O)O ; *NC(CCC(=O)NC(CCC(=O)NC(CCC(*)=O)C(=O)O)C(=O)O)C(=O)O ; *NC1CCC(CC(*)=O)CC1 ; *C(=O)C1CCCN1* ; *C(=O)C1CCCN1C(=O)C1CCCN1C(=O)C1CCCN1*]

wherein the carbonyl group of L³ connects to an amine group of P² to form an amide bond;
D³ is independently selected from: CH₃, C(O)OH, —NR″-chelator, and

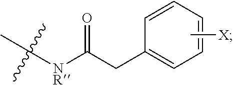

X is halogen;
R³ is an amino acid side chain of a natural amino acid or an amino acid side chain of an unnatural amino acid;
R¹ is an amino acid side chain of a natural amino acid or an amino acid side chain of an unnatural amino acid;
R² is an amino acid side chain of a natural amino acid or an amino acid side chain of an unnatural amino acid;
R³ is an amino acid side chain of a natural amino acid or an amino acid side chain of an unnatural amino acid;
B¹ is C₁₋₆ alkylene;
C¹ is C₁₋₆ alkylene;
A¹ is selected from:

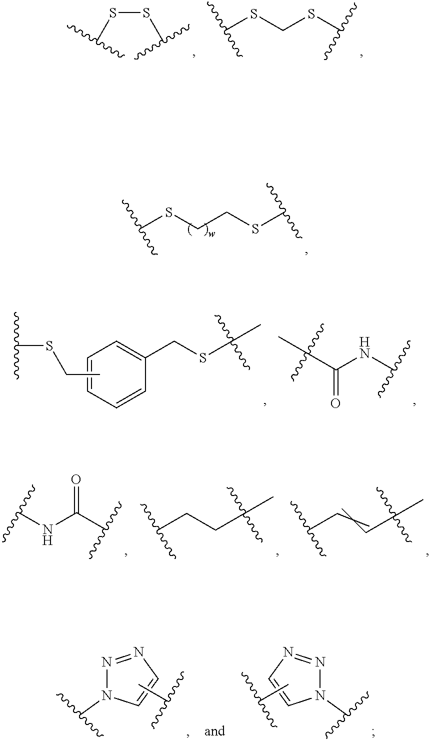

wherein w is selected from 1, 2, or 3;
R⁴ is an amino acid side chain of a natural amino acid or an amino acid side chain of an unnatural amino acid, both of which are optionally substituted with chelator, CH₂C(O)OH, or C(O)(CH₂CH₂O)ₚ(CH₂)₂N(CH₃)₃⁺, wherein the chelator is optionally substituted to the amino acid side chain through a linker;
R⁵ is an amino acid side chain of a natural amino acid or an amino acid side chain of an unnatural amino acid, both of which are optionally substituted with chelator, wherein the chelator is optionally substituted to the amino acid side chain through a linker;
R⁶ is an amino acid side chain of a natural amino acid or an amino acid side chain of an unnatural amino acid;
R⁷ is selected from:
(i) an amino acid side chain of a natural amino acid,
(ii) an amino acid side chain of an unnatural amino acid, or
(iii) selected from the group consisting of:

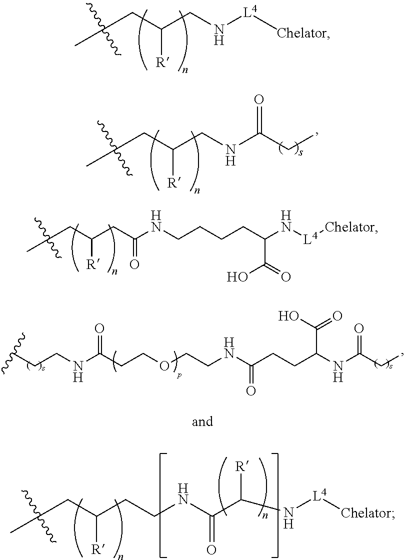

wherein L⁴ is absent or independently selected from:

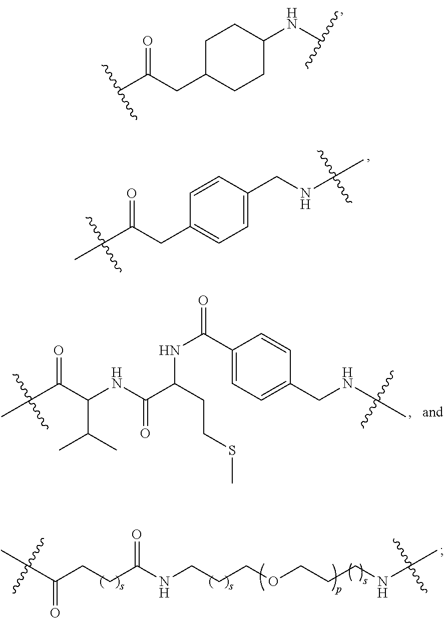

wherein the carbonyl group of L⁴ connects to an amine group of R⁷ to form an amide bond;
R⁸ is an amino acid side chain of a natural amino acid or an amino acid side chain of an unnatural amino acid;
R⁹ is an amino acid side chain of a natural amino acid or an amino acid side chain of an unnatural amino acid;
R¹⁰ is an amino acid side chain of a natural amino acid or an amino acid side chain of an unnatural amino acid;
m is 0 or 1;
each n, q, and u are independently an integer from 0 to 16;
each p is independently an integer from 0 to 24;
each s is independently an integer from 0 to 16;
each t is independently 1, 2, 3, 4, 5, or 6;
R′ is independently selected from H, C(O)OH, (CH₂)OH and NHAc; and
R″ is independently selected from H and CH₃;
wherein either the cyclic peptide does not comprise a chelator, or at least one of R⁴, R⁵, or R⁷ is substituted with a chelator or chelator-containing group.

In an embodiment, the peptide of Formula III is a peptide of Formula III-Ia comprising the amino acid sequence:

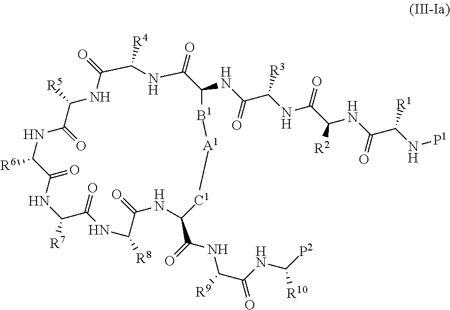

or a pharmaceutically acceptable salt thereof.

In another embodiment, the peptide of Formula III is a peptide of Formula III-Ib comprising the amino acid sequence:

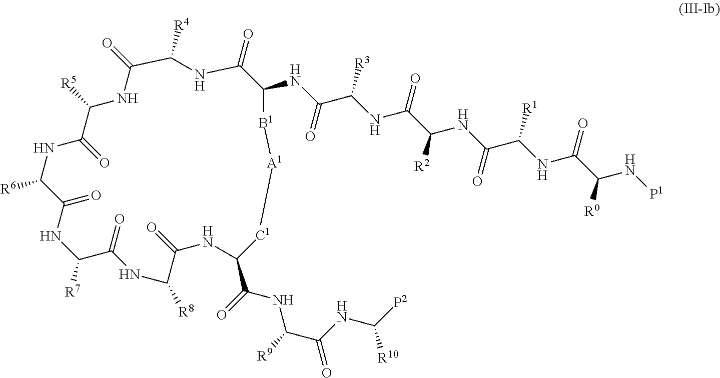

or a pharmaceutically acceptable salt thereof.

In yet another embodiment,
P¹ is selected from Ac,

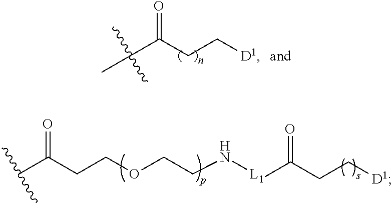

wherein:
D¹ is CH₃ or C(O)OH;
L¹ is absent or

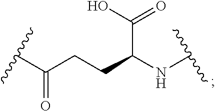 [SMILES: *N[C@@H](CCC(*)=O)C(=O)O]

n and s are independently an integer from 2 to 15; and
p is 8, 9, 10, 11, or 12.

In still another embodiment, P¹ is Ac or CH₃(OCH₂CH₂)₈₋₁₆C(O).

In an embodiment,
P¹ is selected from H, Ac,

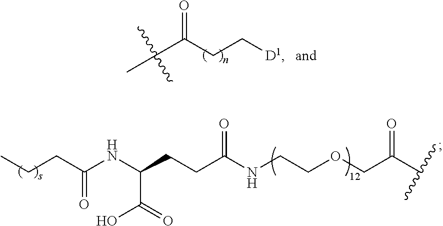

wherein:
n and s are independently 9, 10, 11, 12, or 13; and
D¹ is CH₃ or C(O)OH.

In another embodiment, P² is C(O)NH₂ or C(O)OH.

In yet another embodiment, P² is selected from C(O)NH₂, C(O)OH,

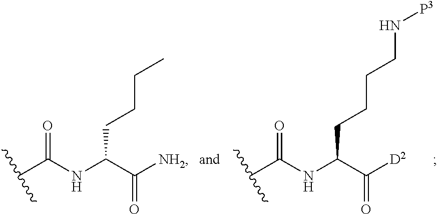

P³ is Ac

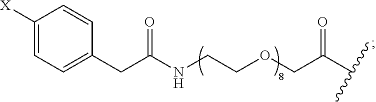

X is halo.

In still another embodiment, the peptide of Formula III is a peptide of Formula III-II comprising the amino acid sequence:

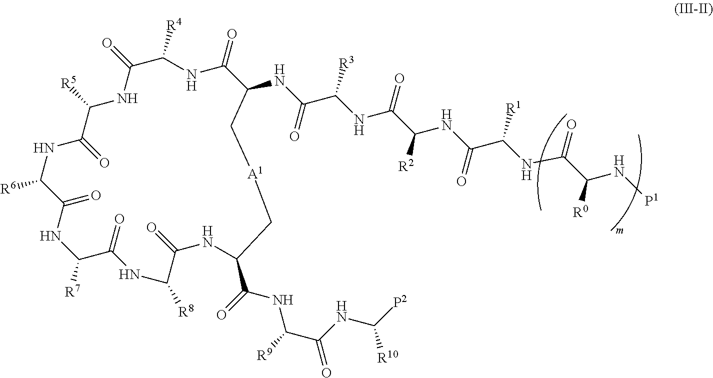

or a pharmaceutically acceptable salt thereof,
wherein P² is C(O)NH₂ or C(O)OH.

In an embodiment, the peptide of Formula III is a peptide of Formula III-III comprising the amino acid sequence:

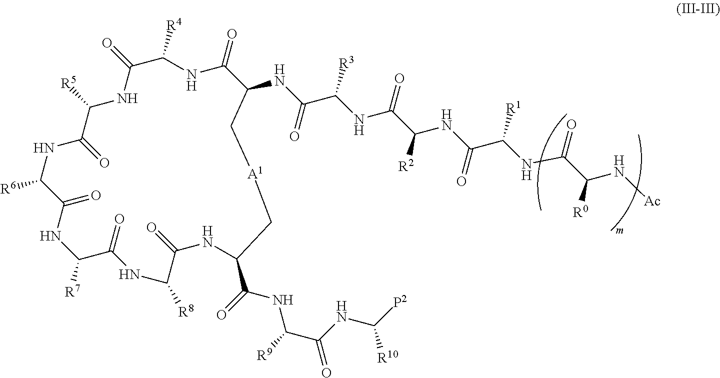

or a pharmaceutically acceptable salt thereof.

In another embodiment, R⁷ is independently selected from the group consisting of

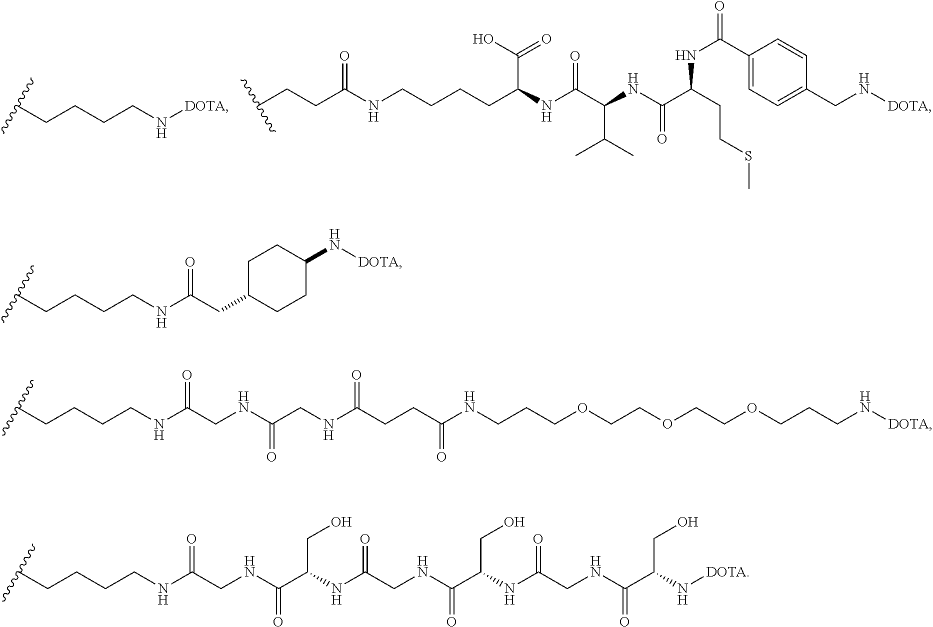

In yet another embodiment,
m is 0;
P¹ is selected from H, Ac,

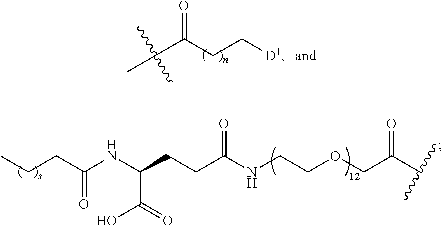

n and s are each independently 9, 10, 11, 12, or 13;
P² is selected from C(O)NH₂, C(O)OH,

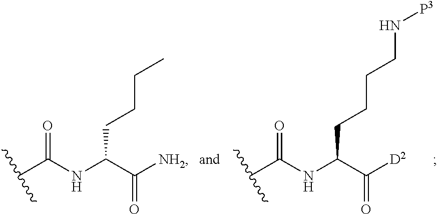

P³ is Ac or

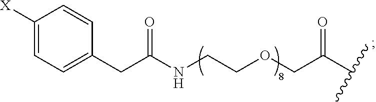

X is halo;
D¹ is CH₃ or C(O)OH;
R¹ is an amino acid side chain selected from the group consisting of Trp, 2Nal, 1 Nal, 4CF₃-Phe, 7Aza-Trp, 1Me-Trp, 50H-Trp, BIP, 5OMe-Trp, 4F-Phe, 3Pya, 4Pya, PAF, MAF, OAF, 5Qui, 7OMe-Trp, 7Me-Trp, 5F-Trp, 7Cl-Trp, D-Ala, Ala, alpha-Me-Trp, and NMe-Trp;
R² is an amino acid side chain selected from the group consisting of Thr, D-Ala, Ala, alpha-Me-Thr, Lys, and NMe-Thr;
R³ an amino acid side chain selected from the group consisting of Ile, Env, CHA, CBA, Nle, Tbg, THPG, Chg, 2Nal, 1Nal, 2CF₃-Phe, 2PhEt-Ala, D-Ala, Ala, Leu, t-Bu-Ala, NMe-Nle, α-tert-amylGly, Allo-Ile, Lys(C12), Lys(C14), Lys(C16), alpha-Me-Ile, and NMe-tBuAla;
A¹ is selected from the group consisting of:

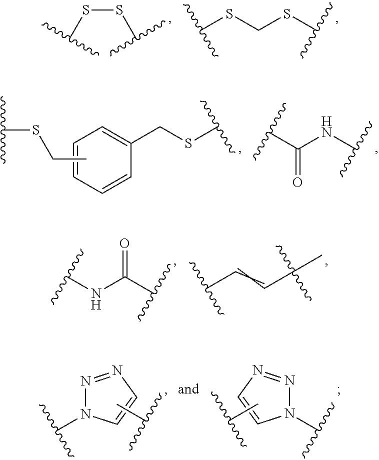

R⁴ is an amino acid side chain selected from the group consisting of Asn, D-Ala, Ala, DAB-4-NHCOC₅H₁₁, DAB-4-NHCOC₇H₁₅, Asp, Ser, Lys(DOTA), Lys, 3-(4-piperidinyl)-Ala, 3-(1-morpholinyl)-Ala, 3Pya, 4Pya, Glu, NMe-Asn, Pip(CH₂CO₂H)Ala, Pip(PegNMe₃)Ala, and Pip(GAE-DOTA)Ala;
R⁵ is an amino acid side chain selected from the group consisting of Asn, Ala, D-Ala, Trp, Asp, Lys, Lys(DOTA), 3Pya, 4Pya, 3-(4-piperidinyl)-Ala, 3-(1-morpholinyl)-Ala, Glu, NMe-Asn, and Ser;
R⁶ is an amino acid side chain selected from the group consisting of Trp, 4CF₃-Phe, 1Me-Trp, 7Aza-Trp, BIP, 2Nal, 1Nal, aMe-Trp, D-Ala, Ala, 4F-Phe, 1Nal, 2Nal, 5F-Trp, 5OMe-Trp, Asn, 50H-Trp, 7Me-Trp, 7MeO-Trp, 7Cl-Trp, and NMe-Trp;
R⁷ is:
(i) an amino acid side chain selected from the group consisting of 3Pya, 4Pya, Lys(Me)3, His, Ala, D-Ala, Gln, Lys, Glu, Arg, Orn, NMe-His, and Ser; or
(ii) selected from the group consisting of:

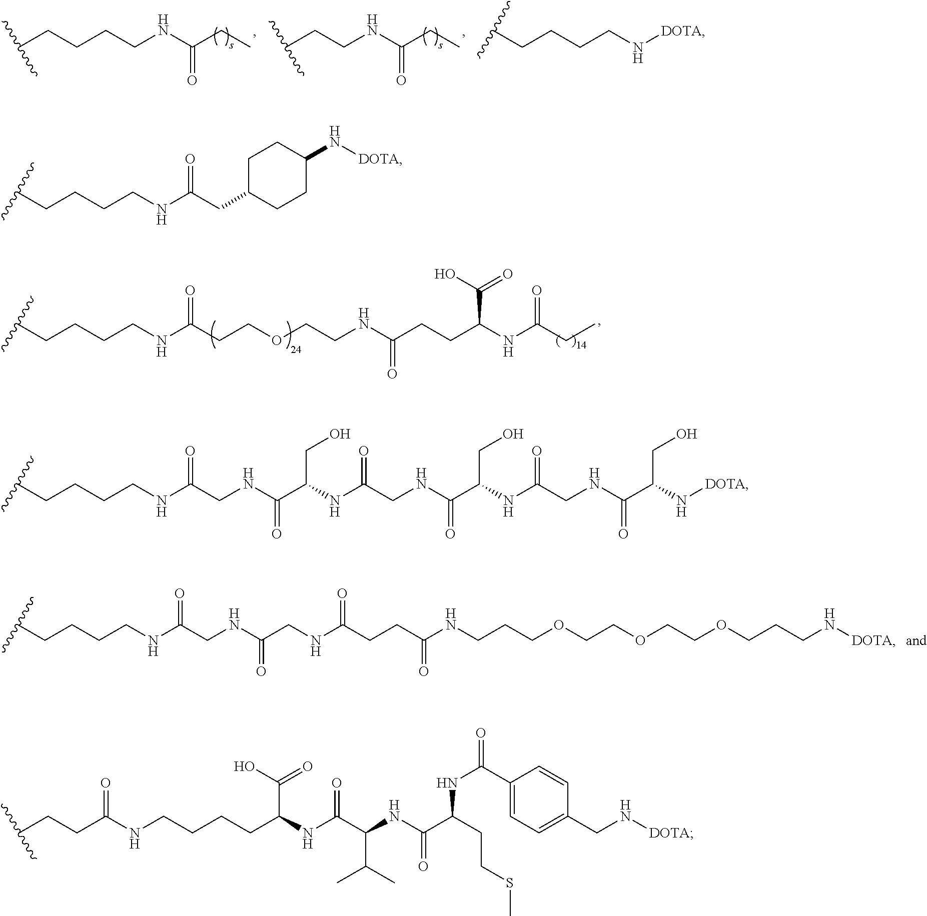

each s is independently 3, 5, 10, 12, or 14;
R⁸ is an amino acid side chain selected from the group consisting of Asp, D-Ala, Ala, Asn, Thr, NMe-Asp, and alpha-Me-Asp;
R⁹ is an amino acid side chain selected from the group consisting of Trp, 7Aza-Trp, 1Me-Trp, D-Ala, Ala, 4F-Phe, 1Nal, 2Nal, 5F-Trp, 5OMe-Trp, alpha-Me-Trp, 7Cl-Trp, 50H-Trp, 7Me-Trp, 7MeO-Trp, and NMe-Trp; and
R¹⁰ is an amino acid side chain selected from the group consisting of Pro, D-Ala, Ala, alpha-Me-Pro, trans4Fluoro-Pro, cis4Fluoro-Pro, trans40H-Pro, cis40H-Pro, Pip, 5,5-diMe-Pro, NMe-Ser, trans4NH2-Pro, cis4NH2-Pro, Sar, Aze, NMe-Ala, NMe-Leu, R-3Me-Aze, alpha-Me-Aze, ACl, and 3Me2-Aze.

In another embodiment,
m is 1;
P¹ is selected from Ac

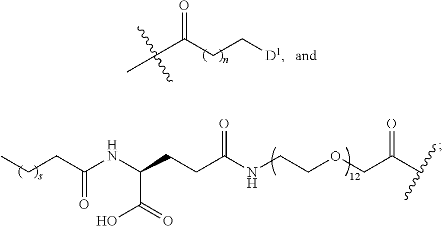

wherein:
n and s are each independently 9, 10, 11, 12, or 13;
P² is selected from C(O)NH₂, C(O)OH,

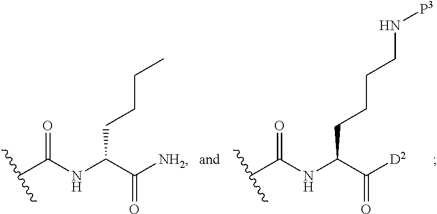

P³ is Ac or

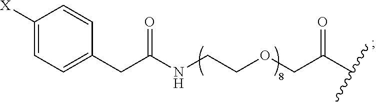

X is halo;
D¹ is CH₃ or C(O)OH;
R⁰ is an amino acid side chain selected from the group consisting of Gly, Met, D-Ala, Ala, Nle, and Nva;
R¹ is an amino acid side chain selected from the group consisting of Trp, 2Nal, 1 Nal, 4CF₃-Phe, 7Aza-Trp, 1Me-Trp, 50H-Trp, BIP, 5OMe-Trp, 4F-Phe, 3Pya, 4Pya, PAF, MAF, OAF, 5Qui, 7OMe-Trp, 7Me-Trp, 5F-Trp, 7Cl-Trp, D-Ala, Ala, alpha-Me-Trp, and NMe-Trp;
R² is an amino acid side chain selected from the group consisting of Thr, D-Ala, Ala, alpha-Me-Thr, Lys, and NMe-Thr;
R³ is an amino acid side chain selected from the group consisting of Ile, Env, CHA, CBA, Nle, Tbg, THPG, Chg, 2Nal, 1Nal, 2CF₃-Phe, 2PhEt-Ala, D-Ala, Ala, Leu, t-Bu-Ala, NMe-Nle, α-tert-amylGly, Allo-Ile, Lys(C12), Lys(C14), Lys(C16), alpha-Me-Ile, and NMe-tBuAla;
A¹ is selected from the group consisting of

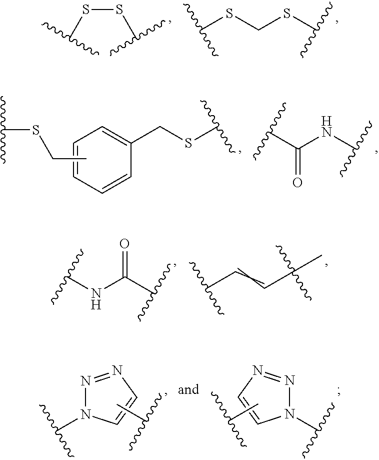

R⁴ is an amino acid side chain selected from the group consisting of Asn, D-Ala, Ala, DAB-4-NHCOC₅H₁₁, DAB-4-NHCOC₇H₁₅, Asp, Ser, Lys(DOTA), Lys, 3-(4-piperidinyl)-Ala, 3-(1-morpholinyl)-Ala, 3Pya, 4Pya, Glu, and NMe-Asn;
R⁵ is an amino acid side chain selected from the group consisting of Asn, Ala, D-Ala, Trp, Asp, Lys, Lys(DOTA), 3Pya, 4Pya, 3-(4-piperidinyl)-Ala, 3-(1-morpholinyl)-Ala, Glu, NMe-Asn, and Ser;
R⁶ is an amino acid side chain selected from the group consisting of Trp, 4CF₃-Phe, 1Me-Trp, 7Aza-Trp, BIP, 2Nal, 1Nal, aMe-Trp, D-Ala, Ala, 4F-Phe, 1Nal, 2Nal, 5F-Trp, 5OMe-Trp, Asn, 50H-Trp, 7Me-Trp, 7MeO-Trp, 7Cl-Trp, and NMe-Trp;
R⁷ is an amino acid side chain selected from the group consisting of 3Pya, 4Pya, Lys(Me)3, His, Ala, D-Ala, Gln, Lys, Glu, Arg, Orn, NMe-His, and Ser; or
R⁷ is selected from the group consisting of

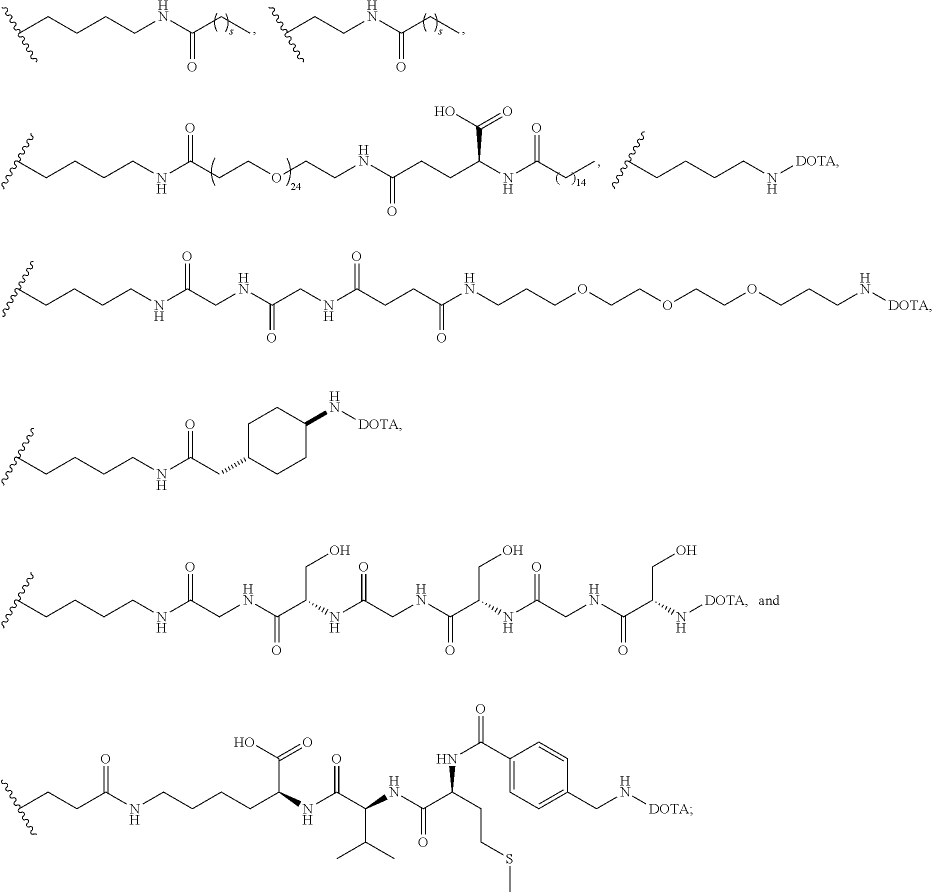

each s is independently 3, 5, 10, 12, or 14;
R⁸ is an amino acid side chain selected from the group consisting of Asp, D-Ala, Ala, Asn, Thr, NMe-Asp, and alpha-Me-Asp;
R⁹ is an amino acid side chain selected from the group consisting of Trp, 7Aza-Trp, 1Me-Trp, D-Ala, Ala, 4F-Phe, 1Nal, 2Nal, 5F-Trp, 5OMe-Trp, alpha-Me-Trp, 7Cl-Trp, 50H-Trp, 7Me-Trp, 7MeO-Trp, and NMe-Trp; and
R¹⁰ is an amino acid side chain selected from the group consisting of Pro, D-Ala, Ala, alpha-Me-Pro, trans4Fluoro-Pro, cis4Fluoro-Pro, trans40H-Pro, cis40H-Pro, Pip, 5,5-diMe-Pro, NMe-Ser, trans4NH2-Pro, cis4NH2-Pro, Sar, Aze, NMe-Ala, NMe-Leu, R-3Me-Aze, alpha-Me-Aze, ACl, and 3Me2-Aze.

In yet another embodiment,
B¹ is CH₂ or C(CH₃)₂; and
C¹ is CH₂ or C(CH₃)₂.

In still another embodiment,
B¹ is CH₂; and
C¹ is CH₂.

In an embodiment, at least one of R⁴, R⁵, or R⁷ is substituted with a chelator or chelator-containing group. In another embodiment, the cyclic peptide does not comprise a chelator.

In yet another embodiment, R⁴ is substituted with chelator. In still another embodiment, R⁵ is substituted with chelator.

In an embodiment, R⁷ is selected from

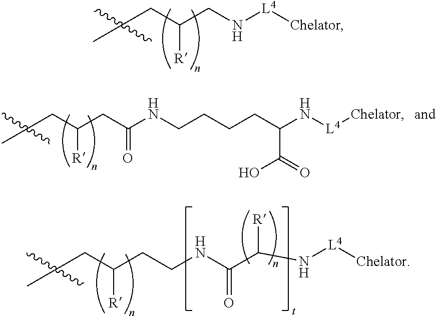

In an embodiment, chelator is independently selected from the group consisting of ethylenediamine tetraacetic acid (EDTA), diethylenetriamine pentaacetic acid (DTPA), 1,4,7,10-tetra-azacylcododecane-N,N′,N″,N′″-tetraacetic acid (DOTA), 6-((16-((6-Carboxypyridin-2-yl)methyl)-1,4,10,13-tetraoxa-7,16-diazacyclooctadecan-7-yl)methyl)-4-isothiocyanatopicolinic acid (Macropa), Macrodipa, 2,2′,2″,2′″-(1,10-dioxa-4,7,13,16-tetraazacyclooctadecane-4,7,13,16-tetrayl)tetraacetic acid) (Crown), 1,4,7,10-Tetraazacyclododecane-1,4,7,10-tetraacetic acid, α-(2-carboxyethyl) (DOTAGA), 1,4,7-Triazacyclononane-N,N′,N″-triacetic acid (NOTA), 1,4,7,10-tetraazacyclododecane-N,N′,N″,N′″-tetraacetic acid (TETA), 1,4,7,10,13-pentaazacyclopentadecane-N,N′,N″,N′″,N″″-pentaacetic acid (PEPA), and 1,4,7,10,13,16-hexaazacyclohexadecane-N,N′,N″,N′″,N″″,N′″″-hexaacetic acid (HEHA).

In another embodiment, chelator is DOTA. In yet another embodiment, chelator is DOTAGA. In still another embodiment, chelator is macrodipa. In an embodiment, chelator is macropa.

In another embodiment, Formula I is substituted by at least one chelator. In yet another embodiment, Formula I is substituted by one chelator. In still another embodiment, Formula I is substituted by at two chelators.

In an embodiment, the peptide comprises the amino acid sequence:

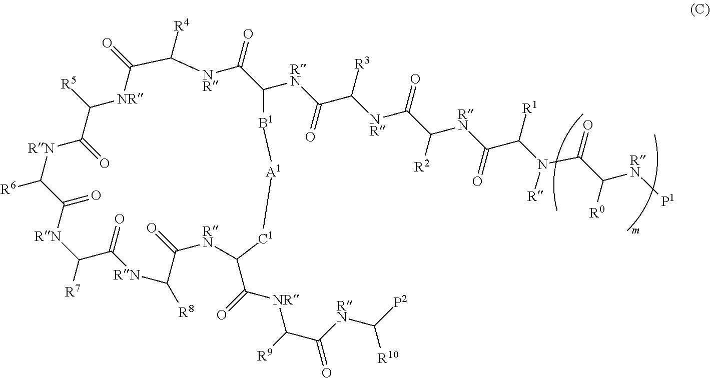

or a pharmaceutically acceptable salt thereof,
wherein the variable definitions are the same as those defined above for Formula III;
   wherein:
each alpha-carbon atom on the peptide backbone is optionally substituted with methyl;
when a variable group R⁰, R¹, R², R³, R⁴, R⁵, R⁶, R⁷, R⁸, R⁹, or R¹⁰ is the side chain of a cyclic amino acid, the corresponding amino acid nitrogen of the peptide backbone forms part of the cyclic group; and
wherein the cyclic peptide optionally comprises a radionuclide.

In an embodiment, the cyclic peptide of Formula III is selected from a peptide in Table 4. In another embodiment, the cyclic peptide of Formula C is selected from a peptide in Table 4.

TABLE 4

| Cpd# | Sequence |
|---|---|
| 1 | Ac-M-W-T-I-C(3)-N-N-W-H-D-C(3)-W-P-CONH2 |
| 2 | Ac-(D-Ala)-W-T-I-C(3)-N-N-W-H-D-C(3)-W-P-CONH2 |
| 3 | Ac-M-(D-Ala)-T-I-C(3)-N-N-W-H-D-C(3)-W-P-CONH2 |
| 4 | Ac-M-W-(D-Ala)-I-C(3)-N-N-W-H-D-C(3)-W-P-CONH2 |
| 5 | Ac-M-W-T-(D-Ala)-C(3)-N-N-W-H-D-C(3)-W-P-CONH2 |
| 6 | Ac-M-W-T-I-C(3)-N-N-(D-Ala)-H-D-C(3)-W-P-CONH2 |
| 7 | Ac-M-W-T-I-C(3)-(D-Ala)-N-W-H-D-C(3)-W-P-CONH2 |
| 8 | Ac-M-W-T-I-C(3)-N-N-W-H-(D-Ala)-C(3)-W-P-CONH2 |
| 9 | Ac-M-W-T-I-C(3)-N-N-W-H-D-C(3)-(D-Ala)-P-CONH2 |
| 10 | Ac-M-W-T-I-C(3)-N-N-W-H-D-C(3)-W-(D-Ala)-CONH2 |
| 11 | Ac-(L-Ala)-W-T-I-C(3)-N-N-W-H-D-C(3)-W-P-CONH2 |
| 12 | Ac-M-(L-Ala)-T-I-C(3)-N-N-W-H-D-C(3)-W-P-CONH2 |
| 13 | Ac-M-W-(L-Ala)-I-C(3)-N-N-W-H-D-C(3)-W-P-CONH2 |
| 14 | Ac-M-W-T-(L-Ala)-C(3)-N-N-W-H-D-C(3)-W-P-CONH2 |
| 15 | Ac-M-W-T-I-C(3)-(L-Ala)-N-W-H-D-C(3)-W-P-CONH2 |
| 16 | Ac-W-T-I-C(3)-N-N-W-H-D-C(3)-W-P-CONH2 |
| 24 | Ac-(7Aza-W)-T-I-C(3)-N-N-W-H-D-C(3)-W-P-CONH2 |
| 25 | Ac-(1Nal)-T-I-C(3)-N-N-W-H-D-C(3)-W-P-CONH2 |
| 26 | Ac-(2Nal)-T-I-C(3)-N-N-W-H-D-C(3)-W-P-CONH2 |
| 27 | Ac-(5OH-W)-T-I-C(3)-N-N-W-H-D-C(3)-W-P-CONH2 |
| 28 | Ac-(5OMe-W)-T-I-C(3)-N-N-W-H-D-C(3)-W-P-CONH2 |
| 29 | Ac-(3Pya)-T-I-C(3)-N-N-W-H-D-C(3)-W-P-CONH2 |
| 30 | Ac-M-W-T-I-DapN3(6)-N-N-W-H-D-Pra(6)-W-P-CONH2 |
| 31 | Ac-M-W-T-I-Pen(3)-N-N-W-H-D-Pen(3)-W-P-CONH2 |
| 32 | Ac-M-W-T-I-C(1)-N-N-W-H-D-C(1)-W-P-CONH2 |
| 33 | Ac-W-T-I-C(3)-N-N-(7Aza-W)-H-D-C(3)-W-P-CONH2 |
| 34 | Ac-M-W-T-I-C(3)-N-N-(L-Ala)-H-D-C(3)-W-P-CONH2 |
| 35 | Ac-M-W-T-I-C(3)-N-N-W-H-D-C(3)-(L-Ala)-P-CONH2 |
| 36 | Ac-M-W-T-I-C(3)-N-N-W-(L-Ala)-D-C(3)-W-P-CONH2 |
| 37 | Ac-M-W-T-I-C(3)-N-N-W-H-(L-Ala)-C(3)-W-P-CONH2 |
| 38 | Ac-M-W-T-I-C(3)-N-(L-Ala)-W-H-D-C(3)-W-P-CONH2 |
| 39 | Ac-M-W-T-I-C(3)-N-(D-Ala)-W-H-D-C(3)-W-P-CONH2 |
| 40 | Ac-M-W-T-I-C(3)-N-N-W-H-D-C(3)-W-(L-Ala)-CONH2 |
| 41 | Ac-M-(1Me-W)-T-I-C(3)-N-N-W-H-D-C(3)-W-P-CONH2 |
| 42 | Ac-M-W-T-I-C(3)-N-N-(1Me-W)-H-D-C(3)-W-P-CONH2 |
| 43 | Ac-M-W-T-I-C(3)-N-N-W-H-D-C(3)-(1Me-W)-P-CONH2 |
| 44 | Ac-M-W-T-(Chg)-C(3)-N-N-W-H-D-C(3)-W-P-CONH2 |
| 45 | Ac-M-W-T-(1Nal)-C(3)-N-N-W-H-D-C(3)-W-P-CONH2 |
| 46 | Ac-M-W-T-(2Nal)-C(3)-N-N-W-H-D-C(3)-W-P-CONH2 |
| 49 | Ac-W-T-I-C(3)-N-N-(4Fluoro-F)-H-D-C(3)-W-P-CONH2 |
| 50 | Ac-W-T-I-C(3)-N-N-(4CF3-F)-H-D-C(3)-W-P-CONH2 |
| 51 | Ac-W-T-I-C(3)-N-N-W-H-D-C(3)-(7Aza-W)-P-CONH2 |
| 52 | Ac-W-T-I-C(3)-N-N-W-H-D-C(3)-(4Fluoro-F)-P-CONH2 |
| 53 | Ac-W-T-I-C(3)-N-N-(BIP)-H-D-C(3)-W-P-CONH2 |
| 71 | Ac-W-T-I-(L-Glu)*-N-N-W-H-D-(L-2,4-Dab)*-W-P-CONH2 (*denotes lactam |
| | between side chains between L-Glu and L-2,4-Dab) |
| 72 | Ac-M-W-T-I-C(3)-N-N-W-(D-Ala)-D-C(3)-W-P-CONH2 |
| 94 | Ac-W-T-(t-Bu-Ala)-C(1)-[3-(4-piperidinyl)-Ala]-N-(2Nal)-Lys(DOTA)-D-C(1)-W-P- |
| | CONH2 |
| 95 | Ac-W-T-I-(L-Asp)*-N-N-W-H-D-(L-2,4-Dab)*-W-P-CONH2 (*denotes lactam |
| | between side chains between L-Asp and L-2,4-Dab) |
| 99 | Ac-W-T-I-(L-Glu)*-N-N-W-H-D-(L-Dap)*-W-P-CONH2 (*denotes lactam between |
| | side chains between L-Glu and L-Dap) |
| 120 | Ac-W-T-I-(L-Asp)*-N-N-W-H-D-(L-Dap)*-W-P-CONH2 (*denotes lactam between |
| | side chains between L-Asp and L-Dap) |
| 153 | Ac-W-T-(NMe-tBuAla)-C(1)-[3-(4-piperidinyl)-Ala]-N-(2Nal)-Lys(DOTA)-D-C(1)-W- |
| | P-CONH2 |
| 214 | Ac-(4Fluoro-F)-T-I-C(3)-N-N-W-H-D-C(3)-W-P-CONH2 |
| 215 | Ac-(4CF3-F)-T-I-C(3)-N-N-W-H-D-C(3)-W-P-CONH2 |
| 216 | Ac-(PAF)-T-I-C(3)-N-N-W-H-D-C(3)-W-P-CONH2 |
| 217 | Ac-(MAF)-T-I-C(3)-N-N-W-H-D-C(3)-W-P-CONH2 |
| 218 | Ac-(OAF)-T-I-C(3)-N-N-W-H-D-C(3)-W-P-CONH2 |
| 236 | Ac-W-T-(NMe-tBuAla)-C(1)-[3-(4-piperidinyl)-Ala]-N-(2Nal)-Lys(DOTA)-D-C(1)-W- |
| | (aMe-P)-CONH2 |
| 247 | Ac-W-T-I-C(3)-Lys(DOTA)-N-(2Nal)-H-D-C(3)-W-P-[D-Lys(DOTA)]-NH2 |
| 248 | Ac-W-T-I-C(3)-N-Lys(DOTA)-(2Nal)-H-D-C(3)-W-P-[D-Lys(DOTA)]-NH2 |
| 249 | Ac-W-T-I-C(3)-Lys(DOTA)-N-(2Nal)-H-D-C(3)-W-P-CONH2 |
| 250 | Ac-W-T-I-C(3)-N-Lys(DOTA)-(2Nal)-H-D-C(3)-W-P-CONH2 |
| 253 | Ac-W-T-I-C(3)-N-N-(2Nal)-Lys(DOTA)-D-C(3)-W-P-[D-Lys(DOTA)]-NH2 |
| 254 | Ac-W-T-I-C(3)-N-N-(2Nal)-Lys(DOTA)-D-C(3)-W-P-CONH2 |
| 300 | Ac-W-T-(NMe-Nle)-C(3)-[3-(4-piperidinyl)-Ala]-N-(2Nal)-Lys(DOTA)-D-C(3)-W-P- |
| | CONH2 |
| 301 | Ac-W-T-(Tbg)-C(3)-[3-(4-piperidinyl)-Ala]-N-(2Nal)-Lys(DOTA)-D-C(3)-W-P- |
| | CONH2 |
| 302 | Ac-W-T-(α-tert-amylGly)-C(3)-[3-(4-piperidinyl)-Ala]-N-(2Nal)-Lys(DOTA)-D-C(3)- |
| | W-P-CONH2 |
| 303 | Ac-W-T-(Allo-Ile)-C(3)-[3-(4-piperidinyl)-Ala]-N-(2Nal)-Lys(DOTA)-D-C(3)-W-P- |
| | CONH2 |
| 304 | Ac-W-T-(Tbg)-C(3)-[3-(1-morpholinyl)-Ala]-N-(2Nal)-Lys(DOTA)-D-C(3)-W-P- |
| | CONH2 (peak 1) |
| 305 | Ac-W-T-(t-Bu-Ala)-C(3)-[3-(1-morpholinyl)-Ala]-N-(2Nal)-Lys(DOTA)-D-C(3)-W-P- |
| | CONH2 (peak 1) |
| 306 | Ac-W-T-(α-tert-amylGly)-C(3)-[3-(1-morpholinyl)-Ala]-N-(2Nal)-Lys(DOTA)-D- |
| | C(3)-W-P-CONH2 (peak 1) |
| 307 | Ac-W-T-(Allo-Ile)-C(3)-[3-(1-morpholinyl)-Ala]-N-(2Nal)-Lys(DOTA)-D-C(3)-W-P- |
| | CONH2 (peak 1) |
| 308 | Ac-W-T-(THPG)-C(3)-[3-(1-morpholinyl)-Ala]-N-(2Nal)-Lys(DOTA)-D-C(3)-W-P- |
| | CONH2 (peak 1) |
| 309 | Ac-W-T-(Tbg)-C(3)-[3-(1-morpholinyl)-Ala]-N-(2Nal)-Lys(DOTA)-D-C(3)-W-P- |
| | CONH2 (peak 2) |
| 310 | Ac-W-T-(t-Bu-Ala)-C(3)-[3-(1-morpholinyl)-Ala]-N-(2Nal)-Lys(DOTA)-D-C(3)-W-P- |
| | CONH2 (peak 2) |
| 311 | Ac-W-T-(α-tert-amylGly)-C(3)-[3-(1-morpholinyl)-Ala]-N-(2Nal)-Lys(DOTA)-D- |
| | C(3)-W-P-CONH2 (peak 2) |
| 312 | Ac-W-T-(Allo-Ile)-C(3)-[3-(1-morpholinyl)-Ala]-N-(2Nal)-Lys(DOTA)-D-C(3)-W-P- |
| | CONH2 (peak 2) |
| 313 | Ac-W-T-(THPG)-C(3)-[3-(1-morpholinyl)-Ala]-N-(2Nal)-Lys(DOTA)-D-C(3)-W-P- |
| | CONH2 (peak 2) |
| 314 | Ac-W-T-(t-Bu-Ala)-C(3)-[3-(4-piperidinyl)-Ala]-N-(2Nal)-Lys(DOTA)-D-C(3)-W-P- |
| | CONH2 |
| 315 | Ac-W-T-(Tbg)-C(3)-N-N-(2Nal)-Lys(DOTA)-D-C(3)-W-P-CONH2 |
| 316 | Ac-W-T-(t-Bu-Ala)-C(3)-N-N-(2Nal)-Lys(DOTA)-D-C(3)-W-P-CONH2 |
| 317 | Ac-W-T-(THPG)-C(3)-N-N-(2Nal)-Lys(DOTA)-D-C(3)-W-P-CONH2 |
| 318 | Ac-W-T-(Chg)-C(3)-N-N-(2Nal)-Lys(DOTA)-D-C(3)-W-P-CONH2 |
| 319 | Ac-W-T-(THPG)-C(3)-[3-(4-piperidinyl)-Ala]-N-(2Nal)-Lys(DOTA)-D-C(3)-W-P- |
| | CONH2 |
| 320 | Ac-W-T-(NMe-Nle)-C(3)-N-N-(2Nal)-Lys(DOTA)-D-C(3)-W-P-CONH2 |
| 321 | Ac-W-T-(α-tert-amylGly)-C(3)-N-N-(2Nal)-Lys(DOTA)-D-C(3)-W-P-CONH2 |
| 322 | Ac-W-T-(Allo-Ile)-C(3)-N-N-(2Nal)-Lys(DOTA)-D-C(3)-W-P-CONH2 |
| 323 | Ac-W-T-(CHA)-C(3)-N-N-(2Nal)-Lys(DOTA)-D-C(3)-W-P-CONH2 |
| 324 | Ac-W-T-(Chg)-C(3)-[3-(4-piperidinyl)-Ala]-N-(2Nal)-Lys(DOTA)-D-C(3)-W-P- |
| | CONH2 |
| 325 | Ac-W-T-(Chg)-C(3)-[3-(1-morpholinyl)-Ala]-N-(2Nal)-Lys(DOTA)-D-C(3)-W-P- |
| | CONH2 |
| 327 | Ac-W-T-(THPG)-C(3)-[3-(4-piperidinyl)-Ala]-N-(2Nal)-Lys(DOTA)-(Ala)-C(3)-W-P- |
| | CONH2 |
| 328 | Ac-W-T-(THPG)-C(3)-[3-(4-piperidinyl)-Ala]-N-(2Nal)-Lys(DOTA)-(Thr)-C(3)-W-P- |
| | CONH2 |
| 331 | Ac-W-T-I-AllyIG(4)-N-N-W-H-D-AllyIG(4)-W-P-CONH2 |
| 332 | Ac-(4Pya)-T-I-C(3)-N-N-W-H-D-C(3)-W-P-CONH2 |
| 333 | Ac-(BIP)-T-I-C(3)-N-N-W-H-D-C(3)-W-P-CONH2 |
| 334 | Ac-W-T-I-C(3)-N-N-W-H-D-C(3)-W-(Sar)-CONH2 |
| 335 | Ac-W-T-I-C(3)-N-N-W-H-D-C(3)-W-(Aze)-CONH2 |
| 336 | Ac-W-T-(Tbg)-C(3)-N-N-W-H-D-C(3)-W-P-CONH2 |
| 337 | Ac-W-T-(Env)-C(3)-N-N-W-H-D-C(3)-W-P-CONH2 |
| 338 | Ac-W-T-(THPG)-C(3)-N-N-W-H-D-C(3)-W-P-CONH2 |
| 339 | Ac-W-T-(Nle)-C(3)-N-N-W-H-D-C(3)-W-P-CONH2 |
| 340 | Ac-W-T-(CHA)-C(3)-N-N-W-H-D-C(3)-W-P-CONH2 |
| 341 | Ac-W-T-(CBA)-C(3)-N-N-W-H-D-C(3)-W-P-CONH2 |
| 342 | Ac-(5Qui)-T-I-C(3)-N-N-W-H-D-C(3)-W-P-CONH2 |
| 343 | Ac-(4Fluoro-F)-T-I-C(3)-N-N-W-H-D-C(3)-W-P-CONH2 |
| 377 | Ac-W-T-(Tbg)-C(3)-[3-(4-piperidinyl)-Ala]-N-(2Nal)-[Glu(gLys(OH)-Val-Met-AmBz- |
| | DOTA)]-D-C(3)-W-P-CONH2 |
| 378 | Ac-W-T-(THPG)-C(3)-[3-(4-piperidinyl)-Ala]-N-(2Nal)-[Glu(gLys(OH)-Val-Met- |
| | AmBz-DOTA)]-D-C(3)-W-P-CONH2 |
| 379 | Ac-W-T-(Chg)-C(3)-[3-(4-piperidinyl)-Ala]-N-(2Nal)-Lys(Chx-DOTA)-D-C(3)-W-P- |
| | CONH2 |
| 380 | Ac-W-T-(Chg)-C(3)-[3-(1-morpholinyl)-Ala]-N-(2Nal)-Lys(Chx-DOTA)-D-C(3)-W-P- |
| | CONH2 |
| 381 | Ac-W-T-(Chg)-C(3)-[3-(4-piperidinyl)-Ala]-N-(2Nal)-[Glu(gLys(OH)-Val-Met-AmBz- |
| | DOTA)]-D-C(3)-W-P-CONH2 |
| 382 | Ac-(NMeW)-T-I-C(3)-N-N-W-H-D-C(3)-W-P-CONH2 |
| 383 | Ac-W-(NMeT)-I-C(3)-N-N-W-H-D-C(3)-W-P-CONH2 |
| 384 | Ac-W-T-I-C(3)-(NMeN)-N-W-H-D-C(3)-W-P-CONH2 |
| 385 | Ac-W-T-I-C(3)-N-(NMeN)-W-H-D-C(3)-W-P-CONH2 |
| 386 | Ac-W-T-I-C(3)-N-N-(NMeW)-H-D-C(3)-W-P-CONH2 |
| 387 | Ac-W-T-I-C(3)-N-N-W-(NMeH)-D-C(3)-W-P-CONH2 |
| 388 | Ac-W-T-I-C(3)-N-N-W-H-(NMeD)-C(3)-W-P-CONH2 |
| 389 | Ac-W-T-I-C(3)-N-N-W-H-D-C(3)-(NMeW)-P-CONH2 |
| 398 | Ac-W-T-(Chg)-C(1)-[3-(4-piperidinyl)-Ala]-N-(2Nal)-Lys(DOTA)-D-C(1)-W-P- |
| | CONH2 |
| 399 | Ac-W-T-(Chg)-C(1)-[3-(4-piperidinyl)-Ala]-N-(2Nal)-Lys(DOTA)-(Thr)-C(1)-W-P- |
| | CONH2 |
| 400 | Ac-W-T-(NMe-Nle)-C(1)-[3-(4-piperidinyl)-Ala]-N-(2Nal)-Lys(DOTA)-D-C(1)-W-P- |
| | CONH2 |
| 401 | Ac-W-T-(Chg)-C(oDBX)-[3-(4-piperidinyl)-Ala]-N-(2Nal)-Lys(DOTA)-D-C(3)-W-P- |
| | CONH2 |
| 402 | Ac-W-T-(Chg)-C(mDBX)-[3-(4-piperidinyl)-Ala]-N-(2Nal)-Lys(DOTA)-D-C(3)-W-P- |
| | CONH2 |
| 404 | Ac-(7Me-Trp)-T-(Chg)-C(3)-[3-(4-piperidinyl)-Ala]-N-(2Nal)-Lys(DOTA)-D-C(3)-W- |
| | P-CONH2 |
| 405 | Ac-(7MeO-Trp)-T-(Chg)-C(3)-[3-(4-piperidinyl)-Ala]-N-(2Nal)-Lys(DOTA)-D-C(3)- |
| | W-P-CONH2 |
| 406 | Ac-W-T-(Chg)-C(3)-[3-(4-piperidinyl)-Ala]-N-(2Nal)-Lys(G-G-TTDS-DOTA)-D-C(3)- |
| | W-P-CONH2 |
| 407 | Ac-W-T-(Chg)-C(3)-[3-(4-piperidinyl)-Ala]-N-(2Nal)-Lys(G-S-G-S-G-S-DOTA)-D- |
| | C(3)-W-P-CONH2 |
| 408 | Ac-(7Me-Trp)-T-(NMe-Nle)-C(3)-[3-(4-piperidinyl)-Ala]-N-(2Nal)-Lys(DOTA)-D- |
| | C(3)-W-P-CONH2 |
| 416 | Ac-W-T-(Chg)-C(1)-[3-(4-piperidinyl)-Ala]-N-(2Nal)-Lys(DOTA)-(Ala)-C(1)-W-P- |
| | CONH2 |
| 417 | Ac-W-T-(Chg)-C(1)-[3-(4-piperidinyl)-Ala]-N-(2Nal)-Lys(DOTA)-D-C(1)-W-P-[D- |
| | Lys(PEG8-plPh))]-CONH2 |
| 418 | Ac-W-T-(Chg)-C(1)-[3-(4-piperidinyl)-Ala]-N-(2Nal)-Lys(DOTA)-D-C(1)-W-P-[D- |
| | Lys(PEG8-pBrPh))]-CONH2 |
| 419 | Ac-W-T-(NMe-tBuAla)-C(3)-[3-(4piperdinyl)-Ala]-N-(2Nal)-Lys(DOTA)-D-C(3)-W- |
| | P-CONH2 |
| 420 | Ac-W-T-(Chg)-C(3)-[3-(4-piperidinyl)-Ala]-N-(2Nal)-Lys(DOTA)-D-C(3)-W-P-[D- |
| | Lys(PEG8-plPh))]-CONH2 |
| 421 | Ac-W-T-(Chg)-C(3)-[3-(4-piperidinyl)-Ala]-N-(2Nal)-Lys(DOTA)-D-C(3)-W-P-[D- |
| | Lys(PEG8-pBrPh))]-CONH2 |
| 422 | Ac-(7MeO-Trp)-T-(NMe-Nle)-C(3)-[3-(4piperdinyl)-Ala]-N-(2Nal)-Lys(DOTA)-D- |
| | C(3)-W-P-CONH2 |
| 423 | Ac-W-T-(t-Bu-Ala)-[D-Cys(1)]-N-N-(2Nal)-Lys(DOTA)-D-C(1)-W-P-CONH2 |
| 424 | Ac-W-T-(t-Bu-Ala)-C(1)-N-N-(2Nal)-Lys(DOTA)-D-[D-Cys(1)]-W-P-CONH2 |
| 425 | Ac-W-T-(Chg)-C(1)-[3-(4-piperidinyl)-Ala]-N-(2Nal)-[Glu(gLys(OH)-Val-Met-AmBz- |
| | DOTA)]-D-C(1)-W-P-CONH2 |
| 428 | Ac-(7Me-Trp)-T-(Chg)-C(1)-[3-(4-piperidinyl)-Ala]-N-(2Nal)-Lys(DOTA)-D-C(1)-W- |
| | P-NH2 |
| 429 | Ac-(7MeO-Trp)-T-(NMe-Nle)-C(1)-[3-(4-piperidinyl)-Ala]-N-(2Nal)-Lys(DOTA)-D- |
| | C(1)-W-P-NH2 |
| 430 | Ac-(7MeO-Trp)-T-(Chg)-C(1)-[3-(4-piperidinyl)-Ala]-N-(2Nal)-Lys(DOTA)-D-C(1)- |
| | W-P-CONH2 |
| 431 | Ac-(7Me-Trp)-T-(NMe-Nle)-C(1)-[3-(4-piperidinyl)-Ala]-N-(2Nal)-Lys(DOTA)-D- |
| | C(1)-W-P-CONH2 |
| 432 | Ac-W-T-(Chg)-C(1)-[3-(4-piperidinyl)-Ala]-N-(2Nal)-Lys(Chx-DOTA)-D-C(1)-W-P- |
| | CONH2 |
| 434 | Ac-W-T-(t-Bu-Ala)-C(3)-[3-(4-piperidinyl)-Ala]-N-(2Nal)-Lys(Chx-DOTA)-D-C(3)-W- |
| | P-NH2 |
| 435 | Ac-W-T-(t-Bu-Ala)-C(1)-[3-(4-piperidinyl)-Ala]-N-(2Nal)-Lys(DOTA)-D-C(1)-W-P- |
| | COOH |
| 436 | Ac-W-T-(t-Bu-Ala)-C(1)-[3-(4-piperidinyl)-Ala]-N-(2Nal)-Lys(DOTA)-D-C(1)-W- |
| | (aMe-P)-COOH |
| 437 | Ac-W-T-(t-Bu-Ala)-C(1)-[3-(4-piperidinyl)-Ala]-N-(2Nal)-Lys(DOTA)-D-C(1)-W- |
| | (aMe-P)-CONH2 |
| 438 | Ac-W-T-(t-Bu-Ala)-C(1)-[3-(4-piperidinyl)-Ala]-N-(2Nal)-Lys(DOTA)-D-C(1)-W-P- |
| | [D-Lys(Ac)]-CONH2 |
| 439 | Ac-W-T-(t-Bu-Ala)-C(1)-[3-(4-piperidinyl)-Ala]-N-(2Nal)-Lys(DOTA)-D-C(1)-W-P- |
| | [D-Lys(Ac)]-COOH |
| 440 | Ac-(L-Nva)-W-T-I-C(3)-N-N-W-H-D-C(3)-W-P-CONH2 |
| 441 | Ac-(L-Nle)-W-T-I-C(3)-N-N-W-H-D-C(3)-W-P-CONH2 |
| 442 | Ac-W-T-(NMe-tBuAla)-C(1)-[3-(4-piperidinyl)-Ala]-N-(2Nal)-Lys(DOTA)-D-C(1)-W- |
| | P-CO2H |
| 443 | Ac-W-T-(NMe-tBuAla)-C(1)-[3-(4-piperidinyl)-Ala]-N-(2Nal)-Lys(DOTA)-D-C(1)-W- |
| | (aMe-P)-CO2H |
| 445 | Ac-W-T-(NMe-tBuAla)-C(1)-[3-(4-piperidinyl)-Ala]-N-(2Nal)-Lys(DOTA)-D-C(1)-W- |
| | P-[D-Lys(Ac)]-CONH2 |
| 446 | Ac-W-T-(NMe-tBuAla)-C(1)-[3-(4-piperidinyl)-Ala]-N-(2Nal)-Lys(DOTA)-D-C(1)-W- |
| | P-[D-Lys(Ac)]-COOH |
| 447 | Ac-W-T-(NMe-tBuAla)-C(1)-[PipA(acetic)]-N-(2Nal)-Lys(DOTA)-D-C(1)-W-P- |
| | CONH2 |
| 448 | Ac-W-T-(NMe-tBuAla)-C(1)-[PipA(Peg)]-N-(2Nal)-Lys(DOTA)-D-C(1)-W-P-CONH2 |
| 449 | Ac-W-T-(NMe-tBuAla)-C(1)-[PipA(acetate-DOTA)]-N-(2Nal)-Lys(DOTA)-D-C(1)- |
| | W-P-CONH2 |

or a pharmaceutically acceptable salt thereof, wherein 0(3) and/or Pen(3) indicate that the two Cysteine residues are involved in cyclic bond formation with disulfide, and wherein C(1) and/or D-Cys(1) indicates that the two Cysteine residues are involved in cyclic bond formation with a thioacetal bridge (S—CH₂—S or methylene cross-linker).

In an embodiment, m is 0 or 1;
P¹ is:

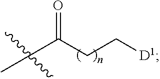

n is 0 to 16;
P² is C(O)NH₂ or C(O)OH;
D¹ is selected from H, OH₃, and C(O)OH;
R¹ is an amino acid side chain selected from the group consisting of Trp, 21Nal, 1 Nal, 4CF₃-Phe, 7Aza-Trp, 1Me-Trp, 50H-Trp, BIP, 5OMe-Trp, 4F-Phe, 3Pya, 4Pya, PAF, MAF, OAF, 5Qui, 7OMe-Trp, 7Me-Trp, 5F-Trp, 7Cl-Trp, D-Ala, Ala, alpha-Me-Trp, and NMe-Trp;
R² is an amino acid side chain selected from the group consisting of Thr, D-Ala, Ala, alpha-Me-Thr, Lys, and NMe-Thr;
R³ an amino acid side chain selected from the group consisting of Ile, Env, CHA, CBA, Nle, Tbg, THPG, Chg, 21Nal, 1Nal, 2CF₃-Phe, 2PhEt-Ala, D-Ala, Ala, Leu, t-Bu-Ala, NMe-Nle, α-tert-amylGly, Allo-Ile, Lys(C12), Lys(C14), Lys(C16), alpha-Me-Ile, and NMe-tBuAla;
A¹ is selected from the group consisting of:

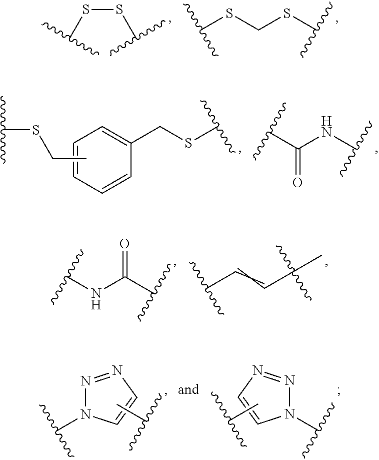

R⁴ is an amino acid side chain selected from the group consisting of Asn, D-Ala, Ala, DAB-4-NHCOC₅H₁₁, DAB-4-NHCOC₇H₁₅, Asp, Ser, Lys(DOTA), Lys, 3-(4-piperidinyl)-Ala, 3-(1-morpholinyl)-Ala, 3Pya, 4Pya, Glu, NMe-Asn, Pip(CH₂CO₂H)Ala, Pip(PegNMe₃)Ala, and Pip(GAE-DOTA)Ala;
R⁵ is an amino acid side chain selected from the group consisting of Asn, Ala, D-Ala, Trp, Asp, Lys, Lys(DOTA), 3Pya, 4Pya, 3-(4-piperidinyl)-Ala, 3-(1-morpholinyl)-Ala, Glu, NMe-Asn, and Ser;
R⁶ is an amino acid side chain selected from the group consisting of Trp, 4CF₃-Phe, 1Me-Trp, 7Aza-Trp, BIP, 2Nal, 1Nal, aMe-Trp, D-Ala, Ala, 4F-Phe, 1Nal, 2Nal, 5F-Trp, 5OMe-Trp, Asn, 50H-Trp, 7Me-Trp, 7MeO-Trp, 7Cl-Trp, and NMe-Trp;
R⁷ is selected from the group consisting of:

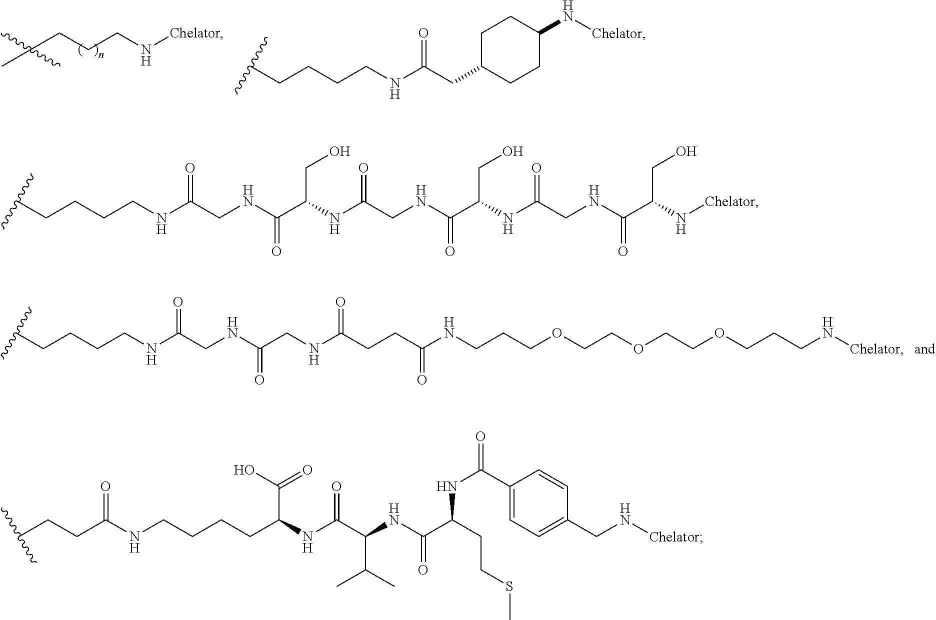

R⁸ is an amino acid side chain selected from the group consisting of Asp, D-Ala, Ala, Asn, Thr, NMe-Asp, and alpha-Me-Asp;
R⁹ is an amino acid side chain selected from the group consisting of Trp, 7Aza-Trp, 1Me-Trp, D-Ala, Ala, 4F-Phe, 1Nal, 2Nal, 5F-Trp, 5OMe-Trp, alpha-Me-Trp, 7Cl-Trp, 50H-Trp, 7Me-Trp, 7MeO-Trp, and NMe-Trp; and
R¹⁰ is an amino acid side chain selected from the group consisting of Pro, D-Ala, Ala, alpha-Me-Pro, trans4Fluoro-Pro, cis4Fluoro-Pro, trans40H-Pro, cis40H-Pro, Pip, 5,5-diMe-Pro, NMe-Ser, trans4NH2-Pro, cis4NH2-Pro, Sar, Aze, NMe-Ala, NMe-Leu, R-3Me-Aze, alpha-Me-Aze, ACl, and 3Me2-Aze.

In another embodiment, A¹ is selected from the group consisting of:

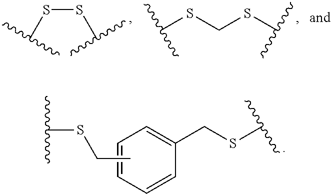

In yet another embodiment, A¹ is:

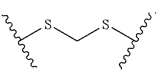 [SMILES: *SCS*]

In still another embodiment, R⁷ is selected from the group consisting of:

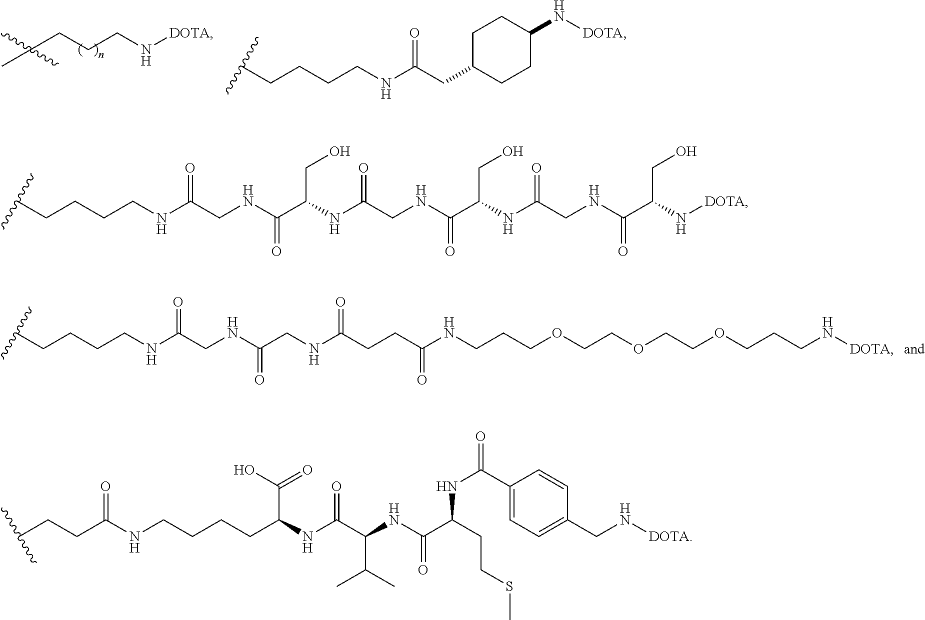

In an embodiment, R⁷ is:

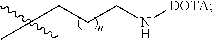

and n is 0 to 6. In yet another embodiment, n is 2.

In still another embodiment, m is 0.

In an embodiment, m is 0 or 1;
P¹ is:

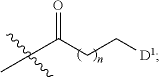

n is 0 to 16;
P² is C(O)NH₂ or C(O)OH;
D¹ is selected from H, CH₃, and C(O)OH;
R¹ is an amino acid side chain selected from the group consisting of Trp, 2Nal, 1 Nal, 7Aza-Trp, 1Me-Trp, 50H-Trp, BIP, 5OMe-Trp, 3Pya, 4Pya, 5Qui, 7OMe-Trp, 7Me-Trp, 5F-Trp, 7Cl-Trp, alpha-Me-Trp, and NMe-Trp;
R² is an amino acid side chain selected from the group consisting of Thr, D-Ala, Ala, alpha-Me-Thr, Lys, and NMe-Thr;
R³ an amino acid side chain selected from the group consisting of Ile, Env, Nle, D-Ala, Ala, Leu, t-Bu-Ala, NMe-Nle, α-tert-amylGly, Allo-Ile, Lys(C12), Lys(C14), Lys(C16), alpha-Me-Ile, and NMe-tBuAla;
A¹ is selected from the group consisting of:

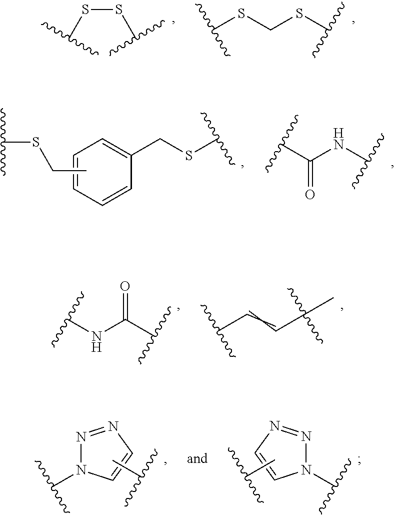

R⁴ is an amino acid side chain selected from the group consisting of Asn, D-Ala, Ala, DAB-4-NHCOC₅H₁₁, DAB-4-NHCOC₇H₁₅, Asp, Ser, Lys(DOTA), Lys, 3-(4-piperidinyl)-Ala, 3-(1-morpholinyl)-Ala, 3Pya, Glu, and NMe-Asn;
R⁵ is an amino acid side chain selected from the group consisting of Asn, Ala, D-Ala, Asp, Lys, Glu, NMe-Asn, and Ser;
R⁶ is an amino acid side chain selected from the group consisting of Trp, 4CF₃-Phe, 1Me-Trp, 7Aza-Trp, BIP, 2Nal, 1Nal, aMe-Trp, 4F-Phe, 1Nal, 2Nal, 5F-Trp, 5OMe-Trp, 50H-Trp, 7Me-Trp, 7MeO-Trp, 7Cl-Trp, and NMe-Trp;
R⁷ is selected from the group consisting of:

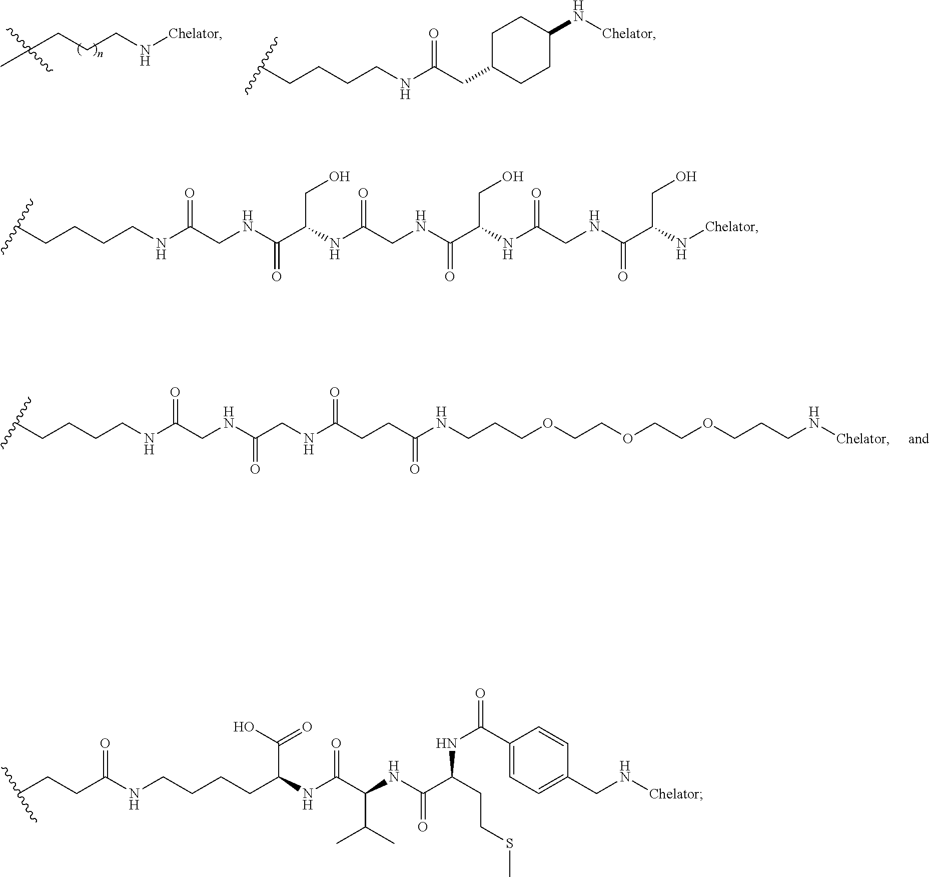

R⁸ is an amino acid side chain selected from the group consisting of Asp, D-Ala, Ala, Asn, Thr, NMe-Asp, and alpha-Me-Asp;
R⁹ is an amino acid side chain selected from the group consisting of Trp, 7Aza-Trp, 1Me-Trp, 1 Nal, 2Nal, 5F-Trp, 5OMe-Trp, alpha-Me-Trp, 7Cl-Trp, 50H-Trp, 7Me-Trp, 7MeO-Trp, and NMe-Trp; and
R¹⁰ is an amino acid side chain selected from the group consisting of Pro, alpha-Me-Pro, trans4Fluoro-Pro, cis4Fluoro-Pro, trans40H-Pro, cis40H-Pro, 5,5-diMe-Pro, trans4NH2-Pro, cis4NH2-Pro, Aze, ACl, and 3Me2-Aze.

In another embodiment, A¹ is selected from the group consisting of:

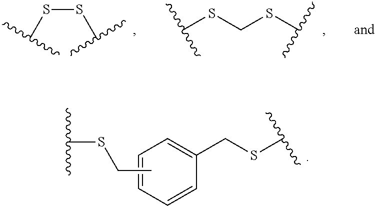

In yet another embodiment, A¹ is:

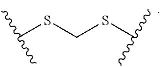 [SMILES: *SCS*]

In still another embodiment, R⁷ is selected from the group consisting of:

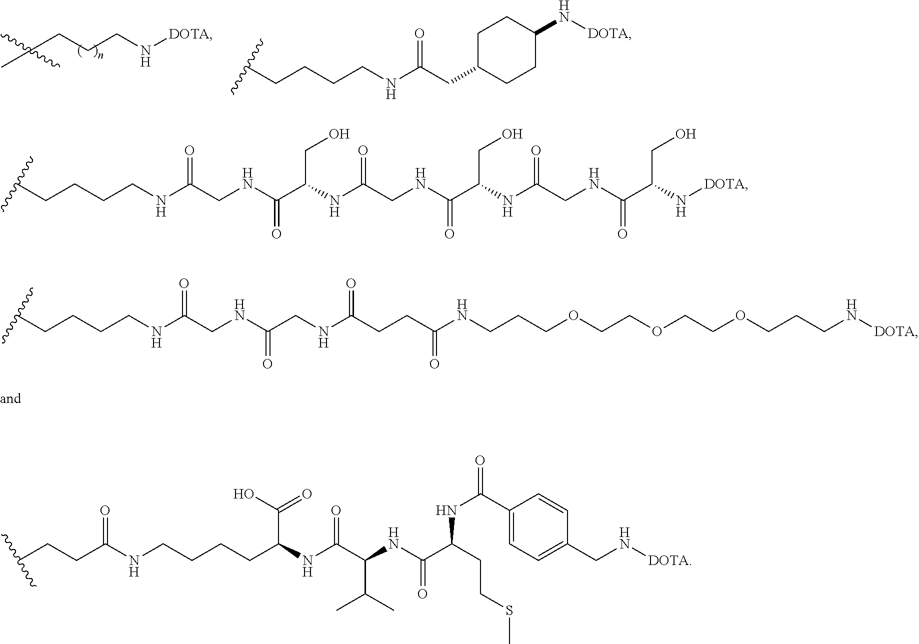

In an embodiment, R⁷ is:

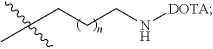

and n is 0 to 6. In yet another embodiment, n is 2.

In still another embodiment, m is 0.

In an embodiment, m is 0;
P¹ is:

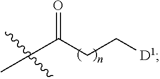

n is 0;
P² is C(O)NH₂ or C(O)OH;
D¹ is H;
R¹ is an amino acid side chain selected from the group consisting of Trp, 7Aza-Trp, 1Me-Trp, 50H-Trp, 5OMe-Trp, 7OMe-Trp, 7Me-Trp, 5F-Trp, 7Cl-Trp, alpha-Me-Trp, and NMe-Trp;
R² is an amino acid side chain selected from the group consisting of Thr, alpha-Me-Thr, Lys, and NMe-Thr;
R³ an amino acid side chain selected from the group consisting of D-Ala, Ala, t-Bu-Ala, and NMe-tBuAla;
A¹ is selected from the group consisting of:

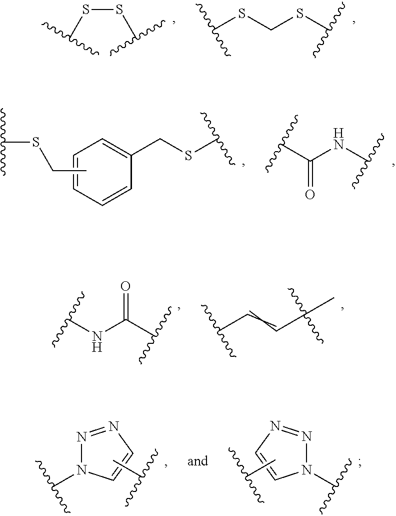

R⁴ is an amino acid side chain selected from 3-(4-piperidinyl)-Ala or 3-(1-morpholinyl)-Ala;
R⁵ is an amino acid side chain selected from the group consisting of Asn, Asp, and NMe-Asn;
R⁶ is an amino acid side chain selected from 2Nal or 1 Nal;
R⁷ is selected from the group consisting of:

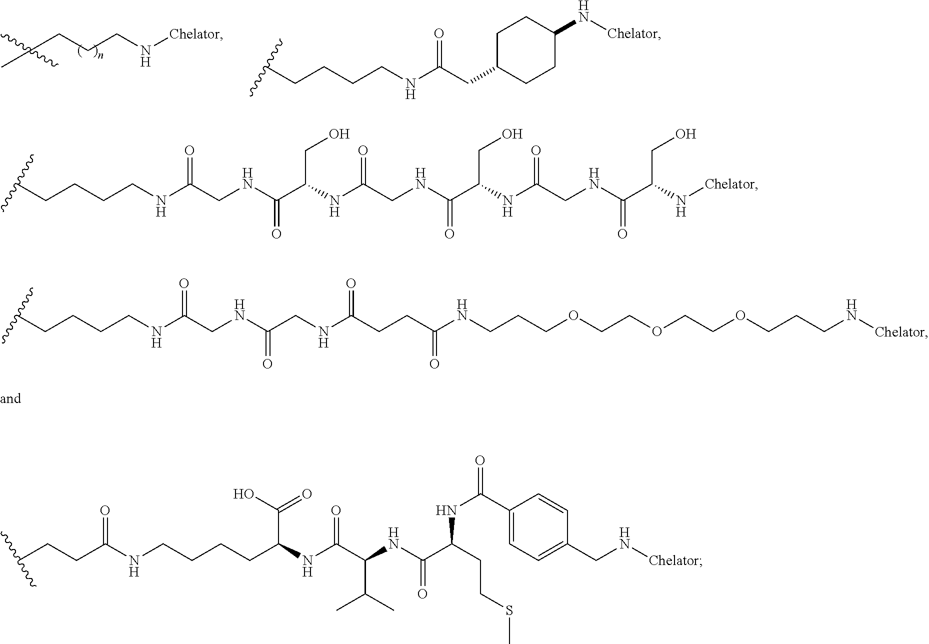

R⁸ is an amino acid side chain selected from the group consisting of Asp, Asn, NMe-Asp, and alpha-Me-Asp;
R⁹ is an amino acid side chain selected from the group consisting of Trp, 7Aza-Trp, 1Me-Trp, 5F-Trp, 5OMe-Trp, alpha-Me-Trp, 7Cl-Trp, 50H-Trp, 7Me-Trp, 7MeO-Trp, and NMe-Trp; and
R¹⁰ is an amino acid side chain selected from the group consisting of Pro, alpha-Me-Pro, trans4Fluoro-Pro, cis4Fluoro-Pro, trans40H-Pro, cis40H-Pro, 5,5-diMe-Pro, trans4NH2-Pro, and cis4NH2-Pro.

In another embodiment,
R¹ is an amino side chain of Trp;
R² is an amino side chain of Thr;
R³ is an amino side chain of NMe-tBuAla;
R⁴ is an amino side chain of 3-(4-piperidinyl)-Ala;
R⁵ is an amino side chain of NMe-Asn;
R⁶ is an amino side chain of 2Nal;
R⁸ is an amino side chain of Asp;
R⁹ is an amino side chain of Trp;
R¹⁰ is an amino side chain of Pro.

In an embodiment, m is 0;
P¹ is:

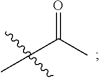 [SMILES: *C(C)=O]

P² is C(O)NH₂ or C(O)OH;
R¹ is an amino acid side chain selected from the group consisting of Trp, 7Aza-Trp, 1Me-Trp, 50H-Trp, 5OMe-Trp, 7OMe-Trp, 7Me-Trp, 5F-Trp, 7Cl-Trp, alpha-Me-Trp, and NMe-Trp;
R² is an amino acid side chain of Lys;
R³ an amino acid side chain selected from the group consisting of D-Ala, Ala, t-Bu-Ala, and NMe-tBuAla;
A¹ is

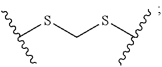 [SMILES: *SCS*]

B¹ is CH₂;
C¹ is CH₂;
R⁴ is an amino acid side chain selected from 3-(4-piperidinyl)-Ala or 3-(1-morpholinyl)-Ala;
R⁵ is an amino acid side chain selected from the group consisting of Asn, Asp, and NMe-Asn;
R⁶ is an amino acid side chain selected from 2Nal or 1 Nal;
R⁷ is

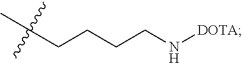

R⁸ is an amino acid side chain selected from the group consisting of Asp, NMe-Asp, and alpha-Me-Asp;
R⁹ is an amino acid side chain selected from the group consisting of Trp, 7Aza-Trp, 1Me-Trp, 5F-Trp, 5OMe-Trp, alpha-Me-Trp, 7Cl-Trp, 50H-Trp, 7Me-Trp, 7MeO-Trp, and NMe-Trp; and
R¹⁰ is an amino acid side chain selected from the group consisting of Pro, alpha-Me-Pro, trans4Fluoro-Pro, cis4Fluoro-Pro, trans40H-Pro, cis40H-Pro, 5,5-diMe-Pro, trans4NH2-Pro, and cis4NH2-Pro.

In an embodiment, the cyclic peptide of Formula III is a peptide of Table 5, or a pharmaceutically acceptable salt thereof.

TABLE 5

| Cpd# | Structure |
|---|---|
| 153 | 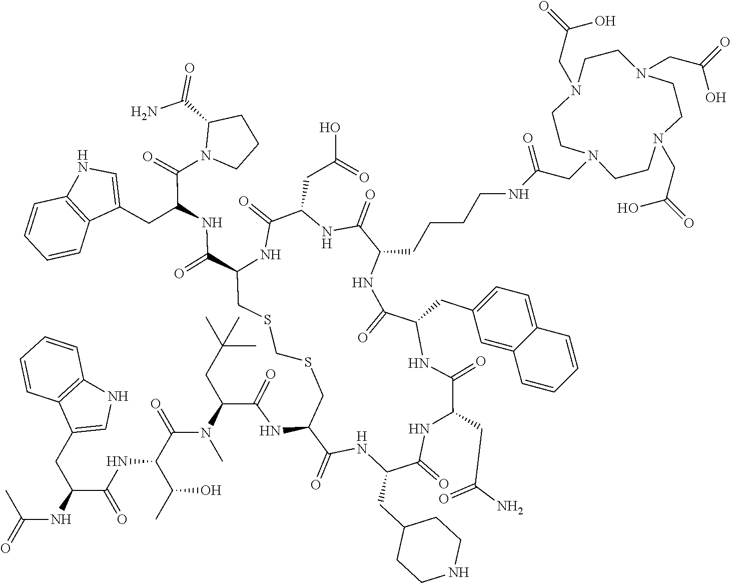 |
| 447 | 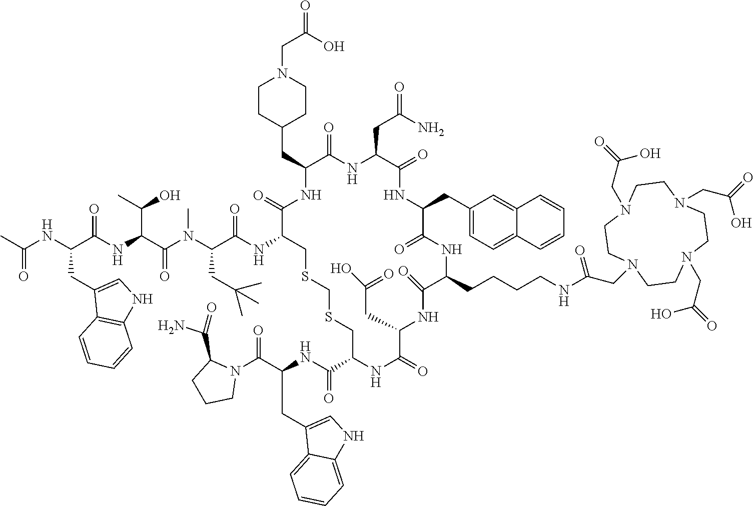 [SMILES: CC(=O)N[C@@H](Cc1c[nH]c2ccccc12)C(=O)N[C@H](C(=O)N(C)[C@@H](CC(C)(C)C)C(=O)N[C@H]1CSCSC[C@@H](C(=O)N[C@@H](Cc2c[nH]c3ccccc23)C(=O)N2CCC[C@H]2C(N)=O)NC(=O)[C@H](CC(=O)O)NC(=O)[C@H](CCCCNC(=O)CN2CCN(CC(=O)O)CCN(CC(=O)O)CCN(CC(=O)O)CC2)NC(=O)[C@H](Cc2ccc3ccccc3c2)NC(=O)[C@H](CC(N)=O)NC(=O)[C@H](CC2CCN(CC(=O)O)CC2)NC1=O)[C@@H](C)O] |

In another embodiment, the cyclic peptide is Compound 496:

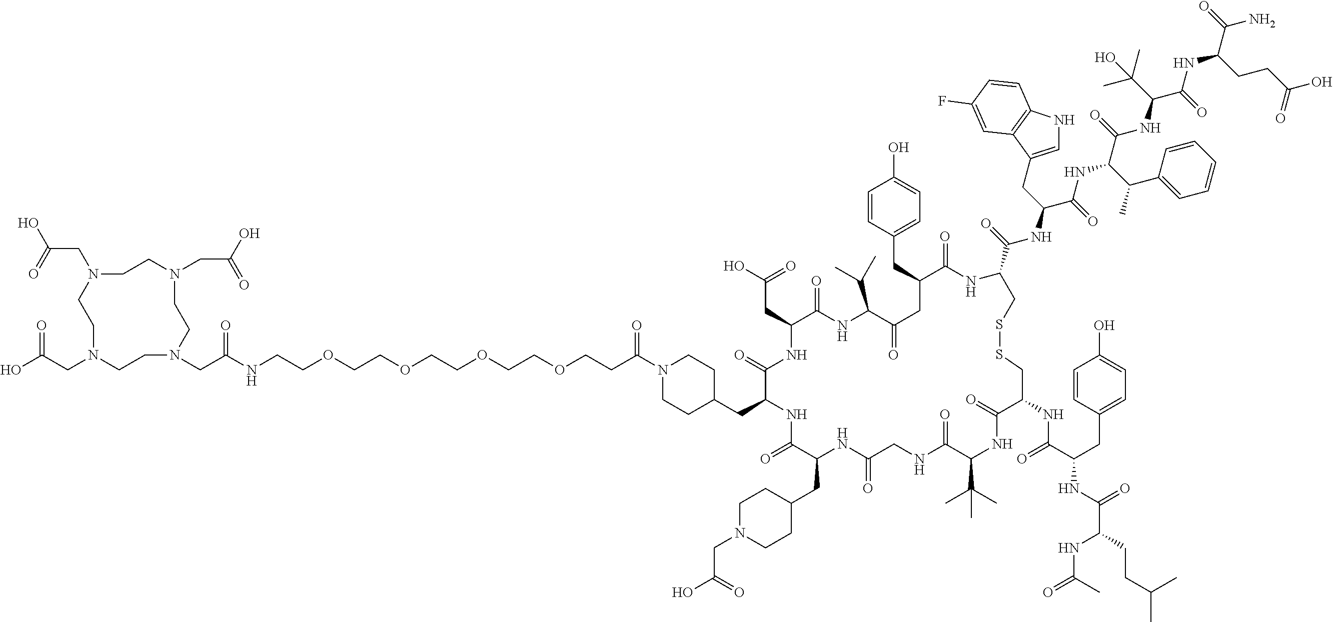 [SMILES: CC(=O)N[C@@H](CCC(C)C)C(=O)N[C@@H](Cc1ccc(O)cc1)C(=O)N[C@H]1CSSC[C@@H](C(=O)N[C@@H](Cc2c[nH]c3ccc(F)cc23)C(=O)N[C@H](C(=O)N[C@H](C(=O)N[C@H](CCC(=O)O)C(N)=O)C(C)(C)O)[C@@H](C)c2ccccc2)NC(=O)[C@H](Cc2ccc(O)cc2)CC(=O)[C@H](C(C)C)NC(=O)[C@H](CC(=O)O)NC(=O)[C@H](CC2CCN(C(=O)CCOCCOCCOCCOCCNC(=O)CN3CCN(CC(=O)O)CCN(CC(=O)O)CCN(CC(=O)O)CC3)CC2)NC(=O)[C@H](CC2CCN(CC(=O)O)CC2)NC(=O)CNC(=O)[C@H](C(C)(C)C)NC1=O]

or a pharmaceutically acceptable salt thereof.

### Radiolabeled Compounds

In a non-limiting embodiment, the radiolabeled compound of the compositions and methods provided herein is selected from a compound of Table 6. In a non-limiting embodiment, the radiolabeled compounds are characterized by a high specific molar activity (i.e., 60-19500 μCi per nmol of compound).

TABLE 6

| Radiolabeled Compounds* | Cpd No. |
|---|---|
| 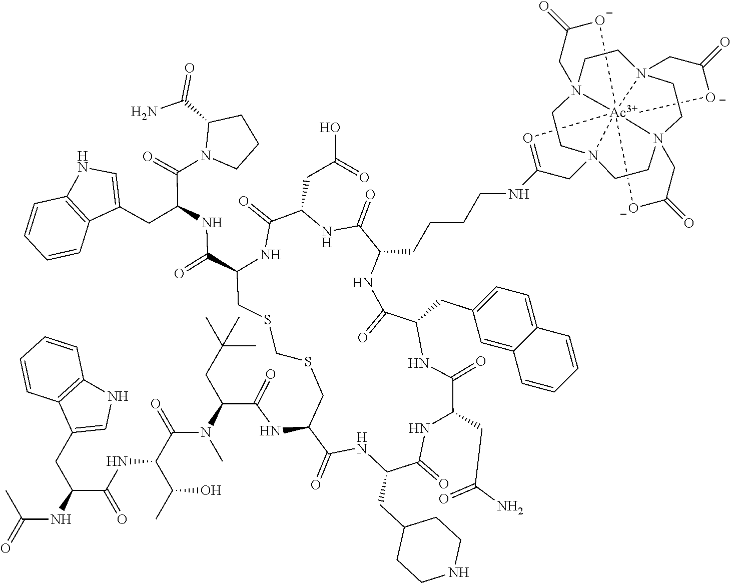 | [²²⁵Ac]Ac- 153 |
| 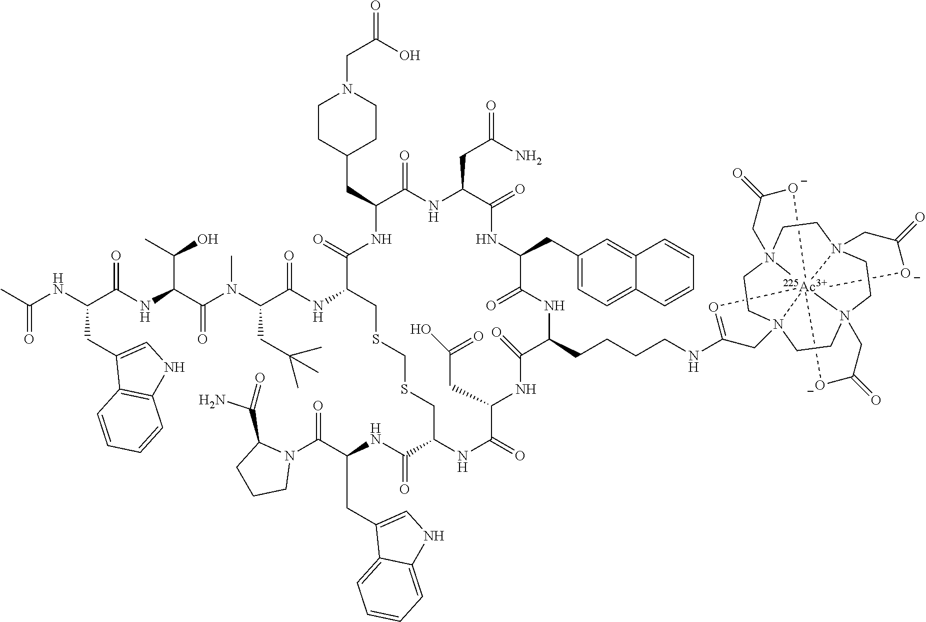 | [²²⁵Ac]Ac- 447 |
| 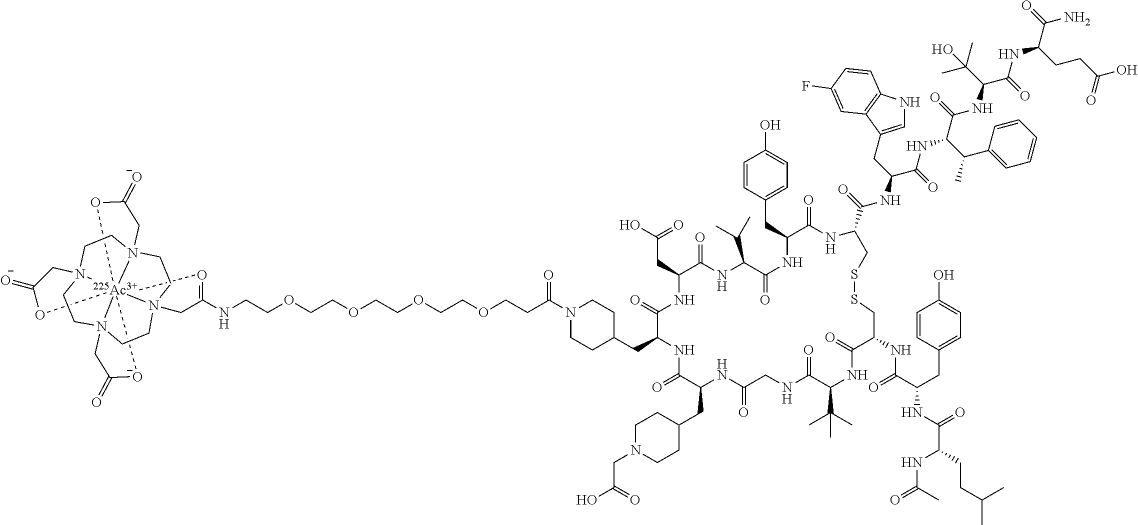 | [²²⁵Ac]Ac- 496 |
| 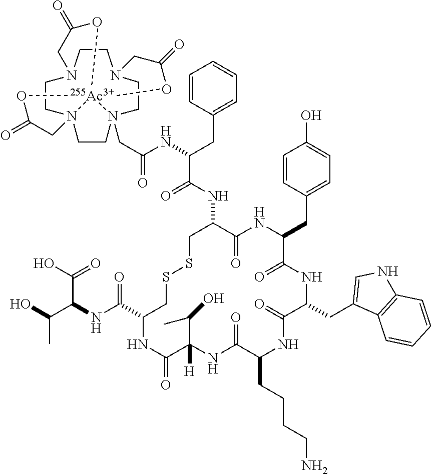 | [²²⁵Ac]Ac- DOTA- TATE |
| 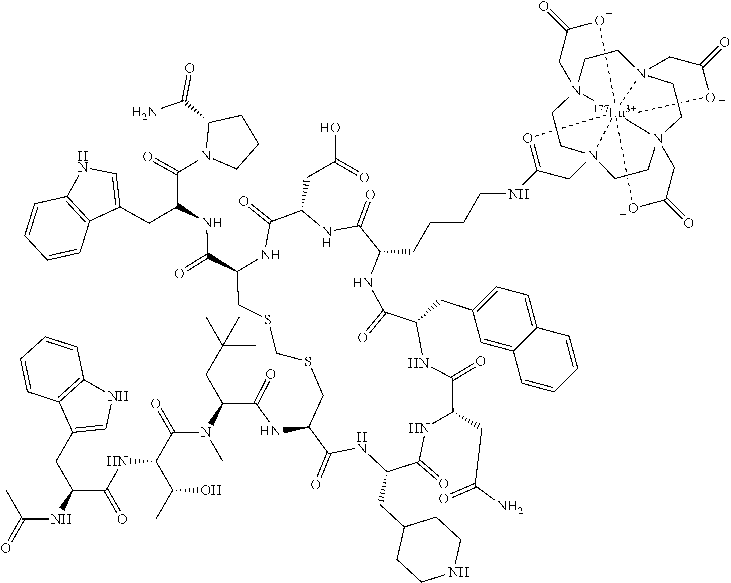 | [¹⁷⁷Lu]Lu- 153 |
| 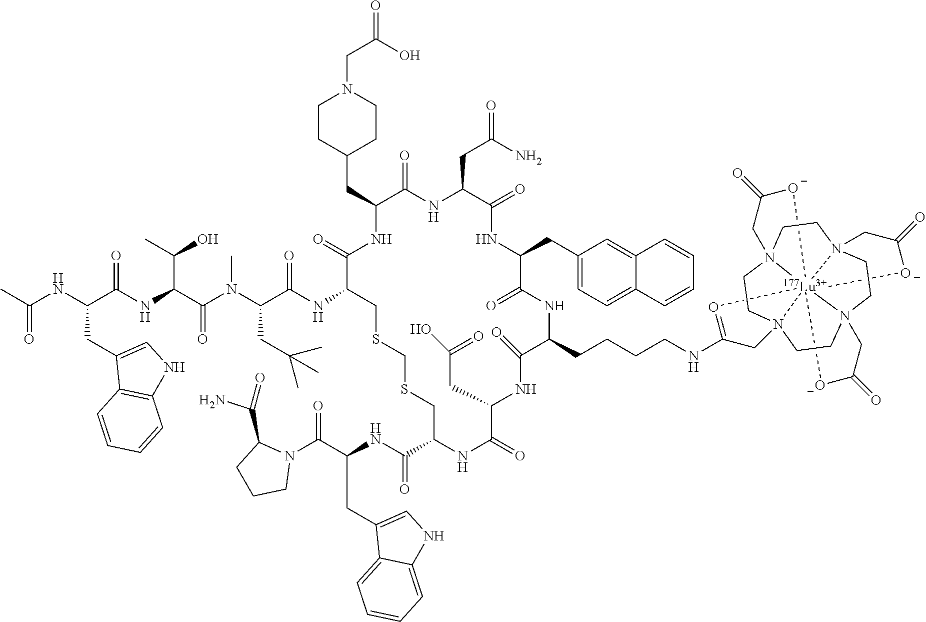 | [¹⁷⁷Lu]Lu- 447 |
| 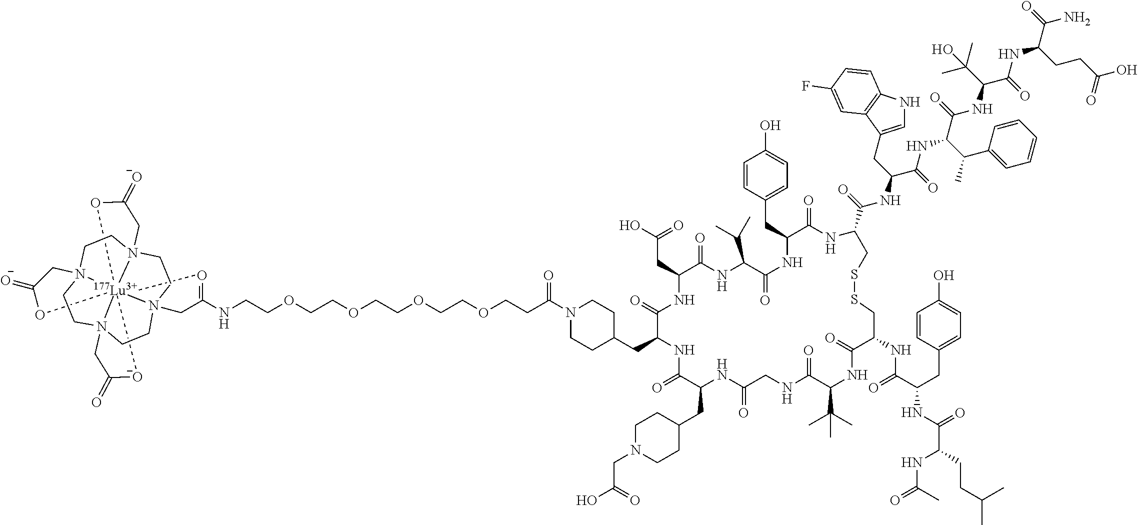 | [¹⁷⁷Lu]Lu- 496 |
| 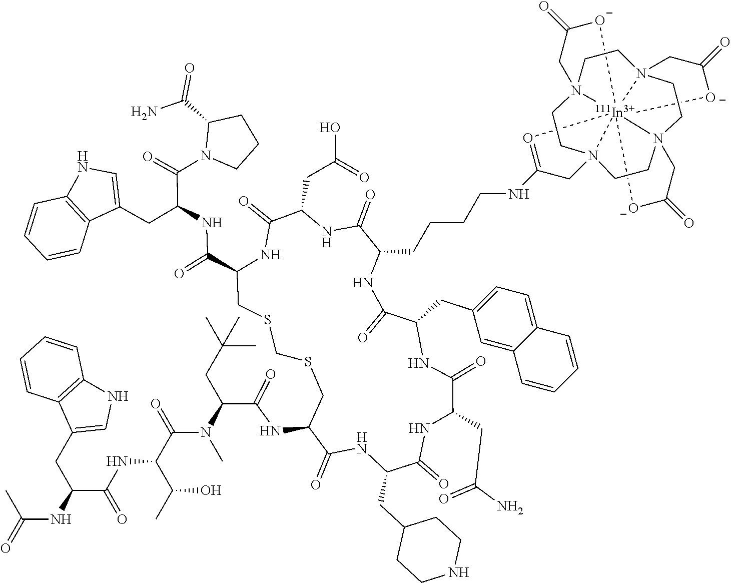 | [¹¹¹In]In- 153 |
| 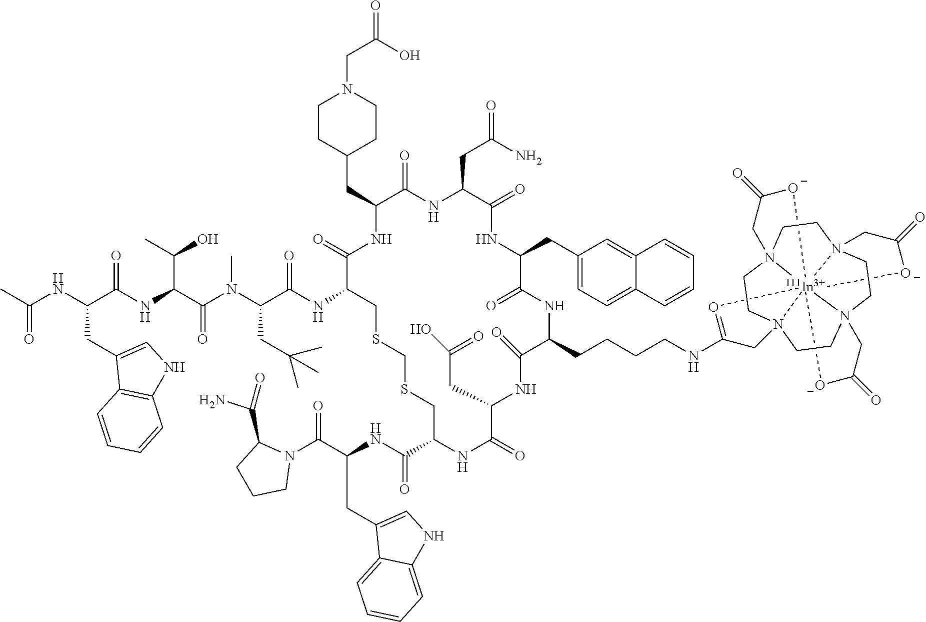 | [¹¹¹In]In- 447 |
| 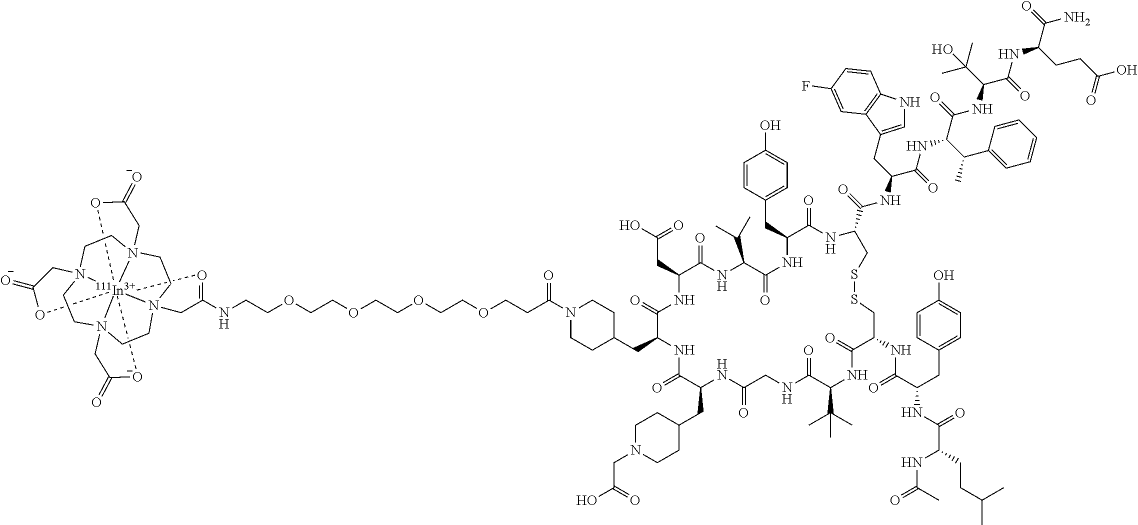 | [¹¹¹In]In- 496 |

*The drawings above represent one way in which the [²²⁵Ac]Ac-DOTA complexes (²²⁵Ac radiolabeled cyclic peptides comprising DOTA) or [¹⁷⁷Lu]Lu-DOTA complexes (¹⁷⁷Lu radiolabeled cyclic peptides comprising DOTA) could be illustrated. The same compound could be drawn, e.g., with a different number of coordination bonds, represented by either dashed or solid lines.

### DOTA-TATE Compositions

The methods described herein can be used to prepare stabilized compositions of radiolabeled DOTA-TATE that exhibit improved RCP over time.

Thus, in an aspect, provided herein is a radiopharmaceutical composition comprising enriched [²²⁵Ac]Ac-DOTA-TATE having a RCP of greater than 98%, wherein [²²⁵Ac]Ac-DOTA-TATE has the structure:

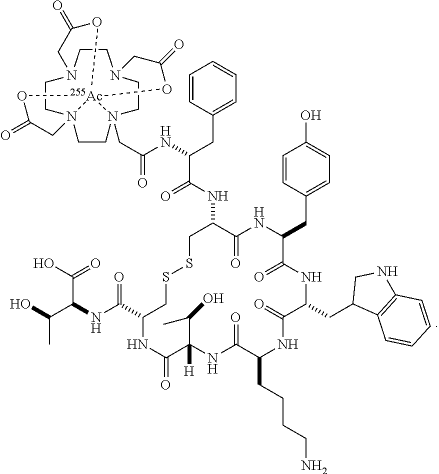

In another aspect, provided herein is a radiopharmaceutical composition comprising enriched [²²⁵Ac]Ac-DOTA-TATE having a RCP of greater than 94%, wherein the RCP is measured by radio-HPLC.

In yet another aspect, provided herein is a radiopharmaceutical composition comprising [²²⁵Ac]Ac-DOTA-TATE, wherein the radiopharmaceutical composition has a RCP of greater than 99% and 5 μCi/nmol specific activity.

In still another aspect, provided herein is a radiopharmaceutical composition comprising [²²⁵Ac]Ac-DOTA-TATE, wherein the radiopharmaceutical composition has a RCP of greater than 95% and 5 μCi/nmol specific activity.

The radiopharmaceutical compositions of the present disclosure exhibit superior stability over previously developed radiopharmaceutical compositions comprising [²²⁵Ac]Ac-DOTA-TATE.

The radionuclide may be a therapeutic radionuclide, diagnostic radionuclide, or both.

Suitable radionuclides include, but are not limited to, auger-electron emitting radionuclides, β-emitting (beta-plus or beta-minus-emitting) radionuclides, and α-emitting (alpha-emitting) radionuclides. The selection of the type of radionuclide may depend on the use of the peptide (e.g., the target of the peptide, such as somatostatin receptor, DLL3, or B7-H3). As will be appreciated by the skilled artisan, several factors may be considered when selecting a radionuclide for use in a peptide that targets, e.g., SSTR2, DLL3, or B7-H3, such as, for example, the half-life, the linear energy transfer, the imaging capabilities, and the emission range in tissue. For example, β-emitting radionuclides typically have a longer emission range in tissue (e.g., 1-5 micrometer) and emit photons in an energy range that is easily imaged, and as such, they may be selected for use in an SSTR2-targeting, a DLL3-targeting or a B7-H3-targeting compound being used for therapeutic, diagnostic, or theragnostic purposes. On the other hand, α-emitting radionuclides have a shorter emission range in tissue (e.g., 50-100 micrometer) and a high potency due to the amount of energy deposited per path length traveled (i.e., linear energy transfer), which is approximately 400 times greater than that of electrons (beta-minus particles) or positrons (beta-plus particles). Thus, α-emitting radionuclides may be selected for therapeutic uses in which high potency of the radionuclide is desired.

Accordingly, in some embodiments, the radionuclide is an α-emitting radionuclide. In other embodiments, the radionuclide is a β-emitting radionuclide. In yet other embodiments, the radionuclide is an auger-electron emitting radionuclide.

### Therapeutic and Diagnostic Methods and Uses

The stabilized compositions and pharmaceutical compositions disclosed herein are useful for imaging a subject. For example, radiolabeled compounds comprising radionuclides such as ¹¹¹In and ¹⁸F may be used as imaging agents for positron emission tomography (PET). Accordingly, provided herein is a composition for use in imaging a subject, such composition being the same as the stabilized composition and pharmaceutical composition described herein in various embodiments. Also provided is a method of imaging a subject, comprising administering to the subject a stabilized composition or pharmaceutical composition as described herein, and obtaining an image of the subject.

The stabilized compositions and pharmaceutical compositions disclosed herein are useful for the treatment of cancer. As described herein, the compositions advantageously exhibit improved stability and consequently maintain high RCP for longer timeframes than previously known formulations. The compositions contain reduced or undetectable levels of unreacted substances such as uncomplexed radionuclides and unlabeled compounds. As such, the compositions are particularly advantageous for the treatment of cancers characterized by low expression of cell-surface receptors targeted by the compounds, which would otherwise be unproductively bound by unlabeled compound. Such cell surface receptors include, for example, DLL3 and B7-H3.

Accordingly, in an aspect, provided herein is a method of treating cancer in a subject in need thereof, comprising administering to the subject a stabilized composition prepared by any one of the methods disclosed herein.

In another aspect, provided herein is a method of treating cancer in a subject in need thereof, comprising administering to the subject any one of the pharmaceutical compositions disclosed herein.

In yet another aspect, provided herein is the use of any one of the pharmaceutical compositions disclosed herein for the manufacture of a medicament for treating cancer.

In still another aspect, provided herein is any one of the pharmaceutical compositions disclosed herein for use in treating cancer.

In an embodiment of the methods, the uses, or the pharmaceutical compositions for use disclosed herein, the cancer is characterized by a cell surface protein that is characterized by an expression of about 1000-100,000 copies per cell. In some embodiments, the cell surface protein is characterized by an expression of about 1000-5000 copies per cell. In some embodiments, the cell surface protein is characterized by an expression of about 3000-5000 copies per cell. In some embodiments, the cell surface protein is characterized by an expression of about 5000-10,000 copies per cell. In some embodiments, the cell surface protein is characterized by an expression of about 10,000-30,000 copies per cell. In some embodiments, the cell surface protein is characterized by an expression of about 20,000-30,000 copies per cell. In another embodiment of the methods, the uses, or the pharmaceutical compositions for use disclosed herein, the cell surface protein is DLL3. In another embodiment of the methods, the uses, or the pharmaceutical compositions for use disclosed herein, the cell surface protein is B7-H3.

In some embodiments, the cancer is a neuroendocrine neoplasm, melanoma, or primary brain cancer. In another embodiment, the neuroendocrine neoplasm is selected from small cell lung cancer (SCLC), medullary thyroid carcinoma (MTC), large cell neuroendocrine cancer (LCNEC), gastroenteropancreatic neuroendocrine carcinoma (GEP NEC), neuroendocrine prostate cancer (NEPC), small cell prostate cancer (SCPC), Merkel cell carcinoma (MCC), neuroendocrine cervical carcinoma, and Grade 3 neuroendocrine tumors (NETs). In some embodiments, the extrapulmonary neuroendocrine carcinoma (NEC) of the cervix. In some embodiment, the small cell lung cancer can be extensive-stage (ES)-SCLC or Limited-stage (LS)-SCLC. In some embodiments, the neuroendocrine prostate cancer (NEPC), can be treatment emergent NEPC.

In some embodiments, the neuroendocrine neoplasm is selected from the group consisting of gastroenteropancreatic neuroendocrine tumor, carcinoid tumor, pheochromocytoma, paraganglioma, medullary thyroid cancer, pulmonary neuroendocrine tumor, thymic neuroendocrine tumor, a carcinoid tumor or a pancreatic neuroendocrine tumor, pituitary adenoma, adrenal gland tumors, Merkel cell carcinoma (MCC), breast cancer, Non-Hodgkin lymphoma, Hodgkin lymphoma, Head & Neck tumor, urothelial carcinoma (bladder), Renal Cell Carcinoma, Hepatocellular Carcinoma, GIST, neuroblastoma, bile duct tumor, cervix tumor, Ewing sarcoma, osteosarcoma, small cell lung cancer (SCLC), prostate cancer, melanoma, meningioma, glioma, medulloblastoma, hemangioblastoma, supratentorial primitive, neuroectodermal tumor, esthesioneuroblastoma, functional carcinoid tumor, insulinoma, gastrinoma, vasoactive intestinal peptide (VIP) oma, glucagonoma, serotoninoma, histaminoma, ACTHoma, pheocromocytoma, and somatostatinoma.

In some embodiments, the cancer is selected from the group consisting of acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor.

In some embodiments, the cancer is selected from the group consisting of primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningeal cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharyngeal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomyosarcoma, multiple myeloma, ovarian cancer, glioblastoma, gastrointestinal stromal tumor, mantle cell lymphoma, and refractory malignancy.

In some embodiments, the cancer is selected from the group consisting of breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, colorectal, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon, rectum, large intestine, rectum, brain and central nervous system, chronic myeloid leukemia (CML), and leukemia.

In some embodiments, the cancer is selected from the group consisting of myeloma, lymphoma, or a cancer selected from gastric, renal, head and neck, oropharyngeal, non-small cell lung cancer (NSCLC), endometrial, hepatocarcinoma, non-Hodgkin's lymphoma, and pulmonary.

In some embodiments, the cancer is selected from the group consisting of prostate cancer, colon cancer, lung cancer, squamous cell cancer of the head and neck, esophageal cancer, hepatocellular carcinoma, melanoma, sarcoma, gastric cancer, pancreatic cancer, ovarian cancer, breast cancer.

In some embodiments, the cancer is selected from the group consisting of tumors, neoplasms, carcinomas, sarcomas, leukemias, lymphomas, and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotropic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodysplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal, and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

In an embodiment, the cancer is a B7-H3-mediated cancer. In another embodiment, the cancer is lung cancer, urothelial cancer, melanoma, squamous cell carcinoma, endometrial cancer, breast cancer, acute myeloid leukemia (AML), gastric cancer, colorectal cancer, prostate cancer, glioma, ovarian cancer, liver cancer, cervical cancer, esophageal cancer, and head and neck cancer. In yet another embodiment, the cancer is small cell lung cancer, urothelial cancer, melanoma, or squamous cell carcinoma.

In yet another embodiment, the cancer is selected from the group consisting of acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor.

In another embodiment, the cancer is selected from the group consisting of primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomysarcoma, multiple myeloma, ovarian cancer, glioblastoma, gastrointestinal stromal tumor, mantle cell lymphoma, and refractory malignancy.

In an embodiment, the cancer is selected from the group consisting of breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, colorectal, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon, rectum, large intestine, rectum, brain and central nervous system, chronic myeloid leukemia (CML), and leukemia.

In another embodiment, the cancer is selected from the group consisting of myeloma, lymphoma, or a cancer selected from gastric, renal, head and neck, oropharangeal, non-small cell lung cancer (NSCLC), endometrial, hepatocarcinoma, non-Hodgkin's lymphoma, and pulmonary.

In an embodiment, the cancer is selected from the group consisting of prostate cancer, colon cancer, lung cancer, squamous cell cancer of the head and neck, esophageal cancer, hepatocellular carcinoma, melanoma, sarcoma, gastric cancer, pancreatic cancer, ovarian cancer, breast cancer.

In an embodiment, the cancer is selected from the group consisting of tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodysplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin syndrome (e.g., medulloblastoma, meningioma, etc.), and liver cancer. Additional exemplary forms of cancer that may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in treating are, for example, colon carcinoma, familial adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

Additional cancers that the compounds described herein may be useful in treating are, for example, colon carcinoma, familial adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngioma, osteosarcoma, chondrosarcoma, myosarcoma, liposarcoma, fibrosarcoma, Ewing sarcoma, and plasmacytoma.

Compositions in accordance with the disclosure are typically formulated in dosage unit form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. The desired dosage may be delivered three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

### EQUIVALENTS AND SCOPE

While various disclosure embodiments have been particularly shown and described in the present disclosure, it will be understood by those skilled in the art that various changes in form and details may be made without departing from the spirit and scope of the embodiments disclosed herein and set forth in the appended claims.

Those skilled in the art will recognize or be able to ascertain using no more than routine experimentation, many equivalents to the specific embodiments described herein. The scope of the present disclosure is not intended to be limited to the above description, but rather is as set forth in the appended claims.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to those of ordinary skill in the art, they may be excluded even if the exclusion is not set forth explicitly herein. Any particular embodiments of compositions disclosed herein can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

The formulations and methods disclosed herein are further illustrated by the following examples, which should not be construed as further limiting. The practice of the present disclosure will employ, unless otherwise indicated, conventional techniques of organic synthesis, cell biology, cell culture, and molecular biology, which are within the skill of the art.

Abbreviations as used herein have respective meanings as follows:

| | |
|---|---|
| Acetic acid | HOAc |
| Acetic anhydride | Ac₂O |
| Acetonitrile | ACN |
| Ammonium acetate | NH₄OAc |
| (7-Azabenzotriazol-1- | PyAOP |
| yloxy)tripyrrolidinophosphonium | |
| hexafluorophosphate | |
| Dichloromethane | DCM |
| Diiodomethane | CH₂I₂ |
| Diisopropylcarbodiimide | DIC |
| Diisopropylethylamine | DIEA |
| Dimethylformamide | DMF |
| Dithiothreitol | DTT |
| Ethanol | EtOH |
| Ethyl (hydroxyimino) | Oxyma |
| Cyanoacetate | |
| Glacial acetic acid | HOAc |
| Hexafluoroisopropanol | HFIP |
| Isopropyl alcohol | IPA |
| Methanol | MeOH |
| Methyl tert-butyl ether | MTBE |
| Nitrogen | N₂ |
| Potassium carbonate | K₂CO₃ |
| Purified water | H₂O |
| Tetrahydrofuran | THF |
| Triethylammonium phosphate | TEAP |
| Trifluoroacetic acid | TFA |
| Triisopropylsilane | TIS |
| Tris(2-carboxyethyl)phosphine | TCEP•HCl |
| hydrochloride | |
| Ultra-high performance liquid | UHPLC |
| chromatography | |

Additional terms as used herein have respective meanings as follows:

| Term | Definition |
|---|---|
| Radiochemical | The proportion of the total radioactivity in the sample, which is |
| purity (RCP) | present as the desired radiolabeled species, reported as a |
| | percentage |
| [²²⁵Ac]Ac | The proportion of chelated [²²⁵Ac]Ac with the desired chelator |
| incorporation | versus unbound in the reaction, reported as a percentage |
| Start of Synthesis | The starting point of synthesis signifies the mixing of key chemical |
| (SOS) | ingredients to enable a reaction mixture and the reaction to follow. |
| End of Synthesis | The generation of the key active product, i.e. radioactive drug |
| (EOS) | substance in-situ as complete. |
| End of Formulation | The end process of generating a final drug product that is |
| (EOF) | formulated and complete. |
| Reverse Phase- | Standard chromatographic system utilizing a non-polar stationary |
| High Performance | phase and a polar mobile phase as eluent under high pressure. |
| Liquid | |
| Chromatography | |
| (RP-HPLC) | |
| Instant Thin Layer | Standard chromatographic system utilizing a thin stationary phase |
| Chromatography | and a mobile phase used to separate compounds based on their |
| (iTLC) | affinity to either phase. |

### Example 1: Synthesis and Structure of Compound 153

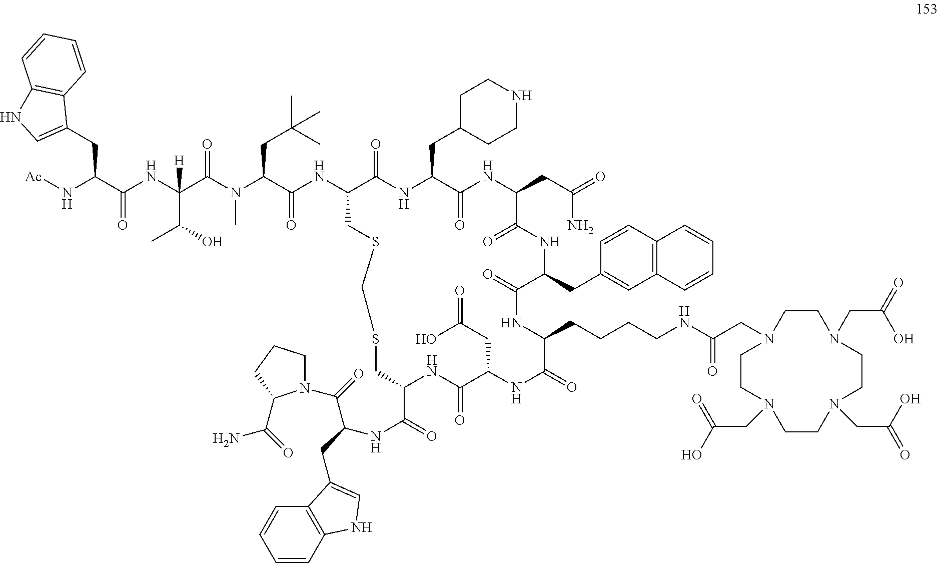 [SMILES: CC(=O)N[C@@H](Cc1c[nH]c2ccccc12)C(=O)N[C@H](C(=O)N(C)[C@@H](CC(C)(C)C)C(=O)N[C@H]1CSCSC[C@@H](C(=O)N[C@@H](Cc2c[nH]c3ccccc23)C(=O)N2CCC[C@H]2C(N)=O)NC(=O)[C@H](CC(=O)O)NC(=O)[C@H](CCCCNC(=O)CN2CCN(CC(=O)O)CCN(CC(=O)O)CCN(CC(=O)O)CC2)NC(=O)[C@H](Cc2ccc3ccccc3c2)NC(=O)[C@H](CC(N)=O)NC(=O)[C@H](CC2CCNCC2)NC1=O)[C@@H](C)O]

Compound 153 has the following sequence: Ac-Trp-Thr-N-Me-Ala(tBu)-Cys-[3-(4-piperidinyl)]Ala⁵-Asn-Nal(2′)-Lys(DOTA)-Asp-Cys¹⁰-Trp-Pro-NH₂, Cys⁴→Cys¹⁰ thioacetal bridge, OAc, wherein the underlined sequence represents the cyclic portion of Compound 153.

### Solid-Phase Peptide Synthesis (SPPS)

The peptide-resin precursor of Compound 153 was manufactured using the general procedure described in the original paper of Merrifield (Merrifield, R. B. 1963. J. Am. Chem. Soc. 85: 2149-2154), with minor modifications. The standard procedures and protocols are well developed and used routinely in the industry.

SPPS was used to sequentially attach amino acids to assemble a peptide anchored onto a solid support (resin) according to the following steps:
Deprotection (removal of the N-terminal Fmoc protecting group);
Washes using DMF;
Coupling of the amino acid derivatives;
Capping/recoupling as applicable;
Acetylation of N-terminal;
Mtt deprotection;
Deprotected peptide on resin neutralization;
DOTA coupling; and
Synthesis completion (washes DMF/IPA).

This cycle was repeated until the desired peptide was assembled. The α-amino group of each amino acid was protected with the base-sensitive 9-fluorenyl-methyloxycarbonyl (Fmoc) group, while side chain functional groups were protected with acid-labile groups, e.g., Boc or Trt derivatives. The peptide was assembled from the C-terminal to the N-terminal.

All of the amino acid derivatives used in the process are commercially available.

Compound 153 was synthesized on Fmoc-Rink-MBHA resin. The Fmoc protecting group was initially removed (i.e., deprotected) from the Fmoc-Rink Amide MBHA resin with DMF and piperidine. The synthesis employed standard Fmoc/DIC SPPS chemistry and the amino acid side chains were protected with acid-labile protecting groups. N-Fmoc deprotection steps were performed using piperidine in DMF. Oxyma-buffered piperidine/DMF was used for the Fmoc removal during the Cys¹⁰ cycle to minimize the possible Trp¹¹-Pro¹² diketopiperazine formation.

Couplings were performed with DIC as the activator and Oxyma as the additive.

Following the coupling of Trp¹ and Fmoc deprotection, acetylation of the N-terminus was performed using acetic anhydride (Ac₂O)/diisopropylethylamine (DIEA)/DMF.

Selective deprotection of the Lys⁸(Mtt) sidechain was performed using hexafluoroisopropanol (HFIP)/triisopropylsilane (TIS)/dichloromethane (DCM).

The tri-tBu-protected 1,4,7,10-tetraazacyclododecane-1,4,7,10-tetraacetic acid (DOTA(OtBu)₃-OH) coupling was performed under basic conditions with (7-Azabenzotriazol-1-yloxy) tripyrrolidinophosphonium hexafluorophosphate (PyAOP)/DIEA.

Completion of these steps resulted in a protected Compound 153 peptide anchored onto the resin.

Qualitative reaction monitoring was performed using the ninhydrin/chloranil tests. These tests measure residual, resin-bound amino groups. A negative test result indicates absence of free amines, i.e., complete coupling, and that the process can then proceed to the next coupling step. A positive test result indicates incomplete coupling; thus, prolonged coupling or re-coupling of the Fmoc amino acid may be performed.

After assembly of the sequence was completed and final deprotection was performed, the peptide on resin was washed with DMF and isopropyl alcohol (IPA) and then dried using a gentle stream of nitrogen (N₂).

The crude peptide was obtained by simultaneous cleavage of the peptide from the resin and removal of the side chain protecting groups through acidolysis using trifluoroacetic acid (TFA), resulting in the C-terminal amide form of the fully deprotected peptide. A global constraint was applied via a thioacetal bridge between the Cysteine residues. A purification scheme using reversed phase-high performance liquid chromatography (RP-HPLC) was used to generate the acetate salt form of Compound 153.

Cleavage of Peptide from Resin and Global Deprotection

Cleavage of the peptide from solid support and global deprotection was performed in a cleavage solution mixture composed of trifluoroacetic acid (TFA), water (H₂O), thioanisole, and dithiothreitol (DTT). The peptide-resin was added to the cold cleavage solution and stirred for 30 min at ambient temperature. The solution was then heated to 40° C. for 2-4 hours to complete deprotection of the DOTA moiety while monitoring the reaction progress by liquid chromatography-mass spectrometry (LC-MS).

The depleted resin is filtered off and rinsed with additional TFA. The filtrate and rinse are combined and concentrated by rotary evaporation under reduced pressure to approximately half the initial volume. The resulting concentrate is precipitated by quick addition to cold 1:1 methyl tert-butyl ether (MTBE):hexanes. The precipitate is filtered, washed with MTBE/hexanes, and dried under vacuum.

The peptide synthesis and cleavage steps are shown in Table 7 below.

TABLE 7

| | | |
|---|---|---|
| Process Step | Coupling status | Material and Solvents |

Fmoc-Rink-MBHA-resin

| | | |
|---|---|---|
| Initial deprotection | ↓ | Piperidine/DMF |
| | H-Rink-MBHA-resin | |
| Synthesis of AAs | ↓ | Coupling: Fmoc-AA- |
| | | OH/DIC/Oxyma/DMF |
| | | Washes DMF |
| | | Deprotection: Piperidine/DMF |
| | | Capping: DIEA/Ac₂O/DMF (Pro¹², |
| | | Cys^{2, 10}) |

Fmoc-Trp(Boc)-Thr(tBu)-N-Me-Ala(tBu)-Cys(Trt)-
[3-(Boc-4-piperidinyl)]Ala-Asn(Trt)-Nal(2′)-
Lys(Mtt)-Asp(tBu)-Cys(Trt)-Trp(Boc)-Pro-Rink-MBHA-Resin

| | | |
|---|---|---|
| N-terminus | ↓ | DIEA/Ac₂O/DMF |
| acetylation | | |

Ac-Trp(Boc)-Thr(tBu)-N-Me-Ala(tBu)-Cys(Trt)-[3-
(Boc-4-piperidinyl)]Ala-Asn(Trt)-Nal(2′)-
Lys(Mtt)-Asp(tBu)-Cys(Trt)-Trp(Boc)-Pro-Rink-MBHA-Resin

| | | |
|---|---|---|
| Mtt deprotection | ↓ | HFIP/TIS/DCM |
| | | Washes: DCM/DIEA/DMF |

Fmoc-Trp(Boc)-Thr(tBu)-N-Me-Ala(tBu)-Cys(Trt)-[3-
(Boc-4-piperidinyl)]Ala-Asn(Trt)-Nal(2′)-
Lys-Asp(tBu)-Cys(Trt)-Trp(Boc)-Pro-Rink-MBHA-Resin

| | | |
|---|---|---|
| DOTA(OtBu)₃ | ↓ | Coupling: DOTA(OtBu)₃- |
| coupling | | OH/PyAOP/DIEA |
| Synthesis | | Synthesis completion: Washes: |
| completion | | DMF/IPA |
| | | Dry: N₂ |

Fmoc-Trp(Boc)-Thr(tBu)-N-Me-Ala(tBu)-Cys(Trt)-
[3-(Boc-4-piperidinyl)]Ala-Asn(Trt)-Nal(2′)-
Lys(DOTA(OtBu)₃)-Asp(tBu)-Cys(Trt)-Trp(Boc)-Pro-Rink-MBHA-Resin

| | | |
|---|---|---|
| Resin cleavage and | ↓ | TFA/H₂O/DTT/thioanisole |
| global deprotection | | |

H-Trp-Thr-N-Me-Ala-Cys-[3-(4-piperidinyl)]Ala-Asn-
Nal(2′)-Lys(DOTA)-Asp-Cys-Trp-Pro-NH₂
↓
Crude Linear Peptide Precursor

### Purification of the Crude Linear Intermediate

The linear intermediate was purified by single stage RP-HPLC. The crude linear intermediate was dissolved in acetic acid (HOAc)/H₂O and stirred at 50° C. for 1 hour to promote decarboxylation of the carbamic acid intermediate (M+44) of the tryptophan residue. The column was equilibrated with acetonitrile (ACN) before loading the solution onto the column. The eluent was collected and analyzed by HPLC. The parameters of the method used for purification of the crude linear intermediate are described below in Table 8. The fraction containing the purified linear intermediate were pooled and lyophilized to yield the linear intermediate as dry powder.

TABLE 8

| Parameter | Description |
|---|---|
| Column | Analogix 75 × 250 mm, C₁₈ 100 Å 15 μm |
| Mobile Phase A | 0.2% TFA/H₂O |
| Mobile Phase B | ACN |
| Flow Rate | 150 mL/min |
| Column Temp | Ambient |
| Gradient | 5% B for 20 min |
| | 5-15% B in 10 min |
| | 15-40% B in 200 min. |

### Solution-Phase Thioacetal Cyclization

The purified linear intermediate peptide was dissolved in 50% THF/H₂O. Potassium carbonate (K₂CO₃) and Tris(2-carboxyethyl)phosphine hydrochloride (TCEP-HCl) in H₂O was added to the peptide solution. The solution was stirred for approximately 30 min. Diiodomethane (CH₂12) was added to the solution followed by K₂CO₃ in H₂O and the mixture was agitated by stirring for 18-24 h. The progress of cyclization was monitored by LC-MS. Upon completion, excess CH₂I₂ and THF was removed by rotary evaporation and the solution was acidified by addition of neat TFA.

### Purification and Counter-Ion Exchange Processes

The crude cyclized Compound 153 from the previous step was purified by a two-step, preparative, reversed phase HPLC procedure, on C₁₈ derivatized silica (Daisogel SP-120-10-ODS-RPS).

The elution from the column was monitored by UV and the fractions obtained were analyzed by an in-process RP-HPLC test. Side fractions that did not meet the purity specification were recycled through the same step of the process or discarded.

### Purification by Preparative Reversed Phase Chromatography (RPC-1)

The crude cyclized Compound 153 was diluted 1:1 with water. The solution was filtered and applied to a preparative HPLC column that was been washed with methanol and equilibrated ACN. The product was eluted using a gradient of 0.1 M triethylammonium phosphate (TEAP) pH 2.25 buffers for mobile phase A and ACN for mobile phase B, with monitoring by UV at 280 nm.

Particular parameters of the RPC-1 purification method are shown below in Table 9.

TABLE 9

| Parameter | Description |
|---|---|
| Mobile phase A | 0.1M TEAP/H₂O |
| Mobile phase B | ACN |
| Flow rate | 110 mL/min |
| Detector λ | 280 nm |
| Gradient | 5-15%; 10 min |
| | 15-40%; 250 min |

### Purification by Preparative Reversed Phase Chromatography (RPC-2)/Lyophilization

In the second stage of the purification, the semi-purified product from the first purification step was diluted 1:1 with water and loaded onto a preparative column, which was equilibrated with 0.2M aqueous HOAc and washed with 0.1M ammonium acetate (NH₄OAc). The product was eluted using a gradient of 0.2M aqueous HOAc for mobile phase A and ACN for mobile phase B, with monitoring by UV at 280 nm. The resulting pure fractions were filtered using a 0.45 um Nylon filter, shell frozen and lyophilized to dryness to give the acetate salt of Compound 153.

Particular parameters of the RPC-2 purification method are shown below in Table 10.

TABLE 10

| Parameter | Description |
|---|---|
| Mobile phase A | 0.2M HOAc/H₂O |
| Mobile phase B | ACN |
| Flow rate | 150 mL/min |
| Detector λ | 280 nm |
| Gradient | 5-40%; 120 min |

### Characterization of Compound 153

Compound 153 was characterized by electrospray ionization mass spectrometry (ESI-MS). Particular molecular ions are shown below in Table 11. identity of the counter ion was determined by reversed-phase high performance liquid chromatography (RP-HPLC). The identity of the acetate counter ion was confirmed by reversed-phase high performance liquid chromatography (RP-HPLC), and the acetic acid content was determined to be 2.6% (w/w), which corresponds to approximately 0.99 equivalents per molecule of Compound 153.

TABLE 11

| | Calculated | Observed | |
|---|---|---|---|
| Molecular ion | mass (Da) | mass (Da) | Comments |
| [M + 2H]²⁺/2 | 1042.5 | 1043.0 | Single ion |
| [M + 3H]³⁺/3 | 695.3 | 696.1 | Single ion |

### Example 2: Preparation of Enriched and Stable RLTs

Chromatographic separation of radiolabeled peptides from non-radiolabeled peptides by chromatography is challenging because the radiolabeled peptide often coelutes with the excess non-radiolabeled peptide starting material. The process outlined below allows for the synthesis and isolation of radioligand therapies enriched in radiolabeled peptide.

### Synthesis of RLTs

Dried Actinium-225, was reconstituted in weak hydrochloric acid and transferred to a pharmaceutical grade glass or reinforced plastic reaction vessel. Low molarity (0.04-0.1M) hydrochloric acid, sodium acetate buffer, and a solution of Compound 153 ascorbic acid, ethanol, and sodium acetate buffer were then prepared.

The HCl, buffer solution, peptide solution, and reconstituted Actinium-225 were combined in a sealed glass container and heated at 90° C. for 15 minutes in a dry heating block heater. Diethylenetriaminepentaacetic acid (DTPA) was then added to quench the reaction and the crude reaction mixture was then analyzed by iTLC (Mobile phase 50 mM EDTA, pH 5.5).

### Purification of RLTs

A 100 μl (small-scale) or a 5.5 mL (large-scale) aliquot of the crude product mixture was manually injected into an HPLC injector-loop large enough to hold the entire volume in a single injection [small scale: Waters XSelect Peptide CSH C18; 130 A; 3.5 um; 4.6×150 mm (PN 186006957) or similar; large scale: Waters XSelect Peptide CSH C18 OBD; 130 A 5 μm; 10×250 mm (PN 186008267), or similar].

In the small-scale purification process, a 25 mM ammonium acetate (mobile phase A) and ethanol (mobile phase B) mobile phase was used. A gradient method of 40% B>47% B over 25 min; 0.5 mL/min; 100 uL injection was applied. The retention time of the purified radiolabeled peptide was simulated using an analogous method with non-radioactive Lanthanum-based peptide (La-153) against non-radiolabeled Compound 153. The purified radiolabeled Compound 153 drug product, [²²⁵Ac]Ac-153, was then diluted to strength, containing less than 10% v/v of ethanol, methionine, sodium ascorbate, and ammonium acetate buffer to mark the End of Synthesis (EOS).

In the large-scale purification process, an isocratic method was used with a mobile phase solvent of 1 mM ammonium acetate, with 43% v/v ethanol. The retention time of the purified radiolabeled peptide was simulated using an analogous method with non-radioactive Lanthanum-based peptide (La-153) against non-radiolabeled Compound 153. After elution, Tween-20 was added. The purified radiolabeled Compound 153 drug product, [²²⁵Ac]Ac-153, was diluted to strength, containing less than 10% v/v of ethanol, methionine, sodium ascorbate, and Tween-20, in an ammonium acetate buffer to mark the End of Synthesis (EOS).

After isolation, the purified radiolabeled Compound 153 drug product, [²²⁵Ac]Ac-153, was passed through a Millipore Millex GV sterile filter or equivalent to mark the End of Formulation (EOF). RCP analysis of the sterile product is then performed using an analytical HPLC equipped with a fraction collector.

High performance liquid chromatography (HPLC) purification and RCP analysis was determined using the two high pressure liquid chromatographs below.
1. Small Scale
   a. Agilent 1260 Infinity II HPLC System
      i. Major Components
      ii. 1260 Infinity II Quaternary Pump
      iii. 1260 Infinity II Vialsampler
      iv. 1260 Infinity II Diode Array Detector WR
      v. 1260 Infinity II Analytical FC
      vi. Flow-RAM with Laura for PET
      vii. PMT/Nal—1″ detector
      viii. Well type Nal/PMT
2. Large Scale
   a. AUTOMATION MODULE by Optimized Radiochemical Applications,
      ORA sprl
      15 rue de la salette
      5600 Neuville
      BELGIUM
   b. AUTOMATION SYSTEM: NEPTIS Mosaic RS® Synthesizer and NEPTIS control system
   c. ATTACHED HPLC with AUTOMATION Module by KNAUER
      Wissenschaftliche Geräte GmbH
      Hegauer Weg 38
      14163 Berlin
      Germany
   d. Isocratic HPLC SYSTEM: P 4.1S with 10 ml pump head and UVD 2.1S
   e. Gradient HPLC SYSTEM: P 6.1L Pump P 6.1L (LPG) with 10 ml pump head.

### Dispensing and Storing RLTs

Bulk drug product [²²⁵Ac]Ac-153 was stored in a seal glass container at low temperature (−20° C., 2-8° C., or 25° C.) prior to dose dispensing. Additional RCP analyses were performed prior to dispensing stored purified, enriched product. After RCP analysis, the purified product is dispensed as a unit-dose or a multi-dose into a Final Product Vial (FPV).

Particular formulations of [²²⁵Ac]Ac-153 with corresponding RCP values are shown below in Table 12, Table 13, Table 14, and Table 15.

TABLE 12

| | Ammonium | Sodium | | RCP (%) | | | | Storage |
|---|---|---|---|---|---|---|---|---|
| Form. | Acetate | Ascorbate | EtOH | | EOS + | EOS + | EOS + | Temp. |
| # | (M) | (mg/mL) | (% v/v) | EOS | 24 h | 72 h | 120 h | (° C.) |
| 1 | 0.1 | 75 | 11.75 | 89.1 | 49.0/ | 51.7 | ND¹ | 2-8 |
| | | | | | 75.1³ | | | |
| 2 | 0.1 | 75 | 11.75 | 91.5 | 67.8 | ND¹ | ND¹ | 2-8 |
| 3 | 0.1 | 100 | TBD⁵ | 89.9 | ND¹ | ND¹ | ND¹ | TBD⁵ |
| 4 | 0.1 | 100 | TBD⁵ | 91.5 | ND¹ | ND¹ | ND¹ | TBD⁵ |
| 5 | 0.1 | 225 | 11.75 | TBD⁵ | ND¹ | TBD⁵ | ND¹ | 2-8 |
| 6 | 0.1 | 300 | 10 | 73.4 | 61.7 | ND¹ | ND¹ | 2-8 |
| 7 | 0.1 | 375 | 11.75 | 81.5 | 90.0 | ND¹ | 78.3 | 2-8 |
| 8 | 0.1 | 500 | TBD⁵ | 81.5 | ND¹ | ND¹ | ND¹ | TBD⁵ |

¹Not determined;
³Synthesis was performed without DTPA;
⁵To be determined.

TABLE 13

| | Ammonium | Sodium | Gentisic | EtOH | RCP (%) | | | | | Storage |
|---|---|---|---|---|---|---|---|---|---|---|
| Form. | Acetate | Ascorbate | Acid | (% | | EOS + | EOS + | EOS + | EOS + | Temp. |
| # | (M) | (mg/mL) | (mg/mL) | v/v) | EOS | 24 h | 96 h | 120 h | 144 h | (° C.) |
| 9 | 0.1 | 375 | 7.5 | 11.75 | 92.9 | 89.3 | TBD⁵ | ND¹ | ND¹ | 2-8 |
| 10 | 0.1 | 375 | 7.5 | 11.75 | 92.9 | 89.3 | 91.9 | 92.6 | ND¹ | 2-8 |
| 11 | 0.1 | 375 | 30 | 11.75 | 95.2 | 92.8 | TBD⁵ | ND¹ | ND¹ | 2-8 |
| 12 | 0.1 | 375 | 30 | 11.75 | 95.2 | 92.8 | 90.8 | — | 90.0 | 2-8 |
| 13 | 0.1 | 500 | 10 | TBD⁵ | 92.9 | ND¹ | ND¹ | ND¹ | ND¹ | TBD⁵ |
| 14 | 0.1 | 500 | 40 | TBD⁵ | 95.2 | ND¹ | ND¹ | ND¹ | ND¹ | TBD⁵ |

¹Not determined;
⁵To be determined.

TABLE 14

| | Ammonium | Sodium | | | RCP (%) | | | | | | Storage |
|---|---|---|---|---|---|---|---|---|---|---|---|
| Form. | Acetate | Ascorbate | Methionine | EtOH | | EOS + | EOS + | EOS + | EOS + | EOS + | Temp. |
| # | (M) | (mg/mL) | (mg/mL) | (% v/v) | EOS | 24 h | 48 h | 72 h | 96 h | 120 h | (° C.) |
| 15 | 0.1 | 75 | 0 | 11.75 | 91.45 | ND¹ | ND¹ | ND¹ | ND¹ | ND¹ | 2-8 |
| 16 | 0.1 | 75 | 0 | 11.75 | 89.81 | 49.0 | ND¹ | 51.7 | ND¹ | ND¹ | 2-8 |
| 17 | 0.1 | 225 | 0 | 11.75 | 89.1 | ND¹ | ND¹ | 83.9 | 90.8 | ND¹ | 2-8 |
| 18 | 0.1 | 375 | 0 | 11.75 | 81.5 | 90.0 | ND¹ | ND¹ | ND¹ | 78.3 | 2-8 |
| 19 | 0.1 | 375 | 7.5 | 11.75 | 83.7 | 89.3 | 90.1 | 95.2 | 94.7 | ND¹ | 2-8 |
| 20 | 0.1 | 150 | 22.5 | 11.75 | 94.0 | 92.8 | ND¹ | ND¹ | ND¹ | ND¹ | 4 |
| 21 | 0.1 | 225 | 22.5 | 11.75 | 97.8 | 92.5 | ND¹ | 92.4 | ND¹ | ND¹ | 25 |
| 22 | 0.1 | 375 | 22.5 | 11.75 | 84.7 | 93.2 | 93.3 | 95.1 | 86.2 | ND¹ | 2-8 |

¹Not determined

TABLE 15

| | Ammonium | Sodium | | | Surfactant |
|---|---|---|---|---|---|
| | Acetate | Ascorbate | Methionine | EtOH | (Tween-20) |
| Form. # | (M) | (mg/mL) | (mg/mL) | (% v/v) | (% w/v) |
| 23 | 0.08 | 50.3 | 24.3 | 9.2 | 0.05 |
| 24 | 0.08 | 162.2 | 24.3 | 9.2 | 0.052 |
| 25 | 0.08 | 251.7 | 24.3 | 9.2 | 0.05 |
| 26 | 0.08 | 251.7 | 24.3 | 9.2 | 0.05 |
| 27 | 0.08 | 251.7 | 24.3 | 9.2 | 0.05 |
| 28 | 0.08 | 251.7 | 24.3 | 9.2 | 0.05 |
| 29 | 0.08 | 251.7 | 24.3 | 9.2 | 0.05 |
| 30 | 0.08 | 251.7 | 24.3 | 9.2 | 0.05 |
| 31 | 0.08 | 251.7 | 24.3 | 9.2 | 0.05 |
| 32 | 0.08 | 251.7 | 24.3 | 9.2 | 0.05 |

| | RCP (%) | | | | | | | | |
|---|---|---|---|---|---|---|---|---|---|
| | | | | | | | | | Storage |
| | | EOF⁶ + | EOF⁶ + | EOF⁶ + | EOF⁶ + | EOF⁶ + | EOF⁶ + | EOF⁶ + | Temp. |
| Form. # | EOF⁶ | 24 h | 48 h | 72 h | 96 h | 120 h | 168 h | 216 h | (° C.) |
| 23 | 97.3 | 97.2 | 96.8 | 95.8 | 95.4 | ND¹ | 94.8 | ND¹ | 2-8 |
| 24 | 92.0 | ND¹ | ND¹ | ND¹ | 25.3 | 64.1 | ND¹ | ND¹ | 4 |
| 25 | 97.3 | 96.9 | 96.8 | 96.4 | 97.5 | ND¹ | ND¹ | ND¹ | 2-8 |
| 26 | 95.5 | 93.2 | 93.2 | ND¹ | ND¹ | 93.7 | ND¹ | ND¹ | 4 |
| 27 | 97.3 | 95.1 | 96.2 | 96.9 | 96.2 | ND¹ | ND¹ | ND¹ | 4 |
| 28 | 97.3 | 96.9 | 96.8 | 96.4 | 97.5 | ND¹ | ND¹ | ND¹ | 4 |
| 29 | 95.5 | 93.2 | 93.2 | ND¹ | ND¹ | 93.7 | ND¹ | 93.4 | 2-8 |
| 30 | 97.3 | 95.1 | 96.2 | 96.9 | 96.2 | ND¹ | ND¹ | ND¹ | 2-8 |
| 31 | 95.5 | 93.2 | 93.2 | ND¹ | ND¹ | 93.7 | ND¹ | 93.4 | 4 |
| 32 | 95.5 | 88.5 | 91.7 | ND¹ | ND¹ | 91.9 | ND¹ | ND¹ | −20 |

¹Not determined;
²Surfactant was added to the formulation prior to the RCP assessment of the EOF + 120 h sample;
⁶EOS for Formulations 24, 31, and 32.

### Example 3: High Specific Activity Radiolabeled Peptides Targeting DLL3

Processes for preparing Compound 153 and Compound 447 disclosed herein can be found, at least, in PCT/US2024/033877, the contents of which are incorporated in their entirety.

### I. High Specific Activity Process and Formulation Development

A process was desired to increase the radioactive payload delivered to antigen expressing tumor cells by removing the competing, non-radiolabeled precursor compound from the injected formulation, therefore increasing the specific activity of the final product. This was achieved by purification of the radiolabeled product through RP-HPLC and stabilization of the isolated radiolabeled product with a stabilizing solution/formulation buffer.

For [²²⁵Ac]Ac-153 and [²²⁵Ac]Ac-447 radiolabeling reactions performed using 1.0-2.1 mCi [²²⁵Ac]AcCl₃ (in 0.04 M HCl), the reaction matrix was selected to contain 0.1 M sodium acetate (pH 5.5), 0.05 mg/mL ascorbic acid, 9.4% (v/v) ethanol, and 1.25 mg Compound 153. These conditions resulted in rapid and reproducible, high Actinium incorporation (>99.0% after 12 minutes of incubation at 90° C.).

### II. [²²⁵Ac]Ac-153 Radiolabeling Development

Radiolabeling methods for chelation of Actinium by Compound 153 was reviewed, including the use of an acetate or ascorbate buffer at a pH range of 5.0-6.0 in the reaction matrix. Drawbacks to ascorbate buffers include their lack of stability, leading to a decrease in Actinium incorporation over time. The use of acetate buffers in combination with anti-radiolytic stabilizers may provide high radiochemical yields at a pH of 5.0-5.5 in the reaction matrix.

The initial radiolabeling reaction was designed to investigate Actinium incorporation into Compound 153 containing a DOTA chelator over time at 90° C. The reaction included use of a sodium acetate buffer (0.1 M, pH 5.13) and was performed at a specific activity of 1.64 mCi/mg. The initial experiment intentionally did not include stabilizers to provide a baseline RCP of the reaction mixture. At the terminal timepoint (40 minutes), the reaction mixture was assessed by RP-HPLC, showing 84.8% RCP. The chromatogram revealed radiolytic impurities immediately preceding the radiolabeled product peak, indicating the need to include stabilizers in the reaction mixture during incubation. The addition of ascorbic acid and ethanol to the reaction mixture was able to reduce the formation of radiolytic products as evidenced by the increased RCP (92.1% vs 84.8%) while maintaining high Actinium incorporation (>99.0%). A higher RCP was desired, and as unbound Actinium was not significantly contributing to the RCP value, additional stabilizing agents were considered to reduce suspected radiolysis.

In addition to ascorbic acid and ethanol, other chemicals were evaluated as radioprotectants. Results detailed in Table 16 showed no substantial change in RCP when initially tested.

TABLE 16
Evaluation of the impact of stabilizers in the [²²⁵Ac]Ac-
153 reaction mixture on RCP of the crude reaction

| Run | #1 | #2 | #3 | #4 |
|---|---|---|---|---|
| Sodium acetate (M) | 0.12 | 0.14 | 0.13 | 0.11 |
| Ascorbic acid (mg/mL) | 0.009 | 0.008 | 0.008 | 0.028 |
| Gentisic acid (mg/mL) | — | — | — | 0.056 |
| L-Methionine (mg/mL) | — | — | — | 0.282 |
| Ethanol (%, v/v) | 8.6 | 8.3 | 8.3 | 17.1 |
| Reaction activity (μCi) | 29.7 | 37.4 | 80.7 | 54.4 |
| Peptide mass (μg) | 4.2 | 2.1 | 6.6 | 5.0 |
| Specific activity (μCi/μg) | 7.0 | 17.6 | 12.3 | 10.9 |
| Reaction volume (μL) | 35 | 48 | 96 | 59 |
| Buffer pH | 5.5 | 5.5 | 5.5 | 5.5 |
| Incubation temperature (° C.) | 90 | 90 | 90 | 90 |
| [²²⁵Ac]Ac | 99.1 | 97.3 | 98.7 | 97.3 |
| Incorporation (HPLC, | | | | |
| % at 15 minutes) | | | | |
| RCP (RP-HPLC, % at 15 minutes) | 92.1 | 90.5 | 89.4 | 92.7 |

### III. [²²⁵Ac]Ac-153 Purification Development

Both analytical and semi-preparative reverse-phase high-performance liquid chromatography (RP-HPLC) conditions were developed to enable isolation of the radiolabeled products in-situ, remove excess of unlabeled precursor, and maintain product stability throughout the purification process. When the isolated product is eluted directly into the formulation buffer, this signifies the End of Synthesis (EOS).

Small scale purifications had been performed on an analytical Agilent 1260 Infinity II HPLC system with an ethanol and 25 mM ammonium acetate gradient method. This solvent system was selected over an acetonitrile-based system due to the toxicity of acetonitrile; the eluted and appropriately diluted product containing ethanol and ammonium acetate is safe for clinical application without the need for an additional buffer exchange. Buffer exchange method or reformulation is typically achieved by trapping the product on a reversed phase cartridge, followed by washing and elution. However, such a procedure results in concentration and decomposition of drug product. An isocratic separation method was desirable for clinical manufacture application and process flow controls, as the ethanol content would be fixed across the entire run time and there would be no possibility of variation in on-line gradient solvent mixing. Initial isocratic development was performed on an analytical Agilent 1260 Infinity II HPLC system equipped with a UV diode array detector, set to 220 nm. As there is no stable isotope of Actinium available, Compound 153 was cold-labeled with Lanthanum (La) and used as a surrogate to simulate the [²²⁵Ac]Ac-153 product. Three factors were considered during optimization of the RP-HPLC purification: minimizing run time, maximizing separation of Compound 153 and La-153, and maintaining a narrow peak shape to minimize product collection time. 39% ethanol produced retention times>60 minutes. 43% ethanol produced good peak-peak separation (˜9.5 min) and a reasonable retention time for La-153 (32.5 min).

Next, the effects of the pH of the ammonium acetate buffer were explored. Two 25 mM ammonium acetate buffers were prepared with a pH of 5.5-6.0 and 4.5-5.0. Neither of the pH adjusted buffers had any appreciable effect on retention time. A product peak shoulder was lessened when run with the pH 5.5-6.0 buffer. When run with the pH 4.5-5.0 buffer, the peak profile dramatically deteriorated with less baseline resolution than the higher pH buffers. A neutral buffer gave the best balance of resolution, peak shape, and ease of production. The previous small-scale tests utilized 25 mM ammonium acetate in pure water that was mixed on-line with pure ethanol to achieve the desired ethanol concentration resulting in an ammonium acetate molarity of the mixed mobile phase running through the column of 14.3 mM. Buffer strengths above and below this value were tested. Increasing the buffer strength to 25 mM with 43% ethanol resulted in reduced retention time and resolution between the two species. Decreasing the buffer strength to 10 mM, 5 mM, and 1 mM showed an increase in resolution, attributed to a reduced retention of Compound 153 and an increased retention of La-153, although the relationship was not linear. The best separation was achieved with 1 mM ammonium acetate, 43% ethanol, resulting in retention times of ˜10 and ˜20 min for Compound 153 and La-153, respectively. Baseline resolution was maintained between Compound 153 and La-153 under a high mass condition of 1 mg.

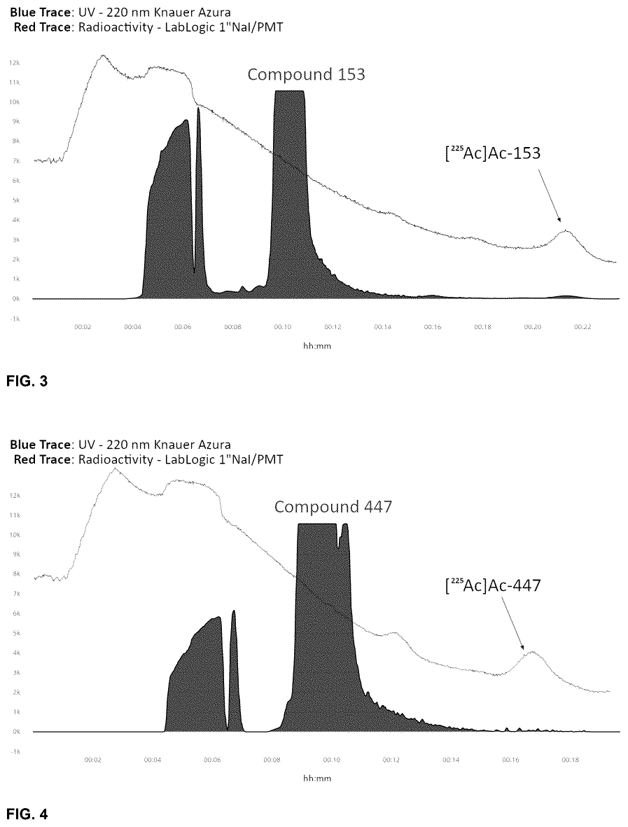

While these tests have demonstrated that 50 μg of La-153 is detectable via UV, a projected full-scale batch of [²²⁵Ac]Ac-labeled compound theoretically contains as little as ˜0.4 μg of labeled peptide. Due to the very low mass expected, UV detection is insufficient for reliable product identification and collection. Despite Actinium being predominantly an alpha emitter, there are beta and weak gamma emissions associated with its decay, along with the decay of its daughters. These decay events can be detected with standard radiodetectors. To facilitate product identification, a LabLogic 1″Nal/PMT probe coupled to a LabLogic Flow-Ram radio-HPLC detector and Agilent 1260 Infinity II HPLC was placed at the UV cell outlet to monitor radioactivity eluting from the column. An initial injection was performed with a mixture of ˜40 μg Compound 153, ˜50 μg La-153, and ˜145 μCi of [²²⁵Ac]Ac-153 to verify that there was a detectable radioactivity signal at a similar retention time to that of La-153. A radioactive signal was observed at the expected product retention time. This was subsequently confirmed to be attributed to [²²⁵Ac]Ac-153 after collection and analysis by HPLC. FIG. 3 shows a typical chromatogram resulting from purification of a [²²⁵Ac]Ac-153 reaction.

Using these conditions, several high activity syntheses/purifications were performed (Table 17).

TABLE 17
Summary of full-scale [²²⁵Ac]Ac-153 results
utilizing optimized purification conditions.

| Run | #1 | #2 | #3 | #4 | #5 | #6 |
|---|---|---|---|---|---|---|
| Reaction Activity (mCi) | 1.47 | 1.82 | 1.12 | 1.66 | 1.02 | 1.20 |
| Product Activity (mCi) | 1.16 | 1.55 | 0.88 | 1.32 | 0.81 | 0.91 |
| [²²⁵Ac]Ac Incorporation, Crude | >99.0 | >99.00 | >99.0 | >99.0 | >99.0 | >99.0 |
| (iTLC, %) | | | | | | |
| RCP (HPLC, %) | 95.5 | 97.3 | 97.3 | 97.3 | 96.9 | 96.2 |
| Radioactive Recovery (%) | 78.9 | 85.1 | 80.3 | 79.6 | 79.1 | 75.6 |

### IV. [²²⁵Ac]Ac-153 Product Formulation Development

One of the key challenges associated with radiopharmaceutical development is the maintenance of drug product stability in storage over its intended shelf-life. Radiolytic degradation of the drug product can occur through two main pathways: direct damage to the molecule due to particles emitted by the incorporated radionuclide and degradation of the molecule due to interaction with radicals formed during the radiolysis of water. Ethanol is used in the [²²⁵Ac]Ac-153 drug product formulation as a result of the RP-HPLC purification. Additional excipients with antioxidant properties were tested to achieve the goal of maintaining RCP of the [²²⁵Ac]Ac-153 drug product at >90% for 96-168 hrs.

### V. Low Activity [²²⁵Ac]Ac-153 Formulation Development (<0.1 mCi)

Excipients were initially screened utilizing low-activity (<0.1 mCi) formulations in ethanol (11.75%, v/v)/ammonium acetate (0.08 M) stored at 2-8° C. Sodium ascorbate concentrations of 75, 225, and 375 mg/mL were ineffective at maintaining a RCP>90% at timepoints up to 120 hrs post-formulation. Utilizing the higher concentration (375 mg/mL) of sodium ascorbate in formulation resulted in the highest RCP over time (90.0% and 78.3% at 24 and 120 hr post-formulation, respectively). This concentration was tested in combination with gentisic acid or L-methionine to evaluate the impact of each stabilizer on product stability over time.

Two concentrations of gentisic acid (7.5 and 30.0 mg/mL) and L-methionine (7.5 and 22.5 mg/mL) were tested in low-activity (<0.1 mCi) formulations containing sodium ascorbate (375 mg/mL), ethanol (11.75%, v/v)/ammonium acetate (0.08 M) stored at 2-8° C. Throughout the screening of these stabilizer concentrations/combinations, it was noted that improvements could be made to the HPLC analysis process that minimizes contamination of the system and enhances the repeatability of results. These process improvements were implemented prior to initiating large-scale development; however, these analytical issues made interpretation of the low-activity results difficult. Anticipating the need for higher amounts of stabilizers in high-activity formulations, high activity (>0.5 mCi) testing was initiated utilizing a high concentration of L-methionine (23.6 mg/mL) in combination with sodium ascorbate.

### VI. High Activity [²²⁵Ac]Ac-153 Formulation Development (>0.5 mCi)

High activity (>0.5 mCi) formulations were tested to achieve RCP of the product>90% at time points up to 168 hrs post-formulation, stored at 2-8° C. Formulations tested contained ammonium acetate (0.08 M), ethanol (9.2%, v/v), L-methionine (23.6 mg/mL), and varying concentrations of sodium ascorbate (50.3-251.7 mg/mL) and Tween-20 (0.00-0.05%, w/v). Formulations containing 251.7 mg/mL sodium ascorbate in combination with 0.05% (w/v) Tween-20, 23.6 mg/mL L-methionine, and 9.2% (v/v) ethanol were effective at maintaining a RCP>90% of the bulk dose 96-216 hr post-formulation. It is hypothesized that the use of Tween-20 allows the amount of sodium ascorbate in formulation to be reduced. Three stability experiments were then conducted containing 50.3 mg/mL sodium ascorbate in combination with L-methionine (23.6 mg/mL), ethanol (9.2% v/v), and Tween-20 (0.05%, w/v). All three stability experiments resulted in an RCP of the product>90% (94.1-95.2%) at 168 hr post formulation. See, Table 18. The final product formulation was selected to contain ammonium acetate (0.08 M), ethanol (9.2%, v/v), L-methionine (23.6 mg/mL), Tween-20 (0.05%, w/v), and sodium ascorbate (50.3 mg/mL).

TABLE 18
Evaluation of sodium ascorbate concentrations and Tween-20 in high activity
formulations of [²²⁵Ac]Ac-153, stored at 2-8° C.

| Run | #1 | #2 | #3 | #4 | #5 | #6 |
|---|---|---|---|---|---|---|
| Ammonium acetate (M) | 0.08 | 0.08 | 0.08 | 0.08 | 0.08 | 0.08 |
| Ethanol (%, v/v) | 9.2 | 9.2 | 9.2 | 9.2 | 9.2 | 9.2 |
| Tween-20 (%, w/v) | 0.00 | 0.05² | 0.05 | 0.05 | 0.05 | 0.05 |
| L-methionine (mg/mL) | 23.6 | 23.6 | 23.6 | 23.6 | 23.6 | 23.6 |
| Sodium ascorbate (mg/mL) | 162.2 | 251.7 | 251.7 | 251.7 | 50.3 | 50.3 |
| Formulation pH | 6.0 | 6.0 | 6.0 | 6.0 | 6.0 | 6.0 |
| Final Product Activity (mCi) | 1.13 | 1.16 | 1.55 | 0.88 | 1.32 | 0.81 |
| Simulated Dose Activity (mCi) | 1.05³ | 0.52 | 1.03 | 0.82 | 1.03 | 0.75³ |
| Radioactive concentration (μCi/μL) | 0.028 | 0.028 | 0.038 | 0.022 | 0.032 | 0.020 |
| Storage temperature (° C.) | 2-8 | 2-8 | 2-8 | 2-8 | 2-8 | 2-8 |

RCP (RP-HPLC, %)

| | | | | | | |
|---|---|---|---|---|---|---|
| T = 0 hr | 92.0 | 95.5 | 97.3 | 97.3 | 97.3 | 96.9 |
| T = 24 hr | — | 93.2 | 95.1 | 96.9 | 97.2 | 96.5 |
| T = 48 hr | — | 93.2 | 96.2 | 96.8 | 96.8 | 94.6 |
| T = 72 hr | — | — | 96.9 | 96.4 | 95.8 | 94.8 |
| T = 96 hr | 25.3¹ | — | 96.2 | 97.5 | 95.4 | 95.6 |
| T = 120 hr | 64.1¹ | 93.7 | — | — | — | — |
| T = 144 hr | — | — | — | — | — | — |
| T = 168 hr | — | — | — | — | 94.8 | 94.1 |
| T = 216 hr | — | 93.4 | — | — | — | — |

¹Sticking of the radiolabeled product in the storage container evidenced by low counts reported by the gamma counter. Tween-20 (0.05%, w/v) added to the formulation just prior to 120 hr sample analysis.
²Tween-20 (0.05%, w/v) added to the formulation just prior to the 24 hr sample analysis
³Simulated dose activities for these instances were derived from decay correcting the Final Product Activity by 24 hours.

### Table 14 vs Table 18: Comparison of Data

In Table 14, the RCPs at 24 h for Formulation #16 (comprising 75 mg/mL sodium ascorbate) and Formulation #18 (comprising 375 mg/mL sodium ascorbate) are 49.0% and 90.0%, respectively. These data show that a high amount of sodium ascorbate (radiolytic stabilizer) has led to high (e.g. >90%) RCP.

The RCPs at 72 h for Formulation #22 in Table 14 (comprising 375 mg/mL sodium ascorbate and no Tween-20) and Formulation #6 in Table 18 (comprising 50.3 mg/mL sodium ascorbate and 0.5 mg/mL Tween 20) are 95.1% and 94.8%, respectively. These data indicate that the presence of polysorbate (Tween 20), even in small amount, enables the amount of radiolytic stabilizer to be reduced significantly without impacting the (high) RCP.

### VII. Sterile Filtration of Formulated Drug Product

After [²²⁵Ac]Ac-153 product collection into the Formulation Buffer, the contents were aspirated into a 30 or 60 mL BD syringe and manually passed through the sterilizing filter into a separate vial (Table 19).

TABLE 19
Radioactive retention and recovery through sterile filtration
of the formulated drug product [²²⁵Ac]Ac-153.

| Run | #1 | #2 | #3 | #4 | #5 | #6 |
|---|---|---|---|---|---|---|
| Starting | 0.897 | 0.830 | 1.335 | 1.035 | 0.818 | 0.925 |
| Product | | | | | | |
| Activity (mCi) | | | | | | |
| Filtered | 0.884 | 0.816 | 1.321 | 1.025 | 0.808 | 0.910 |
| Product | | | | | | |
| Activity (mCi) | | | | | | |
| Syringe | 0.002 | 0.002 | 0.014* | 0.010* | 0.010* | 0.010* |
| Activity | | | | | | |
| (mCi) | | | | | | |
| Filter | 0.008 | 0.007 | | | | |
| Activity (mCi) | | | | | | |
| Filtration | 98.5 | 97.8 | 95.0 | 99.0 | 98.8 | 98.4 |
| Yield (%) | | | | | | |

*Combined activities of syringe and filter.

### VIII. [²²⁵Ac]Ac-447 Radiolabeling Development

The conditions developed for Actinium chelation by Compound 153 and subsequent purification were initially applied to Compound 447 and then optimized. Two reactions were performed using 1.0-1.6 mCi source material (in 0.04 M HCl), resulting in high Actinium incorporation (>99.0% after 12 minutes of incubation at 90° C.).

### IX. [²²⁵Ac]Ac-447 Purification Development

The optimum conditions for the separation of [²²⁵Ac]Ac-447 from excess unlabeled Compound 447 by semi-preparative HPLC were determined. Compound 447 and the Lanthanum surrogate, La-447 exhibited retention times of 3:05 and 6:20 mm:ss, respectively. In order to effectively remove the unlabeled peptide, a methodology similar to that used for Compound 153 was employed to identify mobile phase conditions using the same analytical Agilent 1260 Infinity II HPLC system with a Waters XSelect Peptide CSH C18, 130 Å, 3.5 μm, 4.6×150 mm column, equipped with a UV diode array detector, set to 220 nm. Initial tests were focused on determining an ethanol concentration that would achieve a similar retention of both peptide species as was seen for Compound 153. Ethanol concentrations were varied from 43% to 38% with a 1.75 mM ammonium acetate buffer. Ethanol, 39%, provided comparable separation for Compound 447 as was seen for Compound 153.

Buffer strengths were also explored after fixing the ethanol concentration at 39%. Buffer strengths were tested to provide, after mixing, molarities of 0.5, 0.8, and 1.1 mM. A buffer strength of 0.9 mM was chosen for scale up testing.

Subsequent tests to optimize the purification of Compound 447 were performed on a Waters XSelect Peptide CSH C18 OBD Prep Column 130 Å; 5 μm; 10×250 mm utilizing the Neptis® radiopharmaceutical synthesizer equipped with a Knauer Azura pump set to 3.5 mL/min and a UV cell set to 220 nm for detection. To verify that these HPLC conditions were suitable for full-scale production batches, an injection was performed with a mixture of ˜1300 μg Compound 447, ˜50 μg La-447, and ˜50 μCi of [²²⁵Ac]Ac-447 to verify that there was a detectable radioactivity signal at a similar retention time to that of La-447. A radioactive signal was able to be observed at the expected product retention time (˜17 min). See, Table 20. FIG. 4 shows a typical chromatogram resulting from purification of a high activity [²²⁵Ac]Ac-447 reaction.

High activity reactions were performed and the product was purified and collected as previously described. The resulting solution contained ammonium acetate (0.08 M), ethanol (8.3%, v/v), L-methionine (23.6 mg/mL), Tween-20 (0.05%, w/v), and sodium ascorbate (50.3 mg/mL). This formulation was again successful at maintaining the RCP of the drug product>90.0% for up to 168 hrs. At EOF, the product was kept at room temperature (RT) for the first 24 hr to simulate a room temperature hold during manufacturing between EOF and dispensing. This hold at room temperature had no significant effect on the RCP. After the initial 24 hrs, the product was stored at 2-8° C. (Table 21).

Sterile filtration of formulated [²²⁵Ac]Ac-447 was performed as previously described for [²²⁵Ac]Ac-153.

TABLE 20
Summary of full-scale [²²⁵Ac]Ac-447 results
utilizing optimized purification conditions.

| Run | #1 | #2 | #3 |
|---|---|---|---|
| Reaction Activity (mCi) | 1.62 | 1.04 | 0.67 |
| Product Activity (mCi) | 1.37 | 0.85 | 0.38 |

Results

| | | | |
|---|---|---|---|
| [²²⁵Ac]Ac Incorporation, | >99.0 | >99.0 | 79.4 |
| Crude (iTLC, %) | | | |
| RCP (HPLC, %) | 95.8 | 96.4 | 95.8 |
| Radioactive Recovery (%) | 84.2 | 82.3 | 57.2 |

TABLE 21
Evaluation of optimized formulation with
[²²⁵Ac]Ac-447, stored at 2-8° C.

| Run | #1 | #2 |
|---|---|---|
| Ammonium acetate (M) | 0.08 | 0.08 |
| Ethanol (%, v/v) | 8.3 | 8.3 |
| Tween-20 (%, w/v) | 0.05 | 0.05 |
| L-methionine (mg/mL) | 23.6 | 23.6 |
| Sodium ascorbate (mg/mL) | 50.3 | 50.3 |
| Formulation pH | 6.0 | 6.0 |
| Final Product Activity (mCi) | 1.37 | 0.85 |
| Simulated Dose Activity (mCi) | 1.03 | 0.80 |
| Radioactive concentration (μCi/μL) | 0.033 | 0.021 |
| Storage temperature (° C.) | 2-8 | 2-8 |

RCP (RP-HPLC, %)

| | | |
|---|---|---|
| T = 0 hr | 95.8 | 96.4 |
| T = 24 hr | 95.8 | 96.1 (RT)¹ |
| T = 48 hr | 95.8 | 95.3 |
| T = 72 hr | 95.9 | 95.7 |
| T = 96 hr | 95.3 | 95.2 |
| T = 168 hr | 94.8 | 94.7 |

¹Bulk product held at room temperature for 24 hrs prior to storage at 2-8° C.

### X. Method of Making Stabilized HSA Compositions of ²²⁵Ac-Labeled 153 and 447

Radiosynthesis of the drug substance, i.e., [²²⁵Ac]Ac-153 or [²²⁵Ac]Ac-447, in-situ is conducted with up to 2.1 mCi of Actinium-225 solution in either 0.04 M hydrochloric acid or 0.05 M nitric acid with addition of a 3.15 mL Reagent Solution containing 0.3 mL of Reaction Buffer (1.0 M sodium acetate buffer containing 0.6 mg/mL ascorbic acid), 0.35 mL ethanol, and 1.25 mg (600 nmol) of peptide precursor (Compound 153 or Compound 447) solution in 2.5 mL of metal free water. This reaction mixture is contained in a sealed 10 mL glass vial, transferred to the Neptis® heater, and heated at 90° C. for 12 minutes. After completion of the incubation, the heater is cooled to a setpoint of 40° C. Once the setpoint is reached, the reaction vial is connected to the pre-installed Neptis® cassette. The reaction mixture is then quenched by the automated addition of 2.5 mL of a 0.05 mg/mL DTPA solution in 0.1 M ammonium acetate, pH 5.5.

After the reaction mixture is quenched, it is injected into the semi-preparative HPLC for purification. Product identification is performed utilizing an integrated radiodetector with a typical elution time of ˜17 and 20 minutes ([²²⁵Ac]Ac-Compound 447 and [²²⁵Ac]Ac-153, respectively). Upon triggering product collection, HPLC flow is diverted from waste to a 50 mL formulation vial containing 32.25 mL of Formulation Buffer. Product collection is timed to collect for 2.5 mT at a 3.5 mL/min flowrate, after which flow is diverted back to waste. The final formulated 41 mL of drug product is mixed and sterile filtered using a 33 mm, 0.22 micron Millex-GV sterilizing filter with a DuraporeG PVDF membrane.

### Example 4: Cytotoxicity Studies with HSA [²²⁵Ac]Ac-Compound 447

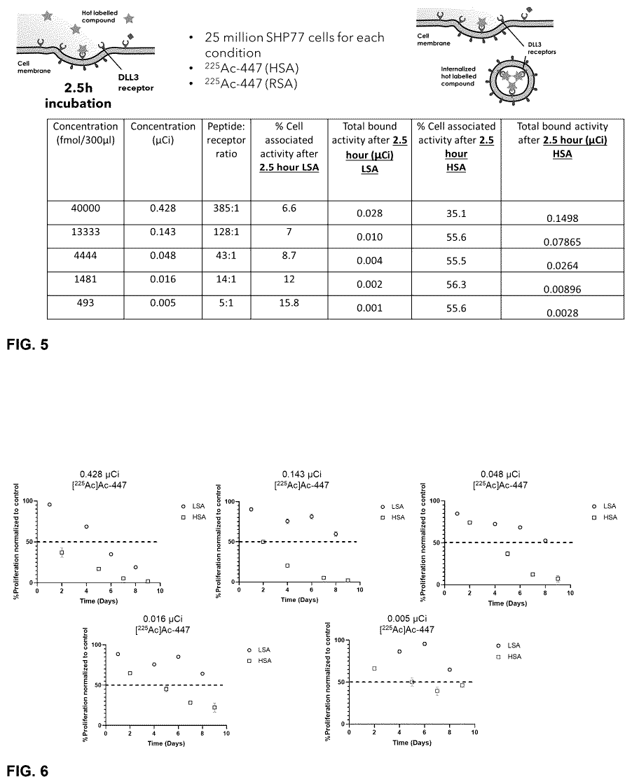

The in vitro cytotoxicity/efficacy of High specific activity (HSA) and Regular (low) (RSA/LSA) specific activity [²²⁵Ac]Ac-447 drug product was studied in SHP-77 cells. SHP-77 cells are a human small cell lung cancer cell line expressing about 3,286 DLL3 cell receptors/cell (FIG. 5 and FIG. 6).

| Materials and Equipment | Manufacturer | Category Number | Lot Number |
|---|---|---|---|
| Bio Safety Cabinet | Thermo Fisher | 1389-M | NA |
| Incubator | Thermo Fisher | 381 | NA |
| Countess Cell Counter | Thermo Fisher | A49893 | NA |
| DMEM (1X) | Gibco | 11965-084 | 2522657 |
| Cell Titer Glow 2.0 | Promega | G9242 | 0000569962 |
| Fetal Bovine Serum | R&D Systems | S11195 | M20193 |
| PBS | Gibco | 10010001 | 2561363 |
| Bovine Serum Albumin | Sigma Aldrich | A9576 | C120003 |
| Glycine | Thermo Fisher | J61855-AK | U091509 |
| Versene (1X) | Thermo Fisher | 15040-066 | NA |
| Tissue Culture | Falcon | 353112 | NA |
| Treated Flask | | | |
| 175 cm2 | | | |
| BioLite 75 cm2 Flask | Thermo Fisher | 130190 | NA |
| 96 well white | Perkin Elmer | 6005680 | NA |
| walled, white | | | |
| bottom, cell | | | |
| culture grade | | | |
| microplates | | | |
| Countess Cell Counting | Thermo Fisher | C10283 | 124B23212 |
| Chamber Slides | | | |
| Trypan blue | Gibco | 15250061 | 2419039 |
| Hidex Sense microplate | Hidex | 425-311 | 321-0302 |
| reader | | | |

### I. Synthesis of Regular (Low) Specific Activity [²²⁵Ac]Ac-447

Compound 447 was radiolabeled with [²²⁵Ac]Ac(NO₃)₃ at a specific activity of 9.6 μCi/nmol peptide in a reaction buffer containing sodium acetate (1.0 M, pH 5.5) with ascorbic acid (0.76 mg/mL) and ethanol (9%, v/v). The reaction mixture was incubated on a thermomixer at 700 rpm and 90° C. for 15 minutes. The product was analyzed by RP-HPLC, and the RCP was determined to be 95.58%. An aliquot of the product was diluted in 1% PBSA to obtain solutions with final concentrations of 0.428 μCi/300 μL, 0.143 μCi/300 μL, 0.048 μCi/300 μL, 0.016 μCi/300 μL, and 0.005 μCi/300 μL before addition to the cells. A control was prepared to contain 32.5 fmol of Compound 447/300 μL.

### II. Synthesis of High Specific Activity [²²⁵Ac]Ac-Compound 447

Compound 447 was radiolabeled with [²²⁵Ac]Ac(NO₃)₃ at a specific activity of 9.6 μCi/nmol peptide in a reaction buffer containing sodium acetate (1.0 M, pH 5.5) with ascorbic acid (0.76 mg/mL) and ethanol (9%, v/v). The reaction mixture was incubated on a thermomixer at 700 rpm and 90° C. for 15 minutes. After completion of the radiolabeling reaction, the non-labeled Compound 447 and other impurities were removed using reversed-phase high performance liquid chromatography (RP-HPLC) (Agilent Infinity II 1260 HPLC equipped with a Waters XSelect Peptide CSH C18 column, fraction collector, and UV-DAD). The product was collected directly into formulation buffer (200 mg/mL sodium ascorbate, 30 mg/mL L-methionine, 0.05% w/v Tween-20, and 0.1 M ammonium acetate). The product was analyzed by RP-HPLC, and the RCP was determined to be 95.58%. An aliquot of the product was diluted in 1% PBSA to a final concentration of 0.428 μCi/300 μL, 0.143 μCi/300 μL, 0.048 μCi/300 μL, 0.016 μCi/300 μL, and 0.005 μCi/300 μL before addition to the cells. A control was prepared to contain 32.5 fmol Compound 447/300 μL.

### III. Cell Preparation, Study Execution, and Analysis

The study was conducted using SHP-77 cells (Table 22).

TABLE 22

| | | Catalogue | Growth | Growth | |
|---|---|---|---|---|---|
| Name | Source | No. | Mode | Conditions | Growth Media |
| SHP-77 | ATCC | CRL-2195 | Mixed | 37° C.; | RPMI 1640 + |
| | | | | 5% CO₂ | 10% FBS |

The cells were cultured in RPMI 1640 media supplemented with 10% fetal bovine serum (FBS) culture media (20 mL) in tissue culture treated T150 flasks at 37° C. and 5% CO₂. After confirming the viability and count of detached cells using Trypan blue, the cells were centrifuged at 4° C. for 5 min (1,000 rpm). The cell pellet obtained was washed once with phosphate buffered saline (PBS) and resuspended in PBS+1% Bovine Serum Albumin (PBSA) to obtain the desired cell concentration (25 million cells/mL).

Cells were incubated with 300 μL of the incubation buffer ([²²⁵Ac]Ac-447 in PBSA) or 300 μL of the incubation buffer (unlabeled 447) for 2.5 hours at 37° C. (Table 23) on a thermomixer at the speed of 700 rpm. See, Table 24. Post incubation, the cells were pelleted and washed 2 times using cold 1% PBSA. The supernatant was discarded, and the cells were then resuspended in 1 mL of media. After confirming the count and viability of the detached cells using Trypan blue, the cells were centrifuged at 4° C. for 5 min (1,200 rpm). The cell pellet obtained was diluted with media to 100,000 cells/100 μL of media. The cells were plated in a 96 well plate each, 6 wells per sample. The plates for the regular (low) specific activity were kept in the incubator and taken out on Day 1, 4, 6 and 8 to be read. The plates for the high specific activity were kept in the incubator and taken out on Day 2, 5, 7 and 9 to be read. To read the plates CTG reagent (100 μL) was added to each treated well and the plate was read in a Hidex plate reader.

TABLE 23
Summary of the Dosing Conditions for HSA and RSA

| Cell Line | Peptide (μCi) in 300 μL | Assay |
|---|---|---|
| SHP-77 | 0.428 | CTG |
| SHP-77 | 0.143 | CTG |
| SHP-77 | 0.048 | CTG |
| SHP-77 | 0.016 | CTG |
| SHP-77 | 0.005 | CTG |
| SHP-77 | 0 | CTG |

TABLE 24
Summary of Experimental Conditions

| | Adherent/ | | | | | | |
|---|---|---|---|---|---|---|---|
| Cell | Suspension/ | Dissociation | Cell | Binding | Incubation | Incubation | Thermomixer |
| Name | Mixed | Method | Number | Media | Time | Temp. | Speed |
| SHP- | Mixed | Versene | 25 | 1% | 2.5 h | 37° C. | 700 rpm |
| 77 | | | million | PBSA | | | |

Abbreviations: PBSA = phosphate buffered saline with 1% bovine serum albumin; rpm = revolutions per minute

Luminescence values were extracted from the Hidex plate reader and the average of 6 replicates at each time and concentration was calculated. Percent proliferation in SHP-77 was calculated by dividing the average luminescence by the luminescence SHP-77 control at each concentration and multiplying by 100.

### Example 5: [¹¹¹In]In-Labeled Peptide Development and Formulation

### I. [¹¹¹In]In-447 Radiolabeling Development

The following section describes the radiochemistry synthesis of [¹¹¹In]In-447, followed by the direct formulation into the final [¹¹¹In]In-447 sterile drug product, that is suitable for clinical application.

Radiosynthesis of active pharmaceutical substance [¹¹¹In]In-447 in-situ is conducted in a 6 ml 0.1 M sodium acetate reaction buffer containing 0.1% ascorbic acid (w/v) and 1% methionine (w/v), with sequential addition of 20 nmol of peptide precursor Compound 447 solution in ˜90 μL of metal free water, and 60 mCi of [¹¹¹In]InCl₃ solution in 0.05 M HCl. This reaction mixture is contained in a sealed 20 mL glass vial, heated at 80° C. for 20 minutes using a heating block, followed by a cooling step at ambient room temperature for 15 minutes.

Immediately upon the synthesis reaction mixture cooling step is completed, [¹¹¹In]In-447 drug product was formulated involving the addition of a 9 mL of formulation buffer (48 mg/mL sodium ascorbate, 10.66 mg/mL ascorbic acid, 24 mg/mL methionine, 0.04% w/v Tween 20, 0.04 M acetate, pH˜4.7) directly into the sealed 20 mL glass vial and mixing with the reaction mixture. The final drug product [¹¹¹In]In-447 formulation solution is sterile filtered using a suitable 0.22 microns (μm) filter. A 10 mL, 40 mCi dose is dispensed with content shown in Table 25.

The target specific activity for [¹¹¹In]In labeled Compound 447 has been tested on similar peptide analogs at 1 mCi/nmol, 2.5 mCi/nmol, or 3 mCi/nmol. The specific activity is calculated based on total radioactivity over the mole/mass of DOTA-peptide.

TABLE 25
Contents and Concentrations of the Dispensed
Final Drug Product Vial

| | | |
|---|---|---|
| Volume | 10 | mL |
| Radioactive Concentration | 4 | mCi/mL |
| Total Activity | 40 | mCi |
| Concentration of 447 | 2.8 | μg/mL |
| Calculated Specific Activity | 1.4 | mCi/μg |
| Concentration of Sodium | 48 | mg/mL |
| Ascorbate | | |
| Concentration of Ascorbic Acid | 10.66 | mg/mL |
| Concentration of Methionine | 24 | mg/mL |
| Concentration of Tween 20 | 0.04% | w/v |

| | |
|---|---|
| Concentration of Acetate | 0.04M |
| pH | 4.7 |

Reaction Buffers with varying pH were first explored in screening experiments with [¹¹¹In]In radiolabeling of Compound 153 (Table 26). Reaction Buffer 4 (0.1 M sodium acetate buffer with pH˜4.1) returned the highest RCP. Ammonium acetate buffer or sodium acetate buffer at a higher pH yielded lower RCP, while 0.2 M sodium acetate buffer at the same pH generated a lower [¹¹¹In]In incorporation rate and RCP.

TABLE 26

| Run | 1 | 2 | 3 | 4 |
|---|---|---|---|---|
| Reaction buffer | pH 3.94 | pH 4.64 | pH 4.15 | pH 4.18 |
| | 0.2M | 0.1M | 0.2M | 0.1M |
| | NaOAc | NaOAc | NaOAc | NaOAc |
| Scale (mCi) | 5.95 | 1.07 | 0.93 | 0.95 |
| Reaction volume | 0.6 | 0.1 | 0.1 | 0.1 |
| (mL) | | | | |
| RCP (HPLC, %) | 92.90 | 93.38 | 93.21 | 94.51 |
| [¹¹¹In]In | 96.46 | 99.16 | 97.11 | 98.38 |
| incorporation rate | | | | |
| (HPLC, %) | | | | |

### II. [¹¹¹In]In-153 Reaction Optimization (Small Scale)

Stabilizers at various concentrations were screened to minimize the radiolytic decomposition during radiolabeling (Table 27A). Initial reactions were on a small scale of 1-3 mCi of starting [¹¹¹In]InCl₃ radioactivity. These reactions were carried out in an Eppendorf vial incubated on an orbital shaker with the setting temperature at 80° C. and shaking speed of 700 RPM. The ascorbic acid concentration was varied ranging from 0.0167 to 0.167 mg/mL, gentisic acid concentration from 0.167-0.2 mg/mL, and methionine at 1.667, 1, or 0.5 mg/mL. In the presence of 9% ethanol, high RCP and high [¹¹¹In]In incorporation rate were observed after 10 min incubation. However, after ethanol was eliminated to simplify the overall process, [¹¹¹In]In incorporation rates dropped to ˜95% in presence of the stabilizers at the same concentrations (Reaction 3 and 5, Table 27A). After elongating the incubation time from 10 min to 20 min, the [¹¹¹In]In incorporation rate increased to 97% (Reaction 6).

Further modification on the concentration of stabilizers improved both the RCP and the [¹¹¹In]In incorporation rate, and a 10 mCi reaction under this reaction condition yielded similar results (Reaction 7 and 9 (Table 27B)).

TABLE 27A
Reaction Stabilizer and Incubation Time Screening for [¹¹¹In]In-153

| Reaction Number | 1 | 2 | 3 | 4 | 5 | 6 | 7 | 8 |
|---|---|---|---|---|---|---|---|---|
| Scale (mCi) | 0.949 | 3 | 1.026 | 0.943 | 1.311 | 1.078 | 1.042 | 10.4 |
| Ascorbic Acid | 0.0167 | 0.167 | 0.0167 | 0.1 | 0.1 | 0.1 | 0.05 | 0.05 |
| (mg/mL) | | | | | | | | |
| Gentistic Acid | 0.167 | 0.167 | 0.167 | 0.2 | 0.2 | 0.2 | 0.1 | 0.1 |
| (mg/mL) | | | | | | | | |
| Methionine | 1.667 | 1.667 | 1.667 | 1 | 1 | 1 | 0.5 | 0.5 |
| (mg/mL) | | | | | | | | |
| Ethanol (%) | 9 | 9 | 0 | 9 | 0 | 0 | 0 | 0 |
| Reaction time | 10 | 10 | 10 | 10 | 10 | 20 | 20 | 20 |
| (min) | | | | | | | | |
| Reaction | 0.1 | 0.3 | 0.1 | 0.1 | 0.1 | 0.1 | 0.1 | 1.0 |
| volume (mL) | | | | | | | | |
| RCP (HPLC, %) | 94.51 | 95.09 | 88.66 | 94.64 | 90.22 | 91.12 | 92.08 | 93.90 |
| [¹¹¹In]In | 98.38 | 99.26 | 94.65 | 99.26 | 95.98 | 97.31 | 98.54 | 99.76 |
| incorporation | | | | | | | | |
| rate (HPLC, %) | | | | | | | | |

Reaction condition: 0.1M sodium acetate buffer, pH ~4.1. Heat at 80° C. for 10 or 20 min, at 700 RPM.

### III. [I¹¹¹In]In-153 Reaction Optimization (Large Scale)

Scaled-up reactions (10 mCi to 70 mCi) were developed (Tables 27B and 270). At these scales, the reactions were performed in a glass vial and incubated by a heat block equipped with a digital temperature control. Ethanol was also eliminated from the reaction. In initial reactions using the small-scale conditions, RCP was lower than the previous smaller scale reaction. The concentrations of methionine and ascorbic acid were increased and gentisic acid was eliminated from the reaction buffer. Under this optimized condition, with 0.1 mg/mL of ascorbic acid and 1 mg/mL of methionine was repeated 6 times at large scale (reaction 11-16, 50 mCi or higher reaction scale) while generating consistent data. The radiochemical purities were consistently over the threshold of 91% with high [¹¹¹In]In incorporation rates.

TABLE 27B
Batch Scaling Data Summary for [¹¹¹In]In-153

| Reaction Number | 9 | 10 | 11 | 12 | 13 | 14 | 15 | 16 |
|---|---|---|---|---|---|---|---|---|
| Scale (mCi) | 19.3 | 20.0 | 50.4 | 49.9 | 49.9 | 49.0 | 48.5 | 68.0 |
| Ascorbic Acid (mg/mL) | 0.05 | 0.05 | 0.1 | 0.1 | 0.1 | 0.1 | 0.1 | 0.1 |
| Gentistic Acid (mg/mL) | 0.1 | 0 | 0 | 0 | 0 | 0 | 0 | 0 |
| Methionine (mg/mL) | 0.5 | 1 | 1 | 1 | 1 | 1 | 1 | 1 |
| Ethanol (%) | 0 | 0 | 0 | 0 | 0 | 0 | 0 | 0 |
| Reaction Volume (mL) | 2.0 | 2.0 | 5.0 | 5.0 | 5.0 | 5.0 | 5.0 | 6.0 |
| RCP (HPLC, %) | 90.11 | 92.77 | 93.3 | 92.51 | 91.34 | 92.08 | 93.69 | 93.58 |
| [¹¹¹In]In incorporation rate | 98.78 | 99.57 | 98.6 | 98.77 | 97.13 | 99.38 | 99.62 | 99.78 |
| (HPLC, %) | | | | | | | | |

### IV. [¹¹¹In]In-447 Reaction Optimization (Large Scale)

Based on the reaction optimization for [¹¹¹In]In-153, the same stabilizers and concentrations were adopted and the radiochemical purities were consistently over 91% with high [¹¹¹In]In incorporation rates (Table 270).

TABLE 27C
Batch Scaling Data Summary for [¹¹¹In]In-447

| Reaction Number | 17 | 18 | 19 | 20 | 21 | 22 | 23 | 24 |
|---|---|---|---|---|---|---|---|---|
| Scale (mCi) | 10.2 | 9.90 | 19.2 | 20.0 | 55.1 | 62.1 | 64.4 | 58.7 |
| Ascorbic Acid (mg/mL) | 0.05 | 0.05 | 0.05 | 0.05 | 0.1 | 0.1 | 0.1 | 0.1 |
| Gentistic Acid (mg/mL) | 0.1 | 0.1 | 0.1 | 0.1 | 0 | 0 | 0 | 0 |
| Methionine (mg/mL) | 0.5 | 0.5 | 0.5 | 0.5 | 1 | 1 | 1 | 1 |
| Ethanol (%) | 0 | 0 | 0 | 0 | 0 | 0 | 0 | 0 |
| Reaction Volume (mL) | 1.0 | 1.0 | 2.0 | 2.0 | 5.4 | 6.2 | 6.2 | 6.6 |
| RCP (HPLC, %) | 91.86 | 93.05 | 94.76 | 92.02 | 95.39 | 94.83 | 94.51 | 94.12 |
| [¹¹¹In]In incorporation rate | 99.38 | 99.24 | 99.59 | 97.37 | 99.72 | 99.76 | 99.63 | 99.22 |
| (HPLC, %) | | | | | | | | |

### V. [I¹¹¹In]In-153 Stability Experiments

The formulation buffer was optimized to maintain high RCP using [¹¹¹In]In-153. Tween 20 was added to the formulation buffer, which is effective in preserving the stability over the period of storage. Gentisic acid was eliminated from the final formulation buffer, and the specific concentration was increased to 4 mCi/mL. The radiolabeled peptide [¹¹¹In]In-153 showed minimal decomposition during over 5-day-storage period, with the possibility to extend to 7 days, and the radiochemical purities were maintained at over 90% over this time period (Table 28). The product is stable at both ambient room temperature and refrigerated at 2-8° C. (Table 28), further supporting long distance shipping at proposed 2-8° C. with excursion temperature to reach ambient room temperature without adversely affecting the product quality.

TABLE 28

| Run | 35 | 36 | 37 | 38 | 39 | 40 | 41 | 42 | 43 | 44 |
|---|---|---|---|---|---|---|---|---|---|---|
| Formulation | 48 mg/mL sodium ascorbate, 10.66 mg/mL ascorbic acid, 24 mg/mL | | | | | | | | | |
| Buffer | methionine, 0.04% w/v Tween 20, 0.04M acetate, pH ~4.7 | | | | | | | | | |
| Dose | 5 | | | | | | 10 | | | |
| Volume | | | | | | | | | | |
| (mL) | | | | | | | | | | |
| Dose | 4 | | | | | | 4 | | | |
| Concentration | | | | | | | | | | |
| (mCi/mL) | | | | | | | | | | |
| Total | 20 | | | | | | 40 | | | |
| activity | | | | | | | | | | |
| (mCi) | | | | | | | | | | |
| RCP, t = 0 | 92.76% | | 92.43% | | 92.81% | | 93.90% | 94.19% | 93.51% | 93.25% |
| Storage | RT | 2-8° C. | RT | 2-8° C. | RT | 2-8° C. | 2-8° C. | 2-8° C. | 2-8° C. | RT |
| condition | | | | | | | | | | |
| RCP, 1-day | 92.11% | 91.88% | — | — | 93.88% | 94.18% | 94.83% | 94.69% | 94.28% | 93.77% |
| storage | | | | | | | | | | |
| RCP, 2-day | 92.75% | 92.28% | — | — | 93.87% | 93.81% | 94.96% | 94.90% | 94.42% | 93.92% |
| storage | | | | | | | | | | |
| RCP, 3-day | 93.28% | 93.61% | 93.13% | 93.42% | — | — | 94.47% | — | 94.94% | 94.01% |
| storage | | | | | | | | | | |
| RCP, 4-day | — | — | 93.15% | 93.38% | — | — | — | — | 94.51% | 94.61% |
| storage | | | | | | | | | | |
| RCP, 5-day | — | — | 93.96% | 93.58% | 94.22% | 93.79% | — | 94.88% | — | — |
| storage | | | | | | | | | | |
| RCP, 6-day | 93.19% | 93.32% | | | | | 93.67% | | | |
| storage | | | | | | | | | | |
| RCP, 7-day | — | — | — | — | — | — | — | — | 93.78% | 93.80% |
| storage | | | | | | | | | | |

### VI. [¹¹¹In]In-447 Process and Formulation Development

The following section describes the radiochemistry synthesis of [¹¹¹In]In-447, followed by the direct formulation into the final [¹¹¹In]In-447 sterile drug product, that is suitable for clinical application when this process is transferred to a GMP clinical manufacture unit and produced under applicable clinical and regulatory guidance. The development leading to this final process, formulation, and references is described in detail in Section VII.

The following represent the final buffers that are chosen. The development process leading to these final buffers is described in Section VII.
Reaction buffer: 0.1 M sodium acetate buffer, pH˜4.1, with 0.1 mg/mL ascorbic acid and 1 mg/mL methionine.
Formulation buffer: 48 mg/mL sodium ascorbate, 10.66 mg/mL ascorbic acid, 24 mg/mL methionine, 0.04% w/v Tween 20, 0.04 M acetate, pH˜4.7.

### VII. Production of [¹¹¹In]In-447 Drug Product

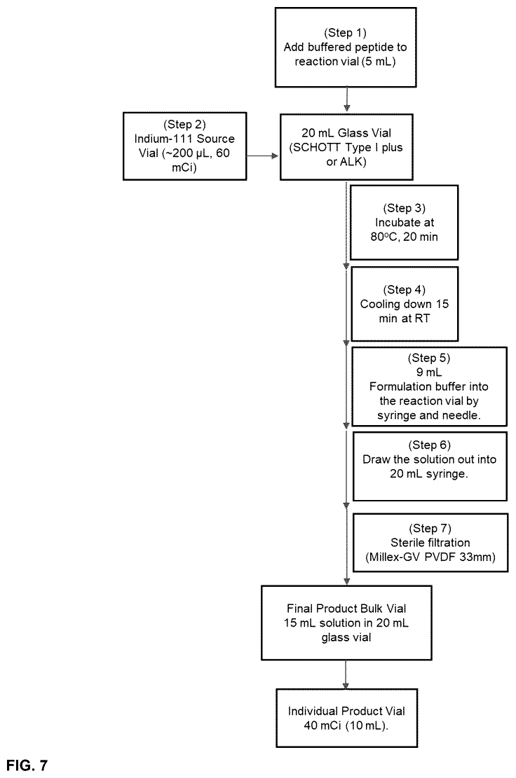

The production of [¹¹¹In]In-447 drug product consists of three (3) parts (FIG. 7);
(i) Radiosynthesis of the active pharmaceutical substance [¹¹¹In]In-447 in FIG. 7; Step 1 through Step 4,
(ii) End of Synthesis (EOS) product formulation in FIG. 7; Step 5 through Step 7, and
(iii) Post-EOF dispensing and Quality Control testing.

Radiosynthesis of active pharmaceutical substance [¹¹¹In]In-447 in-situ is conducted in a 6 ml 0.1 M sodium acetate reaction buffer containing 0.1% ascorbic acid (w/v) and 1% methionine (w/v), with sequential addition of 20 nmol of peptide precursor Compound 447 solution in ˜90 μL of metal free water, and 60 mCi of [¹¹¹In]InCl₃ solution in 0.05 M HCl. This reaction mixture is contained in a sealed 20 mL glass vial, heated at 80° C. for 20 minutes using a heating block, followed by a cooling step at ambient room temperature for 15 minutes.

Immediately upon the synthesis reaction mixture cooling step is completed, [¹¹¹In]In-447 drug product was formulated involving the addition of a 9 mL of formulation buffer directly into the sealed 20 mL glass vial and mixing with the reaction mixture. The final drug product [¹¹¹In]In-447 formulation solution is sterile filtered using a suitable 0.22 microns (μm) filter. A 10 mL, 40 mCi dose is dispensed with content shown in Table 29.

The sterile filter of choice for a production scale of less than 100 ml final formulation volume would ideally be readily commercially available globally, specifically low protein binding, with the compatibility to our aqueous based drug product formula matrix, with up to 10% ethanol, some trace percent of surfactants and broadly have been used with radiopharmaceuticals. A suitable choice is the low protein binding Durapore® PVDF member, a Millex-GV 33 mm sterile filter, CE-marked and suitable for both medical and laboratory applications in and outside the U.S.

Dispensing and QC testing are followed at the End of Formulation (EOF), this involves the dispensing of an appropriate radioactivity dose as per dosing calibration time requested, into a sterile container closure and separate materials sufficient for QC release testing of the drug product. The current production process is designed to provide stability for the longest calibration time of 96 hours, a dispensed dose of 40 mCi.

TABLE 29
Contents and Concentrations of the Dispensed Final Drug Product Vial

| | | |
|---|---|---|
| Volume | 10 | mL |
| Radioactive Concentration | 4 | mCi/mL |
| Total Activity | 40 | mCi |
| Concentration of 447 | 2.8 | μg/mL |
| Calculated Specific Activity | 1.4 | mCi/μg |
| Concentration of Sodium Ascorbate | 48 | mg/mL |
| Concentration of Ascorbic Acid | 10.66 | mg/mL |
| Concentration of Methionine | 24 | mg/mL |
| Concentration of Tween 20 | 0.04% | w/v |

| | |
|---|---|
| Concentration of Acetate | 0.04 M |
| pH | 4.7 |

### VIII. [¹¹¹In]In-496 Radiolabeling and Formulation

Radiosynthesis of active pharmaceutical substance [¹¹¹In]In-496 in situ was conducted in 6 mL of 0.1 M sodium acetate reaction buffer containing 5 mg/mL ascorbic acid, with sequential addition of ˜60 μg (20 nmol) of peptide precursor Cpd 496 solution in ˜60 μL of sodium acetate buffer, and ˜60 mCi of [¹¹¹In]InCl₃ solution in 0.05 M HCl. This reaction mixture was contained in a sealed 20 mL glass vial, heated at 70° C. for 20 minutes using a heating block followed by a cooling step at ambient room temperature for 10 minutes.

Immediately upon completion of the cooling step, [¹¹¹In]In-496 drug substance was formulated by the addition of a 6 mL of formulation buffer and unlabeled peptide solution directly into the 20 mL glass vial containing the reaction mixture. The vial was sealed and mixed.

Contents and Concentrations of [¹¹¹In]In-496

| | |
|---|---|
| Volume (mL) | 8 |
| Radioactive Concentration (mCi/mL) | 5 |
| Total Activity (mCi) | 40 |
| Concentration of Cpd 496 (μg/mL) | 4.5-430 |
| Calculated Specific Activity (mCi/μg) | 1.1 |
| Concentration of Sodium | 50 |
| Ascorbate (mg/ml) | |
| Concentration of Ascorbic Acid (mg/mL) | 7.5 |
| Concentration of Tween-20 (%, w/v) | 0.05 |
| Concentration of Acetate (M) | 0.05 |
| pH | 5.2 |

In order to arrive at the final formulation, shown above, the buffer formulation was optimized with the goal of maintaining the RCP (>90%) throughout the target expiry of 144 hours. Initial conditions (Reaction 1) and optimized conditions (Reactions 2 and 3) are shown below.

| Reaction Number | 1 | |
|---|---|---|
| Formulation | 30 mg/mL sodium ascorbate, | |
| | 20 mg/mL ascorbic acid, 0.05% | |
| | Polysorbate-20 w/v, 0.05M | |
| | acetate | |
| Dose Volume (mL) | 8 | |
| Radioactive Conc. (mCi/mL) | 5 | |
| Total activity (mCi) | 40 | |
| Storage condition | 2-8° C. | |
| RCP, End of Formulation (EOF) | 94.07% | |
| RCP, 24-hour storage | 93.94% | |
| RCP, 43-hour storage | 93.63% | |
| RCP, 116-hour storage | 93.06% | |
| RCP, 143-hour storage | 93.67% | |
| RCP, 169-hour storage | 93.47% | |
| Reaction Number | 2 | 3 |
| Formulation | 50 mg/mL sodium ascorbate, | |
| | 7.5 mg/mL ascorbic acid, 0.05% | |
| | Tween ® 20 w/v, 0.05M acetate. | |
| Dose Volume (mL) | 12 | |
| Radioactive Conc. (mCi/mL) | 5 | |
| Total activity (mCi) | 59.7 | 60.4 |
| Storage condition | 2-8° C. | 2-8° C. |
| RCP, EOF | 96.04% | 96.38% |
| RCP, 24-hour storage | 95.55% | 95.73% |
| RCP, 48-hour storage | 95.82% | 95.98% |
| RCP, 72-hour storage | — | 96.44% |
| RCP, 96-hour storage | — | 95.71% |
| RCP, 120-hour storage | — | — |
| RCP, 144-hour storage | 95.63% | — |
| RCP, 168-hour storage | 95.82% | 96.04% |

The RCP and was determined using a RP-HPLC analysis method, described below. RCP was calculated as the proportion of the total radioactivity in the sample which is present as [¹¹¹In]In-496.

| System | Agilent 1260 Infinity II |
|---|---|
| Column | Waters XBridge C18; 3.5 μm, 4.6 × 250 mm |
| Column temperature | Ambient |
| UV wavelength (nm) | 220/280 |
| Radiodetection | Flow-RAM coupled to 1″ Nal PMT |
| Mobile Phase A | 0.1% (v/v) formic acid/water |
| Mobile Phase B | 0.1% (v/v) formic acid/acetonitrile |
| Flow rate (mL/min) | 0.5 |
| Run time (min) | 35 |
| Retention time, | 16.1 |
| [¹¹¹In]In-496 (min) | |
| Retention time, unbound | 4.6 |
| [¹¹¹In]In (min) | |

Gradient Conditions

| | Mobile | Mobile |
|---|---|---|
| | Phase A | Phase B |
| Time (min) | (%) | (%) |
| 00.00 | 75 | 25 |
| 24.00 | 35 | 65 |
| 28.00 | 10 | 90 |
| 29.00 | 10 | 90 |
| 30.00 | 75 | 25 |

### Example 6: Synthesis and Characterization of [¹⁷⁷Lu]Lu-496

### I. Synthesis and Characterization of Compound 496

### Synthetic Procedure of Fmoc-Pip(CH₂COOtBu)Ala-OH

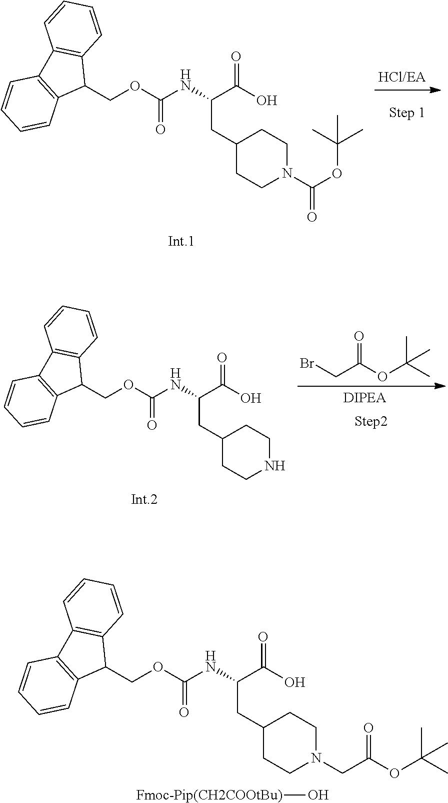

Step 1: The flask (100 mL) was charged with Compound Int. 1 (1 g, 2.02 mmol). 20 mL 4N HCl/EA was added into the flask and stirred at RT for 2 hours. The reaction was concentrated to dryness and carried over to the next step without further purification.

Step 2: The compound Int. 2 dissolved by 20 mL ACN/H₂O=1/1 was added tert-Butyl bromoacetate (0.334 mL, 2.22 mmol, 1.1 equiv.) and DIPEA (0.702 mL, 10.1 mmol, 5 equiv.) sequentially. The mixture was stirred for 2 h at RT. The mixture was filtered with 0.45 μm filter. The filtrate was purified by RP-HPLC. The desired fractions were collected and lyophilized to give the final compound Fmoc-Pip(CH₂COOtBu)-OH (0.789 g) as a white solid.

### Synthetic Procedure of Fmoc-Ala([DOTA-tris(tBuester)]-PEG4-piperidin-4-yl)-OH

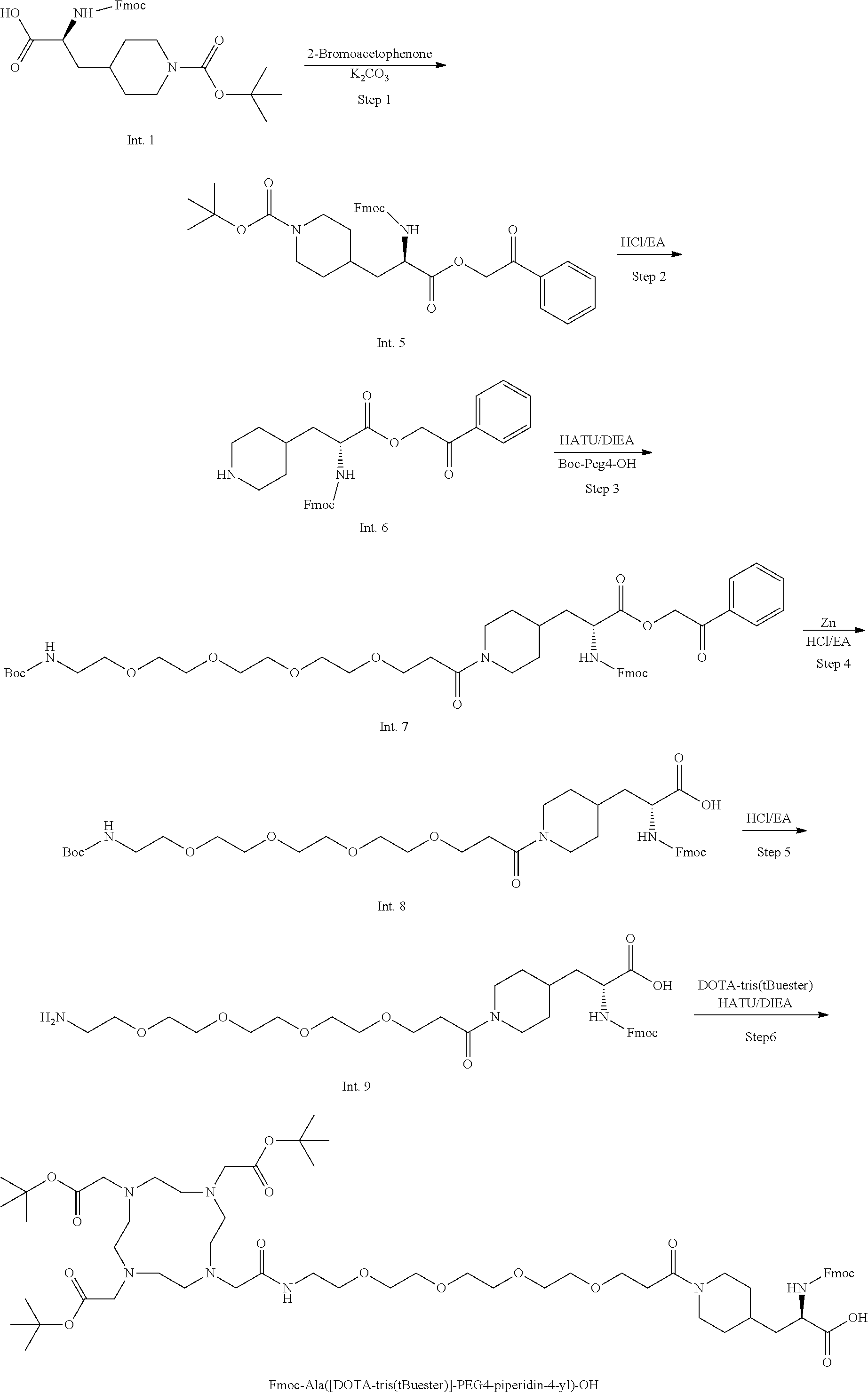

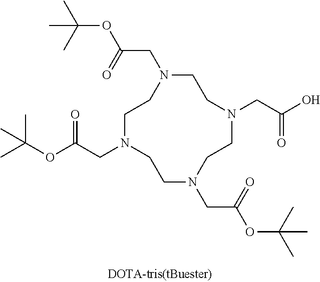 [SMILES: CC(C)(C)OC(=O)CN1CCN(CC(=O)O)CCN(CC(=O)OC(C)(C)C)CCN(CC(=O)OC(C)(C)C)CC1]

Step 1: To a solution of Int. 1 (5 g, 10.11 mmol) in 50 mL DMF was added K₂CO₃ (1.53 g, 11.12 mmol) and 2-Bromoacetophenone (2.2 g, 11.12 mmol) sequentially, the solution was stirred at R.T for 1 h. Upon completion, EA/H₂O (100 mL:200 mL) was added to the above reaction solution, then the EA phase was separated and washed by sat. NaCl solution once. The EA phase was collected and dried by Na₂SO₄ for 1.5 hours and then EA was evaporated to get the crude Int. 5 (6.0 g) without further purification.

Step 2: To a solution of Int. 5 (6 g) was added 120 mL HCl/EA (4N) buffer and stirred for 2 hours at RT. The above solution was evaporated directly to get the Int. 6 and was carried over to the next step without further purification.

Step 3: To a solution of Boc-PEG4-OH 3.7 g (10.11 mmol) and HATU 3.84 g (10.11 mmol) in 20 mL DMF at 0-4° C. was added DIEA and stirred for 5-15 minutes. Then, all of the above Int. 6 was dissolved by 30 mL DMF was added dropwise to the solution and stirred at RT until TLC (DCM:MeOH:AcOH=40:1:0.5) showed the Int. 6 was consumed. Upon completion, EA/H₂O (150 mL:300 mL) was added to the above reaction solution, then the EA phase was separated and washed by sat. NaCl solution once. The EA phase was collected and dried by Na₂SO₄ for half an hour and then EA was evaporated to get the crude Int. 7 (10 g) and carried over to the next step without further purification.

Step 4: To a solution of Int. 7 in 70 mL AcOH/H₂O (9:1) was added Zn powder 4.0 g (60.66 mmol) activated by conc. HCl, and then stirred at RT overnight. Upon completion, the solution was filtrated and the EA filtrate was collected and washed by sat. NaCl solution once. The EA phase was collected and dried by Na₂SO₄ for half an hour and then EA was evaporated to get the crude Int. 8 as oil. Then the Int. 8 was redissolved by 40 mL EA and added a (PE/EA=10:1) solution 500 mL dropwise and stirred overnight to get Int. 8 as a solid (8.0 g).

Step 5: To a solution of Int. 8 (8.0 g) was added 120 mL HCl/EA (4N) buffer and stirred for 2 hour at RT Upon completion, the above solution was concentrated to dryness to give Int. 9 and carried over to the next step without further purification.

Step 6: To a solution of DOTA-tris(tBuester) 5.73 g (10.0 mmol) and HATU 3.8 g (10.0 mmol) in 20 mL DMF at 0-4° C. was added DIEA and stirred for 5-15 minutes. Then, all of the above Int. 9 7.6 g dissolved by 30 mL DMF was added dropwise to the solution and stirred at RT for 2 hours. Additional DOTA-tris(tBuester) 5.73 g (10.0 mmol) was added to the reaction and kept stirring overnight. Upon completion, EA/H₂O (150 mL:300 mL) was added to the above reaction solution, then 5% H₃PO₄ buffer was added to adjust the pH to 3-4, then the EA phase was separated and washed by sat. NaCl solution once. The EA phase was collected and dried by Na₂SO₄ for half an hour and then EA was evaporated to get the crude Compound 8 and then purified by RP-HPLC to afford the title compound as a solid (4.36 g).

### General Procedure A

Peptides were synthesized using the CEM Liberty Blue microwave peptide synthesizer. Standard Fmoc chemistry and couplings were used with Rink Amide resin or Wang Resin.

### Synthesis

1. Deprotection: 20% piperidine in DMF (4 mL) was added to the resin and the mixture was heated to 90° C. for 1 minute. The resin was then washed with 4 mL of DMF 4 times.
2. Coupling: To the microwave reaction vessel was added Fmoc-protected amino acid (5 equiv.), DIC (10 equiv.) in DMF (1 M), and OxymaPure (5 equiv.) in DMF (1 M). The microwave reaction vessel is then heated to 90° C. for 2 minutes and then drained. For a double coupling, this process was repeated. For a triple coupling, this process was repeated two times. (Double coupling were primarily used, single couplings were used for precious building blocks, and triple coupling were used for difficult couplings)
3. Steps 1 and 2 were repeated for the remaining amino acids.
4. Capping: Deprotection protocol from Step 1 was repeated. 4 mL of 10% Ac₂O in DMF was added to the microwave reaction vessel and then heated to 65° C. for 2 minutes. The resin was then washed with 4 mL of DMF 4 times.
5. Orthogonal Deprotection (Dde): The resin is washed with 4 mL of DMF 2 times. To the microwave reaction vessel was added 4 mL of 2% Hydrazine in DMF, which was mixed at room temperature for 30 minutes. The resin is then washed with 4 mL of DMF and additional 5 times.
6. DOTA Coupling: To the microwave reaction vessel was added Fmoc-protected amino acid (5 equiv.), DIC (10 equiv.) in DMF (1M), and OxymaPure (5 equiv.) in DMF (1 M). The microwave reaction vessel is then heated to 90° C. for 4 minutes and then drained. This step is repeated an additional two times.

### Peptide Cleavage, Cyclization and Purification

1. Using the CEM Razor peptide cleavage system, the peptide resin was transferred to a fritted cleavage tube, and 8 mL of cleavage solution (TFA/DODT/H₂O/TIS, 92.5/2.5/2.5/2.5, v/v/v/v) was added to each tube. The peptide resin in cleavage solution was heated to 40° C. for 45 minutes.
2. Each tube is filtered, and the filtrate is collected in centrifuge tubes.
3. The peptide is then precipitated with cold ether (45 mL). The precipitated peptides are then placed on to a centrifuge and allowed to spin for at least 5 minutes. The ether is then decanted from each tube, leaving the peptide at the bottom. This ether precipitation process is then repeated a second time.
4. The peptide pellet is then dissolved in 15 mL of MeCN/H₂O (1/1, v/v), and 0.1 M I₂ in MeOH was added dropwise until a yellow color persisted. The mixture was shaken at room temperature for 10 minutes. The reaction was quenched by adding 0.1 M Na₂S2O₃ in water, dropwise until the yellow color disappeared. The reaction solution is then concentrated under reduced pressure.
5. Purification: The crude peptide within 2% v/v trifluoroacetic acid (TFA) in 1:1 acetonitrile/water was purified by preparative reversed-phase HPLC under acidic conditions, followed by lyophilization to obtain the product.
   1. Column: XSelect Peptide CSH C18, 1.9 cm i.d.×25 cm (5 μm/130 Å)
   2. Mobile phase: Mobile Phase A: 0.05% v/v trifluoroacetic acid in water; Mobile phase B: acetonitrile
   3. Gradient: 10% B for 4 min; 10→20% B over 4 min; 20→30% B over 20 min; 30→95% B over 4 min; 95% B for 2 min. Column temperature: ambient. Flow rate: 24 mL/minute.

### General Procedure B

Peptides were synthesized using the CEM Liberty Blue microwave peptide synthesizer. Standard Fmoc chemistry and couplings were used with 2-Chloro-Trityl Resin

### Resin Loading

Preloaded 2-Chloro-Trityl Resins were used when available. If the preloaded resin was not available, this procedure was used to manually load the resin with the first amino acid.

To a fritted syringe was added 1.0 g of 2-Chlorotrityl Chloride Resin. The resin was washed with 4 mL of DMF three times, followed by washing with 4 mL of DCM three times to swell the resin. In a separate vial, 12 mL of DCM was added, followed by 2 equiv. DIPEA and 1.2 equiv. of the desired amino acid (1^{st} amino acid in sequence). This solution was added to the resin and allowed to shake for 2 hours. The reaction mixture was then drained. The coupling protocol was repeated a second time. After the second coupling, the resin was washed with 4 mL of DMF three times, followed by 4 mL of DCM three times. To a separate vial was added 17 mL DCM, 2 mL of MeOH, and 1 mL of DIPEA. The solution was added to the resin and allowed to shake for 2 h. The reaction mixture was then drained and washed with 4 mL of DCM three times, followed by 4 mL of DMF three times. A solution of 8 mL of 20% Piperidine in DMF was added to the resin and shaken at room temperature for 15 minutes. The reaction mixture was drained and washed with 4 mL of DMF three times and 4 mL of DCM three times. The resin was then placed under vacuum to dry.

### Synthesis

1. Deprotection: 20% piperidine in DMF (4 mL) was added to the resin and the mixture was heated to 75° C. for 3 minutes. The resin was then washed with 4 mL of DMF 4 times.
2. Coupling: To the microwave reaction vessel was added Fmoc-protected amino acid (5 equiv.), DIPEA (10 equiv.) in DMF (1 M), and HATU (5 equiv.) in DMF (0.5 M). The microwave reaction vessel is then heated to 75° C. for 5 minutes and then drained. For a double coupling, this process was repeated. For a triple coupling, this process was repeated two times. (Double coupling were primarily used, single couplings were used for precious building blocks, and triple coupling were used for difficult couplings)
3. Iteration: Steps 1 and 2 were repeated for the remaining amino acids.
4. Capping: Steps 1 and 2 were used with Acetic Acid as the capping reagent.
5. Orthogonal Deprotection (Dde): The resin is washed with 4 mL of DMF 2 times. To the microwave reaction vessel was added 4 mL of 2% Hydrazine in DMF, which was mixed at room temperature for 30 minutes. The resin is then washed with 4 mL of DMF and additional 5 times.
6. If there are amino acids between the deprotection in step 5 and DOTA, those amino acids are installed by repeating steps 1 and 2.
7. DOTA Coupling: To the microwave reaction vessel was added Fmoc-protected amino acid (5 equiv.), DIPEA (10 equiv.) in DMF (1 M), and HATU (5 equiv.) in DMF (0.5 M). The microwave reaction vessel is then heated to 75° C. for 5 minutes and then drained. This step is repeated an additional two times.

### Peptide Cleavage, Cyclization and Purification

1. Using the CEM Razor peptide cleavage system, the peptide resin was transferred to a fritted cleavage tube, and 8 mL of cleavage solution (TFA/DODT/H₂O/TIS, 92.5/2.5/2.5/2.5, v/v/v/v) was added to each tube. The peptide resin in cleavage solution was heated to 40° C. for 45 minutes.
2. Each tube is filtered, and the filtrate is collected in centrifuge tubes.
3. The peptide is then precipitated with cold ether (45 mL). The precipitated peptides are then placed on to a centrifuge and allowed to spin for at least 5 minutes. The ether is then decanted from each tube, leaving the peptide at the bottom. This ether precipitation process is then repeated a second time.
4. The peptide pellet is then dissolved in 15 mL of MeCN/H₂O (1/1, v/v), and 0.1 M I₂ in MeOH was added dropwise until a yellow color persisted. The mixture was shaken at room temperature for 10 minutes. The reaction was quenched by adding 0.1 M Na₂S2O₃ in water, dropwise until the yellow color disappeared. The reaction solution is then concentrated under reduced pressure.
5. Purification: The crude peptide within 2% v/v trifluoroacetic acid (TFA) in 1:1 acetonitrile/water was purified by preparative reversed-phase HPLC under acidic conditions, followed by lyophilization to obtain the product.
   1. Column: XSelect Peptide CSH C18, 1.9 cm i.d.×25 cm (5 μm/130 Å)
   2. Mobile phase: Mobile Phase A: 0.05% v/v trifluoroacetic acid in water; Mobile phase B: acetonitrile
   3. Gradient: 10% B for 4 min; 10→20% B over 4 min; 20→30% B over 20 min; 30→95% B over 4 min; 95% B for 2 min. Column temperature: ambient. Flow rate: 24 mL/minute.

Rink Amide MBHA Resin (0.1 mmol, 0.353 g, Sub: 0.284 mmol/g) was swelled in DMF (10 mL) for 2 h, then 20% Piperidine in DMF (10 mL) was added into the resin. The mixture was kept at room temperature for 30 minutes while a stream of nitrogen bubbled through it. The mixture was filtered and the peptidyl resin was washed with DMF (5*10 mL). Fmoc-DGlu(OtBu)-OH (0.2 mmol, 0.083 g), DMF (1.5 mL), NMM (0.4 mmol, 0.044 mL) and HATU (0.19 mmol, 0.72 g) were added into the resin sequentially. The suspension was kept at room temperature for 1 hour while a stream of nitrogen bubbled through it. The coupling was monitored by ninhydrine test until its completion. The mixture was filtered and the peptidyl resin was washed with DMF (3*10 mL). 20% Piperidine in DMF (10 mL) was added into the resin to remove the Fmoc group. The following amino acids were coupled to the resin bound peptide sequentially as follows:

Synthesis Table

| | Reaction | | | Reaction | Deprotection | |
|---|---|---|---|---|---|---|
| Amino Acid | Solvent | Reagents | | Time | reagents and time | |
| Fmoc-Val(3-OH)-OH | DMF | HATU | NMM | 60 min | 20% | 25 |
| 0.2 mmol | (1.5 mL) | 0.19 | 0.4 | | Pip/DMF 10 | min |
| | | mmol | mmol | | mL | |
| Fmoc-(2S,3S)-2- | DMF | HATU | NMM | 58 min | 20% | 30 |
| amino-3- | (1.5 mL) | 0.19 | 0.4 | | Pip/DMF 10 | min |
| phenylbutanoic acid | | mmol | mmol | | mL | |
| 0.2 mmol | | | | | | |
| Fmoc-Trp(5-F)-OH | DMF | HATU | NMM | 55 min | 20% | 30 |
| 0.2 mmol | (1.5 mL) | 0.19 | 0.4 | | Pip/DMF 10 | min |
| | | mmol | mmol | | mL | |
| Fmoc-Cys(Trt)-OH | DMF | HATU | NMM | 60 min | 20% | 25 |
| 0.3 mmol | (1.5 mL) | 0.285 | 0.6 | | Pip/DMF 10 | min |
| | | mmol | mmol | | mL | |
| Fmoc-Tyr(tBu)-OH | DMF | HATU | NMM | 59 min | 20% | 30 |
| 0.3 mmol | (1.5 mL) | 0.285 | 0.6 | | Pip/DMF 10 | min |
| | | mmol | mmol | | mL | |
| Fmoc-Val-OH | DMF | HBTU | NMM | 48 min | 20% | 30 |
| 0.3 mmol | (1.5 mL) | 0.285 | 0.6 | | Pip/DMF 10 | min |
| | | mmol | mmol | | mL | |
| Fmoc-Asp(OtBu)-OH | DMF | HBTU | NMM | 40 min | 20% | 26 |
| 0.3 mmol | (1.5 mL) | 0.285 | 0.6 | | Pip/DMF 10 | min |
| | | mmol | mmol | | mL | |
| Fmoc-Ala([DOTA- | DMF | HATU | NMM | 60 min | 20% | 28 |
| tris(tBuester)]-PEG4- | (1.5 mL) | 0.142 | 0.3 | | Pip/DMF 10 | min |
| piperidin-4-yl)-OH | | mmol | mmol | | mL | |
| 0.15 mmol | | | | | | |
| Fmoc- | DMF | HATU | NMM | 50 min | 20% | 30 |
| Pip(CH2COOtBu)Ala- | (1.5 mL) | 0.19 | 0.4 | | Pip/DMF 10 | min |
| OH | | mmol | mmol | | mL | |
| 0.2 mmol | | | | | | |
| Fmoc-Gly-OH | DMF | HBTU | NMM | 55 min | 20% | 30 |
| 0.3 mmol | (1.5 mL) | 0.285 | 0.6 | | Pip/DMF 10 | min |
| | | mmol | mmol | | mL | |
| Fmoc-Tbg-OH | DMF | HATU | NMM | 60 min | 20% | 24 |
| 0.2 mmol | (1.5 mL) | 0.19 | 0.4 | | Pip/DMF 10 | min |
| | | mmol | mmol | | mL | |
| Fmoc-Cys(Trt)-OH | DMF | HBTU | NMM | 50 min | 20% | 30 |
| 0.3 mmol | (1.5 mL) | 0.285 | 0.6 | | Pip/DMF 10 | min |
| | | mmol | mmol | | mL | |
| Fmoc-Tyr(tBu)-OH | DMF | HBTU | NMM | 50 min | 20% | 30 |
| 0.3 mmol | (1.5 mL) | 0.285 | 0.6 | | Pip/DMF 10 | min |
| | | mmol | mmol | | mL | |
| Fmoc-homoLeu-OH | DMF | HATU | NMM | 50 min | 20% | 30 |
| 0.3 mmol | (1.5 mL) | 0.285 | 0.6 | | Pip/DMF 10 | min |
| | | mmol | mmol | | mL | |
| Ac₂O | DMF | N/A | NMM | 30 min | N/A | N/A |
| 1 mL | (8.4 mL) | | 0.6 mL | | | |

After the peptidyl resin was assembled, it was washed with DCM (3*10 mL) and MeOH (3*10 mL). The resin was dried under vacuum overnight.

### Cleavage

Cleavage E solution (TFA:Thioanisole:phenol:EDT:H₂O=87.5:5:2.5:2.5:2.5, 10 mL) was added into peptidyl resin (0.782 g) under the protection of nitrogen. The mixture was shaken for 2.5 hours at room temperature. Cold ether (60 mL) was added into the filtrate, the peptide was precipitated by centrifugation (4000 revolutions/minute). The precipitate pellet was washed with ether (60 mL) twice. Then the crude was dried under vacuum overnight to give the crude as a solid.

### Oxidation

0.129 g crude was dissolved by 5 mL ACN, 10 mL H₂O and 15 mL AcOH. Then 2 mL of I₂ in methanol (10 mg/mL) was added into the mixture and the mixture was stirred for 5 minutes at RT. Ascorbic acid was added into the solution while slow magnetic stirring until the solution changed to colorless. The solution was filtered with 0.45 μm filter.

### Purification

### Step 1

The filtrate was purified by using a Hanbon 300 connected to Phenomenex Luna™ C18 100 Å; 10 μm; 21*250 mm HPLC column. Method: gradient window of solvent B from 38% to 68% over 60 minutes at a flow rate of 15 mL/minute (solvent A: 4 g/L Ammonium acetate in water; solvent B: 4 g/L Ammonium acetate in 70% ACN/H₂O). The purification fractions with purity more than 91% were collected.

### Step 2

The buffer from step 1 with purity more than 91% was purified further by using a Hanbon 300 connected to Phenomenex Luna™ C18 100 Å; 10 μm; 21*250 mm HPLC column. Method: gradient window of solvent B from 38% to 68% over 60 minutes at a flow rate of 15 mL/minute (solvent A: 0.1% TFA in water; solvent B: 0.1% TFA in 80% ACN/H₂O). The solution was collected and lyophilized to give the final peptide as a solid.

Table 30 below shows LCMS analysis for the Compound 496.

TABLE 30

| | Molecular | | observed | |
|---|---|---|---|---|
| Cpd# | Weight | exact mass | mass | comment |
| 496 | 2711.12 | 2709.28 | 904.40 | [M + 3H]/3 |

### II. Synthesis of [¹⁷⁷Lu]Lu-496

[¹⁷⁷Lu]LuCl₃ was received from venders in HCl solution. For every mCi of [¹⁷⁷Lu]LuCl₃ added to the reaction vial (1.5 mL Eppendorf vial), ammonium acetate buffer (0.2 M, pH 4.9, 100 μL, containing 1% w/v ascorbic acid and 6% v/v ethanol) and peptide conjugate (1 nmol) was added. The pH of the solution was determined to be approximately 5 by using pH strips. The reaction vial was incubated at 80° C., 700 RPM for 17-20 minutes. A sample from the reaction was analyzed by RP-HPLC using an Agilent Infinity II 1260 HPLC to determine reaction completion and RCP. HPLC conditions: Waters XBridge BEH C18 Column, 130 Å, 3.5 μm, 4.6 mm×250 mm; mobile phase: Solvent A=water (with 0.1% formic acid), solvent B=acetonitrile (with 0.1% formic acid). Gradients: 25-45% B in 10 minutes, 45-65% B in 12 minutes, 65-90% in 6 minutes at a flow rate of 0.5 mL/minute. Required amounts of the product were formulated in 1% PBSA for cell studies.

### Example 7: High Specific Activity Radiolabeled Compounds for B7-H3

### I. High Specific Activity Production of [¹⁷⁷Lu]Lu-496

Representative reactions were carried out at a radioactivity scale range of 25-107 mCi in Eppendorf tubes and heating support using a small heater such as an orbital heater. Reaction volumes range from 650 μL to 1031 μL. The reaction buffer was an acetate buffer containing ascorbic acid as stabilizer. Reactions were heated at 80° C. setpoint for 20 min. Post heating and cooled reaction mixture was quenched with DTPA solution and injected into the analytical HPLC or Neptis semi-prep HPLC for purification. The specific purification method will vary based on the system used for purification and are described below. Fractions were collected into Formulation Buffer at the retention time of the [¹⁷⁷Lu]Lu-496 product. An aliquot of the formulated solution was analyzed by using RP-HPLC (Table 31). RCP (RCP) was reported based on HPLC analysis, and the specific activity was calculated based on the specific concentration and the concentration of peptide mass.

TABLE 31
Analytical HPLC method for RCP and peptide mass determination

| System | Agilent 1260 Infinity II |
|---|---|
| Column | Waters XBridge C18; 3.5 μm, 4.6 × 250 mm |
| Column temperature | Ambient |
| UV wavelength (nm) | 220/280 |
| Radiodetection | Flow-RAM coupled to 1.5″ Nal PMT |
| Mobile Phase A | 0.1% (v/v) formic acid/water |
| Mobile Phase B | 0.1% (v/v) formic acid/acetonitrile |
| Flow rate (mL/min) | 0.5 |
| Run time (min) | 35 |

Gradient Conditions

| | Mobile | Mobile |
|---|---|---|
| Time (min) | Phase A (%) | Phase B (%) |
| 00.00 | 75 | 25 |
| 24.00 | 35 | 65 |
| 28.00 | 10 | 90 |
| 29.00 | 10 | 90 |
| 30.00 | 75 | 25 |

### II. Radiolabeling and Purification with Analytical HPLC

To a 1.5 mL Eppendorf vial, 0.6 mL of 0.1 M Sodium acetate buffer containing 5 mg/mL ascorbic acid, 60 μL of ethanol, 223 μL of Compound 496 solution (0.298 nmol/μL) and 103 μL of Lu-177 chloride solution were added sequentially. The specific activity was 1.6 mCi/nmol for radiolabeling. The vial was assayed by a dose calibrator with Lu-1177 calibration number and the activity was 106.8 mCi. The reaction vial was heated at 80° C. C setpoint for 20 minutes. After the incubation was finished, the reaction was quenched with 30 μL of 10 mg/mL DTPA solution. 900 μL of the mixture (91.2 mCi) was injected into HPLC for purification with the purification method below.

Purification method: Waters XSelect CSH C18 4.6×152 mm, 3.5 μm; Mobile phase: 70% 1.5 mM NH4OAc in water, and 30% ethanol. Isocratic condition. Flow rate=0.5 mL/min. Column temp=50° C. Run time=35 min. Flow-RAM coupled to 1″ Nal PMT was used for detection of the radioactive product

Fractions were collected at 18:54-20:00 and 20:00-21:00 and the activities were 74.9 mCi and 2.15 mCi, respectively. Fraction 1 was diluted with formulation buffer to the final volume of 5 mL. The formulation buffer consisted of 75 mg/mL sodium acetate, 7.5 mg/mL ascorbic acid, 0.75% Tween 20, and 0.75 mg/mL DTPA in water. The concentration of the diluted Fraction 1 was 14.98 mCi/mL. The final drug product formulation contained 50 mg/mL sodium ascorbate, 5 mg/mL ascorbic acid, 0.05% tween-20 and 0.05 mg/mL DTPA. The RCP was 96.36% after formulation, and the specific activity was measured at 19.0 mCi/nmol. The sample was stored at 2-8° C. The RCP was 95.69% after 2-day storage. The results are summarized in the following tables.

| Radiolabeled Compound | HSA [¹⁷⁷Lu]Lu-496 |
|---|---|
| RCP | 96.4% |
| Radioactive Concentration (mCi/mL) | 8.69 |
| Drug Mass Concentration (μg/mL) | 1.2 |
| Total activity (mCi) | 40.5 |
| Total volume (mL) | 4.66 |
| Storage condition | 2-8° C. |

| | Sodium ascorbate 50 mg/mL, |
|---|---|
| | Ascorbic acid 5 mg/mL, Tween-20 |
| HSA [¹⁷⁷Lu]Lu-496 Formulation | 0.05%, Ethanol content 10% |
| RCP, EOF after | 96.36 |
| purification (HPLC, %) | |
| ¹⁷⁷Lu incorporation, EOF | 99.72 |
| after purification (HPLC, %) | |
| RCP, 2 day storage | 95.69 |
| 2-8° C. (HPLC, %) | |
| ¹⁷⁷Lu incorporation, | 99.44 |
| 2 day storage 2-8° C. (HPLC, %) | |

### III. Purification with Neptis Semi-Prep HPLC

To a 1.5 mL Eppendorf vial, 0.2 mL of 0.1 M Sodium acetate buffer containing 5 mg/mL ascorbic acid, 0.6 mL 0.1 M Sodium acetate buffer, 50 μL of ethanol, 94 μL of Compound 496 solution (0.424 nmol/μL) and 87 μL of Lu-177 chloride solution were added sequentially. The vial was assayed by a dose calibrator with Lu-177 calibration number and the activity was 70.1 mCi. The reaction vial was heated at 80° C. for 15 minutes. After the incubation was finished, the reaction was quenched with 10 μL of 10 mg/mL DTPA solution. All the mixture (65 mCi) was injected by the automated Neptis system equipped with an HPLC system for purification.

Purification method: Waters XSelect CSH C18 4.6×250 mm, 5 μm; Mobile phase: 66% 5 mM ammonium acetate; 34% ethanol; isocratic, Column temperature=ambient temperature. Flow rate=1 mL/min. Flow-RAM coupled to 1″ Nal PMT was used for detection of the radioactive product.

Fraction was collected between 19:30-22:00 for 2.5-minute collection. The activity collected was 42.3 mCi. The product collection vial was prefilled with 1 mL of stabilization buffer (100 mg/mL sodium ascorbate, 10 mg/mL ascorbic acid, 0.1% tween-20). After collection, 5 mL of formulation buffer (50 mg/mL sodium ascorbate, 5 mg/mL ascorbic acid, 0.05% tween-20) was added to the collection vial to yield a final volume at 8.5 mL. The RCP was 98.97%.

### IV. Efficacy Study with [¹⁷⁷Lu]Lu-496 in a CDX Mouse Model (NCI-H1650 and SHP77)

The objective of this study was to compare the efficacy of RSA and HSA [¹⁷⁷Lu]Lu-496 drug products in a tumor model of human lung small cell lung cancer using SHP77 cells, which exhibit low receptor expression (˜27 k B7-H3 receptors/cell), and in NCI-H1650 cells, which exhibit relatively high receptor expression (˜212 k B7-H3 receptors/cell). A cell line-derived xenograft (CDX) mouse model is a research tool that involves implanting human tumor cells into an immunodeficient mouse to evaluate the efficacy of a cancer therapy. The chosen cell lines are good models to explore efficacy as they represent low end and high end of B7-H3 expression for CDX models.

All mice were sorted into study groups based on caliper estimation of tumor burden. The mice were distributed to ensure that the mean tumor burden for all groups was within 10% of the overall mean tumor burden for the study population. Particular details of the study are shown in the following table.

| | | | | | | Treatment |
|---|---|---|---|---|---|---|
| Group | N | Treatment | Dose | ROA | Regimen | (days) |
| 1 | 10 | Vehicle | 200 μL/injection | IV | QDx1 | 13 |
| 2 | 10 | [¹⁷⁷Lu] Lu-496 (RSA) | 1400 μCi/animal | IV | QDx1 | 13 |
| | | (1.4 mCi at | | | | |
| | | 200 uL/injection) | | | | |
| 3 | 10 | [¹⁷⁷Lu] Lu-496 (HSA) | 1400 μCi/animal | IV | QDx1 | 13 |
| | | (1.4 mCi at | | | | |
| | | 200 uL/injection) | | | | |
| 4 | 10 | [¹⁷⁷Lu] Lu-496 (RSA) | 700 μCi/animal | IV | QDx1 | 13 |
| | | (0.7 mCi at | | | | |
| | | 100 uL/injection) | | | | |
| 5 | 10 | [¹⁷⁷Lu] Lu-496 (HSA) | 700 μCi/animal | IV | QDx1 | 13 |
| | | (0.7 mCi at | | | | |
| | | 100 uL/injection) | | | | |

The RSA drug products were characterized as having a specific molar activity of about 1.4 mCi/nmol, whereas the HSA drug products were characterized as having a specific molar activity of about 15.5 mCi/nmol (Table 32).

TABLE 32

| Dose | Specific activity | Total mass | % labeled/% unlabeled |
|---|---|---|---|
| (mCi) | (mCi/nmol) | dose (ug) | peptide |
| 1.4 mCi | 15.5 | 0.19 | 80%/20% |
| (HSA) | | | |
| 1.4 mCi | 1.37 | 2.1 | 7.1%/92.9% |
| (RSA) | | | |
| 0.7 mCi | 15.5 | 0.095 | 80%/20% |
| (HSA) | | | |
| 0.7 mCi | 1.37 | 1.05 | 7.1%/92.9% |
| (RSA) | | | |

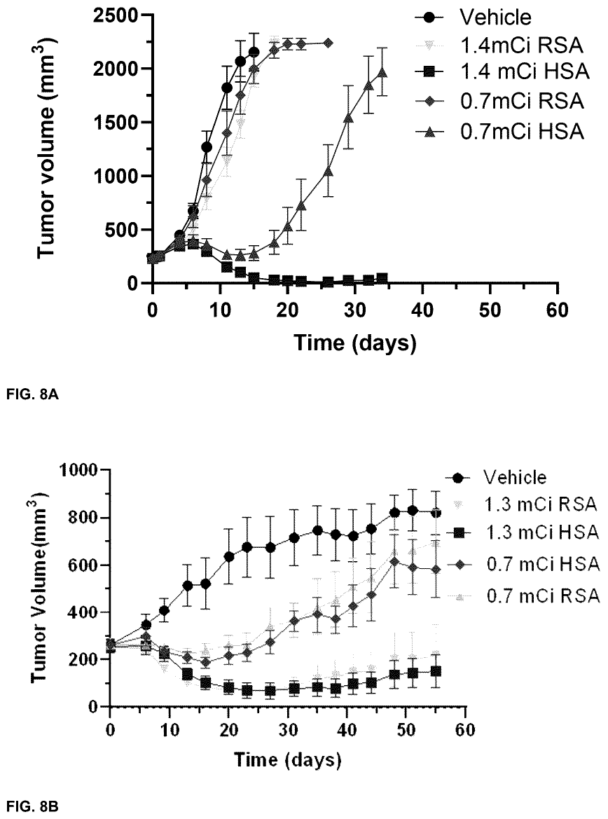

Treatment of animals having SHP77 tumors with [¹⁷⁷Lu]Lu-496 (HSA) at 1400 μCi/animal (1.4 mCi/animal) or 700 μCi/animal (0.7 mCi/animal) (Groups 3 and 5) produced dose-dependent anti-cancer activity. High dose treatment in Group 3 produced a Day 24 regression value of 28% and 80.0% incidences of complete regressions and tumor-free survivors. Low dose treatment in Group 5 produced a Day 24 median ΔT/ΔC value of 1%, an increase in time to progression of 163.6%, and 10.0% incidence of complete regressions. Treatment with [¹⁷⁷Lu]Lu-496 (RSA) at 1400 μCi/animal or 700 μCi/animal (Groups 2 and 4) produced median .T/.C values of 46% and 68% and ITP values of 27.2% and 9.0%, respectively. See, FIG. 8A.

As shown in FIG. 8B, equivalent efficacy was observed with HSA vs RSA approaches for higher the expressing model (NCI-H1650).

### Example 8: HSA [²²⁵Ac]Ac-496

### I. Small-Scale High Specific Activity Production of [²²⁵Ac]Ac-496

[²²⁵Ac]Ac source: Actinium-225 nitrate dissolved in 0.04 M hydrochloric acid to a radioactive concentration of 2-20 mCi/mL.

Reaction Buffer: 1 M sodium acetate pH 5-6 with a variable concentration of ascorbic acid such that the total reaction volume contains an ascorbic acid concentration of 5 mg/mL.

Peptide Solution: Compound 496 dissolved in 0.1 M sodium acetate pH 5-6 buffer to a concentration of 0.592-0.690 nmol/μL.

Formulation Buffer: 0.1 M ammonium acetate buffer pH 5.5-6.0 containing sodium ascorbate, L-methionine, and Tween-20 such that the concentrations in the radiolabeled product formulation are 50 mg/mL, 23.6 mg/mL, and 0.05% w/v respectively.

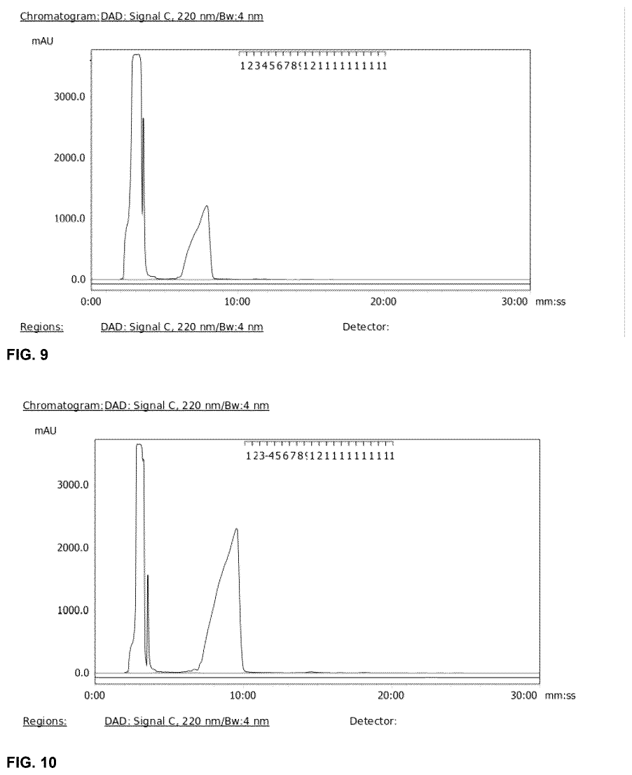

Procedure (Table 33 through Table 37): Ac-225 source was added to a 1.5 mL Eppendorf Lo-Bind microcentrifuge tube. Reaction Buffer was added to the vial at a volume of 0.5-0.6 times the volume of Ac-225 source added. Ethanol was added to the vial such that the concentration in the total reaction volume is 9.4-10.0% v/v. Peptide Solution was added to the vial such that the targeted specific molar activity was 4.9 μCi/nmol. The reaction mixture was incubated at 90° C. for 15 minutes on a USA Scientific Mixer HC at 700 RPM. The reaction was then allowed to briefly cool before RP-HPLC purification. The reaction was transferred to an HPLC vial containing 5 μL of 0.05 mg/mL DTPA in a 0.1 M ammonium acetate buffer pH 5.5. The reaction vial was rinsed with an aliquot of Formulation Buffer and transferred to the HPLC vial such that the total volume in the HPLC vial was ˜110 μL. The vial contents were injected onto the HPLC. HPLC eluate was collected in 0.5 ml fractions between 10 and 20 minutes into vials that were preloaded with Formulation Buffer. The collected fractions were assayed for activity and the fractions containing the desired radiolabeled product identified (FIG. 9 and FIG. 10). These fractions were analyzed via RP-HPLC and iTLC for RCP. The radiolabeled product was then stored at 2-8C.

TABLE 33
Reaction Summaries.

| | Run #1 | Run #2 |
|---|---|---|
| Ac-225 Activity (μCi) | 70 | 200 |
| Total Reaction Volume (μL) | 79.5 | 84.1 |
| Reaction Sodium Acetate Concentration | 0.23 | 0.14 |
| (M) | | |
| Reaction Peptide Concentration (nmol/μL) | 0.18 | 0.49 |
| Reaction Radioactivity Concentration | 0.88 | 2.38 |
| (μCi/μL) | | |
| Targeted Specific Molar Activity (μCi/nmol) | 4.9 | 4.9 |
| Isotope Incorporation via iTLC (%) | 99.25 | >99 |

TABLE 34
Results Summaries.

| ELN | Run #1 | Run #2 |
|---|---|---|
| Product Activity (μCi) | 44 | 156 |
| Sodium Ascorbate (mg/mL) | 50 | 50 |
| Methionine (mg/mL) | 23.6 | 23.6 |
| Tween-20 (% w/v) | 0.05 | 0.05 |
| Ethanol (% v/v) | 7.5 | 7.5 |
| Ammonium Acetate (M) | 0.08 | 0.08 |
| Product Radioactive | 0.04 | 0.05 |
| Concentration (μCi/μL) | | |
| Radioactive Yield (%) | 63 | 78 |
| RCP via RP-HPLC (%) | 95.00 | 96.16 |
| RCP via iTLC (%) | 99.80 | >99 |
| 24 hr - RCP via | 94.60 | N/A |
| RP-HPLC (%) | | |
| 24 hr - RCP via | 99.90 | N/A |
| iTLC (%) | | |

TABLE 35
iTLC Conditions.

| | |
|---|---|
| Mobile phase | 50 mM EDTA; pH 5.4 |
| Stationary Phase | Agilent Silica Gel Impregnated |
| | Microfiber Paper (p/n: SGI0001) |
| Radiodetection | Eckert & Ziegler AR2000¹ |

¹Post-development, iTLC papers are stored until secular equilibrium between Ac-225 and its daughter radioactive isotopes is achieved (>6 hours). They are then scanned using the AR2000.

TABLE 36
Analytical RP-HPLC Conditions.

| | |
|---|---|
| System | Analytical HPLC (HPLC04) |
| Mobile phase A | 0.1% Formic acid in water |
| Mobile phase B | 0.1% Formic acid in acetonitrile |
| Column | Waters XBridge C18; 3.5 μm, 4.6 × |
| | 250 mm (p/n: 186003943) |
| Flow rate (mL/min) | 0.5 |
| Running conditions | Gradient - 25%-75% B, 0-23 min |
| | 90% B, 23-28 min |
| Radiodetection | Perkin Elmer Wizard2 2470 Gamma Counter¹ |

¹Fractions were stored until secular equilibrium between Ac-225 and its daughter radioactive isotopes was achieved (>6 hours). Fractions were then analyzed using the gamma counter and a chromatogram was reconstructed from the CPM values as a function of time.

TABLE 37
Purification RP-HPLC Conditions.

| | |
|---|---|
| System | Analytical HPLC (HPLC01) |
| Mobile phase A | 70% 1.5 mM ammonium acetate |
| Mobile phase B | 30% ethanol |
| Column | Waters XSelect CSH C18; 3.5 μm, 4.6 × |
| | 150 mm (p/n: 186006957) |
| Column Temp | 50 |
| (° C.) | |
| Flow rate | 0.5 |
| (mL/min) | |

### II. Large-Scale High Specific Activity Production of [²²⁵Ac]Ac-496

[²²⁵Ac]Ac source: Actinium-225 nitrate dissolved in 0.04 M hydrochloric acid to a radioactive concentration of 1 mCi/mL.

Reaction Buffer: 1 M sodium acetate pH 5-6 with a variable concentration of ascorbic acid such that the total reaction volume contains an ascorbic acid concentration of 5 mg/mL.

Peptide Solution: Compound 496 dissolved in 0.1 M sodium acetate pH 5-6 buffer to a concentration of 0.665 nmol/μL.

Formulation Buffer: 0.1 M ammonium acetate buffer pH 5.5-6.0 containing sodium ascorbate, L-methionine, and Tween-20 such that the concentrations in the radiolabeled product formulation are 50 mg/mL, 23.6 mg/mL, and 0.05% w/v respectively.

Procedure (Table 38 through Table 40): Ac-225 source was added to a 10 mL ALK vial. Reaction Buffer was added to the vial such that the volume added was 0.5 times the volume of Ac-225 source added. Ethanol was added to the vial such that the concentration in the total reaction volume was 9-10% v/v. Peptide Solution was added to the vial such that the targeted specific molar activity was ˜5 μCi/nmol. The vial was sealed and crimped and placed into the Neptis heater. The vial was incubated at 90° C. for 12 minutes and then cooled with compressed air to 40° C. prior to purification. The reaction mixture was pulled from the reaction vial via a syringe driver. The vial was then washed with a buffered DTPA solution which was subsequently drawn into the syringe and mixed with the reaction mixture. The syringe then pushed the reaction mixture into the HPLC injection loop to begin purification. A 1″ Na/I probe was used to detect radioactivity in the eluate post-column and the radiolabeled product was identified by radioactive signal. Product collection was triggered by the operator. During product collection, flow was diverted from waste into a pre-loaded 50 mL ALK vial containing 26.25 mL of Formulation Buffer. The product was then analyzed via RP-HPLC and iTLC for RCP. See above section for methods. The radiolabeled product was stored at 2-8° C.

TABLE 38
Neptis Reaction Summary.

| | |
|---|---|
| Ac-225 Activity (μCi) | 1598 |
| Total Reaction Volume | 3182 |
| (μL) | |
| Reaction Sodium Acetate | 0.27 |
| Concentration (M) | |
| Reaction Peptide | 0.10 |
| Concentration (nmol/μL) | |
| Reaction Radioactivity | 0.50 |
| Concentration (μCi/μL) | |
| Targeted Specific Molar | 5.0 |
| Activity (μCi/nmol) | |
| Isotope Incorporation | 97.85 |
| via iTLC (%) | |

TABLE 39
Neptis Results Summary.

| | |
|---|---|
| Product Activity (μCi) | 1320 |
| Sodium Ascorbate | 50.0 |
| (mg/mL) | |
| Methionine (mg/mL) | 23.6 |
| Tween-20 (% w/v) | 0.05 |
| Ethanol (% v/v) | 8.75 |
| Ammonium Acetate (M) | 0.076 |
| Product Radioactive | 0.038 |
| Concentration (μCi/μL) | |
| Radioactive Yield (%) | 83 |
| RCP via RP-HPLC (%) | 97.14 |
| RCP via iTLC (%) | 99.88 |
| 48 hr - RCP via | 96.89 |
| RP-HPLC (%) | |
| 48 hr - RCP via | 99.80 |
| iTLC (%) | |
| 192 hr - RCP via | 95.15 |
| RP-HPLC (%) | |
| 192 hr - RCP via | 99.21 |
| iTLC (%) | |

TABLE 40
Neptis Purification Conditions.

| | |
|---|---|
| Mobile phase | 3.25 mM ammonium acetate, 35% |
| | (v/v) ethanol |
| Column | Waters XSelect Peptide CSH C18; |
| | 130 Å, 5 μm, 10 × 250 mm |
| | (p/n: 1806008267) |
| Column Temp (° C.) | Uncontrolled Ambient |
| Flow rate (mL/min) | 3.5 |
| Running conditions | Isocratic |
| Radiodetection | Flow-RAM with 1″ Na/I PMT |

### III. Stability Comparison Versus Regular Specific Activity [²²⁵Ac]Ac-496

Radiolabeling in this example proceeded as described in the above section, but without purification (Table 41 and Table 42). The reaction mixture was split into two equivalent formulations of differing activity and radioactive concentration. Ethanol was introduced into the final formulations by way of the formulation buffer. Formulations were stored in a 50 mL ALK vial and in the 10 mL ALK vial used for the reaction. Samples were then taken from each formulation for RP-HPLC and iTLC analysis. The two formulations were stored at 2-8° C.

TABLE 41
Reaction Summary - Regular Specific Activity - 5 μCi/nmol.

| | |
|---|---|
| Ac-225 Activity (μCi) | 2040 |
| Total Reaction Volume (μL) | 4052 |
| Reaction Sodium Acetate | 0.26 |
| Concentration (M) | |
| Reaction Peptide | 0.10 |
| Concentration (nmol/μL) | |
| Reaction Radioactivity | 0.50 |
| Concentration (μCi/μL) | |
| Targeted Specific Molar | 5.1 |
| Activity (μCi/nmol) | |

TABLE 42
Results Summaries - Regular Specific Activity - 5 μCi/nmol.

| | Run #1 | Run #2 |
|---|---|---|
| Product Radioactive | 0.04 | 0.11 |
| Concentration (μCi/μL) | | |
| Product Activity (μCi) | 1278 | 735 |
| Ascorbic Acid (mg/mL) | 0.40 | 1.1 |
| Sodium Ascorbate (mg/mL) | 50.1 | 49.4 |
| Methionine (mg/mL) | 23.7 | 23.3 |
| Tween-20 (% w/v) | 0.05 | 0.05 |
| Ethanol (% v/v) | 8.75 | 8.77 |
| Ammonium Acetate (M) | 0.079 | 0.079 |
| RCP via RP-HPLC (%) | 95.95 | 96.07 |
| RCP via iTLC (%) | 98.29 | 98.98 |
| 48 hr - RCP via | 95.21 | 95.14 |
| RP-HPLC (%) | | |
| 48 hr - RCP via | 98.31 | 98.25 |
| iTLC (%) | | |
| 192 hr - RCP via | 93.53 | 91.80 |
| RP-HPLC (%) | | |
| 192 hr - RCP via | N/A | N/A |
| iTLC (%) | | |

### Example 9. In Vivo Evaluation of the Anti-Tumor Activity of [²²⁵Ac]Ac-496 in a Mouse CDX Model of Lung Adenocarcinoma

### I. Summary

B7-H3 (CD276) is a transmembrane glycoprotein belonging to the B7 family of checkpoint molecules that is highly expressed in multiple solid tumors including breast, lung, melanoma, prostate, and esophageal. Compound 496 is a macrocyclic peptide developed by the inventors that binds to B7-H3 (41 g), the predominant form of B7-H3 expressed by tumor cells, with high affinity combined with selectivity over the 21 g isoform.

Compound 496 was labeled with the alpha particle-emitting radionuclide ²²⁵Ac to produce [²²⁵Ac]Ac-496. NCI-H1650 cells (derived from a human NSCLC) were inoculated subcutaneously in mice (female, athymic, nude-Foxn1^{m}). When the tumor volumes reached approximately 200-300 mm³ mice were randomized into groups with similar mean tumor volumes and injected via the tail vein with a single bolus of either vehicle or [²²⁵Ac]Ac-496. Two separate studies were carried out. In study 1, the two groups were vehicle control, and [²²⁵Ac]Ac-496 (1.4 μCi; n=10 for both). In study 2, the three groups were vehicle control (n=8), and 0.35 and 0.7 μCi of [²²⁵Ac]Ac-496 (n=7 for both). Tumor volume and body weight data were collected throughout the study in a blinded fashion.

The study showed that treatment with 1.3, 0.7, and 0.35 μCi/animal of [²²⁵Ac]Ac-496 resulted in tumor regression in a dose-dependent manner and prolonged survival compared with vehicle control groups. In study 1, median survival for the control group was 29.5 days while the median survival for the 1.3 μCi/animal group was not reached before end of study (day 56). Of the mice in the group treated with 1.3 μCi [²²⁵Ac]Ac-496, 80% survived to the end of the study with mean tumor volumes of 76.99±30.31 mm³ on day 56. In study 2, median survival for control and 0.35 μCi groups were 20 days and 44 days respectively while median survival of the 0.7 μCi group was not reached before end of study (day 55). Of mice in the group treated with 0.35 μCi of [²²⁵Ac]Ac-496, 43% survived till the end of study with mean tumor volumes of 403.14±66.16 mm³, while 86% of mice treated with 0.7 μCi survived till the end of the study with mean tumor volumes of 322.75±61.92 mm³. The effects of treatment on survival extension were statistically significant for both studies (p<0.05; log-rank (Mantel-Cox) test).

A single bolus i.v. injection of [²²⁵Ac]Ac-496 led to statistically significant, dose-dependent tumor growth inhibition and an extension of survival in an in vivo model of non-small cell lung cancer. Tumor regression was transient at lower doses, while at the highest dose, the regression was sustained.

### II. Radiolabeling

[²²⁵Ac]Ac(NO₃)₃ was dissolved in 0.04 M hydrochloric acid at a concentration of 20 mCi/mL when provided as the dry nitrate form, or the isotope was provided solubilized in 0.1 M hydrochloric acid at ˜2 mCi/mL and used as-is. Actinium was added to a reaction vial and the activity assayed. Sodium acetate buffer (1.0 M pH 5.5-6.0, ascorbic acid such that the total reaction concentration was 5 mg/mL) was added at a 0.5-0.6:1 ratio to actinium volume. Ethanol was added such that the total reaction concentration was 9-10% v/v. Compound 496 was added in sufficient quantity to achieve a specific activity of ˜5 μCi/nmol. The reaction was then incubated on a thermomixer at 700 rpm and 90° C. for 15 minutes. After completion of the radiolabeling reaction, the non-labeled Compound 496 and other impurities were removed using RP-HPLC (Agilent Infinity II 1260 HPLC equipped with a Waters XSelect Peptide CSH C18 column, fraction collector, and UV-diode array detector [DAD]). The product was collected directly into formulation buffer (66.67 mg/mL sodium ascorbate, 31.47 mg/mL L-methionine, 0.067% Tween-20, and 0.1 M ammonium acetate). Formulations used for animal dosing are summarized below (Table 43).

TABLE 43
Formulation Summary of [²²⁵Ac]Ac-496

| Reaction No. | 1 | 2 |
|---|---|---|
| Purpose of Reaction | Used for | Used for |
| | Study 1 | Study 2 |
| RCP (%) | 94.6 | 96.2 |
| 1.4 μCi/100 μL, Formulation Activity (μCi) | 13.8 | 20.5 |
| 1.4 μCi/100 μL, Formulation Mass (mg) | 994.73 | 1435.63 |
| 1.4 μCi/μL, Formulation Concentration (μCi/mg) | 0.014 | 0.014 |
| 0.7 μCi/100 μL, Formulation Activity (μCi) | — | 6.9 |
| 0.7 μCi/100 μL, Formulation Mass (mg) | — | 984.43 |
| 0.7 μCi/μL, Formulation Concentration (μCi/mg) | — | 0.007 |
| 0.35 μCi/100 μL, Formulation Activity (μCi) | — | 7.3 |
| 0.35 μCi/100 μL, Formulation Mass (mg) | — | 2090.62 |
| 0.35 μCi/μL, Formulation Concentration (μCi/mg) | — | 0.0035 |

### III. In Vivo Tumor Models

Athymic (nude) mice (homozygous for Foxn1^{nu} mutation: Taconic Biosciences, Inc., Germantown, NY) were used as in vivo hosts of the NCI-H1650-expressing subcutaneous tumors as their deficient immune system allows human cancer cells to grow into tumors. Following acclimation after delivery at 6 weeks of age from the external vendor the mice were entered into the study when they were around 7-9 weeks old.

The cells were washed with phosphate-buffered saline (PBS), treated with trypsin to remove them from the surface of the tissue culture flasks, and concentrated by centrifugation at 1,200 rpm at 4° C. for 5 min. before being resuspended in fresh cell medium. Viability was tested using Trypan Blue (>90%) and the cells were counted using a Countess 3 automated cell counter. They were then re-centrifuged at 1,200 rpm at 4° C. for 5 min., and then resuspended at a concentration of 50 million cells/mL in a 1:1 mixture of PBS and Geltrex® and kept on ice until subcutaneous inoculation. Tumor cells (5 million in 100 μL) were injected subcutaneously using a syringe with a 29-gauge needle. Tumors grew over the subsequent 2 weeks before inclusion in efficacy studies.

### IV. In Vivo Efficacy Studies

These studies were carried out as two separate studies. The first study included a control group (treated with vehicle only) and a group treated with 1.3 μCi of [²²⁵Ac]Ac-496. The second study comprised a control group, a group treated with 0.7 μCi of [²²⁵Ac]Ac-496 and a group treated with 0.35 μCi of [²²⁵Ac]Ac-496.

For the first study, the mice were sorted into 2 groups with similarly sized tumors: 269±62 and 265±71 mm3 (mean±SD) for the control and 1.3 μCi [²²⁵Ac]Ac-496 treated groups, respectively. Mice were injected via the tail vein with: vehicle (formulation buffer; control group, n=10); or [²²⁵Ac]Ac-496 (n=10; injected activity of 1.29±0.07 μCi; mean±SD) on day 0.

For the second study, the mice were sorted into 3 groups with similarly sized tumors: 252±104, 243±70 and 270±104 mm3 (mean±SD) for the control, 0.7 and 0.35 μCi [²²⁵Ac]Ac-496 treated groups, respectively. Mice were injected via the tail vein with: vehicle (formulation buffer; control group, n=8); or 0.7 μCi of [²²⁵Ac]Ac-496 (n=7; injected activity of 0.71±0.01 μCi mean±SD); or 0.35 μCi of [²²⁵Ac]Ac-496 (n=7; injected activity of 0.35±0.01 μCi).

Following injection, mice were returned to their cages and monitored daily. Individual body weights and tumor volumes were measured twice a week by a researcher who was blinded to the treatments. Tumors were measured in three dimensions using calipers and the tumor volume was calculated using the formula V=π/6×L×W×H as previously described (Tomayko and Reynolds 1989). Mice were removed from the study and euthanized based on clinical signs including body weight loss of more than 20%, hunched appearance, inappetence, ulceration of the tumor, or tumor growth beyond 1,000 mm3. The decision to euthanize based on clinical signs (e.g., a large tumor was impeding ambulation and therefore probably feeding etc.) was that of the blinded observer and was thereby separated from treatment group and was made objectively. Data was analyzed using GraphPad Prism version 9.5.1.

### V. Effect of Treatment on Tumor Volume and Survival Extension

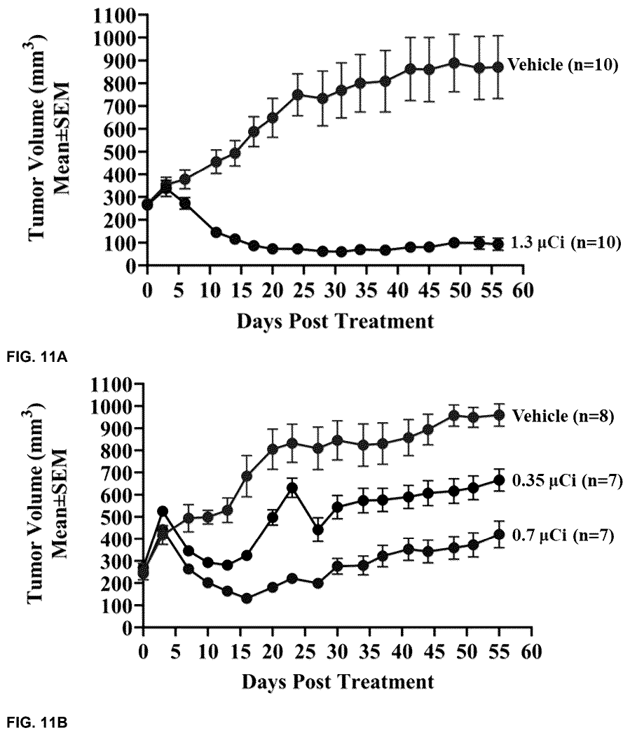

Treatment with [²²⁵Ac]Ac-496 resulted in tumor regression for all mice compared with those in the control groups as is shown in FIG. 11.

In study 1, treatment with [²²⁵Ac]Ac-496 at 1.3 μCi resulted in robust and sustained tumor regression (FIG. 11A). Median survival for vehicle group was 29.5 days while the median survival for 1.3 μCi group was not reached before end of study on day 56 (p=0.0203, log-rank (Mantel-Cox) test)). The maximum mean tumor volume of 733.5±155.5 mm³ 1.3 μCi was reached in vehicle treated group on day 28 while the mean tumor volume on day 28 for 1.3 μCi [²²⁵Ac]Ac-496 treatment group was 38.15±7.33 mm³. One tumor in the vehicle-treated group spontaneously regressed and another tumor reached 535 mm³ by the end of the study. Two mice from the 1.3 μCi [²²⁵Ac]Ac-496 treatment group were removed on days 3 and 17 due to ulceration not considered to be related to [²²⁵Ac]Ac-496 treatment.

In study 2, treatment with [²²⁵Ac]Ac-496 at 0.7 μCi and 0.35 μCi resulted in dose-dependent transient tumor regression (FIG. 11B). The maximum mean tumor volume of 732.4±101.5 mm³ was reached in vehicle treated group on day 20 while the mean tumor volume on day 20 for 0.7 μCi and 0.35 μCi [²²⁵Ac]Ac-496 treatment group were 180.2±18.8 mm3 and 497.3±34.8 mm³ respectively. Median survival for vehicle and 0.35 μCi groups were 20 days and 44 days respectively while median survival 0.7 μCi group was not reached by end of study ((p=0.0122, log-rank (Mantel-Cox) test). Treatment with [²²⁵Ac]Ac-496 at 0.7 μCi resulted in robust tumor regression in all mice, with 29% of tumor rebounding 20 days post treatment. Of the mice treated with 0.7 μCi, 80% survived till the end of the study with mean tumor volumes of 322.8±61.9 mm³ (day 55). In the group treated with 0.35 μCi, tumor regression was initially observed but tumors started to regrow by day 20. Nearly half (43%) of the mice in this group treated survived at the end of study with mean tumor volumes of 403.1±66.2 mm³. Tumor ulceration led to 29% of the mice in this group leaving the study.

### Example 10: Radiochemistry Process and Formulation Development of High Specific Activity [²²⁵Ac]Ac-DOTA-TATE

DOTA-TATE can be purchased from commercial sources.

### I. [²²⁵Ac]Ac Radiolabeling of DOTA-TATE

Reagents consisted of a 1.0 M sodium acetate buffer pH 5.5-6.0, DOTA-TATE precursor dissolved in metal free water or 0.1 M sodium acetate buffer pH 5.5 to a concentration of 0.4-1.3 nmol/μL, and Actinium-225 source material dissolved in 0.04-0.1 M hydrochloric acid at a radioconcentration of 24.0-8.2 μCi/μL. The above reagents were mixed in various proportions such that the final concentrations present in the reaction mixture ranged from 0.04-0.15 M sodium acetate, 0.30-0.96 nmol/μL DOTA-TATE, and 1.19-3.16 μCi/μL Actinium-225. This mixture was incubated at 90° C. for 15 min and shaken at 700 RPM. After the 15 min had elapsed, the reaction was allowed to cool briefly, quenched with a solution of 0.05 mg/mL DTPA, and injected into RP-HPLC for purification as described in section II below, in 100 μL aliquots as needed. See, FIG. 13. Reaction conditions are summarized in Table 44. In Run #1, the injected activity was 74 μCi and the recovered activity was 67 μCi, resulting in a purification yield of 91% and a process yield of 80%. In Run #2, the injected activity was 541 μCi and the recovered activity was 370 μCi, resulting in a purification yield of 68% and a process yield of 58%. In Run #3, the injected activity was 76 μCi and the recovered activity was 55 μCi, resulting in a purification yield of 73% and a process yield of 67%.

TABLE 44
Summary of initial [²²⁵Ac]Ac-DOTA-
TATE radiolabeling reaction conditions

| Run | #1 | #2 | #3 |
|---|---|---|---|
| Net sodium acetate (M) | 0.04 | 0.11 | 0.15 |
| Reaction activity (μCi) | 83.5 | 632 | 82.2 |
| Peptide mass (nmol) | 25 | 191 | 20.7 |
| Specific activity (μCi/nmol) | 3.3 | 3.3 | 4.0 |
| Reaction volume (μL) | 59 | 200 | 69 |
| Reaction pH | 5.0-5.5 | 4.5-5.0 | 5.0-5.5 |
| Incubation | 90 | 90 | 90 |
| temperature (° C.) | | | |
| [²²⁵Ac]Ac Incorporation (iTLC, %) | | | |
| T = 15 min | 99 | 99.2 | >99 |

### II. Isocratic RP-HPLC Purification Development

Isocratic RP-HPLC purification development was performed using an Agilent 1260 Infinity II system equipped with a diode array detector with detection at 220 nm. Separations were performed on a Waters XSelect Peptide CSH C18 column (130 Å, 3.5 μm, 4.6×150 mm). A single buffer strength was tested, 1.5 mM ammonium acetate, along with two different ethanol concentrations, 20% and 30% at 0.5 mL/min. These solvent compositions yielded buffer strengths on-column of 1.20 and 1.05 mM respectively.

Internally produced DOTA-TATE and [^{nal}La]La-DOTA-TATE were dissolved in water to a concentration of 0.47 and 0.36 nmol/uL respectively. Co-injections were performed by mixing the compounds in equivalent volumes for 5 uL injections.

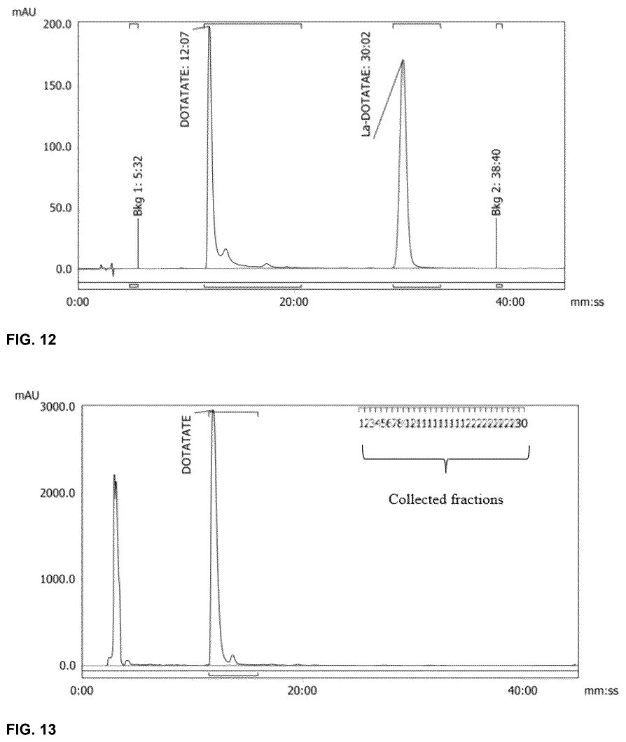

Initial testing with 20% ethanol yielded very favorable results with good resolution and peak shape of both species. Several co-injections were performed to verify results and a DOTA-TATE injection performed to confirm peak identity. See, FIG. 12.

### III. Formulation Development

A formulation buffer consisting of ˜50 mg/mL sodium ascorbate, ˜24 mg/mL L-methionine, ˜0.05% Tween-20, and ˜75 mM ammonium acetate and ˜5% v/v ethanol was selected using the optimization strategy described above, and was able to maintain an RCP>90% over 288 hr. During HPLC purification, fractions were collected into 2 mL HPLC vials pre-loaded with 0.75 mL of formulation buffer. Each fraction was collected for 30 seconds, yielding ˜0.25 mL of collected eluant and ˜1 mL total fraction volume post-collection.

A study was conducted to determine stability differences between the high specific activity formulation, and a reduced specific activity formulation (5 μCi/nmol). A precise quantification of the specific molar activity of the high specific activity formulation is unavailable due to inability to detect peptide mass at the mass loading used. Post-purification and after secular equilibrium was re-established, an aliquot of high specific activity formulation was diluted with sufficient precursor DOTA-TATE mass to achieve the 5 μCi/nmol specific activity. Samples were taken at the time points indicated in the table below and analyzed via iTLC and RP-HPLC for RCP.

Both formulations maintained a high RCP of >90% up to the tested 288 hr. See Table 45 and Table 46.

TABLE 45
Summary of RSA vs HSA [²²⁵Ac]Ac-DOTA-TATE % RCP
up to 288 hr via iTLC stored at 2-8° C.

| | |
|---|---|
| #1 | #2 |

| | High | Regular Specific | High |
|---|---|---|---|
| | Specific | Activity | Specific |
| Run | Activity | (5 μCi/nmol) | Activity |
| Activity Stored (μCi) | 58.4 | 186.2 | 183.2 |
| Radioconcentration | 0.058 | 0.037 | 0.037 |
| (μCi/μL) | | | |

RCP (iTLC, %)

| | | | |
|---|---|---|---|
| T = EOS | 99.9 | 99.9 | 99.9 |
| T = 24 hr | 99.4 | 99.9 | 98.8 |
| T = 48 hr | — | 99.9 | 99.8 |
| T = 120 hr | — | 99.7 | 99.2 |
| T = 144 hr | 98.5 | — | — |
| T = 168 hr | 98.7 | 99.6 | 98.6 |
| T = 288 hr | 98.8 | 99.3 | 98.5 |

TABLE 46
Summary of RSA vs HSA [²²⁵Ac]Ac-DOTA-TATE % RCP
up to 288 hr via RP-HPLC stored at 2-8° C.

| | |
|---|---|
| #1 | #2 |

| | High | Regular Specific | High |
|---|---|---|---|
| | Specific | Activity | Specific |
| Run | Activity | (5 μCi/nmol) | Activity |
| Activity Stored (μCi) | 58.4 | 186.2 | 183.2 |
| Radioconcentration | 0.058 | 0.037 | 0.037 |
| (μCi/μL) | | | |

RCP (RP-HPLC, %)

| | | | |
|---|---|---|---|
| T = EOS | 93.4 | 96.2 | 96.2 |
| T = 24 hr | 95.0 | 96.3 | 95.2 |
| T = 48 hr | — | 96.1 | 94.7 |
| T = 120 hr | — | 96.4 | 94.4 |
| T = 144hr | 95.0 | — | — |
| T = 168 hr | 94.8 | 96.1 | 94.4 |
| T = 288 hr | 92.9 | 95.6 | 94.4 |

### IV. Determination of RCP

The analytical conditions used for HPLC and iTLC analysis are described below in Table 48 and Table 49, respectively.

TABLE 48
Analytical HPLC conditions.

| | |
|---|---|
| System | Agilent 1260 Infinity II |
| Column | Waters XBridge C18; 3.5 μm, 4.6 × 250 mm |
| UV wavelength (nm) | 220 |
| Radiodetection | Gamma count fractions >6 hr post-collection |
| | (Perkin Elmer Wizard2 2470) |
| Fraction collection | 12 second time-slices |
| Mobile Phase A | 0.1% (v/v) formic acid/water |
| Mobile Phase B | 0.1% (v/v) formic acid/acetonitrile |
| Flow Rate (mL/min) | 0.5 |
| Run time (min) | 20 |
| Retention time, | 11.0-11.2 |
| [²²⁵Ac]Ac-DOTA-TATE (min) | |
| Retention time, | 4-5 |
| [²²⁵Ac]Ac-DTPA¹ (min) | |

Gradient Conditions

| Time (min) | Mobile Phase A (%) | Mobile Phase B (%) |
|---|---|---|
| 00.00 | 80 | 20 |
| 01.00 | 80 | 20 |
| 15.00 | 50 | 50 |
| 15.01 | 10 | 90 |
| 19.99 | 10 | 90 |
| 20.00 | 80 | 20 |

¹DTPA chelated [²²⁵Ac]Ac in analyses is representative of unbound [²²⁵Ac]Ac in the reaction/formulation mixtures.

TABLE 49
Analytical iTLC conditions.

| Stationary Phase | Agilent Silica Gel Impregnated |
|---|---|
| | Microfiber Paper (20 × 100 mm) |
| Mobile Phase | 50 mM EDTA; pH 5.4 |
| Radiodetection | E&Z AR2000 scan >6 |
| | hr post-development |
| Elution, [²²⁵Ac]Ac-DOTA-TATE(mm) | 0-75 |
| Elution, [²²⁵Ac]Ac-DTPA¹ (mm) | 75-150 |

¹DTPA chelated [²²⁵Ac]Ac in analyses is representative of unbound [²²⁵Ac]Ac in the reaction/formulation mixtures.

### V. HSA [²²⁵Ac]Ac-DOTA-TATE: Comparison of Formulation and Stability

A study was conducted to compare the stability profile of a [²²⁵Ac]Ac-DOTA-TATE formulation prepared according to the methods described herein with a formulation prepared according to a previously known method. The known method involves the chelation of radioisotope ions with an excess quantity of chelate ligands (e.g., DOTA-peptides) in a buffer with stabilizers/radioprotectants. This method results in a low specific activity radioactive product containing mostly unlabeled chelate ligands and impurities derived from a radiolabeling reaction. In contrast, the methods described herein remove excess unlabeled chelate ligands as well as impurities produced during the radiolabeling reaction conditions.

The known method is disclosed in Example 13 of U.S. Pat. No. 11,819,556 and involves heating a mixture of [²²⁵Ac]Ac and DOTA-TATE to yield the product [²²⁵Ac]Ac-DOTA-TATE having a targeted specific molar activity of 5 μCi/nmol. The [²²⁵Ac]Ac-labeled DOTA-TATE was then mixed with a formulation buffer consisting of sodium ascorbate, DTPA, and saline to provide a product solution having a radioactive concentration of 0.033 μCi/μL, which was then assessed by iTLC at different time points (Table 50).

TABLE 50

| | Percentage of [²²⁵Ac]Ac in [²²⁵Ac]Ac-DOTA-TATE | | | | | | |
|---|---|---|---|---|---|---|---|
| | 0 | 24 | 48 | 72 | 96 | 120 | 144 |
| | hours | hours | hours | hours | hours | hours | hours |
| 1 mCi | 99.21% | 98.87% | 97.21% | 96.54% | Not | 95.52% | 94.81% |
| Formulation | | | | | measured | | |

The comparator formulation of [²²⁵Ac]Ac-DOTA-TATE was prepared and formulated in sodium ascorbate, L-methionine, Tween-20, 75 mM ammonium acetate, and ethanol, as described above, to afford a product solution having a specific molar activity of 5 μCi/nmol and a radioactive concentration of 0.033 μCi/μL. The product solution was divided into three portions, the stability of which were assessed by iTLC. See, Table 45.

Although the methods and pharmaceutical compositions of the disclosure have been described in some detail by way of illustration and example for purposes of clarity of understanding, one of ordinary skill in the art will appreciate that certain changes and modifications may be practiced within the scope of the appended claims. In addition, each reference, including all of the U.S. patents, U.S. patent application publications, U.S. patent applications, foreign patents, foreign patent applications and non-patent publications referred to in this specification are incorporated herein by reference, in their entirety, to the extent not inconsistent with the present description. Where a conflict exists between the instant application and a reference provided herein, the instant application shall dominate.

1. A method of making a stabilized composition of a radiolabeled compound, comprising:
a. eluting a mixture comprising the radiolabeled compound and a corresponding unlabeled compound through a chromatography column, thereby separating the radiolabeled compound from the unlabeled compound;
b. collecting an eluate containing the separated radiolabeled compound in a vessel containing a stabilizing solution to afford the stabilized composition of the radiolabeled compound.

2-4. (canceled)

5. The method of claim 1, wherein eluting the mixture comprising the radiolabeled compound and unlabeled compound through a chromatography column comprises the use of mobile phase solvents, wherein the mobile phase solvents are pharmaceutically acceptable.

6. (canceled)

7. The method of claim 1, wherein the molar ratio of radiolabeled compound to unlabeled compound, prior to separation, is in the range of about 1:5 to about 1:15000.

8-15. (canceled)

16. The method of claim 1, wherein the stabilizing solution comprises one or more components selected from the group consisting of L-methionine, a polysorbate, ascorbic acid or a pharmaceutically acceptable salt thereof, and acetic acid or a pharmaceutically acceptable salt thereof.

17-18. (canceled)

19. The method of claim 16, wherein the mass ratio of ascorbic acid or a pharmaceutically acceptable salt thereof to polysorbate in the stabilizing solution is from 50:1 to 150:1.

20. The method of claim 16, wherein the ascorbic acid or a pharmaceutically acceptable salt thereof is present in the stabilizing solution in an amount of ≤100 mg/mL.

21. The method of claim 16, wherein the polysorbate is present in the stabilizing solution in an amount of ≤1 mg/mL.

22-42. (canceled)

43. The method of claim 1, wherein the radiolabeled compound comprises: (i) a peptide comprising a chelator, and (ii) a radioisotope.

44-50. (canceled)

51. The method of claim 1, wherein the stabilized composition has a specific molar activity in the range of 60-19500 μCi per nmol compound.

52. The method of claim 1, wherein the stabilized composition has a specific molar activity in the range of 15-19 mCi per nmol compound.

53-57. (canceled)

58. The method of claim 43, wherein the mixture comprising the radiolabeled compound and unlabeled compound is prepared by combining the radioisotope and a reagent solution comprising the peptide.

59-64. (canceled)

65. The method of claim 58, wherein the reagent solution further comprises a reaction buffer.

66. The method of claim 65, wherein the reaction buffer comprises a stabilizer comprising ascorbic acid or a pharmaceutically acceptable salt thereof.

67-69. (canceled)

70. The method of claim 58, wherein the reagent solution further comprises ethanol.

71. (canceled)

72. The method of claim 58, wherein the reagent solution does not comprise a polysorbate.

73-77. (canceled)

78. A pharmaceutical composition comprising a radiolabeled compound and a stabilizing solution,
wherein the stabilizing solution comprises ascorbic acid or a pharmaceutically acceptable salt thereof and a polysorbate, and one or more components selected from the group consisting of ethanol, L-methionine, selenomethionine, histidine, melatonin, acetic acid or a pharmaceutically acceptable salt thereof, benzyl alcohol, p-aminobenzoic acid or a pharmaceutically acceptable salt thereof, cysteamine, 5-amino-2-hydroxybenzoic acid or a pharmaceutically acceptable salt thereof, nicotinic acid or a pharmaceutically acceptable salt thereof, nicotinamide, cysteine, monothioglycerol, sodium bisulfite, sodium metabisulfite, gentisic acid, and inositol, and
wherein the mass ratio of ascorbic acid or a pharmaceutically acceptable salt thereof to polysorbate in the pharmaceutical composition is from 50:1 to 150:1.

79-83. (canceled)

84. The pharmaceutical composition of claim 78, wherein the ascorbic acid or a pharmaceutically acceptable salt thereof is present in the pharmaceutical composition in an amount of ≤100 mg/mL.

85. The pharmaceutical composition of claim 78, wherein the polysorbate is present in the pharmaceutical composition in an amount of ≤1 mg/mL.

86. (canceled)

87. The pharmaceutical composition of claim 78, wherein the pharmaceutical composition comprises L-methionine.

88. The pharmaceutical composition of claim 78, wherein the pharmaceutical composition comprises acetic acid or a pharmaceutically acceptable salt thereof.

89. (canceled)

90. The pharmaceutical composition of claim 78, wherein the pharmaceutical composition comprises ethanol.

91-99. (canceled)

100. The pharmaceutical composition of claim 78, wherein the radiolabeled compound comprises: (i) a peptide comprising a chelator, and (ii) a radioisotope.

101-112. (canceled)

113. The pharmaceutical composition of claim 78, wherein the pharmaceutical composition comprises sodium ascorbate in a concentration of about 40 mg/mL to about 60 mg/mL, L-methionine in a concentration of about 10 mg/mL to about 30 mg/mL, polyoxyethylene (20) sorbitan monolaurate in an amount of from about 0.01% (w/v) to about 0.1% (w/v), ethanol in an amount of from about 1% (v/v) to about 10% (v/v), and acetate in a concentration of about 0.01 M to about 0.1 M.

114-122. (canceled)

123. The pharmaceutical composition of claim 78, comprising a radiolabeled compound having a specific molar activity in the range of 60-19500 μCi per nmol.

124. (canceled)

125. The pharmaceutical composition of claim 78, wherein the radiolabeled peptide has a specific molar activity in the range of from 15-19 mCi per nmol peptide.

126. (canceled)

127. A pharmaceutical composition prepared using the method as defined in claim 1.

128. A pharmaceutical composition comprising:
a radiolabeled compound comprising (i) a peptide comprising a chelator, and (ii) a radioisotope, wherein radioisotope is chelated with the chelator;
ascorbic acid or a pharmaceutically acceptable salt thereof; and
a polysorbate;
wherein the pharmaceutical composition has a radiochemical purity (RCP) of >90% after 72 hours at 2-8° C.

129-140. (canceled)

141. The pharmaceutical composition of claim 78, wherein the pharmaceutical composition comprises:
a radiolabeled compound comprising (i) a peptide comprising a DOTA chelator and (ii) a radioisotope which is ²²⁵Ac, wherein the ²²⁵Ac is chelated with the DOTA chelator;
sodium ascorbate, which is present in an amount of ≤100 mg/mL; and
polyoxyethylene (20) sorbitan monolaurate, which is present in an amount of ≤1 mg/mL;
wherein the pharmaceutical composition has an RCP>90% after 72 hours.

142-143. (canceled)

144. The pharmaceutical composition of claim 78, wherein the pharmaceutical composition comprises:
a radiolabeled compound comprising (i) a peptide comprising a DOTA chelator and (ii) a radioisotope which is ¹⁷⁷Lu, wherein the ¹⁷⁷Lu is chelated with the DOTA chelator;
sodium ascorbate, which is present in an amount of ≤100 mg/mL; and
polyoxyethylene (20) sorbitan monolaurate, which is present in an amount of ≤1 mg/mL;
wherein the pharmaceutical composition has an RCP>90% after 72 hours.

145. A method of treating cancer in a subject in need thereof, comprising administering to the subject a stabilized composition prepared by the method as defined in claim 1.

146-147. (canceled)

148. A method of treating cancer in a subject in need thereof, comprising administering to the subject the pharmaceutical composition of claim 78.

149-150. (canceled)

151. The method of claim 145, wherein the cancer comprises a cell surface protein characterized by an expression of 1000-100,000 copies per cell.
